(19) United States
(12) Patent Application Publication
(10) Pub. No.: US 2016/0016907 A1
(43) Pub. Date: Jan. 21, 2016

(54) PYRIDINE DERIVATIVES AS MUSCARINIC M1 RECEPTOR POSITIVE ALLOSTERIC MODULATORS

(71) Applicant: PFIZER INC., NEW YORK, NY (US)

(72) Inventors: MICHAEL AARON BRODNEY, NEWTON, MA (US); JENNIFER ELIZABETH DAVOREN, CAMBRIDGE, MA (US); MICHELLE RENEE GARNSEY, PROVIDENCE, RI (US); LEI ZHANG, AUBURNDALE, MA (US); STEVEN VICTOR O'NEIL, EAST LYME, CT (US)

(73) Assignee: PFIZER INC., NEW YORK, NY (US)

(21) Appl. No.: 14/799,621

(22) Filed: Jul. 15, 2015

Related U.S. Application Data
(60) Provisional application No. 62/142,691, filed on Apr. 3, 2015.
(60) Provisional application No. 62/026,087, filed on Jul. 18, 2014.

(51) Int. Cl. C07D 213/81; C07D 417/14; C07D 401/06; C07D 401/10; C07D 401/14; C07D 405/14; C07D 413/14

(52) U.S. Cl. CPC C07D 213/81; C07D 405/14; C07D 417/14; C07D 413/14; C07D 401/10; C07D 401/14; C07D 401/06

(57) ABSTRACT

The present invention provides, in part, compounds of Formula I:

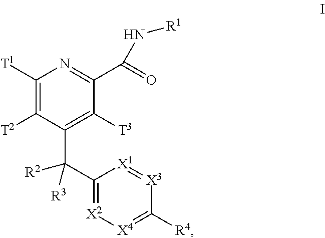

N-oxides thereof, and pharmaceutically acceptable salts of the compounds or N-oxides; processes for the preparation of; intermediates used in the preparation of; and compositions containing such compounds, N-oxides, or salts, and their uses for treating M1-mediated (or M1-associated) disorders including, e.g., Alzheimer's disease, schizophrenia (e.g., its cognitive and negative symptoms), pain, addiction, and a sleep disorder.

### FIELD OF THE INVENTION

The present invention generally relates to novel pyridine derivatives, which are muscarinic M1 receptor modulators (e.g. positive allosteric modulators), salts thereof, pharmaceutically compositions thereof, and uses thereof in the treatment of M1-mediated diseases and disorders such as Alzheimer's disease.

### BACKGROUND OF THE INVENTION

Alzheimer's disease is a common neurodegenerative disease affecting the elderly, resulting in progressive memory impairment, loss of language and visuospatial skills, and behavior deficits. Characteristics of the disease include degeneration of cholinergic neurons in the cerebral cortex, hippocampus, basal forebrain, and other regions of the brain; neurofibrillary tangles; and accumulation of the amyloid β peptide (Aβ). AR is a 39-43 amino acid produced in the brain by processing of the beta-amyloid precursor protein (APP) by the beta-amyloid protein cleaving enzyme (“beta secretase” or “BACE”) and gamma-secretase. The processing leads to accumulation of AR in the brain.

Cholinergic neurotransmission involves the binding of acetylcholine either to the nicotinic acetylcholine receptor (nAChR) or to the muscarinic acetylcholine receptor (mAChR). It has been hypothesized that cholinergic hypofunction contributes to the cognitive deficits of patients suffering from Alzheimer's disease. Consequently, acetyl cholinesterase inhibitors, which inhibit acetylcholine hydrolysis, have been approved in the United States for use in treating cognitive impairments of Alzheimer's disease patients. While acetyl cholinesterase inhibitors have provided some cognitive enhancement in Alzheimer's disease patients, the therapy has not been shown to change the underlying disease pathology.

A second potential pharmacotherapeutic target to counteract cholinergic hypofunction is the activation of muscarinic receptors. Muscarinic receptors are prevalent throughout the body. Five distinct muscarinic receptors (M1-M5) have been identified in mammals. In the central nervous system, muscarinic receptors are involved in cognitive, behavior, sensory, motor, and autonomic functions. The muscarinic M1 receptor, which is prevalent in the cerebral cortex, hippocampus, and striatum, has been found to have a major role in cognitive processing and is believed to have a role in the pathophysiology of Alzheimer's disease. See Eglen et al, TRENDS in Pharmacological Sciences, 2001, 22:8, 409-414. In addition, unlike acetyl cholinesterase inhibitors, which are known to provide only symptomatic treatment, M1 agonists also have the potential to treat the underlying disease mechanism of Alzheimer's disease. The cholinergic hypothesis of Alzheimer's disease is linked to both β-amyloid and hyperphosphorylated tau protein. Formation of β-amyloid may impair the coupling of the muscarinic receptor with G-proteins. Stimulation of the M1 muscarinic receptor has been shown to increase formation of the neuroprotective sAPPα fragment, thereby preventing the formation of the AR peptide. Thus, M1 agonists may alter APP processing and enhance αAPPs secretion. See Fisher, Jpn J Pharmacol, 2000, 84:101-112.

The M1/M4 muscarinic agonist xanomeline was found to improve all three of the major symptom domains in schizophrenic patients, including positive, negative, and cognitive symptoms, was found to reduce psychotic symptoms in patients with Alzheimer's disease. See Shekhar A, et. al, “Selective muscarinic receptor agonist xanomeline as a novel treatment approach for schizophrenia,” Am J Psychiatry, 2008 August; 165(8):1033-9; see also Bodick N C, et. al, “Effects of xanomeline, a selective muscarinic receptor agonist, on cognitive function and behavioral symptoms in Alzheimer disease,” Arch Neurol. 1997, April, 54(4):465-73. Moreover, M1 ligands (such as agonists) may be useful for treating neuropathic pain and addiction (such as substance addiction, e.g., cocaine addiction). See Martino G, et. al, “The M1/M4 preferring agonist xanomeline is analgesic in rodent models of chronic inflammatory and neuropathic pain via central site of action,” Pain, 2011, December, 152(12):2852-60; and Thomsen M, et. al, “Attenuation of cocaine's reinforcing and discriminative stimulus effects via muscarinic M1 acetylcholine receptor stimulation,” J Pharmacol Exp Ther. 2010, 332(3):959-69.

M1 muscarinic acetylcholine receptor (mAChR) activation was shown to reduce rapid eye movement (REM) sleep latency and slow wave sleep (SWS) duration in comparison with placebo. See Nissen C, et. al, “M1 muscarinic acetylcholine receptor agonism alters sleep without affecting memory consolidation,” J Cogn Neurosci. 2006 November; 18(11):1799-807; and Nissen C, et. al, “Differential effects of the muscarinic M1 receptor agonist RS-86 and the acetylcholine-esterase inhibitor donepezil on REM sleep regulation in healthy volunteers,” Neuropsychopharmacology. 2006 June; 31(6):1294-300.

Dry mouth is a frequent side effect of muscarinic receptor antagonists, while selective activators of M1 muscarinic receptors increase salivary secretion in mice, rats, and humans. See Eglen R M et al., 1999, “Muscarinic receptor ligands and their therapeutic potential,” Curr Opin Chem Biol 3: 426-32; and Gautam D et al., 2004, “Cholinergic stimulation of salivary secretion studied with M1 and M3 muscarinic receptor single- and double-knockout mice.”

However, M1 ligands that have been developed and studied for Alzheimer's disease have produced side effects common to other muscarinic receptor ligands, such as sweating, nausea and diarrhea, See Spalding et al, Mol Pharmacol, 2002, 61:6, 1297-1302.

The muscarinic receptors are known to contain one or more allosteric sites, which may alter the affinity with which muscarinic ligands bind to the primary binding or orthosteric sites. See e.g., S. Lazareno et al, Mol Pharmacol, 2002, 62:6, 1491-1505; and S. Lazareno et al, Mol Pharmacol, 2000, 58, 194-207. Muscarinic M1 positive allosteric modulators may be useful for treating M1-mediated diseases and disorders (e.g., Alzheimer's disease and schizophrenia). See e.g. US2012252808 and US2013059860.

New or improved agents that modulate muscarinic M1 receptors (such as M1 positive allosteric modulators) are needed for developing new and more effective pharmaceuticals to treat M1-mediated diseases and disorders such as Alzheimer's disease and others described herein.

### SUMMARY OF THE INVENTION

The present invention provides, in part, a compound of Formula I:

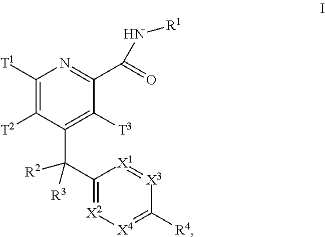

or an N-oxide thereof, or a pharmaceutically acceptable salt of the compound or the N-oxide, wherein:

R¹ is selected from the group consisting of C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted one or more independently selected R⁵, and wherein each of the C₁₋₈ alkyl, C₃₋₁₀ cycloalkyl, 4- to 10-membered heterocycloalkyl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is further optionally substituted one or more oxo;

each of R² and R³ is independently selected from the group consisting of H, halogen (e.g. F or Cl), OH, methyl, and methoxy, wherein each of the methyl and methoxy is optionally substituted with one or more substituents each independently selected from OH and halogen;

R⁴ is selected from the group consisting of H, halogen, OR⁶, CN, C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted with one or more independently selected R⁷, and wherein each of the C₁₋₈ alkyl, C₃₋₁₀ cycloalkyl, 4- to 10-membered heterocycloalkyl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is further optionally substituted one or more oxo;

T¹ is selected from the group consisting of H, halogen, —N(R^{c})₂, —NR^{e}R^{f}, —CN, C₁₋₆ alkyl, C₂₋₆ alkenyl, C₂₋₆ alkynyl, C₃₋₆ cycloalkyl, (Cₘ cycloalkyl)-C₁₋₂ alkyl-, and C₁₋₆ alkoxy, wherein each of the C₁₋₆ alkyl, C₂₋₆ alkenyl, C₂₋₆ alkynyl, Cₘ cycloalkyl, (C₃₋₆ cycloalkyl)-C₁₋₂ alkyl-, and C₁₋₆ alkoxy of T¹ is optionally substituted with one or more substituents independently selected from the group consisting of halogen, —CN, —C(═O)C₁₋₄ alkyl, —C(═O)OH, —C(═O)O—C₁₋₄ alkyl, —C(═O)NHC₁₋₄ alkyl, —C(═O)N(C₁₋₄ alkyl)₂, oxo, —OH, —OC(═O)—C₁₋₄ alkyl, —OC(═O)O—C₁₋₄ alkyl, —NH₂, —NH(C₁₋₄ alkyl), —N(C₁₋₄ alkyl)₂, —NHC(═O)C₁₋₄ alkyl, —NHC(═O)OC₁₋₄ alkyl, —NHC(═O)NHC₁₋₄ alkyl, and C₁₋₄ alkoxy, and wherein R^{e} and R^{f} together with the N atom to which they are attached form a 4- to 7-membered heterocycloalkyl optionally substituted with one or more substituents each independently selected from the group consisting of halogen, —OH, oxo, —C(═O)H, —C(═O)OH, —C(═O)—C₁₋₄ alkyl, —C(═O)—NH₂, —C(═O)—N(C₁₋₄ alkyl)₂, —ON, C₁-4 alkyl, C₁₋₄ alkoxy, C₁₋₄ hydroxylalkyl, C₁₋₄ haloalkyl, and C₁₋₄ haloalkoxy;

T² is selected from the group consisting of halogen, N(R^{c})₂, —NR^{e}R^{f}, —ON, C₁₋₆ alkyl, C₂₋₆ alkenyl, C₂₋₆ alkynyl, C₃₋₆ cycloalkyl, (Cₘ cycloalkyl)-C₁₋₂ alkyl-, and C₁₋₆ alkoxy, where in each of the C₁₋₆ alkyl, C₂₋₆ alkenyl, C₂₋₆ alkynyl, C₃₋₆ cycloalkyl, (C₃₋₆ cycloalkyl)-C₁₋₂ alkyl-, and C₁₋₆ alkoxy of T² is optionally substituted with one or more substituents independently selected from the group consisting of halogen, —CN, —C(═O)C₁₋₄ alkyl, —C(═O)OH, —C(═O)O—C₁₋₄ alkyl, —C(═O)NHC₁₋₄ alkyl, —C(═O)N(C₁₋₄ alkyl)₂, oxo, —OH, —OC(═O)—C₁₋₄ alkyl, —OC(═O)O—C₁₋₄ alkyl, —NH₂, —NH(C₁₋₄ alkyl), —N(C₁₋₄ alkyl)₂, —NHC(═O)C₁₋₄ alkyl, —NHC(═O)OC₁₋₄ alkyl, —NHC(═O)NHC₁₋₄ alkyl, and C₁₋₄ alkoxy;

T³ is selected from the group consisting of H, halogen, CH₃, and C₁ fluoroalkyl; each of X¹, X², X³, and X⁴ is independently selected from the group consisting of CR⁹ and N, provided that at most two of X¹, X², X³, and X⁴ are N;

each R⁵ is independently selected from the group consisting of halogen, —OH, —NO₂, —SF₅, C₁₋₆ alkyl, C₁₋₆ haloalkyl, C₁₋₆ alkoxy, C₁₋₆ haloalkoxy, C₂₋₆ alkenyl, C₂₋₆ alkynyl, C₃₋₇ cycloalkyl, a 4- to 10-membered heterocycloalkyl, —N(R^{a})(R^{b}), —N(R^{c})(C(═O)R^{d}), —C(═O)—N(R^{a})(R^{b}), —C(═O)—R^{d}, —C(═O)—OR^{d}, —OC(═O)—R^{d}, —N(R^{c})(S(═O)₂R^{d}), —S(═O)₂—N(R^{a})(R^{b}), —SR^{d}, and —OR^{d}, wherein each of the C₁₋₆ alkyl, C₃₋₇ cycloalkyl, and heterocycloalkyl is optionally substituted with one or more substituents each independently selected from the group consisting of halogen, —CN, —OH, C₁₋₄ alkyl, C₁₋₄ alkoxy, C₁₋₄ haloalkyl, C₁₋₄ haloalkoxy, C₃₋₆ cycloalkyl, —N(R^{a})(R^{b}), —N(R^{c})(C(═O)R^{d}), —C(═O)—OR^{d}, —C(═O)H, —C(═O)R^{d}, —C(═O)N(R^{a})(R^{b}), —N(R^{c})(S(═O)₂R^{d}), —S(═O)₂—N(R^{a})(R^{b}), —SR^{d}, and —OR^{d};

R⁶ is selected from the group consisting of H, C₁₋₅ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted with one or more substituents independently selected from the group consisting of halogen, —CN, —C(═O)C₁₋₄ alkyl, —C(═O)OH, —C(═O)O—C₁₋₄ alkyl, —C(═O)NHC₁₋₄ alkyl, —C(═O)N(C₁₋₄ alkyl)₂, oxo, —OH, —OC(═O)—C₁₋₄ alkyl, —OC(═O)O—C₁₋₄ alkyl, —NH₂, —NH(C₁₋₄ alkyl), —N(C₁₋₄ alkyl)₂, —NHC(═O)C₁₋₄ alkyl, —NHC(═O)OC₁₋₄ alkyl, —NHC(═O)NHC₁₋₄ alkyl, and C₁₋₄ alkoxy;

each R⁷ is independently selected from the group consisting of halogen, —OH, —NO₂, —CN, —SF₅, C₁₋₆ alkyl, C₁₋₆ haloalkyl, C₁₋₆ haloalkoxy, C₂₋₆ alkenyl, C₂₋₆ alkynyl, C₃₋₇ cycloalkyl, a 4- to 10-membered heterocycloalkyl, —N(R^{a})(R^{b}), —N(R^{c})(C(═O)R^{d}), —C(═O)—N(R^{a})(R^{b}), —C(═O)—R^{d}, —C(═O)—OR^{d}, —OC(═O)—R^{d}, —N(R^{c})(S(═O)₂R^{d}), —S(═O)₂—N(R^{a})(R^{b}), —SR^{d}, and —OR^{d}, wherein each of the C₁₋₆ alkyl, C₃₋₇ cycloalkyl, and heterocycloalkyl is optionally substituted with one or more substituents each independently selected from the group consisting of halogen, —CN, —OH, C₁₋₄ alkyl, C₁₋₄ alkoxy, C₁₋₄ haloalkyl, C₁₋₄ haloalkoxy, Cₘ cycloalkyl, —N(R^{a})(R^{b}), —N(R^{c})(C(═O)R^{d}), —C(═O)—OR^{d}, —C(═O)H, —C(═O)R^{d}, —C(═O)N(R^{a})(R^{b}), —N(R^{c})(S(═O)₂R^{d}), —S(═O)₂—N(R^{a})(R^{b}), —SR^{d}, and —OR^{d};

each R⁹ is independently selected from the group consisting of H, halogen, —OH, —NO₂, —CN, —SF₅, C₁₋₆ alkyl, C₁₋₆ haloalkyl, C₁₋₆ haloalkoxy, C₂₋₆ alkenyl, C₂₋₆ alkynyl, C₃₋₇ cycloalkyl, a 4- to 10-membered heterocycloalkyl, —N(R^{a})(R^{b}), —N(R^{c})(C(═O)R^{d}), —C(═O)—N(R^{a})(R^{b}), —C(═O)—R^{d}, —C(═O)—OR^{d}, —OC(═O)—R^{d}, —N(R^{c})(S(═O)₂R^{d}), —S(═O)₂—N(R^{a})(R^{b}), —SR^{d}, and —OR^{d}, wherein each of the C₁₋₆ alkyl, C₃₋₇ cycloalkyl, and heterocycloalkyl is optionally substituted with one or more substituents each independently selected from the group consisting of halogen, —CN, —OH, C₁₋₄ alkyl, C₁₋₄ alkoxy, C₁₋₄ haloalkyl, C₁₋₄ haloalkoxy, Cₘ cycloalkyl, —N(R^{a})(R^{b}), —N(R^{c})(C(═O)R^{d}), —C(═O)—OR^{d}, —C(═O)H, —C(═O)R^{d}, —C(═O)N(R^{a})(R^{b}), —N(R^{c})(S(═O)₂R^{d}), —S(═O)₂—N(R^{a})(R^{b}), —SR^{d}, and —OR^{d};

each R^{a} is independently H, C₁₋₄ alkyl, C₁₋₄ haloalkyl, C₃₋₇ cycloalkyl, or (C₃₋₇ cycloalkyl)-C₁₋₄ alkyl-;

each R^{b} is independently H or selected from the group consisting of C₁₋₄ alkyl, C₁-4 haloalkyl, C₃₋₇ cycloalkyl, a 4- to 10-membered heterocycloalkyl, C₆₋₁₀ aryl, a 5- to 10-membered heteroaryl, (C₃₋₇ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the selections from the group is optionally substituted with one or more substituents each independently selected from the group consisting of —OH, —CN, C₁₋₄ alkyl, C₃₋₇ cycloalkyl, C₁₋₄ hydroxylalkyl, —S—C₁₋₄ alkyl, —C(═O)H, —C(═O)—C₁₋₄ alkyl, —C(═O)—O—C₁₋₄ alkyl, —C(═O)—NH₂, —C(═O)—N(C₁₋₄ alkyl)₂, C₁₋₄ haloalkyl, C₁₋₄ alkoxy, and C₁₋₄ haloalkoxy;

or R^{a} and R^{b} together with the N atom to which they are attached form a 4- to 10-membered heterocycloalkyl or a 5- to 10-membered heteroaryl, each optionally substituted with one or more substituents each independently selected from the group consisting of halogen, —OH, oxo, —C(═O)H, —C(═O)OH, —C(═O)—C₁₋₄ alkyl, —C(═O)—NH₂, —C(═O)—N(C₁₋₄ alkyl)₂, —CN, C₁₋₄ alkyl, C₃₋₆ cycloalkyl, (C₃₋₆ cycloalkyl)-C₁₋₂ alkyl-, C₁₋₄ alkoxy, C₁₋₄ hydroxylalkyl, C₁₋₄ haloalkyl, and C₁₋₄ haloalkoxy;

each R^{c} is independently selected from the group consisting of H, C₁₋₄ alkyl, C₃₋₇ cycloalkyl, and (C₃₋₇ cycloalkyl)-C₁₋₄ alkyl-;

each R^{d} is independently selected from the group consisting of C₁₋₆ alkyl, C₃₋₇ cycloalkyl, a 4- to 14-membered heterocycloalkyl, C₆₋₁₀ aryl, a 5- to 10-membered heteroaryl, (C₃₋₇ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the selections from the group is optionally substituted with one or more substituents each independently selected from the group consisting of halogen, —CF₃, —CN, —OH, oxo, —S—C₁₋₄ alkyl, C₁₋₄ alkyl, C₁₋₄ haloalkyl, C₂₋₆ alkenyl, C₂₋₆ alkynyl, C₃₋₇ cycloalkyl, C₁₋₄ alkoxy, and C₁₋₄ haloalkoxy; and

R^{e} and R^{f} of the NR^{e}R^{f} of T², together with the N atom to which they are attached form a 4- to 7-membered heterocycloalkyl optionally substituted with one or more substituents each independently selected from the group consisting of halogen, —OH, oxo, —C(═O)H, —C(═O)OH, —C(═O)—C₁₋₄ alkyl, —C(═O)—NH₂, —C(═O)—N(C₁₋₄ alkyl)₂, —CN, C₁-4 alkyl, C₃₋₆ cycloalkyl, (C₃₋₆ cycloalkyl)-C₁₋₂ alkyl-, C₁₋₄ alkoxy, C₁₋₄ hydroxylalkyl, C₁₋₄ haloalkyl, and C₁₋₄ haloalkoxy.

In some embodiments, when R¹ is optionally substituted (4- to 10-membered heterocycloalkyl)-C₁₋₄ alkyl-, then the 4- to 10-membered heterocycloalkyl moiety comprises one oxygen ring-form atom.

In some embodiments, each of R² and R³ is independently selected from the group consisting of H, halogen (e.g. F or Cl), methyl, C₁ fluoroalkyl, methoxy, and C₁ fluoroalkoxy. In some further embodiments, each of R² and R³ is independently selected from the group consisting of H, halogen (e.g. F or Cl), methyl, and C₁ fluoroalkyl. In some yet further embodiments, each of R² and R³ is independently selected from the group consisting of H and halogen (e.g. F or Cl).

In some embodiments, each of R² and R³ is independently selected from the group consisting of H and F.

In some embodiments, each of R² and R³ is independently selected from the group consisting of H, halogen (e.g. F or Cl), methyl, and C₁ fluoroalkyl; and R⁴ is selected from the group consisting of halogen, OR⁶, CN, C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted with one or more independently selected R⁷, and wherein each of the C₁₋₈ alkyl, C₃₋₁₀ cycloalkyl, 4- to 10-membered heterocycloalkyl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is further optionally substituted one or more oxo. In some further embodiments, each of R² and R³ is independently selected from the group consisting of H and halogen (e.g. F or Cl). In some yet further embodiments, each of R² and R³ is independently selected from the group consisting of H and F.

In some embodiments:

R¹ is selected from the group consisting of C₁₋₈ alkyl, C₃₋₁₀ cycloalkyl, 4- to 10-membered heterocycloalkyl, 5- to 10-membered heteroaryl, (C₃₋₁₀ cycloalkyl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, 5- to 10-membered heteroaryl, (C₃₋₁₀ cycloalkyl)-C₁₋₄ alkyl-, and (5- to 10-membered heteroaryl)-C₁₋₄ alkyl- is optionally substituted one or more independently selected R⁵, and wherein each of the C₁₋₈ alkyl, C₃₋₁₀ cycloalkyl, 4- to 10-membered heterocycloalkyl, (C₃₋₁₀ cycloalkyl)-C₁₋₄ alkyl-, and (5- to 10-membered heteroaryl)-C₁₋₄ alkyl- is further optionally substituted one or more oxo; and

each R⁵ is independently selected from the group consisting of halogen, —OH, —CN, C₁₋₆ alkyl, C₁₋₆ haloalkyl, C₁₋₆ hydroxylalkyl, C₁₋₆ alkoxy, and C₁₋₆ haloalkoxy.

In some embodiments:

R¹ is R²¹, —CH₂—R²¹, R²², —CH₂—R²², R²³, —CH₂—R²³, R²⁴, or R²⁵;

R²¹ is C₃₋₇ cycloalkyl optionally substituted with 1, 2, or 3 substituents each independently selected from halogen, —OH, C₁₋₂ hydroxylalkyl, C₁₋₂ alkoxy, and C₁₋₂ haloalkoxy;

R²² is 4- to 8-membered heterocycloalkyl optionally substituted with 1, 2, or 3 substituents each independently selected from halogen, —OH, C₁₋₂ hydroxylalkyl, C₁₋₂ alkoxy, and C₁₋₂ haloalkoxy, and wherein one of the ring-forming atoms of the 4- to 8-membered heterocycloalkyl is an oxygen atom and the rest of the ring-forming atoms are carbon atoms;

R²³ is 5- or 6-membered heteroaryl optionally substituted with 1, 2, or 3 substituents each independently selected from halogen, —OH, C₁₋₂ hydroxylalkyl, C₁₋₂ alkyl, C₁₋₂ haloalkyl, C₁₋₂ alkoxy, and C₁₋₂ haloalkoxy,

R²⁴ is a moiety of Formula a-1:

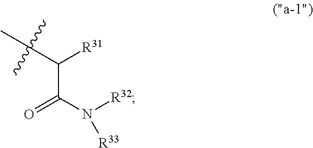

R²⁵ is a moiety of Formula a-2:

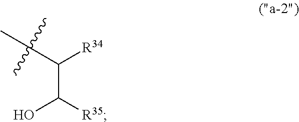

R³¹ is H or C₁₋₄ alkyl;

R³² is H or C₁₋₄ alkyl;

R³³ is H or C₁₋₄ alkyl;

or R³¹ and R³², together with the intervening moiety of C—C(═O)—N(R³³)— to which they are attached, form a 4-10 membered heterocycloalkyl optionally substituted with 1, 2, or 3 substituents each independently selected from halogen, —OH, C₁₋₂ hydroxylalkyl, C₁₋₂ alkyl, C₁₋₂ haloalkyl, C₁₋₂ alkoxy, and C₁₋₂ haloalkoxy,

R³⁴ is H or C₁₋₄ alkyl; and

R³⁵ is H or C₁₋₄ alkyl.

In some embodiments, R¹ is C₃₋₇ cycloalkyl optionally substituted with 1, 2, or 3 substituents each independently selected from halogen (e.g. fluoro), —OH, C₁₋₂ hydroxylalkyl, C₁₋₂ alkoxy, and C₁₋₂ haloalkoxy. In some further embodiments, R¹ is C₄₋₇ cycloalkyl substituted with 1, 2, or 3 substituents each independently selected from halogen (e.g. fluoro), —OH, C₁₋₂ hydroxylalkyl, C₁₋₂ alkoxy, and C₁₋₂ haloalkoxy.

In some embodiments, R¹ is 4- to 7-membered heterocycloalkyl, wherein one of the ring-forming atoms of the 4- to 7-membered heterocycloalkyl is an oxygen atom and the rest of the ring-forming atoms are carbon atoms [the 4- to 7-membered heterocycloalkyl can be, for example, oxetanyl (e.g. oxetan-2-yl), tetrahydrofuran (e.g. tetrahydrofuran-2-yl), or tetrahydropyranyl (e.g., tetrahydro-2H-pyran-4-yl)]; and wherein the 4- to 7-membered heterocycloalkyl is optionally substituted with 1, 2, or 3 substituents each independently selected from halogen (e.g. fluoro), —OH, C₁₋₂ hydroxylalkyl, C₁₋₂ alkoxy, and C₁₋₂ haloalkoxy.

In some embodiments R¹ is selected from the group consisting of C₄₋₇ cycloalkyl and 4- to 7-membered heterocycloalkyl, wherein each of the C₄₋₇ cycloalkyl and 4- to 7-membered heterocycloalkyl is substituted with one OH, and wherein one of the ring-forming atoms of the 4- to 7-membered heterocycloalkyl is an oxygen atom and the rest of the ring-forming atoms are carbon atoms.

In some embodiments, R¹ is a moiety of Formula b-1 or b-2:

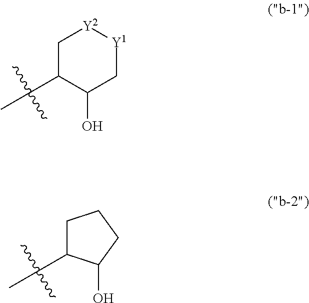

wherein each of Y¹ and Y² is independently 0 or CH₂, provided that at most one of Y¹ and Y² is O. In some further embodiments, the OH group in Formula b-1 or b-2 is trans to the NH—C(═O) moiety of Formula I.

In some embodiments, R¹ is a moiety of Formula b-1. In some further embodiments, one of Y¹ and Y² is O and the other is CH₂. In some yet further embodiments, the OH group in Formula b-1 is trans to the NH—C(═O) moiety of Formula I.

In some embodiments, R¹ is a moiety of Formula b-1; Y¹ is O; and Y² is CH₂. In some further embodiments, the OH group in Formula b-1 is trans to the NH—C(═O) moiety of Formula I.

In some embodiments, R¹ is a moiety of Formula b-1; Y¹ and Y² are both CH₂. In some further embodiments, the OH group in Formula b-1 is trans to the NH—C(═O) moiety of Formula I.

In some embodiments, R¹ is a moiety of Formula b-1; Y¹ and Y² are both CH₂; and the OH group in Formula b-1 is cis to the NH—C(═O) moiety of Formula I.

In some embodiments, R¹ is a moiety of Formula b-2. In some further embodiments, the OH group in Formula b-1 is trans to the NH—C(═O) moiety of Formula I.

In some embodiments, R¹ is a moiety of Formula b-3, b-4, b-5, or b-6:

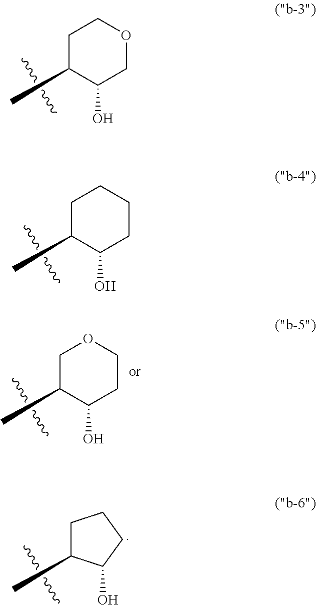

In some embodiments, R¹ is a moiety of Formula b-3.

In some embodiments R¹ is a moiety of Formula b-4.

In some embodiments, R¹ is C₄₋₇ cycloalkyl substituted with one or more (e.g. 1, 2, 3, or 4) halogen (e.g. fluoro). In some further embodiments, R¹ is C₅₋₆ cycloalkyl substituted with one or more (e.g. 1, 2, 3, or 4) halogen (e.g. fluoro). In some yet further embodiments, R¹ is cylcohexyl substituted with one or more (e.g. 1, 2, 3, or 4) halogen (e.g. fluoro). In some still further embodiments, R¹ is 2,2-difluorocyclohexan-1-yl.

In some embodiments, R¹ is C₅₋₆ cycloalkyl substituted with two fluoro wherein the two fluoro are substituted on a same carbon ring-forming atom of the C₅₋₆ cycloalkyl. In some further embodiments, R¹ is 3,3-difluorocyclopentyl or 2,2-difluorocyclohexan-1-yl.

In some embodiments, T¹ is selected from the group consisting of H, halogen, —CN, C₁₋₄ alkyl, and C₁₋₄ haloalkyl, C₁₋₄ alkoxy, and C₁₋₄ haloalkoxy. In some further embodiments, T¹ is selected from the group consisting of H, halogen (e.g., Cl), C₁₋₂ alkyl, and C₁₋₂ haloalkyl.

In some embodiments, T¹ is H, Cl, methyl, or C₁ fluoroalkyl. In some further embodiments, T¹ is H, Cl, or methyl.

In some embodiments, T¹ is H, C₁₋₂ alkyl, or C₁₋₂ haloalkyl. In some further embodiments, T¹ is H or C₁₋₂ alkyl. In yet further embodiments, T¹ is H or methyl. In still further embodiments, T¹ is H.

In some embodiments, T² is selected from the group consisting of Cl, —CN, C₁₋₄ alkyl, C₁₋₄ alkoxy, C₁₋₄ haloalkyl, and C₁₋₄ haloalkoxy. In some further embodiments, T² is selected from the group consisting of Cl, C₁₋₄ alkyl, C₁₋₄ alkoxy, C₁₋₄ haloalkyl, and C₁-4 haloalkoxy. In some yet further embodiments, T² is selected from the group consisting of C₁₋₄ alkyl, C₁₋₄ alkoxy, C₁₋₄ haloalkyl, and C₁₋₄ haloalkoxy.

In some embodiments, T² is selected from the group consisting of Cl, C₁₋₂ alkyl, C₁₋₂ haloalkyl, C₁₋₂ alkoxy, and C₁₋₂ haloalkoxy. In some further embodiments, T² is selected from the group consisting of C₁₋₂ alkyl, C₁₋₂ haloalkyl, C₁₋₂ alkoxy, and C₁₋₂ haloalkoxy.

In some embodiments, T² is selected from the group consisting of Cl, C₁₋₂ alkyl, and C₁₋₂ haloalkyl. In some further embodiments, T² is C₁₋₂ alkyl or C₁₋₂ haloalkyl. In some yet further embodiments, T² is C₁₋₂ alkyl or C₁₋₂ fluoroalkyl.

In some embodiments, T² is Cl, methyl, or C₁ fluoroalkyl.

In some embodiments, T² is methyl or C₁ fluoroalkyl.

In some embodiments, T² is CI or methyl.

In some embodiments, T² is methyl.

In some embodiments, T² is C₁ fluoroalkyl (i.e., CF₃, CHF₂, or CH₂F).

In some embodiments, T² is C₁₋₂ alkoxy or C₁₋₂ haloalkoxy. In some further embodiments, T² is C₁₋₂ alkoxy or C₁₋₂ fluoroalkoxy. In some yet further embodiments, T² is methoxy or C₁ fluoroalkoxy.

In some embodiments, T² is methoxy.

In some embodiments, T² is C₁ fluoroalkoxy (i.e., OCF₃, OCHF₂, or OCH₂F).

In some embodiments, T³ is selected from the group consisting of H, F, Cl, and methyl. In some further embodiments, T³ is selected from the group consisting of H, F, and methyl.

In some embodiments, T³ is H or methyl.

In some further embodiments, T³ is selected from the group consisting of H, Cl, and methyl.

In some embodiments, T³ is H or F.

In some embodiments, T³ is H.

In some embodiments, one of R² and R³ is H, and the other of R² and R³ is H or F.

In some embodiments, each of R² and R³ is H.

In some embodiments, one of R² and R³ is H, and the other of R² and R³ is F.

In some embodiments, 0 or 1 of X¹, X², X³, and X⁴ is N and each of the rest of X¹, X², X³, and X⁴ is CR⁹.

In some embodiments, each of X¹, X², X³, and X⁴ is independently CR⁹.

In some embodiments, 1 of X¹, X², X³, and X⁴ is N and each of the rest of X¹, X², X³, and X⁴ is independently CR⁹.

In some embodiments, each of X¹, X², and X³ is CR⁹ and X⁴ is N.

In some embodiments, each each R⁹ is independently selected from the group consisting of H, halogen, —ON, optionally substituted C₁₋₄ alkyl, optionally substituted C₃₋₆ cycloalkyl, optionally substituted C₃₋₆ cycloalkyl-C₁₋₂ alkyl-, and optionally substituted C₁₋₄ alkoxy.

In some embodiments, each R⁹ is independently H, halogen, C₁₋₄ alkyl, C₁₋₄ haloalkyl, C₁₋₄ alkoxy, or C₁₋₄ haloalkoxy. In some further embodiments, each R⁹ is independently H, halogen, C₁₋₂ alkyl, C₁₋₂ haloalkyl, C₁₋₂ alkoxy, or C₁₋₂ haloalkoxy. In some yet further embodiments, each R⁹ is independently H, or C₁₋₂ alkyl (e.g. methyl).

In some embodiments, each R⁹ is H.

In some embodiments, R⁴ is selected from the group consisting of halogen, C₁₋₆ alkoxy, C₁₋₆ halolkoxy, and 5- to 10-membered heteroaryl, wherein the 5- to 10-membered heteroaryl is optionally substituted with one or more independently selected R⁷; and each R⁷ is independently selected from the group consisting of halogen, —CN, optionally substituted C₁₋₄ alkyl, optionally substituted C₃₋₆ cycloalkyl, optionally substituted C₃₋₆ cycloalkyl-C₁₋₂ alkyl-, and optionally substituted C₁₋₄ alkoxy. In some further embodiments, R⁴ is 5- to 10-membered heteroaryl optionally substituted with one or more independently selected R⁷. In some yet further embodiments, R⁴ is 5- to 6-membered heteroaryl optionally substituted with one or more independently selected R⁷. In some still further embodiments, R⁴ is 5-membered heteroaryl optionally substituted with one or more independently selected R⁷.

In some embodiments, R⁴ is 5-membered heteroaryl optionally substituted with 1 or 2 independently selected R⁷, wherein the 5-membered heteroaryl comprises one nitrogen ring-forming atom and one heteroatom ring-forming atom that is selected from nitrogen, oxygen, and sulfur. In some further embodiments, R⁴ is selected from pyrazolyl, oxazoly, and thiazolyl, each of the selections is optionally substituted with one or more independently selected R⁷.

In some further embodiments, R⁴ is selected from pyrazolyl, oxazoly, and thiazolyl, each of the selections is optionally substituted with 1 or 2 substituents each independently selected from the group consisting of halogen, —CN, OH, C₁₋₂ alkyl, C₁₋₂ haloalkyl, C₁₋₂ alkoxy, and C₁₋₂ haloalkoxy. In some further embodiments, R⁴ is selected from pyrazolyl, oxazoly, and thiazolyl, each of the selections is optionally substituted with 1 or 2 substituents each independently selected from the group consisting of OH, C₁₋₂ alkyl, C₁₋₂ haloalkyl, C₁₋₂ alkoxy, and C₁₋₂ haloalkoxy. In some yet further embodiments, R⁴ is selected from pyrazolyl, oxazoly, and thiazolyl, each of the selections is optionally substituted with 1 or 2 substituents each independently selected from the group consisting of C₁₋₂ alkyl, C₁₋₂ haloalkyl, C₁₋₂ alkoxy, and C₁₋₂ haloalkoxy.

In some embodiments, each R⁷ is independently selected from the group consisting of OH, halogen, —CN, C₁₋₄ alkyl, C₁₋₄ alkoxy, C₃₋₄ cycloalkyl, —O—C₃₋₄ cycloalkyl, —CH₂—C₃₋₄ cycloalkyl, and —O—CH₂—C₃₋₄ cycloalkyl, wherein each of the C₁₋₄ alkyl, C₁₋₄ alkoxy, C₃₋₄ cycloalkyl, —O—C₃₋₄ cycloalkyl, —CH₂—C₃₋₄ cycloalkyl, and —O—CH₂—C₃₋₄ cycloalkyl is optionally substituted with one or more substituents each independently selected from the group consisting of halogen, OH, C₁₋₂ alkyl, C₁₋₂ alkoxy, C₁₋₂ haloalkyl, and C₁₋₂ haloalkoxy.

In some embodiments, each R⁷ is independently selected from the group consisting of OH, halogen, —CN, C₁₋₄ alkyl, C₁₋₄ haloalkyl, C₁₋₄ alkoxy, and C₁₋₄ haloalkoxy.

In some embodiments, each R⁷ is independently selected from the group consisting of OH, halogen, —CN, C₁₋₂ alkyl, C₁₋₂ haloalkyl, C₁₋₂ alkoxy, and C₁₋₂ haloalkoxy.

In some embodiments, each R⁷ is independently selected from the group consisting of halogen, —CN, C₁₋₂ alkyl, C₁₋₂ haloalkyl, C₁₋₂ alkoxy, and C₁₋₂ haloalkoxy.

In some embodiments, each R⁷ is independently selected from the group consisting of halogen, —CN, C₁₋₂ alkyl, and C₁₋₂ haloalkyl.

In some embodiments, each R⁷ is independently selected from the group consisting of OH, C₁₋₂ alkyl, C₁₋₂ haloalkyl, C₁₋₂ alkoxy, and C₁₋₂ haloalkoxy. In some embodiments:

R⁴ is a moiety of Formula c-1, c-2, c-3, c-4, c-5, or c-6:

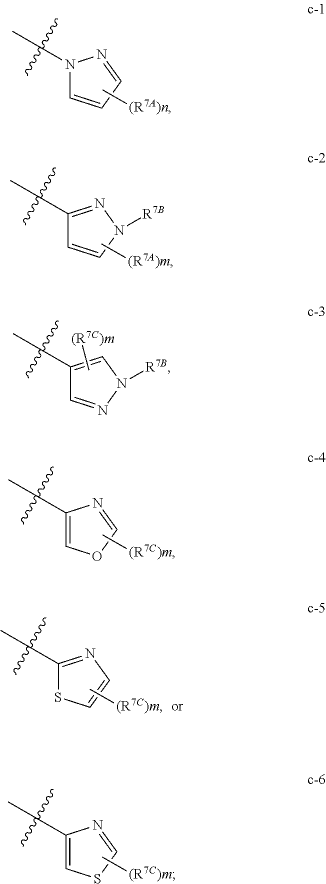

each R^{7A} is independently halogen, —CN, —OH, C₁₋₂ alkyl, C₁₋₂ haloalkyl, C₁₋₂ alkoxy, or C₁₋₂ haloalkoxy;

R^{7B} is C₁₋₂ alkyl;

each R^{7C} is independently C₁₋₂ alkyl, C₁₋₂ haloalkyl, C₁₋₂ alkoxy, or C₁₋₂ haloalkoxy;

n is 0, 1, 2, or 3; and

m is 0, 1, or 2.

In some embodiments, R⁴ is a moiety of Formula c-1.

In some embodiments, R⁴ is a moiety of Formula c-4. In some embodiments, R⁴ is a moiety of Formula c-6.

In some embodiments, R¹ is selected from the group consisting of C₄₋₇ cycloalkyl and 4- to 7-membered heterocycloalkyl, wherein each of the C₄₋₇ cycloalkyl and 4- to 7-membered heterocycloalkyl is substituted with one OH, and wherein one of the ring-forming atoms of the 4- to 7-membered heterocycloalkyl is an oxygen atom and the rest of the ring-forming atoms are carbon atoms; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H, F, Cl, and methyl; one of R² and R³ is H, and the other of R² and R³ is H or F; 0 or 1 of X¹, X², X³, and X⁴ is N and each of the rest of X¹, X², X³, and X⁴ is CR⁹; each R⁹ is independently H, halogen, C₁₋₄ alkyl, C₁₋₄ haloalkyl, C₁₋₄ alkoxy, or C₁₋₄ haloalkoxy; R⁴ is 5- to 6-membered heteroaryl optionally substituted with one or more independently selected R⁷; and each R⁷ is selected from the group consisting of halogen, —CN, C₁₋₄ alkyl, C₁₋₄ haloalkyl, C₁₋₄ alkoxy, and C₁₋₄ haloalkoxy. In some further embodiments, R¹ is a moiety of Formula b-1 or b-2. In some yet further embodiments, the OH group in Formula b-1 or b-2 is trans to the NH—C(═O) moiety of Formula I.

In some embodiments, R¹ is a moiety of Formula b-1 [e.g., wherein either (a) Y¹ is O and Y² is CH₂ or (b) Y¹ is CH₂ and Y² is CH₂]; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H, F, Cl, and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; R⁴ is 5- to 6-membered heteroaryl optionally substituted with one or more independently selected R⁷; and each R⁷ is selected from the group consisting of halogen, —CN, C₁₋₄ alkyl, C₁₋₄ haloalkyl, C₁₋₄ alkoxy, and C₁₋₄ haloalkoxy. In some further embodiments, the OH group in Formula b-1 is trans to the NH—C(═O) moiety of Formula I. In some yet further embodiments, the moiety of Formula b-1 is a moiety of Formula b-3 or b-4.

In some embodiments, R¹ is a moiety of Formula b-3, b-4, b-5, or b-6 (e.g., a moiety of Formula b-3 or b-4); T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H, F, Cl, and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; R⁴ is 5- to 6-membered heteroaryl optionally substituted with one or more independently selected R⁷; and each R⁷ is selected from the group consisting of halogen, —CN, C₁₋₄ alkyl, C₁₋₄ haloalkyl, C₁₋₄ alkoxy, and C₁₋₄ haloalkoxy. In some further embodiments, R⁴ is 5-membered heteroaryl (e.g. pyrazolyl, oxazoly, or thiazolyl) optionally substituted with one or more independently selected R⁷, and wherein the 5-membered heteroaryl comprises one nitrogen ring-forming atom and one heteroatom ring-forming atom that is selected from nitrogen, oxygen, and sulfur. In some yet further embodiments, R⁴ is a moiety of Formula c-1, c-2, c-3, c-4, c-5, or c-6 (e.g. a moiety of Formula c-1, c-4, or c-6).

In some embodiments, R¹ is a moiety of Formula b-3; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H, F, Cl, and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; R⁴ is 5- to 6-membered heteroaryl optionally substituted with one or more independently selected R⁷; and each R⁷ is selected from the group consisting of halogen, —CN, C₁₋₄ alkyl, C₁₋₄ haloalkyl, C₁₋₄ alkoxy, and C₁₋₄ haloalkoxy. In some further embodiments, R⁴ is 5-membered heteroaryl (e.g. pyrazolyl, oxazoly, or thiazolyl) optionally substituted with one ore more independently selected R⁷, and wherein the 5-membered heteroaryl comprises one nitrogen ring-forming atom and one heteroatom ring-forming atom that is selected from nitrogen, oxygen, and sulfur. In some yet further embodiments, R⁴ is a moiety of Formula c-1, c-2, c-3, c-4, c-5, or c-6 (e.g. a moiety of Formula c-1, c-4, or c-6).

In some embodiments, R¹ is a moiety of Formula b-4; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H, F, Cl, and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; R⁴ is 5- to 6-membered heteroaryl optionally substituted with one or more independently selected R⁷; and each R⁷ is selected from the group consisting of halogen, —CN, C₁₋₄ alkyl, C₁₋₄ haloalkyl, C₁₋₄ alkoxy, and C₁₋₄ haloalkoxy. In some further embodiments, R⁴ is 5-membered heteroaryl (e.g. pyrazolyl, oxazoly, or thiazolyl) optionally substituted with one or more independently selected R⁷, and wherein the 5-membered heteroaryl comprises one nitrogen ring-forming atom and one heteroatom ring-forming atom that is selected from nitrogen, oxygen, and sulfur. In some yet further embodiments, R⁴ is a moiety of Formula c-1, c-2, c-3, c-4, c-5, or c-6 (e.g. a moiety of Formula c-1, c-4, or c-6).

In some embodiments, R¹ is a moiety of Formula b-3, b-4, or b-5; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, c-3, c-4, c-5, or c-6 (e.g. a moiety of Formula c-1, c-4, or c-6). In some further embodiments, T¹ is H, methyl, Cl, or C₁ fluoroalkyl. In some yet further embodiments, T² is C₁₋₂ alkyl or C₁₋₂ haloalkyl. In some still further embodiments, both R² and R³ are H.

In some embodiments, R¹ is a moiety of Formula b-3; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, c-3, c-4, c-5, or c-6 (e.g. a moiety of Formula c-1, c-4, or c-6). In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-4; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, c-3, c-4, c-5, or c-6 (e.g. a moiety of Formula c-1, c-4, or c-6). In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-3 or b-4; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, or c-3 (e.g. c-1). In some further embodiments, T¹ is H, methyl, Cl, or C₁ fluoroalkyl. In some yet further embodiments, T² is C₁₋₂ alkyl or C₁₋₂ haloalkyl(e.g. C₁₋₂ fluoroalkyl). In some still further embodiments, both R² and R³ are H.

In some embodiments, R¹ is a moiety of Formula b-3; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, or c-3 (e.g. c-1). In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-3; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1. In some further embodiments, T¹ is H; T³ is H; and T² is C₁₋₂ alkyl or C₁₋₂ haloalkyl (e.g. C₁₋₂ fluoroalkyl). In some yet further embodiments, each of X¹, X², X³, and X⁴ is CR⁹. In some still further embodiments, both R² and R³ are H.

In some embodiments, R¹ is a moiety of Formula b-3; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-4, c-5, or c-6. In some further embodiments, T¹ is H and T³ is H. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-3; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-4. In some further embodiments, T¹ is H; T³ is H; and T² is C₁₋₂ alkyl or C₁₋₂ haloalkyl (e.g. C₁₋₂ fluoroalkyl). In some yet further embodiments, each of X¹, X², X³, and X⁴ is CR⁹. In some still further embodiments, both R² and R³ are H.

In some embodiments, R¹ is a moiety of Formula b-3; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-6. In some further embodiments, T¹ is H; T³ is H; and T² is C₁₋₂ alkyl or C₁₋₂ haloalkyl (e.g. C₁₋₂ fluoroalkyl). In some yet further embodiments, each of X¹, X², X³, and X⁴ is CR⁹. In some still further embodiments, both R² and R³ are H.

In some embodiments, R¹ is a moiety of Formula b-4; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, or c-3. In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-4; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-4. In some further embodiments, T¹ is H; T³ is H; and T² is C₁₋₂ alkyl or C₁₋₂ haloalkyl (e.g. C₁₋₂ fluoroalkyl). In some yet further embodiments, each of X¹, X², X³, and X⁴ is CR⁹. In some still further embodiments, both R² and R³ are H.

In some embodiments, R¹ is a moiety of Formula b-4; T¹ is selected from the group consisting of H, Cl, C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-6. In some further embodiments, T¹ is H; T³ is H; and T² is C₁₋₂ alkyl or C₁₋₂ haloalkyl (e.g. C₁₋₂ fluoroalkyl). In some yet further embodiments, each of X¹, X², X³, and X⁴ is CR⁹. In some still further embodiments, both R² and R³ are H.

In some embodiments, R¹ is a moiety of Formula b-3; T¹ is selected from the group consisting of H, methyl, and CI; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, c-3, c-4, c-5, or c-6 (e.g. a moiety of Formula c-1, c-4, or c-6). In some further embodiments, T¹ is H or methyl; and T² is C₁₋₂ alkyl or C₁₋₂ haloalkyl. In some yet further embodiments, T¹ is H. In some still further embodiments, both R² and R³ are H.

In some embodiments, R¹ is a moiety of Formula b-4; T¹ is selected from the group consisting of H, methyl, and CI; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, c-3, c-4, c-5, or c-6 (e.g. a moiety of Formula c-1, c-4, or c-6). In some further embodiments, T¹ is H or methyl; and T² is C₁₋₂ alkyl or C₁₋₂ haloalkyl. In some yet further embodiments, T¹ is H. In some still further embodiments, both R² and R³ are H.

In some embodiments, R¹ is a moiety of Formula b-3; T¹ is selected from the group consisting of H, methyl, and CI; T² is selected from the group consisting of Cl, C₁₋₂ alkyl, C₁₋₂ haloalkyl, C₁₋₂ alkoxy, and C₁₋₂ haloalkoxy (e.g. C₁₋₂ fluoroalkoxy); T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹ (e.g., each of X¹, X², X³, and X⁴ is CR⁹); each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, or c-3 (e.g. c-1). In some further embodiments, T¹ is H or methyl; and T² is C₁₋₂ alkyl or C₁₋₂ haloalkyl. In some yet further embodiments, T¹ is H. In some still further embodiments, both R² and R³ are H.

In some embodiments, R¹ is a moiety of Formula b-3; T¹ is selected from the group consisting of H, Cl, C₁₋₂ alkyl, and C₁₋₂ fluoroalkyl; T² is selected from the group consisting of Cl, C₁₋₂ alkyl, and C₁₋₂ fluoroalkyl; T³ is H; one of R² and R³ is H, and the other of R² and R³ is H or F; 0 or 1 of X¹, X², X³, and X⁴ is N and each of the rest of X¹, X², X³, and X⁴ is CR⁹; each R⁹ is independently H, halogen, C₁₋₄ alkyl, C₁₋₄ haloalkyl, C₁-4 alkoxy, or C₁₋₄ haloalkoxy; and R⁴ is a moiety of Formula c-1, c-2, or c-3 (e.g., c-1). In some further embodiments, T¹ is H or methyl; and T² is C₁₋₂ alkyl or C₁₋₂ fluoroalkyl. In some yet further embodiments, T¹ is H; and T² is C₁₋₂ alkyl or C₁₋₂ fluoroalkyl. In some still further embodiments, both R² and R³ are H.

In some embodiments, R¹ is a moiety of Formula b-3; T¹ is selected from the group consisting of H, Cl, C₁₋₂ alkyl, and C₁₋₂ fluoroalkyl; T² is selected from the group consisting of Cl, C₁₋₂ alkyl, and C₁₋₂ fluoroalkyl; T³ is H; one of R² and R³ is H, and the other of R² and R³ is H or F; each of X¹, X², X³, and X⁴ is CR⁹; each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-1, c-2, or c-3 (e.g, c-1). In some further embodiments, T¹ is H or methyl; and T² is C₁₋₂ alkyl or C₁₋₂ fluoroalkyl. In some yet further embodiments, T¹ is H; and T² is C₁₋₂ alkyl or C₁₋₂ fluoroalkyl. In still further embodiments, one of R² and R³ is H, and the other of R² and R³ is F.

In some embodiments, R¹ is a moiety of Formula b-3; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹; each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-4, c-5, or c-6. In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-3; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹; each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-4. In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, R¹ is a moiety of Formula b-3; T¹ is selected from the group consisting of H, halogen (e.g. Cl), C₁₋₂ alkyl, and C₁₋₂ haloalkyl; T² is selected from the group consisting of Cl, C₁₋₂ alkyl, C₁₋₂ haloalkyl, C₁₋₂ alkoxy, and C₁₋₂ haloalkoxy; T³ is selected from the group consisting of H and methyl; one of R² and R³ is H, and the other of R² and R³ is H or F; 0 or 1 of X¹, X², X³, and X⁴ is N and each of the rest of X¹, X², X³, and X⁴ is CR⁹; each R⁹ is independently H, halogen, C₁₋₄ alkyl, C₁₋₄ haloalkyl, C₁₋₄ alkoxy, or C₁₋₄ haloalkoxy; and R⁴ is a moiety of Formula c-6. In some further embodiments, T¹ is H, methyl, Cl, or C₁ fluoroalkyl. In some yet further embodiments, T² is C₁₋₂ alkyl or C₁₋₂ haloalkyl (e.g. C₁₋₂ fluoroalkyl). In some still further embodiments, both R² and R³ are H.

In some embodiments, the invention also provides one or more of the compounds or N-oxides described in Examples 1-72 in the Examples section of the subject application, or pharmaceutically acceptable salts of the compounds or the N-oxides.

In some embodiments, the present invention provides a compound or N-oxide selected from the group consisting of:
4-[2-fluoro-4-(1-methyl-1H-pyrazol-4-yl)benzyl]-N-[3-hydroxytetrahydro-2H-pyran-4-yl]-5-methylpyridine-2-carboxamide (e.g., its trans diastereoisomers);
N-[3-hydroxytetrahydro-2H-pyran-4-yl]-5-methyl-4-[4-(1,3-thiazol-2-yl)benzyl]pyridine-2-carboxamide (e.g., its trans diastereoisomers);
N-[3-hydroxytetrahydro-2H-pyran-4-yl]-5-methyl-4-[4-(1,3-thiazol-5-yl)benzyl]pyridine-2-carboxamide (e.g., its trans diastereoisomers);
N-[3-hydroxytetrahydro-2H-pyran-4-yl]-5-methyl-4-[4-(1-methyl-1H-pyrazol-3-yl)benzyl]pyridine-2-carboxamide (e.g., its trans diastereoisomers);
N-[3-hydroxytetrahydro-2H-pyran-4-yl]-5-methyl-4-[4-(1,3-thiazol-4-yl)benzyl]pyridine-2-carboxamide (e.g., its trans diastereoisomers);
N-[3-hydroxytetrahydro-2H-pyran-4-yl]-5-methyl-4-[4-(1H-pyrazol-1-yl)benzyl]pyridine-2-carboxamide (e.g., its trans diastereoisomers);
N-[3-hydroxytetrahydro-2H-pyran-4-yl]-5-methyl-4-[4-(2-methyl-1,3-oxazol-4-yl)benzyl]pyridine-2-carboxamide (e.g., its trans diastereoisomers);
N-[2-hydroxycyclohexyl]-5-methyl-4-[4-(1H-pyrazol-1-yl)benzyl]pyridine-2-carboxamide (e.g., its cis diastereoisomers);
5-chloro-N-[3-hydroxytetrahydro-2H-pyran-4-yl]-6-methyl-4-[4-(1H-pyrazol-1-yl)benzyl]pyridine-2-carboxamide (e.g., its trans diastereoisomers);
N-[3-hydroxytetrahydro-2H-pyran-4-yl]-5-methoxy-4-[4-(1H-pyrazol-1-yl)benzyl]pyridine-2-carboxamide (e.g., its trans diastereoisomers);
5-chloro-N-[2-hydroxycyclohexyl]-6-methyl-4-[4-(1H-pyrazol-1-yl)benzyl]pyridine-2-carboxamide (e.g., its trans diastereoisomers);
N-[(2-hydroxycyclohexyl]-5-methyl-4-[4-(1H-pyrazol-1-yl)benzyl]pyridine-2-carboxamide (e.g., its trans diastereoisomers);
N-[(2-hydroxycyclohexyl]-5-methyl-4-[4-(2-methyl-1,3-oxazol-4-yl)benzyl]pyridine-2-carboxamide (e.g., its trans diastereoisomers);
N-[2-hydroxycyclopentyl]-5-methyl-4-[4-(1H-pyrazol-1-yl)benzyl]pyridine-2-carboxamide (e.g., its trans diastereoisomers);
N-(2,2-difluorocyclohexyl)-5-methyl-4-[4-(1H-pyrazol-1-yl)benzyl]pyridine-2-carboxamide;
5-chloro-N-[3-hydroxytetrahydro-2H-pyran-4-yl]-4-[4-(1H-pyrazol-1-yl)benzyl]pyridine-2-carboxamide (e.g., its trans diastereoisomers);
N-[3-hydroxytetrahydro-2H-pyran-4-yl]-5-methyl-4-[4-(1H-pyrazol-1-yl)benzyl]pyridine-2-carboxamide 1-oxide (e.g., its trans diastereoisomers);
5-(difluoromethyl)-N-[3-hydroxytetrahydro-2H-pyran-4-yl]-4-[4-(1H-pyrazol-1-yl)benzyl]pyridine-2-carboxamide (e.g., its trans diastereoisomers),
4-{fluoro[4-(1H-pyrazol-1-yl)phenyl]methyl}-N-[3-hydroxytetrahydro-2H-pyran-4-yl]-5-methylpyridine-2-carboxamide (e.g., its trans diastereoisomers);
N-[3-Hydroxytetrahydro-2H-pyran-4-yl]-5-methoxy-4-[4-(2-methyl-1,3-oxazol-4-yl)benzyl]pyridine-2-carboxamide (e.g., its trans diastereoisomers);
N-[3-hydroxytetrahydro-2H-pyran-4-yl]-5-methoxy-4-[4-(1,3-thiazol-4-yl)benzyl]pyridine-2-carboxamide (e.g., its trans diastereoisomers); and
N-[3-hydroxytetrahydro-2H-pyran-4-yl]-5-methyl-4-[4-(2-methyl-1,3-thiazol-4-yl)benzyl]pyridine-2-carboxamide (e.g., its trans diastereoisomers),
or an N-oxide thereof, or a pharmaceutically acceptable salt of the compound or N-oxide.

In some embodiments, the present invention provides a compound or N-oxide selected from the group consisting of:
4-[2-fluoro-4-(1-methyl-1H-pyrazol-4-yl)benzyl]-N-[(3R,4S)-3-hydroxytetrahydro-2H-pyran-4-yl]-5-methylpyridine-2-carboxamide;
N-[(3R,4S)-3-hydroxytetrahydro-2H-pyran-4-yl]-5-methyl-4-[4-(1,3-thiazol-2-yl)benzyl]pyridine-2-carboxamide;
N-[(3,4-trans)-3-hydroxytetrahydro-2H-pyran-4-yl]-5-methyl-4-[4-(1,3-thiazol-5-yl)benzyl]pyridine-2-carboxamide, ENT-2;
N-[(3R,4S)-3-hydroxytetrahydro-2H-pyran-4-yl]-5-methyl-4-[4-(1-methyl-1H-pyrazol-3-yl)benzyl]pyridine-2-carboxamide;
N-[(3R,4S)-3-hydroxytetrahydro-2H-pyran-4-yl]-5-methyl-4-[4-(1,3-thiazol-4-yl)benzyl]pyridine-2-carboxamide;
N-[(3R,4S)-3-hydroxytetrahydro-2H-pyran-4-yl]-5-methyl-4-[4-(1H-pyrazol-1-yl)benzyl]pyridine-2-carboxamide;
(+)-N-[(3,4-trans)-3-hydroxytetrahydro-2H-pyran-4-yl]-5-methyl-4-[4-(2-methyl-1,3-oxazol-4-yl)benzyl]pyridine-2-carboxamide;
(−)-N-[(1,2-cis)-2-hydroxycyclohexyl]-5-methyl-4-[4-(1H-pyrazol-1-yl)benzyl]pyridine-2-carboxamide;
5-chloro-N-[(3R,4S)-3-hydroxytetrahydro-2H-pyran-4-yl]-6-methyl-4-[4-(1H-pyrazol-1-yl)benzyl]pyridine-2-carboxamide;
N-[(3R,4S)-3-hydroxytetrahydro-2H-pyran-4-yl]-5-methoxy-4-[4-(1H-pyrazol-1-yl)benzyl]pyridine-2-carboxamide;
5-chloro-N-[(1S,2S)-2-hydroxycyclohexyl]-6-methyl-4-[4-(1H-pyrazol-1-yl)benzyl]pyridine-2-carboxamide;
N-[(1S,2S)-2-hydroxycyclohexyl]-5-methyl-4-[4-(1H-pyrazol-1-yl)benzyl]pyridine-2-carboxamide;
N-[(1S,2S)-2-hydroxycyclohexyl]-5-methyl-4-[4-(2-methyl-1,3-oxazol-4-yl)benzyl]pyridine-2-carboxamide;
N-[trans-2-hydroxycyclopentyl]-5-methyl-4-[4-(1H-pyrazol-1-yl)benzyl]pyridine-2-carboxamide;
N-(2,2-difluorocyclohexyl)-5-methyl-4-[4-(1H-pyrazol-1-yl)benzyl]pyridine-2-carboxamide, ENT-2;
5-chloro-N-[(3R,4S)-3-hydroxytetrahydro-2H-pyran-4-yl]-4-[4-(1H-pyrazol-1-yl)benzyl]pyridine-2-carboxamide;
N-[(3R,4S)-3-hydroxytetrahydro-2H-pyran-4-yl]-5-methyl-4-[4-(1H-pyrazol-1-yl)benzyl]pyridine-2-carboxamide 1-oxide;
5-(difluoromethyl)-N-[(3R,4S)-3-hydroxytetrahydro-2H-pyran-4-yl]-4-[4-(1H-pyrazol-1-yl)benzyl]pyridine-2-carboxamide;
4-{(R)-fluoro[4-(1H-pyrazol-1-yl)phenyl]methyl}-N-[(3R,4S)-3-hydroxytetrahydro-2H-pyran-4-yl]-5-methylpyridine-2-carboxamide;
N-[(3R,4S)-3-Hydroxytetrahydro-2H-pyran-4-yl]-5-methoxy-4-[4-(2-methyl-1,3-oxazol-4-yl)benzyl]pyridine-2-carboxamide;
N-[(3R,4S)-3-hydroxytetrahydro-2H-pyran-4-yl]-5-methoxy-4-[4-(1,3-thiazol-4-yl)benzyl]pyridine-2-carboxamide; and
N-[(3R,4S)-3-hydroxytetrahydro-2H-pyran-4-yl]-5-methyl-4-[4-(2-methyl-1,3-thiazol-4-yl)benzyl]pyridine-2-carboxamide,
or an N-oxide thereof, or a pharmaceutically acceptable salt of the compound or N-oxide.

In some embodiments, the present invention provides a compound that is N-[3-hydroxytetrahydro-2H-pyran-4-yl]-5-methyl-4-[4-(1,3-thiazol-4-yl)benzyl]pyridine-2-carboxamide {e.g., N-[(3R,4S)-3-hydroxytetrahydro-2H-pyran-4-yl]-5-methyl-4-[4-(1,3-thiazol-4-yl)benzyl]pyridine-2-carboxamide}, or an N-oxide thereof, or a pharmaceutically acceptable salt of the compound or the N-oxide.

In some embodiments, the present invention provides a compound that is N-[3-hydroxytetrahydro-2H-pyran-4-yl]-5-methyl-4-[4-(1H-pyrazol-1-yl)benzyl]pyridine-2-carboxamide {e.g., N-[(3R,4S)-3-hydroxytetrahydro-2H-pyran-4-yl]-5-methyl-4-[4-(1H-pyrazol-1-yl)benzyl]pyridine-2-carboxamide}, or an N-oxide thereof, or a pharmaceutically acceptable salt of the compound or the N-oxide.

In some embodiments, the present invention provides a compound that is N-[3-hydroxytetrahydro-2H-pyran-4-yl]-5-methyl-4-[4-(2-methyl-1,3-oxazol-4-yl)benzyl]pyridine-2-carboxamide {e.g., (+)-N-[(3,4-trans)-3-hydroxytetrahydro-2H-pyran-4-yl]-5-methyl-4-[4-(2-methyl-1,3-oxazol-4-yl)benzyl]pyridine-2-carboxamide}, or an N-oxide thereof, or a pharmaceutically acceptable salt of the compound or the N-oxide.

In some embodiments, the present invention provides a compound that is N-[3-hydroxytetrahydro-2H-pyran-4-yl]-5-methoxy-4-[4-(1H-pyrazol-1-yl)benzyl]pyridine-2-carboxamide {e.g., N-[(3R,4S)-3-hydroxytetrahydro-2H-pyran-4-yl]-5-methoxy-4-[4-(1H-pyrazol-1-yl)benzyl]pyridine-2-carboxamide}, or an N-oxide thereof, or a pharmaceutically acceptable salt of the compound or the N-oxide.

In some embodiments, the present invention provides a compound that is 4-{fluoro[4-(1H-pyrazol-1-yl)phenyl]methyl}-N-[3-hydroxytetrahydro-2H-pyran-4-yl]-5-methylpyridine-2-carboxamide {e.g., 4-{(R)-fluoro[4-(1H-pyrazol-1-yl)phenyl]methyl}-N-[(3R,4S)-3-hydroxytetrahydro-2H-pyran-4-yl]-5-methylpyridine-2-carboxamide}, or an N-oxide thereof, or a pharmaceutically acceptable salt of the compound or the N-oxide.

In some embodiments, the present invention provides a compound that is N-[3-Hydroxytetrahydro-2H-pyran-4-yl]-5-methoxy-4-[4-(2-methyl-1,3-oxazol-4-yl)benzyl]pyridine-2-carboxamide {e.g., N-[(3R,4S)-3-Hydroxytetrahydro-2H-pyran-4-yl]-5-methoxy-4-[4-(2-methyl-1,3-oxazol-4-yl)benzyl]pyridine-2-carboxamide}, or an N-oxide thereof, or a pharmaceutically acceptable salt of the compound or the N-oxide.

In some embodiments, the present invention provides a compound that is N-[3-hydroxytetrahydro-2H-pyran-4-yl]-5-methoxy-4-[4-(1,3-thiazol-4-yl)benzyl]pyridine-2-carboxamide {e.g., N-[(3R,4S)-3-hydroxytetrahydro-2H-pyran-4-yl]-5-methoxy-4-[4-(1,3-thiazol-4-yl)benzyl]pyridine-2-carboxamide}, or an N-oxide thereof, or a pharmaceutically acceptable salt of the compound or the N-oxide.

In some embodiments, the present invention provides a compound that is N-[3-hydroxytetrahydro-2H-pyran-4-yl]-5-methyl-4-[4-(2-methyl-1,3-thiazol-4-yl)benzyl]pyridine-2-carboxamide {e.g., N-[(3R,4S)-3-hydroxytetrahydro-2H-pyran-4-yl]-5-methyl-4-[4-(2-methyl-1,3-thiazol-4-yl)benzyl]pyridine-2-carboxamide}, or an N-oxide thereof, or a pharmaceutically acceptable salt of the compound or the N-oxide.

The present invention includes any subset of any embodiment described herein.

The present invention includes combinations of two or more embodiments described hereinabove, or any subset thereof.

The present invention further provides the compound of Formula I or an N-oxide thereof or a pharmaceutically acceptable salt of the compound or the N-oxide (including all embodiments and combinations of two or more embodiments described herein or any subcombination thereof) for use in treating an M1-mediated (or M1-associated) disorder described herein.

The present invention further provides use of the compound of Formula I or an N-oxide thereof or a pharmaceutically acceptable salt of the compound or the N-oxide (including all embodiments and combinations of two or more embodiments described herein or any subcombination thereof) for treating an M1-mediated (or M1-associated) disorder described herein.

The present invention further provides a method for treating an M1-mediated (or M1-associated) disorder in a patient (e.g. a mammal such as a human) comprising administering to patient a therapeutically effective amount of the compound of Formula I or an N-oxide thereof or a pharmaceutically acceptable salt of the compound or the N-oxide (including all embodiments and combinations of two or more embodiments described herein or any subcombination thereof).

The present invention further provides use of the compound of Formula I or an N-oxide thereof or a pharmaceutically acceptable salt of the compound or the N-oxide (including all embodiments and combinations of two or more embodiments described herein or any subcombination thereof) in manufacturing a medicament for use in treating an M1-mediated (or M1-associated) disorder described herein.

The compound of Formula I or an N-oxide thereof or a pharmaceutically acceptable salt of the compound or the N-oxide of present invention is an M-1 modulator (e.g., an M-1 positive allosteric modulator). Thus, the present invention further provides a method for modulating an activity of M1 receptor (either in vitro or in vivo, for example, modulating via a positive allosteric site of the M1 receptor), comprising contacting (including incubating) the M1 receptor with a compound of Formula I, or an N-oxide thereof, or a pharmaceutically acceptable salt thereof of the compound or the N-oxide (such as one selected from Examples 1-72 herein) described herein.

The amount of the compound of Formula I or an N-oxide thereof or a pharmaceutically acceptable salt of the foregoing used in any one of the methods of the present invention is effective in modulating an activity of M1 receptor (e.g. via a positive allosteric site of the M1 receptor).

M1-mediated (or M1-associated) disorders include, for example, Alzheimer's disease, schizophrenia or psychosis, a cognitive disorder (e.g. mild cognitive impairment), addiction (e.g. substance addiction such as addiction to opioids, cocaine, or alcohol), pain (e.g. acute pain, inflammatory pain, and neuropathic pain), and a sleep disorder (such as those related to REM sleep regulation, for example, those related to REM sleep onset). Additional M1-mediated (or M1-associated) disorders or conditions that may be treated by the compounds of the invention include, dry mouth, a cognitive disorder (e.g. mild cognitive impairment), Parkinson's Disease, dyskinesia, pulmonary hypertension, chronic obstructive pulmonary disease (COPD), asthma, urinary incontinence, glaucoma, Trisomy 21 (Down Syndrome), cerebral amyloid angiopathy, dementia (e.g. degenerative dementia), Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes, autism, and atherosclerosis. See e.g. U.S. Pat. No. 8,664,234.

Schizophrenia or psychosis for which the compounds, N-oxide thereof, and pharmaceutically acceptable salts of the foregoing of the invention may be useful includes one or more of the following conditions: schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthesia, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both the positive and the negative symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse); delirium, amnestic disorders, or age related cognitive decline.

Potential sleep disorders for which the compounds, N-oxide thereof, and pharmaceutically acceptable salts of the foregoing of the invention may be useful include: enhancing sleep quality; improving sleep quality; augmenting sleep maintenance; increasing the value which is calculated from the time that a subject sleeps divided by the time that a subject is attempting to sleep; decreasing sleep latency or onset (the time it takes to fall asleep); decreasing difficulties in falling asleep; increasing sleep continuity; decreasing the number of awakenings during sleep; decreasing nocturnal arousals; decreasing the time spent awake following the initial onset of sleep; increasing the total amount of sleep; reducing the fragmentation of sleep; altering the timing, frequency or duration of REM sleep bouts; altering the timing, frequency or duration of slow wave (i.e. stages 3 or 4) sleep bouts; increasing the amount and percentage of stage 2 sleep; promoting slow wave sleep; enhancing EEG-delta activity during sleep; increasing daytime alertness; reducing daytime drowsiness; treating or reducing excessive daytime sleepiness; insomnia; hypersomnia; narcolepsy; interrupted sleep; sleep apnea; wakefulness; nocturnal myoclonus; REM sleep interruptions; jet-lag; shift workers' sleep disturbances; dyssomnias; night terror; insomnias associated with depression, emotional/mood disorders, as well as sleep walking and enuresis, and sleep disorders which accompany aging; Alzheimer's sundowning; conditions associated with circadian rhythmicity as well as mental and physical disorders associated with travel across time zones and with rotating shift-work schedules; conditions due to drugs which cause reductions in REM sleep as a side effect; syndromes which are manifested by non-restorative sleep and muscle pain or sleep apnea which is associated with respiratory disturbances during sleep; and conditions which result from a diminished quality of sleep.

Pain disorders for which the compounds, N-oxide thereof, and pharmaceutically acceptable salts of the foregoing of the invention may be useful include neuropathic pain (such as postherpetic neuralgia, nerve injury, the “dynias”, e.g., vulvodynia, phantom limb pain, root avulsions, painful diabetic neuropathy, painful traumatic mononeuropathy, painful polyneuropathy); central pain syndromes (potentially caused by virtually any lesion at any level of the nervous system); postsurgical pain syndromes (eg, postmastectomy syndrome, postthoracotomy syndrome, stump pain); bone and joint pain (osteoarthritis), repetitive motion pain, dental pain, cancer pain, myofascial pain (muscular injury, fibromyalgia); perioperative pain (general surgery, gynecological), chronic pain, dysmennorhea, as well as pain associated with angina, and inflammatory pain of varied origins (e.g. osteoarthritis, rheumatoid arthritis, rheumatic disease, teno-synovitis and gout), headache, migraine and cluster headache, headache, primary hyperalgesia, secondary hyperalgesia, primary allodynia, secondary allodynia, or other pain caused by central sensitization.

The compounds, N-oxides thereof, and pharmaceutically acceptable salts of the foregoing of the invention may be used to decrease tolerance and/or dependence to opioid treatment of pain, and for treatment of withdrawal syndrome of e.g., alcohol, opioids, and cocaine.

The term “therapeutically effective amount” as used herein refers to that amount of the compound (including an N-oxide thereof or a pharmaceutically acceptable salt of the compound or the N-oxide) being administered which will relieve to some extent one or more of the symptoms of the disorder being treated. In reference to the treatment of an M1-mediated disorder (e.g., Alzheimer's disease or schizophrenia), a therapeutically effective amount refers to that amount which has the effect of relieving to some extent (or, for example, eliminating) one or more symptoms associated with the M1-mediated disorder (e.g., positive, negative, or cognitive symptom of schizophrenia; or psychotic symptom of Alzheimer's disease).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined herein. The term “treating” also includes adjuvant and neo-adjuvant treatment of a subject.

As used herein, the term “n-membered” where n is an integer typically describes the number of ring-forming atoms in a moiety where the number of ring-forming atoms is n. For example, pyridine is an example of a 6-membered heteroaryl ring and thiophene is an example of a 5-membered heteroaryl group.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to include C₁ alkyl (methyl), C₂ alkyl (ethyl), C₃ alkyl, C₄ alkyl, C₅ alkyl, and C₆ alkyl. For another example, the term “a 5- to 10-membered heteroaryl group” is specifically intended to include any 5-, 6-, 7-, 8-, 9- or 10-membered heteroaryl group.

As used herein, the term “alkyl” is defined to include saturated aliphatic hydrocarbons including straight chains and branched chains. In some embodiments, the alkyl group has 1 to 20 carbon atoms, 1 to 10 carbon atoms, 1 to 6 carbon atoms, or 1 to 4 carbon atoms. For example, the term “C₁₋₆ alkyl,” as well as the alkyl moieties of other groups referred to herein (e.g., C₁₋₆alkoxy) refers to linear or branched radicals of 1 to 6 carbon atoms (e.g., methyl, ethyl, n-propyl, isopropyl, n-butyl, isobutyl, sec-butyl, tert-butyl, n-pentyl, or n-hexyl). For yet another example, the term “C₁₋₄ alkyl” refers to linear or branched aliphatic hydrocarbon chains of 1 to 4 carbon atoms; the term “C₁₋₃ alkyl” refers to linear or branched aliphatic hydrocarbon chains of 1 to 3 carbon atoms; the term “C₁₋₂ alkyl” refers to linear or branched aliphatic hydrocarbon chains of 1 to 2 carbon atoms; and the term “C₁ alkyl” refers to methyl. An alkyl group optionally can be substituted by one or more (e.g. 1 to 5) suitable substituents.

As used herein, the term “alkenyl” refers to aliphatic hydrocarbons having at least one carbon-carbon double bond, including straight chains and branched chains having at least one carbon-carbon double bond. In some embodiments, the alkenyl group has 2 to 20 carbon atoms, 2 to 10 carbon atoms, 2 to 6 carbon atoms, 3 to 6 carbon atoms, or 2 to 4 carbon atoms. For example, as used herein, the term “C₂₋₆ alkenyl” means straight or branched chain unsaturated radicals (having at least one carbon-carbon double bond) of 2 to 6 carbon atoms, including, but not limited to, ethenyl, 1-propenyl, 2-propenyl (allyl), isopropenyl, 2-methyl-1-propenyl, 1-butenyl, 2-butenyl, and the like. An alkenyl group optionally can be substituted by one or more (e.g. 1 to 5) suitable substituents. When the compounds of Formula I contain an alkenyl group, the alkenyl group may exist as the pure E form, the pure Z form, or any mixture thereof.

As used herein, the term “alkynyl” refers to aliphatic hydrocarbons having at least one carbon-carbon triple bond, including straight chains and branched chains having at least one carbon-carbon triple bond. In some embodiments, the alkynyl group has 2 to 20, 2 to 10, 2 to 6, or 3 to 6 carbon atoms. For example, as used herein, the term “C₂₋₆ alkynyl” refers to straight or branched hydrocarbon chain alkynyl radicals as defined above, having 2 to 6 carbon atoms. An alkynyl group optionally can be substituted by one or more (e.g. 1 to 5) suitable substituents.

As used herein, the term “cycloalkyl” refers to saturated or unsaturated, non-aromatic, monocyclic or polycyclic (such as bicyclic) hydrocarbon rings (e.g., monocyclics such as cyclopropyl, cyclobutyl, cyclopentyl, cyclohexyl, cycloheptyl, cyclooctyl, cyclononyl, or bicyclics including spiro, fused, or bridged systems (such as bicyclo[1.1.1]pentanyl, bicyclo[2.2.1]heptanyl, bicyclo[3.2.1]octanyl or bicyclo[5.2.0]nonanyl, decahydronaphthalenyl, etc.). The cycloalkyl group has 3 to 15 carbon atoms. In some embodiments the cycloalkyl may optionally contain one, two or more non-cumulative non-aromatic double or triple bonds and/or one to three oxo groups. In some embodiments, the bicycloalkyl group has 6 to 14 carbon atoms. For example, the term “C₃₋₁₄ cycloalkyl” refers to saturated or unsaturated, non-aromatic, monocyclic or polycyclic (such as bicyclic) hydrocarbon rings of 3 to 14 ring-forming carbon atoms (e.g., cyclopropyl, cyclobutyl, cyclopentyl, cyclohexyl, bicyclo[1.1.1]pentanyl, or cyclodecanyl); and the term “C₃₋₇ cycloalkyl” refers to saturated or unsaturated, non-aromatic, monocyclic or polycyclic (such as bicyclic) hydrocarbon rings of 3 to 7 ring-forming carbon atoms (e.g., cyclopropyl, cyclobutyl, cyclopentyl, cyclohexyl, bicyclo[1.1.1]pentan-1-yl, or bicyclo[1.1.1]pentan-2-yl). For another example, the term “C₃₋₆ cycloalkyl” refers to saturated or unsaturated, non-aromatic, monocyclic or polycyclic (such as bicyclic) hydrocarbon rings of 3 to 6 ring-forming carbon atoms. For yet another example, the term “C₃₋₄ cycloalkyl” refers to cyclopropyl or cyclobutyl. Also included in the definition of cycloalkyl are moieties that have one or more aromatic rings (including aryl and heteroaryl) fused to the cycloalkyl ring, for example, benzo or thienyl derivatives of cyclopentane, cyclopentene, cyclohexane, and the like (e.g., 2,3-dihydro-1H-indene-1-yl, or 1H-inden-2(3H)-one-1-yl). The cycloalkyl group optionally can be substituted by 1 or more (e.g., 1 to 5) suitable substituents.

As used herein, the term “aryl” refers to all-carbon monocyclic or fused-ring polycyclic aromatic groups having a conjugated pi-electron system. The aryl group has 6 or 10 carbon atoms in the ring(s). Most commonly, the aryl group has 6 carbon atoms in the ring. For example, as used herein, the term “C₆₋₁₀ aryl” means aromatic radicals containing from 6 to 10 carbon atoms such as phenyl or naphthyl. The aryl group optionally can be substituted by 1 or more (e.g., 1 to 5) suitable substituents.

As used herein, the term “heteroaryl” refers to monocyclic or fused-ring polycyclic aromatic heterocyclic groups with one or more heteroatom ring members (ring-forming atoms) each independently selected from O, S and N in at least one ring. The heteroaryl group has 5 to 14 ring-forming atoms, including 1 to 13 carbon atoms, and 1 to 8 heteroatoms selected from O, S, and N. In some embodiments, the heteroaryl group has 5 to 10 ring-forming atoms including one to four heteroatoms. The heteroaryl group can also contain one to three oxo or thiono (i.e. ═S) groups. In some embodiments, the heteroaryl group has 5 to 8 ring-forming atoms including one, two or three heteroatoms. For example, the term “5-membered heteroaryl” refers to a monocyclic heteroaryl group as defined above with 5 ring-forming atoms in the monocyclic heteroaryl ring; the term “6-membered heteroaryl” refers to a monocyclic heteroaryl group as defined above with 6 ring-forming atoms in the monocyclic heteroaryl ring; and the term “5- or 6-membered heteroaryl” refers to a monocyclic heteroaryl group as defined above with 5 or 6 ring-forming atoms in the monocyclic heteroaryl ring. For another example, term “5- or 10-membered heteroaryl” refers to a monocyclic or bicyclic heteroaryl group as defined above with 5, 6, 7, 8, 9 or 10 ring-forming atoms in the monocyclic or bicyclic heteroaryl ring. A heteroaryl group optionally can be substituted by 1 or more (e.g., 1 to 5) suitable substituents. Examples of monocyclic heteroaryls include those with 5 ring-forming atoms including one to three heteroatoms or those with 6 ring-forming atoms including one, two or three nitrogen heteroatoms. Examples of fused bicyclic heteroaryls include two fused 5- and/or 6-membered monocyclic rings including one to four heteroatoms.

Examples of heteroaryl groups include pyridinyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl (e.g., pyrazol-1-yl, pyrazol-3-yl, pyrazol-4-yl), tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, 1H-imidazo[4,5-c]pyridinyl, imidazo[1,2-a]pyridinyl, 1H-pyrrolo[3,2-c]pyridinyl, imidazo[1,2-a]pyrazinyl, imidazo[2,1-c][1,2,4]triazinyl, imidazo[1,5-a]pyrazinyl, imidazo[1,2-a]pyrimidinyl, 1H-indazolyl, 9H-purinyl, imidazo[1,2-a]pyrimidinyl, [1,2,4]triazolo[1,5-a]pyrimidinyl, [1,2,4]triazolo[4,3-b]pyridazinyl, isoxazolo[5,4-c]pyridazinyl, isoxazolo[3,4-c]pyridazinyl, pyridone, pyrimidone, pyrazinone, pyrimidinone, 1H-imidazol-2(3H)-one, 1H-pyrrole-2,5-dione, 3-oxo-2H-pyridazinyl, 1H-2-oxo-pyrimidinyl, 1H-2-oxo-pyridinyl, 2,4(1H,3H)-dioxo-pyrimidinyl, 1H-2-oxo-pyrazinyl, and the like. The heteroaryl group optionally can be substituted by 1 or more (e.g., 1 to 5) suitable substituents.

As used herein, the term “heterocycloalkyl” refers to a monocyclic or polycyclic [including 2 or more rings that are fused together, including spiro, fused, or bridged systems, for example, a bicyclic ring system], saturated or unsaturated, non-aromatic 4- to 15-membered ring system (such as a 4- to 14-membered ring system, 4- to 12-membered ring system, 5- to 10-membered ring system, 4- to 7-membered ring system, 4- to 6-membered ring system, or 5- to 6-membered ring system), including 1 to 14 ring-forming carbon atoms and 1 to 10 ring-forming heteroatoms each independently selected from O, S and N. The heterocycloalkyl group can also optionally contain one or more oxo or thiono (i.e. ═S) groups. For example, the term “4- to 12-membered heterocycloalkyl” refers to a monocyclic or polycyclic, saturated or unsaturated, non-aromatic 4- to 12-membered ring system that comprises one or more ring-forming heteroatoms each independently selected from O, S and N; and the term “4- to 10-membered heterocycloalkyl” refers to a monocyclic or polycyclic, saturated or unsaturated, non-aromatic 4- to 10-membered ring system that comprises one or more ring-forming heteroatoms each independently selected from O, S and N. For another example, the term “4- to 6-membered heterocycloalkyl” refers to a monocyclic or polycyclic, saturated or unsaturated, non-aromatic 4- to 6-membered ring system that comprises one or more ring-forming heteroatoms each independently selected from O, S and N; and the term “5- to 6-membered heterocycloalkyl” refers to a monocyclic or polycyclic, saturated or unsaturated, non-aromatic 5- to 6-membered ring system that comprises one or more ring-forming heteroatoms each independently selected from O, S and N. Also included in the definition of heterocycloalkyl are moieties that have one or more aromatic rings (including aryl and heteroaryl) fused to the nonaromatic heterocycloalkyl ring, for example pyridinyl, pyrimidinyl, thiophenyl, pyrazolyl, phthalimidyl, naphthalimidyl, and benzo derivatives of the nonaromatic heterocycloalkyl rings. The heterocycloalkyl group optionally can be substituted by 1 or more (e.g., 1 to 5) suitable substituents.

Examples of such heterocycloalkyl rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, quinuclidinyl, chromanyl, isochromanyl, benzoxazinyl, 2-oxaspiro[3.3]heptyl {e.g. 2-oxaspiro[3.3]hept-6-yl}, 7-azabicyclo[2.2.1]heptan-1-yl, 7-azabicyclo[2.2.1]heptan-2-yl, 7-azabicyclo[2.2.1]heptan-7-yl, 2-azabicyclo[2.2.1]heptan-3-on-2-yl, 3-azabicyclo[3.1.0]hexanyl, 3-azabicyclo[4.1.0]heptanyl and the like. Further examples of heterocycloalkyl rings include tetrahydrofuran-2-yl, tetrahydrofuran-3-yl, tetrahydropyranyl (e.g. tetrahydro-2H-pyran-4-yl), imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, 1,3-oxazolidin-3-yl, 1,4-oxazepan-1-yl, isothiazolidinyl, 1,3-thiazolidin-3-yl, 1,2-pyrazolidin-2-yl, 1,2-tetrahydrothiazin-2-yl, 1,3-thiazinan-3-yl, 1,2-tetrahydrodiazin-2-yl, 1,3-tetrahydrodiazin-1-yl, 1,4-oxazin-4-yl, oxazolidinonyl, 2-oxo-piperidinyl (e.g., 2-oxo-piperidin-1-yl), 2-oxoazepan-3-yl, and the like. Some examples of aromatic-fused heterocycloalkyl groups include indolinyl, isoindolinyl, isoindolin-1-one-3-yl, 5,7-dihydro-6H-pyrrolo[3,4-b]pyridin-6-yl, 6,7-dihydro-5H-pyrrolo[3,4-d]pyrimidin-6-yl, 4,5,6,7-tetrahydrothieno[2,3-c]pyridine-5-yl, 5,6-dihydrothieno[2,3-c]pyridin-7(4H)-one-5-yl, 1,4,5,6-tetrahydropyrrolo[3,4-c]pyrazol-5-yl, and 3,4-dihydroisoquinolin-1(2H)-one-3-yl groups. The heterocycloalkyl group is optionally substituted by 1 or more (e.g., 1 to 5) suitable substituents. Examples of heterocycloalkyl groups include 5- or 6-membered monocyclic rings and 9- or 10-membered fused bicyclic rings.

As used herein, the term “halo” or “halogen” group is defined to include fluorine, chlorine, bromine or iodine.

As used herein, the term “haloalkyl” refers to an alkyl group having one or more halogen substituents (up to perhaloalkyl, i.e., every hydrogen atom of the alkyl group has been replaced by a halogen atom). For example, the term “C₁₋₆ haloalkyl” refers to a C₁₋₆ alkyl group having one or more halogen substituents (up to perhaloalkyl, i.e., every hydrogen atom of the alkyl group has been replaced by a halogen atom). For another example, the term “C₁₋₄ haloalkyl” refers to a C₁₋₄ alkyl group having one or more halogen substituents (up to perhaloalkyl, i.e., every hydrogen atom of the alkyl group has been replaced by a halogen atom); the term “C₁₋₃ haloalkyl” refers to a C₁₋₃ alkyl group having one or more halogen substituents (up to perhaloalkyl, i.e., every hydrogen atom of the alkyl group has been replaced by a halogen atom); and the term “C₁₋₂ haloalkyl” refers to a C₁₋₂ alkyl group (i.e. methyl or ethyl) having one or more halogen substituents (up to perhaloalkyl, i.e., every hydrogen atom of the alkyl group has been replaced by a halogen atom). For yet another example, the term “C₁ haloalkyl” refers to a methyl group having one, two, or three halogen substituents. Examples of haloalkyl groups include CF₃, C₂F₅, CHF₂, CH₂F, CH₂CF₃, CH₂Cl and the like.

As used herein, the term “alkoxy” or “alkyloxy” refers to an —O-alkyl group. For example, the term “C₁₋₆ alkoxy” or “C₁₋₆ alkyloxy” refers to an —O—(C₁₋₆ alkyl) group; and the term “C₁₋₄ alkoxy” or “C₁₋₄ alkyloxy” refers to an —O—(C₁ alkyl) group; For another example, the term “C₁₋₂ alkoxy” or “C₁₋₂ alkyloxy” refers to an —O—(C₁₋₂ alkyl) group. Examples of alkoxy include methoxy, ethoxy, propoxy (e.g., n-propoxy and isopropoxy), tert-butoxy, and the like. The alkoxy or alkyloxy group optionally can be substituted by 1 or more (e.g., 1 to 5) suitable substituents.

As used here, the term “haloalkoxy” refers to an —O-haloalkyl group. For example, the term “C₁₋₆ haloalkoxy” refers to an —O—(C₁₋₆ haloalkyl) group. For another example, the term “C₁₋₄ haloalkoxy” refers to an —O—(C₁₋₄ haloalkyl) group; and the term “C₁₋₂ haloalkoxy” refers to an —O—(C₁₋₂ haloalkyl) group. For yet another example, the term “C₁ haloalkoxy” refers to a methoxy group having one, two, or three halogen substituents. An example of haloalkoxy is —OCF₃ or OCHF₂.

As used herein, the term “cycloalkoxy” or “cycloalkyloxy” refers to an —O— cycloalkyl group. For example the term “C₃₋₇ cycloalkoxy” or “C₃₋₇ cycloalkyloxy” refers to an —O—(C₃₋₇ cycloalkyl) group. For another example, the term “C₃₋₆ cycloalkoxy” or “C₃₋₆ cycloalkyloxy” refers to an —O—(C₃₋₆ cycloalkyl) group. Examples of cycloalkoxy include C₃₋₆ cycloalkoxy (e.g., cyclopropoxy, cyclobutoxy, cyclopentoxy, cyclohexanoxy, and the like). The cycloalkoxy or cycloalkyloxy group optionally can be substituted by 1 or more (e.g., 1 to 5) suitable substituents.

As used here, the term “C₆₋₁₀ aryloxy” refers to an O—(C₆₋₁₀ aryl) group. An example of a C₆₋₁₀ aryloxy group is —O-phenyl [i.e., phenoxy]. The C₆₋₁₀ aryloxy y group optionally can be substituted by 1 or more (e.g., 1 to 5) suitable substituents.

As used herein, the term “fluoroalkyl” refers to an alkyl group having one or more fluorine substituents (up to perfluoroalkyl, i.e., every hydrogen atom of the alkyl group has been replaced by fluorine). For example, the term “C₁₋₂ fluoroalkyl” refers to a C₁₋₂ alkyl group having one or more fluorine substituents (up to perfluoroalkyl, i.e., every hydrogen atom of the C₁₋₂ alkyl group has been replaced by fluorine). For another example, the term “C₁ fluoroalkyl” refers to a C₁ alkyl group (i.e., methyl) having 1, 2, or 3 fluorine substituents). Examples of fluoroalkyl groups include CF₃, C₂F₅, CH₂CF₃, CHF₂, CH₂F, and the like.

As used here, the term “fluoroalkoxy” refers to an —O-fluoroalkyl group. For example, the term “C₁₋₂ fluoroalkoxy” refers to an —O—C₁₋₂ fluoroalkyl group. For another example, the term “C₁ fluoroalkoxy” refers to a methoxy group having one, two, or three fluorine substituents. An example of C₁ fluoroalkoxy is —OCF₃ or OCHF₂.

As used herein, the term “hydroxylalkyl” or “hydroxyalkyl” refers to an alkyl group having one or more (e.g., 1, 2, or 3) OH substituents. The term “C₁₋₆ hydroxylalkyl” or “C₁₋₆ hydroxyalkyl” refers to a C₁₋₆ alkyl group having one or more (e.g., 1, 2, or 3) OH substituents. The term “C₁₋₄ hydroxylalkyl” or “C₁₋₄ hydroxyalkyl” refers to a C₁₋₄ alkyl group having one or more (e.g., 1, 2, or 3) OH substituents; the term “C₁₋₃ hydroxylalkyl” or “C₁₋₃ hydroxyalkyl” refers to a C₁₋₃ alkyl group having one or more (e.g., 1, 2, or 3) OH substituents; and the term “C₁₋₂ hydroxylalkyl” or “C₁₋₂ hydroxyalkyl” refers to a C₁₋₂ alkyl group having one or more (e.g., 1, 2, or 3) OH substituents. An example of hydroxylalkyl is —CH₂OH or —CH₂CH₂OH.

As used herein, the term “oxo” refers to ═O. When an oxo is substituted on a carbon atom, they together form a carbonyl moiety [—C(═O)—]. When an oxo is substituted on a sulfur atom, they together form a sulfinyl moiety [—S(═O)—]; when two oxo groups are substituted on a sulfur atom, they together form a sulfonyl moiety [—S(═O)₂—].

As used herein, the term “thiono” refers to ═S. When an thiono is substituted on a carbon atom, they together form moiety of [—C(═S)—].

As used herein, the term “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group (up to that every hydrogen atom on the designated atom or moiety is replaced with a selection from the indicated substituent group), provided that the normal valency of the designated atom or moiety is not exceeded, and that the substitution results in a stable compound. For example, if a methyl group (i.e., CH₃) is optionally substituted, then up to 3 hydrogen atoms on the carbon atom can be replaced with substituent groups.

As used herein, the term “optionally substituted C₁₋₄ alkyl” refers to C₁₋₄ alkyl optionally substituted by one or more (e.g. 1 to 5) substituents each independently selected from the group consisting of —OH, halogen, —CN, —NH₂, —NH(C₁₋₄ alkyl), —N(C₁₋₄ alkyl)₂, C₁₋₄ alkoxy, and C₁₋₄ haloalkoxy.

As used herein, the term “optionally substituted C₃₋₆ cycloalkyl” refers to C₃₋₄ cycloalkyl optionally substituted by one or more (e.g. 1 to 5) substituents each independently selected from the group consisting of —OH, halogen, —CN, —NH₂, —NH(C₁₋₄ alkyl), —N(C₁₋₄ alkyl)₂, C₁₋₄ alkyl, C₁₋₄ haloalkyl, C₁₋₄ hydroxylalkyl, C₁₋₄ alkoxy, and C₁₋₄ haloalkoxy.

As used herein, the term “optionally substituted C₃₋₆ cycloalkyl-C₁₋₂ alkyl-” refers to C₃₋₆ cycloalkyl-C₁₋₂ alkyl- optionally substituted by one or more (e.g. 1 to 5) substituents each independently selected from the group consisting of —OH, halogen, —CN, —NH₂, —NH(C₁₋₄ alkyl), —N(C₁₋₄ alkyl)₂, C₁₋₄ alkyl, C₁₋₄ haloalkyl, C₁₋₄ hydroxylalkyl, C₁-4 alkoxy, and C₁₋₄ haloalkoxy.

As used herein, the term “optionally substituted C₁₋₄ alkoxy” refers to C₁₋₄ alkoxy optionally substituted by one or more (e.g. 1 to 5) substituents each independently selected from the group consisting of —OH, halogen, —CN, —NH₂, —NH(C₁₋₄ alkyl), —N(C₁₋₄ alkyl)₂, C₁₋₄ alkoxy, and C₁₋₄ haloalkoxy.

As used herein, unless specified, the point of attachment of a substituent can be from any suitable position of the substituent. For example, piperidinyl can be piperidin-1-yl (attached through the N atom of the piperidinyl), piperidin-2-yl (attached through the C atom at the 2-position of the piperidinyl), piperidin-3-yl (attached through the C atom at the 3-position of the piperidinyl), or piperidin-4-yl (attached through the C atom at the 4-position of the piperidinyl). For another example, pyridinyl (or pyridyl) can be 2-pyridinyl (or pyridin-2-yl), 3-pyridinyl (or pyridin-3-yl), or 4-pyridinyl (or pyridin-4-yl).

When a bond to a substituent is shown to cross a bond connecting two atoms in a ring, then such substituent may be bonded to any of the ring-forming atoms in that ring that are substitutable (i.e., bonded to one or more hydrogen atoms), unless otherwise specifized or otherwise implicit from the context. For example, as shown in Formula c-4 below, one R⁷ (wherein m is 1) may be bonded to either of the two ring carbon atoms each of which bears a hydrogen atom (but not shown).

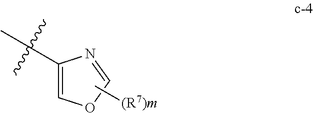

When a substituted or optionally substituted moiety is described without indicating the atom via which such moiety is bonded to a substituent, then the substituent may be bonded via any appropriate atom in such moiety. For example in a substituted arylalkyl, a substituent on the arylalkyl [e.g., (C₆₋₁₀ aryl)-C₁₋₄ alkyl-] can be bonded to any carbon atom on the alkyl part or on the aryl part of the arylalkyl. Combinations of substituents and/or variables are permissible only if such combinations result in stable compounds.

As noted above, the compounds of Formula I (or N-oxides thereof) may exist in the form of pharmaceutically acceptable salts such as acid addition salts and/or base addition salts of the compounds of Formula I. The phrase “pharmaceutically acceptable salt(s)”, as used herein, unless otherwise indicated, includes acid addition or base salts which may be present in the compounds of Formula I (or N-oxides thereof).

Pharmaceutically acceptable salts of the compounds of Formula I (or N-oxides thereof) include the acid addition and base salts thereof.

Suitable acid addition salts are formed from acids which form non-toxic salts. Examples include the acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulfate and hemicalcium salts.

For a review on suitable salts, see “Handbook of Pharmaceutical Salts: Properties, Selection, and Use” by Stahl and Wermuth (Wiley-VCH, 2002). Methods for making pharmaceutically acceptable salts of compounds of Formula I are known to one of skill in the art.

As used herein the terms “Formula I” or “Formula I or an N-oxide thereof or a pharmaceutically acceptable salt of the compound or N-oxide” are defined to include all forms of the compound of Formula I or N-oxide thereof, including hydrates, solvates, isomers (including for example rotational stereoisomers), crystalline and non-crystalline forms, isomorphs, polymorphs, metabolites, and prodrugs thereof.

As it is known to the person skilled in the art, amine compounds (i.e., those comprising one or more nitrogen atoms), for example tertiary amines, can form N-oxides (also known as amine oxides or amine N-oxides). An N-oxide has the formula of (R¹⁰⁰R²⁰⁰R³⁰⁰)N⁺—O⁻ wherein the parent amine (R¹⁰⁰R²⁰⁰R³⁰⁰)N can be for example, a tertiary amine (for example, each of R¹⁰⁰, R²⁰⁰, R³⁰⁰ is independently alkyl, arylalkyl, aryl, heteroaryl, or the like), a heterocyclic or heteroaromatic amine [for example, (R¹⁰⁰R²⁰⁰R³⁰⁰)N together forms 1-alkylpiperidine, 1-alkylpyrrolidine, 1-benzylpyrrolidine, or pyridine]. For instance, an imine nitrogen, especially heterocyclic or heteroaromatic imine nitrogen, or pyridine-type nitrogen

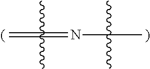 [SMILES: *N=*]

atom [such as a nitrogen atom in pyridine, pyridazine, or pyrazine], can be N-oxidized to form the N-oxide comprising the group

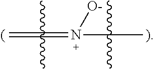

Thus, a compound according to the present invention comprising one or more nitrogen atoms (e.g., an imine nitrogen atom) may be capable of forming an N-oxide thereof (e.g., mono-N-oxides, bis-N-oxides or multi-N-oxides, or mixtures thereof depending on the number of nitrogen atoms suitable to form stable N-oxides).

As used herein, the term “N-oxide(s)” refer to all possible, and in particular all stable, N-oxide forms of the amine compounds (e.g., compounds comprising one or more imine nitrogen atoms) described herein, such as mono-N-oxides (including different isomers when more than one nitrogen atom of an amine compound can form a mono-N-oxide) or multi-N-oxides (e.g., bis-N-oxides), or mixtures thereof in any ratio.

Compounds of Formula I and their salts described herein further include N-oxides thereof.

In the description herein below, unless otherwise specified, compounds of Formula I (or compounds of the invention) include N-oxides thereof and salts of the compounds or the N-oxides.

Compounds of Formula I may exist in a continuum of solid states ranging from fully amorphous to fully crystalline. The term ‘amorphous’ refers to a state in which the material lacks long-range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from apparent solid to a material with liquid properties occurs, which is characterised by a change of state, typically second order (glass transition′). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

Compounds of Formula I may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of Formula I may exist as clathrates or other complexes (e.g., co-crystals). Included within the scope of the invention are complexes such as clathrates, drug-host inclusion complexes wherein the drug and host are present in stoichiometric or non-stoichiometric amounts. Also included are complexes of the compounds of Formula I containing two or more organic and/or inorganic components, which may be in stoichiometric or non-stoichiometric amounts. The resulting complexes may be ionized, partially ionized, or non-ionized. Co-crystals are typically defined as crystalline complexes of neutral molecular constituents that are bound together through non-covalent interactions, but could also be a complex of a neutral molecule with a salt. Co-crystals may be prepared by melt crystallization, by recrystallization from solvents, or by physically grinding the components together; see O. Almarsson and M. J. Zaworotko, Chem. Commun. 2004, 17, 1889-1896. For a general review of multi-component complexes, see J. K. Haleblian, J. Pharm. Sci. 1975, 64, 1269-1288.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

The invention also relates to prodrugs of the compounds of Formula I. Thus certain derivatives of compounds of Formula I which may have little or no pharmacological activity themselves can, when administered into or onto the body, be converted into compounds of Formula I having the desired activity, for example, by hydrolytic cleavage. Such derivatives are referred to as “prodrugs”. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985), or in Prodrugs: Challenges and Reward, 2007 edition, edited by Valentino Stella, Ronald Borchardt, Michael Hageman, Reza Oliyai, Hans Maag, Jefferson Tilley, pages 134-175 (Springer, 2007).

Moreover, certain compounds of Formula I may themselves act as prodrugs of other compounds of Formula I.

Also included within the scope of the invention are metabolites of compounds of Formula I, that is, compounds formed in vivo upon administration of the drug.

The compounds of Formula I include all stereoisomers and tautomers. Stereoisomers of Formula I include cis and trans isomers, optical isomers such as R and S enantiomers, diastereomers, geometric isomers, rotational isomers, atropisomers, and conformational isomers of the compounds of Formula I, including compounds exhibiting more than one type of isomerism; and mixtures thereof (such as racemates and diastereomeric pairs). Also included are acid addition or base addition salts wherein the counterion is optically active, for example, D-lactate or L-lysine, or racemic, for example, DL-tartrate or DL-arginine.

In some embodiments, the compounds of Formula I (including salts thereof) may have asymmetric carbon atoms. The carbon-carbon bonds of the compounds of Formula I may be depicted herein using a solid line (-) a wavy line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that only the stereoisomer shown is meant to be included. The use of a wavy line to depict bonds to asymmetric carbon atoms is meant to indicate that the stereochemistry is unknown (unless otherwise specified). It is possible that compounds of Formula I may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of Formula I can exist as enantiomers and diastereomers or as racemates and mixtures thereof. The use of a solid line to depict bonds to one or more asymmetric carbon atoms in a compound of Formula I and the use of a solid or dotted wedge to depict bonds to other asymmetric carbon atoms in the same compound is meant to indicate that a mixture of diastereomers is present.

In some embodiments, the compounds of Formula I may exist in and/or be isolated as atropisomers (e.g., one or more atropenantiomers). Those skilled in the art would recognize that atropisomerism may exist in a compound that has two or more aromatic rings (for example, two aromatic rings linked through a single bond). See e.g., Freedman, T. B. et al., Absolute Configuration Determination of Chiral Molecules in the Solution State Using Vibrational Circular Dichroism. Chirality 2003, 15, 743-758; and Bringmann, G. et al., Atroposelective Synthesis of Axially Chiral Biaryl Compounds. Angew. Chem., Int. Ed. 2005, 44, 5384-5427.

When any racemate crystallizes, crystals of different types are possible. One type is the racemic compound (true racemate) wherein one homogeneous form of crystal is produced containing both enantiomers in equimolar amounts. Another type is a racemic mixture or conglomerate wherein two forms of crystal are produced in equal or different molar amounts each comprising a single enantiomer.

The compounds of Formula I may exhibit the phenomena of tautomerism and structural isomerism. For example, the compounds of Formula I may exist in several tautomeric forms, including the enol and imine form, the amide and imidic acid form, and the keto and enamine form and geometric isomers and mixtures thereof. All such tautomeric forms are included within the scope of the compounds of Formula I. Tautomers may exist as mixtures of a tautomeric set in solution. In solid form, usually one tautomer predominates. Even though one tautomer may be described, the present invention includes all tautomers of the compounds of Formula I. For example, when one of the following two tautomers is disclosed herein, those skilled in the art would readily recognize the other tautomer.

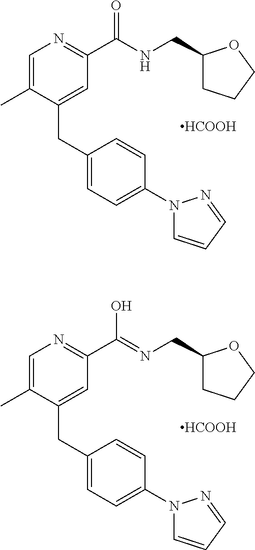 [SMILES: Cc1cnc(C(=O)NC[C@@H]2CCCO2)cc1Cc1ccc(-n2cccn2)cc1 ; "·HCOOH" ; Cc1cnc(/C(O)=N/C[C@@H]2CCCO2)cc1Cc1ccc(-n2cccn2)cc1 ; "·HCOOH"]

The present invention includes all pharmaceutically acceptable isotopically-labelled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula I, for example, those incorporating a radioactive isotope, are useful in drug and/or substrate tissue distribution studies. The radioactive isotopes tritium, i.e., ³H, and carbon-14, i.e., ¹⁴O, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron-emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples and Preparations using an appropriate isotopically-labeled reagent in place of the non-labeled reagent previously employed.

The present invention also provides compositions (e.g., pharmaceutical compositions) comprising a novel compound of Formula I in the second aspect of the invention. Accordingly, in one embodiment, the invention provides a pharmaceutical composition comprising (a therapeutically effective amount of) a novel compound of Formula I and optionally comprising a pharmaceutically acceptable carrier. In one further embodiment, the invention provides a pharmaceutical composition comprising (a therapeutically effective amount of) a compound of Formula I, optionally comprising a pharmaceutically acceptable carrier and, optionally, at least one additional medicinal or pharmaceutical agent (such as an antipsychotic agent or anti-schizophrenia agent described below). In one embodiment, the additional medicinal or pharmaceutical agent is an anti-schizophrenia agent as described below.

The pharmaceutically acceptable carrier may comprise any conventional pharmaceutical carrier or excipient. Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus for oral administration, tablets containing various excipients, such as citric acid, may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream or for rectal administration as a suppository.

Exemplary parenteral administration forms include solutions or suspensions of active compounds in sterile aqueous solutions, for example, aqueous propylene glycol or dextrose solutions. Such dosage forms may be suitably buffered, if desired.

The pharmaceutical composition may be in unit dosage forms suitable for single administration of precise dosages. One of ordinary skill in the art would appreciate that the composition may be formulated in sub-therapeutic dosage such that multiple doses are envisioned.

In one embodiment the composition comprises a therapeutically effective amount of a compound of Formula I and a pharmaceutically acceptable carrier.

Compounds of Formula I are M1 modulators (e.g. M1 allosteric modulators or M1 positive allosteric modulators). In some embodiments, a compound of Formula I is an M1 modulator [binding to (having affinity for) M1 receptors in the presence and/or absence of Ach and activating and/or potentiating M1 receptors in the presence and/or absence of ACh], for example, an M1 positive allosteric modulator (potentiator). In some embodiments, the Inflection Point of a compound of Formula I with respect to M1 receptor (as an M1 positive allosteric modulator) in the presence of an EC₁₀-EC₃₀ concentration of ACh is less than about 10 μM, 5 μM, 2 μM, 1 μM, 500 nM, 200 nM, 100 nM, 50, 40, 30, 20, 10, 5, 2, or 1 nM as determined by the method in Example AA described herein below.

Administration of the compounds of Formula I may be effected by any method that enables delivery of the compounds to the site of action. These methods include, for example, enteral routes (e.g., oral routes, buccal routes, sublabial routes, sublingual routes), oral routes, intranasal routes, inhaled routes, intraduodenal routes, parenteral injection (including intravenous, subcutaneous, intramuscular, intravascular or infusion), intrathecal routes, epidural routes, intracerebral routes, intracerbroventricular routes, topical, and rectal administration.

In one embodiment of the present invention, the compounds of Formula I may be administered/effected by oral routes.

Dosage regimens may be adjusted to provide the optimum desired response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specifications for the dosage unit forms of the invention are dictated by a variety of factors such as the unique characteristics of the therapeutic agent and the particular therapeutic or prophylactic effect to be achieved. In one embodiment of the present invention, the compounds of Formula I may be used to treat humans.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent is well-known in the relevant art and would be understood to be encompassed by the skilled artisan once provided the teachings disclosed herein.

The amount of the compound of Formula I administered will be dependent on the subject being treated, the severity of the disorder or condition, the rate of administration, the disposition of the compound and the discretion of the prescribing physician. Generally, an effective dosage is in the range of about 0.0001 to about 50 mg per kg body weight per day, for example about 0.01 to about 10 mg/kg/day, in single or divided doses. For a 70 kg human, this would amount to about 0.007 mg to about 3500 mg/day, for example about 0.7 mg to about 700 mg/day. In some instances, dosage levels below the lower limit of the aforesaid range may be more than adequate, while in other cases still larger doses may be employed without causing any harmful side effect, provided that such larger doses are first divided into several small doses for administration throughout the day.

As used herein, the term “combination therapy” refers to the administration of a compound of Formula I or a pharmaceutically acceptable salt thereof together with an at least one additional pharmaceutical or medicinal agent (e.g., an anti-schizophrenia agent), either sequentially or simultaneously.

The present invention includes the use of a combination of a compound of Formula I (including an N-oxide thereof or a salt of the compound or the N-oxide) and one or more additional pharmaceutically active agent(s). If a combination of active agents is administered, then they may be administered sequentially or simultaneously, in separate dosage forms or combined in a single dosage form. Accordingly, the present invention also includes pharmaceutical compositions comprising an amount of: (a) a first agent comprising a compound of Formula I (including an N-oxide thereof or a pharmaceutically acceptable salt of the compound or the N-oxide); (b) a second pharmaceutically active agent; and (c) a pharmaceutically acceptable carrier, vehicle or diluent.

Various pharmaceutically active agents may be selected for use in conjunction with the compounds of Formula I, depending on the disease, disorder, or condition to be treated. Pharmaceutically active agents that may be used in combination with the compositions of the present invention include, without limitation:

(i) acetylcholinesterase inhibitors such as donepezil hydrochloride (ARICEPT, MEMAC); or Adenosine A_{2A} receptor antagonists such as Preladenant (SCH 420814) or SCH 412348;

(ii) amyloid-β (or fragments thereof), such as Aβ₁₋₁₅ conjugated to pan HLA DR-binding epitope (PADRE) and ACC-001 (Elan/Wyeth);

(iii) antibodies to amyloid-β (or fragments thereof), such as bapineuzumab (also known as AAB-001) and AAB-002 (Wyeth/Elan);

(iv) amyloid-lowering or -inhibiting agents (including those that reduce amyloid production, accumulation and fibrillization) such as colostrinin and bisnorcymserine (also known as BNC);

(v) alpha-adrenergic receptor agonists such as clonidine (CATAPRES);

(vi) beta-adrenergic receptor blocking agents (beta blockers) such as carteolol;

(vii) anticholinergics such as amitriptyline (ELAVIL, ENDEP);

(viii) anticonvulsants such as carbamazepine (TEGRETOL, CARBATROL);

(ix) antipsychotics, such as lurasidone (also known as SM-13496; Dainippon Sumitomo);

(x) calcium channel blockers such as nilvadipine (ESCOR, NIVADIL);

(xi) catechol O-methyltransferase (COMT) inhibitors such as tolcapone (TASMAR);

(xii) central nervous system stimulants such as caffeine;

(xiii) corticosteroids such as prednisone (STERAPRED, DELTASONE);

(xiv) dopamine receptor agonists such as apomorphine (APOKYN);

(xv) dopamine receptor antagonists such as tetrabenazine (NITOMAN, XENAZINE, dopamine D2 antagonist such as Quetiapine);

(xvi) dopamine reuptake inhibitors such as nomifensine maleate (MERITAL);

(xvii) gamma-aminobutyric acid (GABA) receptor agonists such as baclofen (LIORESAL, KEMSTRO);

(xviii) histamine 3 (H₃) antagonists such as ciproxifan;

(xix) immunomodulators such as glatiramer acetate (also known as copolymer-1; COPAXONE);

(xx) immunosuppressants such as methotrexate (TREXALL, RHEUMATREX);

(xxi) interferons, including interferon beta-1a (AVONEX, REBIF) and interferon beta-1b (BETASERON, BETAFERON);

(xxii) levodopa (or its methyl or ethyl ester), alone or in combination with a DOPA decarboxylase inhibitor (e.g., carbidopa (SINEMET, CARBILEV, PARCOPA));

(xxiii)N-methyl-D-aspartate (NMDA) receptor antagonists such as memantine (NAMENDA, AXURA, EBIXA);

(xxiv) monoamine oxidase (MAO) inhibitors such as selegiline (EMSAM);

(xxv) muscarinic receptor (particularly M1 subtype) agonists such as bethanechol chloride (DUVOID, URECHOLINE);

(xxvi) neuroprotective drugs such as 2,3,4,9-tetrahydro-1H-carbazol-3-one oxime;

(xxvii) nicotinic receptor agonists such as epibatidine;

(xxviii) norepinephrine (noradrenaline) reuptake inhibitors such as atomoxetine (STRATTERA);

(xxix) phosphodiesterase (PDE) inhibitors, for example, PDE9 inhibitors such as BAY 73-6691 (Bayer AG) and PDE 10 (e.g. PDE10A) inhibitors such as papaverine;

(xxx) other PDE inhibitors including (a) PDE1 inhibitors (e.g., vinpocetine), (b) PDE2 inhibitors (e.g., erythro-9-(2-hydroxy-3-nonyl)adenine (EHNA)), (c) PDE4 inhibitors (e.g., rolipram), and (d) PDE5 inhibitors (e.g., sildenafil (VIAGRA, REVATIO));

(xxxi) quinolines such as quinine (including its hydrochloride, dihydrochloride, sulfate, bisulfate and gluconate salts);

(xxxii) β-secretase inhibitors such as WY-25105;

(xxxiii) γ-secretase inhibitors such as LY-411575 (Lilly);

(xxxiv) serotonin (5-hydroxytryptamine) 1A (5-HT_{1A}) receptor antagonists such as spiperone;

(xxxv) serotonin (5-hydroxytryptamine) 4 (5-HT₄) receptor agonists such as PRX-03140 (Epix);

(xxxvi) serotonin (5-hydroxytryptamine) 6 (5-HT₆) receptor antagonists such as mianserin (TORVOL, BOLVIDON, NORVAL);

(xxxvii) serotonin (5-HT) reuptake inhibitors such as alaproclate, citalopram (CELEXA, CIPRAMIL);

(xxxviii) trophic factors, such as nerve growth factor (NGF), basic fibroblast growth factor (bFGF; ERSOFERMIN), neurotrophin-3 (NT-3), cardiotrophin-1, brain-derived neurotrophic factor (BDNF), neublastin, meteorin, and glial-derived neurotrophic factor (GDNF), and agents that stimulate production of trophic factors, such as propentofylline;

and the like.

The compound of Formula I (including an N-oxide thereof and a salt of the compounds or the N-oxide) is optionally used in combination with another active agent. Such an active agent may be, for example, an atypical antipsychotic or an anti-Parkinson's disease agent or an anti-Alzheimer's agent. Accordingly, another embodiment of the invention provides methods of treating an M1-mediated disorder (e.g., a neurological and psychiatric disorder associated with M1), comprising administering to a mammal an effective amount of a compound of Formula I (including an N-oxide thereof or a pharmaceutically acceptable salt of the compound or the N-oxide) and further comprising administering another active agent.

As used herein, the term “another active agent” refers to any therapeutic agent, other than the compound of Formula I (including or a pharmaceutically acceptable salt thereof) that is useful for the treatment of a subject disorder. Examples of additional therapeutic agents include antidepressants, antipsychotics (such as anti-schizophrenia), anti-pain, anti-Parkinson's disease agents, anti-LID (levodopa-induced dyskinesia), anti-Alzheimer's and anti-anxiety agents. Examples of particular classes of antidepressants that can be used in combination with the compounds of the invention include norepinephrine reuptake inhibitors, selective serotonin reuptake inhibitors (SSRIs), NK-1 receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, and atypical antidepressants. Suitable norepinephrine reuptake inhibitors include tertiary amine tricyclics and secondary amine tricyclics. Examples of suitable tertiary amine tricyclics and secondary amine tricyclics include amitriptyline, clomipramine, doxepin, imipramine, trimipramine, dothiepin, butriptyline, iprindole, lofepramine, nortriptyline, protriptyline, amoxapine, desipramine and maprotiline. Examples of suitable selective serotonin reuptake inhibitors include fluoxetine, fluvoxamine, paroxetine, and sertraline. Examples of monoamine oxidase inhibitors include isocarboxazid, phenelzine, and tranylcyclopramine. Examples of suitable reversible inhibitors of monoamine oxidase include moclobemide. Examples of suitable serotonin and noradrenaline reuptake inhibitors of use in the present invention include venlafaxine. Examples of suitable atypical anti-depressants include bupropion, lithium, nefazodone, trazodone and viloxazine. Examples of anti-Alzheimer's agents include Dimebon, NMDA receptor antagonists such as memantine; and cholinesterase inhibitors such as donepezil and galantamine. Examples of suitable classes of anti-anxiety agents that can be used in combination with the compounds of the invention include benzodiazepines and serotonin 1A (5-HT1A) agonists or antagonists, especially 5-HT1A partial agonists, and corticotropin releasing factor (CRF) antagonists. Suitable benzodiazepines include alprazolam, chlordiazepoxide, clonazepam, chlorazepate, diazepam, halazepam, lorazepam, oxazepam, and prazepam. Suitable 5-HT1A receptor agonists or antagonists include buspirone, flesinoxan, gepirone, and ipsapirone. Suitable atypical antipsychotics include paliperidone, bifeprunox, ziprasidone, risperidone, aripiprazole, olanzapine, and quetiapine. Suitable nicotine acetylcholine agonists include ispronicline, varenicline and MEM 3454. Anti-pain agents include pregabalin, gabapentin, clonidine, neostigmine, baclofen, midazolam, ketamine and ziconotide. Examples of suitable anti-Parkinson's disease agents include L-DOPA (or its methyl or ethyl ester), a DOPA decarboxylase inhibitor (e.g., carbidopa (SINEMET, CARBILEV, PARCOPA), an Adenosine A_{2A} receptor antagonist [e.g., Preladenant (SCH 420814) or SCH 412348], benserazide (MADOPAR), α-methyldopa, monofluoromethyldopa, difluoromethyldopa, brocresine, or m-hydroxybenzylhydrazine), a dopamine agonist [such as apomorphine (APOKYN), bromocriptine (PARLODEL), cabergoline (DOSTINEX), dihydrexidine, dihydroergocryptine, fenoldopam (CORLOPAM), lisuride (DOPERGIN), pergolide (PERMAX), piribedil (TRIVASTAL, TRASTAL), pramipexole (MIRAPEX), quinpirole, ropinirole (REQUIP), rotigotine (NEUPRO), SKF-82958 (GlaxoSmithKline), and sarizotan], a monoamine oxidase (MAO) inhibitor [such as selegiline (EMSAM), selegiline hydrochloride (L-deprenyl, ELDEPRYL, ZELAPAR), dimethylselegilene, brofaromine, phenelzine (NARDIL), tranylcypromine (PARNATE), moclobemide (AURORIX, MANERIX), befloxatone, safinamide, isocarboxazid (MARPLAN), nialamide (NIAMID), rasagiline (AZILECT), iproniazide (MARSILID, IPROZID, IPRONID), CHF-3381 (Chiesi Farmaceutici), iproclozide, toloxatone (HUMORYL, PERENUM), bifemelane, desoxypeganine, harmine (also known as telepathine or banasterine), harmaline, linezolid (ZYVOX, ZYVOXID), and pargyline (EUDATIN, SUPIRDYL)], a catechol O-methyltransferase (COMT) inhibitor [such as tolcapone (TASMAR), entacapone (COMTAN), and tropolone], an N-methyl-D-aspartate (NMDA) receptor antagonist [such as amantadine (SYMMETREL)], anticholinergics [such as amitriptyline (ELAVIL, ENDEP), butriptyline, benztropine mesylate (COGENTIN), trihexyphenidyl (ARTANE), diphenhydramine (BENADRYL), orphenadrine (NORFLEX), hyoscyamine, atropine (ATROPEN), scopolamine (TRANSDERM-SCOP), scopolamine methylbromide (PARMINE), dicycloverine (BENTYL, BYCLOMINE, DIBENT, DILOMINE, tolterodine (DETROL), oxybutynin (DITROPAN, LYRINEL XL, OXYTROL), penthienate bromide, propantheline (PRO-BANTHINE), cyclizine, imipramine hydrochloride (TOFRANIL), imipramine maleate (SURMONTIL), lofepramine, desipramine (NORPRAMIN), doxepin (SINEQUAN, ZONALON), trimipramine (SURMONTIL), and glycopyrrolate (ROBINUL)], or a combination thereof. Examples of anti-schizophrenia agents include ziprasidone, risperidone, olanzapine, quetiapine, aripiprazole, asenapine, blonanserin, or iloperidone. Some additional “another active agent” examples include rivastigmine (Exelon), Clozapine, Levodopa, Rotigotine, Aricept, Methylphenidate, memantine. milnacipran, guanfacine, bupropion, and atomoxetine.

As noted above, the compounds of Formula I may be used in combination with one or more additional anti-schizophrenia agents which are described herein. When a combination therapy is used, the one or more additional anti-schizophrenia agents may be administered sequentially or simultaneously with the compound of the invention. In one embodiment, the additional anti-schizophrenia agent is administered to a mammal (e.g., a human) prior to administration of the compound of the invention. In another embodiment, the additional anti-schizophrenia agent is administered to the mammal after administration of the compound of the invention. In another embodiment, the additional anti-schizophrenia agent is administered to the mammal (e.g., a human) simultaneously with the administration of the compound of the invention (or an N-oxide thereof or a pharmaceutically acceptable salt of the foregoing).

The invention also provides a pharmaceutical composition for the treatment of schizophrenia in a mammal, including a human, which comprises an amount of a compound of Formula I (including an N-oxide thereof or a salt of the compound or the N-oxide), as defined above (including hydrates, solvates and polymorphs of said compound or pharmaceutically acceptable salts thereof), in combination with one or more (for example one to three) anti-schizophrenia agents such as ziprasidone, risperidone, olanzapine, quetiapine, aripiprazole, asenapine, blonanserin, or iloperidone, wherein the amounts of the active agent and the combination when taken as a whole are therapeutically effective for treating schizophrenia.

The invention also provides a pharmaceutical composition for treating an M1-mediated (or M1-associated) disease or disorder in a mammal, including a human, which comprises an amount of a compound of Formula I (including an N-oxide thereof or a salt of the compound or the N-oxide), as defined above (including hydrates, solvates and polymorphs of said compound N-oxide or a pharmaceutically acceptable salt of the foregoing), in combination with one or more (for example one to three) other agents for treating the M1-mediated (or M1-associated) disease or disorder, wherein the amount of the active agents and the combination when taken as a whole are therapeutically effective for treating the M1-mediated (or M1-associated) disease or disorder.

It will be understood that the compounds of Formula I depicted above are not limited to a particular stereoisomer (e.g. enantiomer or atropisomer) shown, but also include all stereoisomers and mixtures thereof.

### DETAILED DESCRIPTION OF THE INVENTION

Compounds of the invention, including N-oxides thereof and salts of the compounds or N-oxides, can be prepared using known organic synthesis techniques and can be synthesized according to any of numerous possible synthetic routes.

The reactions for preparing compounds of the invention can be carried out in suitable solvents, which can be readily selected by one of skill in the art of organic synthesis. Suitable solvents can be substantially non-reactive with the starting materials (reactants), the intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin layer chromatography (TLC).

Compounds of Formula I and intermediates thereof may be prepared according to the following reaction schemes and accompanying discussion. Unless otherwise indicated, R¹, R², R³, R⁴, T¹, T², T³, X¹, X², X³, X⁴ and structural Formula I in the reaction schemes and discussion that follow are as defined above. In general, the compounds of this invention may be made by processes which include processes analogous to those known in the chemical arts, particularly in light of the description contained herein. Certain processes for the manufacture of the compounds of this invention and intermediates thereof are provided as further features of the invention and are illustrated by the following reaction schemes. Other processes are described in the experimental section. The schemes and examples provided herein (including the corresponding description) are for illustration only, and not intended to limit the scope of the present invention.

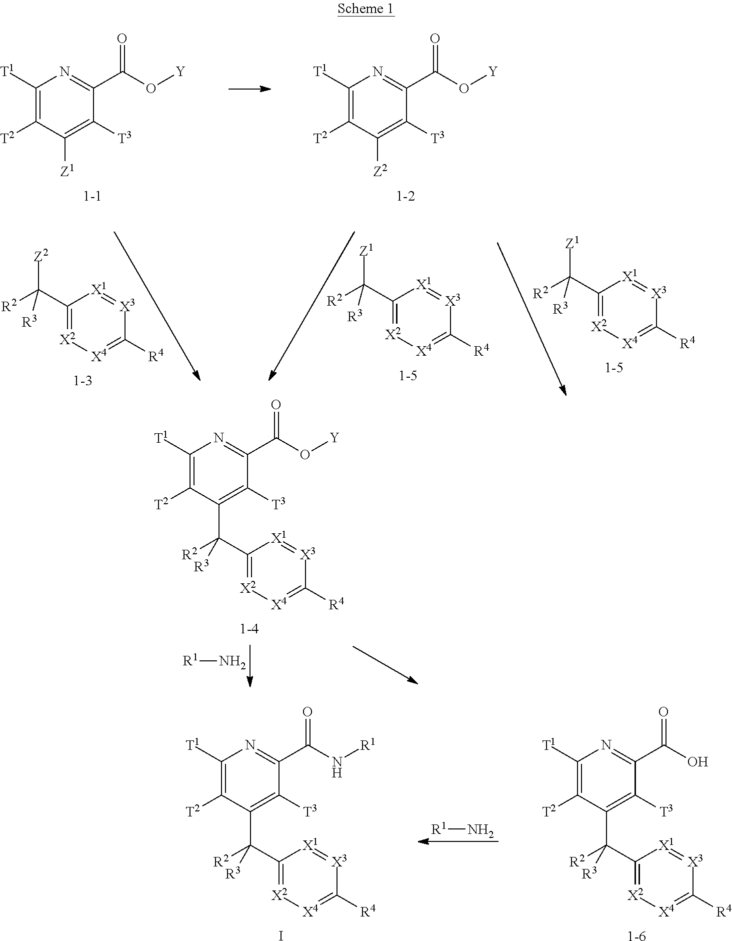

Scheme 1 refers to preparation of compounds of Formula I. Referring to Scheme 1, compounds of Formula 1-1, 1-2, 1-3 and 1-5 [where Z¹ is a halogen (e.g. Cl, Br or I), Z² is a boronic ester (e.g. 4,4,5,5-tetramethyl-1,3,2-dioxaborolan-2-yl) or boronic acid and Y is a simple alkyl (e.g. methyl, ethyl)] are either commercially available or can be obtained by the methods described herein. A compound of Formula 1-4 can be made by coupling a compound of Formula 1-1 and 1-3 under suitable conditions such as a Suzuki reaction [A. Suzuki, J. Organomet. Chem. 1999, 576, 147-168; N. Miyaura and A. Suzuki, Chem. Rev. 1995, 95, 2457-2483; A. F. Littke et al., J. Am. Chem. Soc. 2000, 122, 4020-4028]. The coupling can be accomplished, for example, by heating a mixture of a compound of Formula 1-1 and 1-3 in the presence of a base (such as K₂CO₃), a metal catalyst [such as a palladium catalyst, e.g Pd(dppf)Cl₂], in an appropriate solvent (such as 1,4-dioxane). Alternatively, a compound of Formula 1-1 can be converted to a compound of Formula 1-2 (wherein Z² is defined as above). For example, this reaction can be accomplished by reacting a compound of Formula 1-1 (wherein Z¹ is halogen such as Br) with 4,4,4′,4′,5,5,5′,5′-octamethyl-2,2′-bi-1,3,2-dioxaborolane, a suitable base (such as potassium acetate), and a palladium catalyst {such as [1,1′-bis(diphenylphosphino)ferrocene]dichloropalladium(II)} in a suitable solvent such as toluene. A compound of Formula 1-2 can then be coupled with a compound of Formula 1-5 following similar conditions described above to give a compound of Formula 1-4. The alkyl ester moiety of Compound 1-4 can subsequently be hydrolyzed to a compound of Formula 1-6 in the presence of a suitable base (e.g. NaOH). Alternatively, a compound of Formula 1-6 can be prepared by the directly coupling of a compound of Formula 1-2 and a compound of Formula 1-5 in the presence of an aqueous base (e.g. NaOH) and a metal catalyst [such as a palladium catalyst, e.g. Pd(PPh₃)₄] in an appropriate solvent (e.g. Acetonitrile) at elevated temperature. Subsequently a compound of Formula I can be prepared by coupling of a compound of Formula of 1-6 with an amine (R¹—NH₂) by amidation methods well known to those skilled in the art. For example, the reaction can be accomplished in the presence of a base (e.g. Et₃N) and a peptide coupling agent (e.g. HATU) in an appropriate solvent (e.g. dichloromethane) at an appropriate temperature (e.g. ambient temperature). Alternatively, a compound of Formula I can be prepared directly from an ester of Formula 1-4 by reacting it with an amine (R¹—NH₂) in the presence of a base (e.g. 1,3,4,6,7,8-Hexahydro-2H-pyrimido[1,2-a]pyrimidine) in an appropriate solvent (e.g. N,N-dimethylformamide) at an appropriate temperature (e.g. at an elevated temperature).

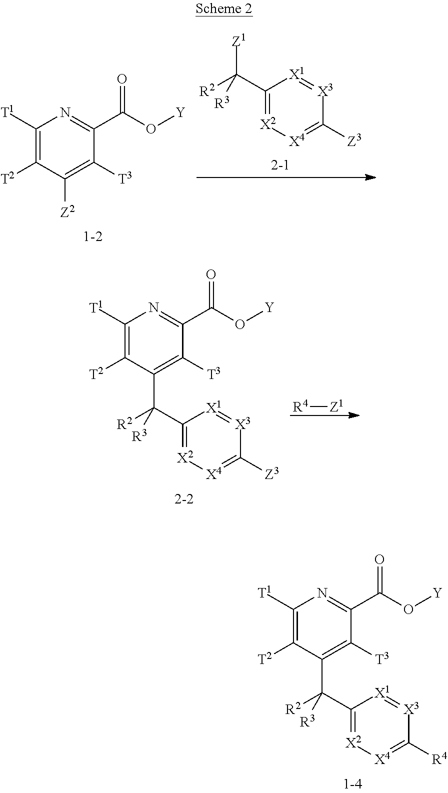

Scheme 2 refers to preparation of intermediates of Formula 1-4. Referring to Scheme 2, a compound of Formula 2-2 can be obtained by coupling of a compound of Formula 1-2 with a compound of Formula 2-1 [wherein Z¹ can be, for example, a halogen (e.g. Cl, Br or I) and Z³ can be, for example, 6-methyl-1,3,6,2-dioxazaborocane-4,8-dione] under suitable conditions such as a Suzuki reaction [A. Suzuki, J. Organomet. Chem. 1999, 576, 147-168; N. Miyaura and A. Suzuki, Chem. Rev. 1995, 95, 2457-2483; A. F. Littke et al., J. Am. Chem. Soc. 2000, 122, 4020-4028]. The coupling can be accomplished, for example, by heating a mixture of a compound of Formula 1-2 and 2-1 in the presence of a base (such as KF), a metal catalyst [such as a palladium catalyst, e.g Pd(PPh₃)₄], in an appropriate solvent (such as Acetonitrile). A compound of Formula 2-2 can then be coupled to a compound of Formula R⁴—Z¹ under Suzuki reaction conditions such as those already described to furnish an intermediate of Formula 1-4, which can be used in Scheme 1 to give compounds of Formula I.

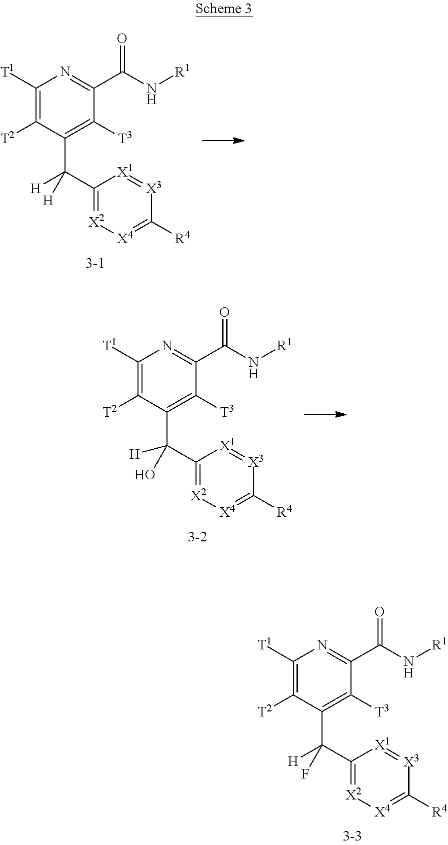

Scheme 3 refers to a 3-step preparation of a compound of Formula 3-3 (which is a specific compound of Formula I wherein one of R² and R³ is H and the other is F) from a compound of Formula 3-1 (which is a specific compound of Formula I wherein both R² and R³ are H). Benzylic bromination of a compound of formula 3-1 by a brominating agent such as N-Bromosuccinimide (NBS) in the presense of a radical initator such as Azobisisobutyronitrile (AIBN) followed by hydrolysis under aqueous conditions will furnish an intermediate benzylic hydroxyl compound of formula 3-2. Conversion of the hydroxyl group of the compound of formula 3-2 into a leaving group followed by treatment with a fluorinating agent (e.g. a HF-amine complex such as HF-pyridine or triethylamine trihydrofluoride) will give a compound of formula 3-3. This conversion can be accomplished, for example, by treating the compound of formula 3-2 with with an activating agent such as 1,1,2,2,3,3,4,4,4-Nonafluorobutane-1-sulfonyl fluoride in the presence of triethylamine trihydrofluoride.

Additional starting materials and intermediates useful for making the compounds of the present invention can be obtained from chemical vendors such as Sigma-Aldrich or can be made according to methods described in the chemical art.

Those skilled in the art can recognize that in all of the Schemes described herein, if there are functional (reactive) groups present on a part of the compound structure such as a substituent group, for example R¹, R², R³, R⁴, T¹, T², T³, X¹, X², X³, X⁴, etc., further modification can be made if appropriate and/or desired, using methods well known to those skilled in the art. For example, a —CN group can be hydrolyzed to afford an amide group; a carboxylic acid can be converted to an amide; a carboxylic acid can be converted to an ester, which in turn can be reduced to an alcohol, which in turn can be further modified. For another example, an OH group can be converted into a better leaving group such as a methanesulfonate, which in turn is suitable for nucleophilic substitution, such as by a cyanide ion (CN⁻). For another example, an —S— can be oxidized to —S(═O)— and/or —S(═O)₂—. For yet another example, an unsaturated bond such as C═C or CEO can be reduced to a saturated bond by hydrogenation. In some embodiments, a primary amine or a secondary amine moiety (present on a substituent group such as R³, R⁴, R⁹, R¹⁰, etc.) can be converted to an amide, sulfonamide, urea, or thiourea moiety by reacting it with an appropriate reagent such as an acid chloride, a sulfonyl chloride, an isocyanate, or a thioisocyanate compound. One skilled in the art will recognize further such modifications. Thus, a compound of Formula I having a substituent that contains a functional group can be converted to another compound of Formula I having a different substituent group.

Similarly, those skilled in the art can also recognize that in all of the schemes described herein, if there are functional (reactive) groups present on a substituent group such as R³, R⁴, R⁹, R¹⁰, etc., these functional groups can be protected/deprotected in the course of the synthetic scheme described here, if appropriate and/or desired. For example, an OH group can be protected by a benzyl, methyl, or acetyl group, which can be deprotected and converted back to the OH group in a later stage of the synthetic process. For another example, an NH₂ group can be protected by a benzyloxycarbonyl (Cbz) or Boc group; conversion back to the NH₂ group can be carried out at a later stage of the synthetic process via deprotection.

As used herein, the term “reacting” (or “reaction” or “reacted”) refers to the bringing together of designated chemical reactants such that a chemical transformation takes place generating a compound different from any initially introduced into the system. Reactions can take place in the presence or absence of solvent.

Compounds of Formula I may exist as stereoisomers, such as atropisomers, racemates, enantiomers, or diastereomers. Conventional techniques for the preparation/isolation of individual enantiomers include chiral synthesis from a suitable optically pure precursor or resolution of the racemate using, for example, chiral high-performance liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of Formula I (and chiral precursors thereof) may be obtained in enantiomerically enriched form using chromatography, typically HPLC, on an asymmetric resin with a mobile phase consisting of a hydrocarbon, typically heptane or hexane, containing from 0% to 50% 2-propanol, typically from 2% to 20%, and from 0% to 5% of an alkylamine, typically 0.1% diethylamine. Concentration of the eluate affords the enriched mixture. Stereoisomeric conglomerates may be separated by conventional techniques known to those skilled in the art. See, e.g., Stereochemistry of Organic Compounds by E. L. Eliel and S. H. Wilen (Wiley, New York, 1994), the disclosure of which is incorporated herein by reference in its entirety. Suitable stereoselective techniques are well known to those of ordinary skill in the art.

Where a compound of Formula I contains an alkenyl or alkenylene (alkylidene) group, geometric cis/trans (or Z/E) isomers are possible. Cis/trans isomers may be separated by conventional techniques well known to those skilled in the art, for example, chromatography and fractional crystallization. Salts of the present invention can be prepared according to methods known to those of skill in the art.

The compounds of Formula I that are basic in nature are capable of forming a wide variety of salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the basic compounds of this invention can be prepared by treating the basic compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, isonicotinic acid, lactic acid, pantothenic acid, bitartric acid, ascorbic acid, 2,5-dihydroxybenzoic acid, gluconic acid, saccharic acid, formic acid, methanesulfonic acid, ethanesulfonic acid, benzenesulfonic acid, p-toluenesulfonic acid, and pamoic [i.e., 4,4′-methanediylbis(3-hydroxynaphthalene-2-carboxylic acid)] acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as ethanesulfonic acid, or the like.

Those compounds of Formula I that are acidic in nature are capable of forming base salts with various pharmacologically acceptable cations. Examples of such salts include the alkali metal or alkaline earth metal salts, and particularly the sodium and potassium salts. These salts are all prepared by conventional techniques. The chemical bases which are used as reagents to prepare the pharmaceutically acceptable base salts of this invention are those which form non-toxic base salts with the acidic compounds of Formula I. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, for example under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are, for example, employed in order to ensure completeness of reaction and maximum yields of the desired final product.

Pharmaceutically acceptable salts of compounds of Formula I (including compounds of Formula Ia or Ib) may be prepared by one or more of three methods:

(i) by reacting the compound of Formula I with the desired acid or base;

(ii) by removing an acid- or base-labile protecting group from a suitable precursor of the compound of Formula I or by ring-opening a suitable cyclic precursor, for example, a lactone or lactam, using the desired acid or base; or

(iii) by converting one salt of the compound of Formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

Polymorphs can be prepared according to techniques well-known to those skilled in the art, for example, by crystallization.

When any racemate crystallizes, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture may have almost identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, New York, 1994).

The invention also includes isotopically labeled compounds of Formula I wherein one or more atoms is replaced by an atom having the same atomic number, but an atomic mass or mass number different from the atomic mass or mass number usually found in nature. Isotopically labeled compounds of Formula I (or pharmaceutically acceptable salts thereof or N-oxides thereof) can generally be prepared by conventional techniques known to those skilled in the art or by processes analogous to those described herein, using an appropriate isotopically labeled reagent in place of the non-labeled reagent otherwise employed.

Prodrugs in accordance with the invention can, for example, be produced by replacing appropriate functionalities present in the compounds of Formula I with certain moieties known to those skilled in the art as ‘pro-moieties’ as described, for example, in Design of Prodrugs by H. Bundgaard (Elsevier, 1985).

The compounds of Formula I should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention (or pharmaceutically acceptable salts thereof) and methods for their preparation will be readily apparent to those skilled in the art. Such compositions and methods for their preparation may be found, for example, in Remington's Pharmaceutical Sciences, 19th Edition (Mack Publishing Company, 1995).

The compounds of the invention (including N-oxides thereof and pharmaceutically acceptable salts of the foregoing) may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropyl methyl cellulose) and typically comprise a carrier, for example, water, ethanol, polyethylene glycol, propylene glycol, methyl cellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described by Liang and Chen, Expert Opinion in Therapeutic Patents 2001, 11, 981-986.

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 weight % to 25 weight %, for example, from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulfate. Lubricants generally comprise from 0.25 weight % to 10 weight %, for example, from 0.5 weight % to 3 weight % of the tablet.

Other possible ingredients include anti-oxidants, colorants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt-congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of Formula I, a film-forming polymer, a binder, a solvent, a humectant, a plasticizer, a stabilizer or emulsifier, a viscosity-modifying agent and a solvent. Some components of the formulation may perform more than one function.

The compound of Formula I (or pharmaceutically acceptable salts thereof or N-oxides thereof) may be water-soluble or insoluble. A water-soluble compound typically comprises from 1 weight % to 80 weight %, more typically from 20 weight % to 50 weight %, of the solutes. Less soluble compounds may comprise a smaller proportion of the composition, typically up to 30 weight % of the solutes. Alternatively, the compound of Formula I may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavorings and flavor enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al., Pharmaceutical Technology On-line, 25(2), 1-14 (2001). The use of chewing gum to achieve controlled release is described in WO 00/35298.

The compounds of the invention (including N-oxides thereof and pharmaceutically acceptable salts of the foregoing) may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (for example to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of Formula I (including N-oxides thereof and pharmaceutically acceptable salts of the foregoing) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(DL-lactic-coglycolic acid) (PLGA) microspheres.

The compounds of the invention (including N-oxides thereof and pharmaceutically acceptable salts of the foregoing) may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated. See e.g., Finnin and Morgan, J. Pharm. Sci. 1999, 88, 955-958.

Other means of topical administration include delivery by electroporation, iontophoresis, phonophoresis, sonophoresis and microneedle or needle-free (e.g., Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention (including N-oxides thereof and pharmaceutically acceptable salts of the foregoing) can also be administered intranasally or by inhalation, typically in the form of a dry powder (either alone; as a mixture, for example, in a dry blend with lactose; or as a mixed component particle, for example, mixed with phospholipids, such as phosphatidylcholine) from a dry powder inhaler, as an aerosol spray from a pressurized container, pump, spray, atomizer (for example an atomizer using electrohydrodynamics to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or hydroxypropyl methyl cellulose), blisters and cartridges for use in an inhaler or insufflator may be formulated to contain a powder mix of the compound of the invention, a suitable powder base such as lactose or starch and a performance modifier such as L-leucine, mannitol, or magnesium stearate. The lactose may be anhydrous or in the form of the monohydrate. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 μL to 100 μL. A typical formulation may comprise a compound of Formula I or a pharmaceutically acceptable salt thereof, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.01 to 100 mg of the compound of Formula I. The overall daily dose will typically be in the range 1 μg to 200 mg, which may be administered in a single dose or, more usually, as divided doses throughout the day.

The compounds of the invention (including N-oxides thereof and pharmaceutically acceptable salts of the foregoing) may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention (including N-oxides thereof and pharmaceutically acceptable salts of the foregoing) may also be administered directly to the eye or ear, typically in the form of drops of a micronized suspension or solution in isotonic, pH-adjusted, sterile saline. Other formulations suitable for ocular and aural administration include ointments, gels, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropyl methyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention (including N-oxides thereof and pharmaceutically acceptable salts of the foregoing) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e., as a carrier, diluent, or solubilizer. Most commonly used for these purposes are alpha-, beta- and gamma-cyclodextrins, examples of which may be found in International Patent Applications Nos. WO 91/11172, WO 94/02518 and WO 98/55148.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of such compound or prodrug and a second compound as described above. The kit comprises means for containing the separate compositions such as a container, a divided bottle or a divided foil packet. Typically the kit comprises directions for the administration of the separate components. The kit form is particularly advantageous when the separate components are for example administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. In some embodiments, the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. For example, the dispenser is equipped with a memory aid, so as to further facilitate compliance with the regimen. An example of such a memory aid is a mechanical counter which indicates the number of daily doses that has been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results. Additional compounds within the scope of this invention may be prepared using the methods illustrated in these Examples, either alone or in combination with techniques generally known in the art. In the following Examples and Preparations, “DMSO” means dimethyl sulfoxide, “N” where referring to concentration means Normal, “M” means molar, “mL” means milliliter, “mmol” means millimoles, “μmol” means micromoles, “eq.” means equivalent, “° C.” means degrees Celsius, “MHz” means megahertz, “HPLC” means high-performance liquid chromatography.

### EXAMPLES

The following illustrate the synthesis of various compounds of the present invention. Additional compounds within the scope of this invention may be prepared using the methods illustrated in these Examples, either alone or in combination with techniques generally known in the art.

Experiments were generally carried out under inert atmosphere (nitrogen or argon), particularly in cases where oxygen- or moisture-sensitive reagents or intermediates were employed. Commercial solvents and reagents were generally used without further purification. Anhydrous solvents were employed where appropriate, generally AcroSeal® products from Acros Organics or DriSolv® products from EMD Chemicals. In other cases, commercial solvents were passed through columns packed with 4 Å molecular sieves, until the following QC standards for water were attained: a) <100 ppm for dichloromethane, toluene, N,N-dimethylformamide and tetrahydrofuran; b) <180 ppm for methanol, ethanol, 1,4-dioxane and diisopropylamine. For very sensitive reactions, solvents were further treated with metallic sodium, calcium hydride or molecular sieves, and distilled just prior to use. Products were generally dried under vacuum before being carried on to further reactions or submitted for biological testing. Mass spectrometry data is reported from either liquid chromatography-mass spectrometry (LCMS), atmospheric pressure chemical ionization (APCI) or gas chromatography-mass spectrometry (GCMS) instrumentation. Chemical shifts for nuclear magnetic resonance (NMR) data are expressed in parts per million (ppm, 6) referenced to residual peaks from the deuterated solvents employed. In some examples, chiral separations were carried out to separate enantiomers of certain compounds of the invention (in some examples, the separated enantiomers are designated as ENT-1 and ENT-2, according to their order of elution). In some examples, the optical rotation of an enantiomer was measured using a polarimeter. According to its observed rotation data (or its specific rotation data), an enantiomer with a clockwise rotation was designated as the (+)-enantiomer and an enantiomer with a counter-clockwise rotation was designated as the (−)-enantiomer. Racemic compounds can optionally be indicated by the presence of (+/−) adjacent to the structure; in these cases, indicated stereochemistry represents the relative (rather than absolute) configuration of the compound's substituents.

Reactions proceeding through detectable intermediates were generally followed by LCMS, and allowed to proceed to full conversion prior to addition of subsequent reagents. For syntheses referencing procedures in other Examples or Methods, reaction conditions (reaction time and temperature) may vary. In general, reactions were followed by thin-layer chromatography or mass spectrometry, and subjected to work-up when appropriate. Purifications may vary between experiments: in general, solvents and the solvent ratios used for eluents/gradients were chosen to provide appropriate R_{f}s or retention times.

### PREPARATIONS

Preparations below describe preparations of P1-P3 that can be used as starting materials/intermediates for preparation of certain examples of compounds of the invention.

### Preparation P1

### (3R,4S)-4-Aminotetrahydro-2H-pyran-3-ol, N-acetyl-D-phenylalanine salt (P1)

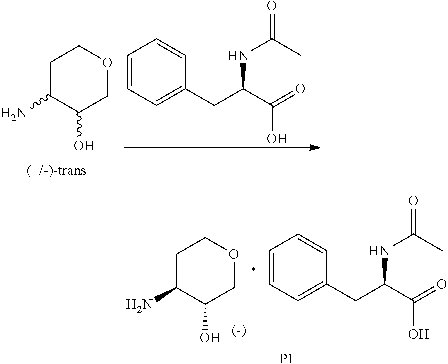

trans-4-Aminotetrahydro-2H-pyran-3-ol (30.0 g, 256 mmol) and N-acetyl-D-phenylalanine (99%, 53.6 g, 256 mmol) were suspended in ethanol (3 L), equally divided between two flasks. The mixtures were heated at reflux until they became homogeneous; at this point the volume in each flask had been reduced to approximately 1.3 L. After the solutions had cooled to room temperature, the precipitates were isolated via filtration and washed with ethanol to provide a white solid (38 g). This material was suspended in ethanol (900 mL) and heated at reflux for 30 minutes, during which time the volume was reduced to approximately 800 mL. The mixture was cooled first to room temperature, and then in an ice bath for 30 minutes, whereupon the solid was collected via filtration to provide the product as a white solid. Yield: 36.0 g, 111 mmol, 43%. ¹H NMR (400 MHz, DMSO-d₆) δ 7.62 (d, J=7.8 Hz, 1H), 7.11-7.26 (m, 5H), 4.15-4.24 (m, 1H), 3.71-3.82 (m, 2H), 3.20-3.35 (m, 2H), 3.04 (dd, J=13.6, 4.8 Hz, 1H), 2.93 (dd, J=10.5, 10.3 Hz, 1H), 2.71-2.86 (m, 2H), 1.79-1.87 (m, 1H), 1.75 (s, 3H), 1.39-1.52 (s, 1H).

Another sample of P1, synthesized in the same manner, was found to have a negative (−) rotation; upon reaction with benzyl carbonochloridate and sodium bicarbonate, the resulting benzyl [(3R,4S)-3-hydroxytetrahydro-2H-pyran-4-yl]carbamate was found to exhibit >99.5% purity upon chiral supercritical fluid chromatography analysis (Column: Phenomenex Lux Amylose-2, 5 μm; Gradient: 5% to 60% methanol in carbon dioxide). The indicated absolute stereochemistry for P1 was assigned in accordance with that determined by single crystal X-ray analysis of Example 9, as P1 was used in the synthesis of 9 in Route 2 of Example 9.

The indicated absolute stereochemistry of P1 was assigned also based on an X-ray crystal structure determination (see below) carried out on a sample of P1 prepared in the same manner described herein above and recrystallized from acetone/water.

### Single Crystal X-Ray Analysis of P1

Data collection was performed on a Bruker APEX diffractometer at −150° C. Data collection consisted of omega and phi scans.

The structure was solved by direct methods using SHELX software suite in the space group P2₁. The structure was subsequently refined by the full-matrix least squares method. All non-hydrogen atoms were found and refined using anisotropic displacement parameters.

During refinement, residual electron density was noted along an infinite channel along the b axis of the structure. These residuals were modeled as half occupied water molecules.

The hydrogen atoms located on nitrogen and oxygen were found from the Fourier difference map and refined freely. The remaining hydrogen atoms were placed in calculated positions and were allowed to ride on their carrier atoms. The final refinement included isotropic displacement parameters for all hydrogen atoms.

The absolute stereochemistry of the 4-aminotetrahydro-2H-pyran-3-ol was determined in relation to the known stereocenter of N-acetyl-D-phenylalanine

The final R-index was 4.9%. A final difference Fourier revealed no missing or misplaced electron density.

Pertinent crystal, data collection and refinement are summarized in Table P1-1. Atomic coordinates, bond lengths, bond angles, and displacement parameters are listed in Tables P1-2 to P1-5.

### Software and References

SHELXTL, Version 5.1, Bruker AXS, 1997.
PLATON, A. L. Spek, J. Appl. Cryst. 2003, 36, 7-13.
MERCURY, C. F. Macrae, P. R. Edington, P. McCabe, E. Pidcock, G. P. Shields, R. Taylor, M. Towler, and J. van de Streek, J. Appl. Cryst. 2006, 39, 453-457.
OLEX2, O. V. Dolomanov, L. J. Bourhis, R. J. Gildea, J. A. K. Howard, and H. Puschmann, J. Appl. Cryst. 2009, 42, 339-341.

TABLE P1-1
Crystal data and structure refinement for P1.

| Empirical formula | C₁₆H₂₄N₂O₅•2H₂O | |
|---|---|---|
| Formula weight | 324.38 • 36.02 | |
| Temperature | 123(2) K | |
| Wavelength | 1.54178 Å | |
| Crystal system | Monoclinic | |
| Space group | P2(1) | |
| Unit cell dimensions | a = 10.4727(3) Å | α = 90°. |
| | b = 5.9576(2) Å | β = 103.2600(10)°. |
| | c = 15.1165(5) Å | γ = 90°. |

| | |
|---|---|
| Volume | 918.01(5) Å³ |
| Z | 2 |
| Density (calculated) | 1.304 Mg/m³ |
| Absorption coefficient | 0.856 mm⁻¹ |
| F(000) | 388 |
| Crystal size | 0.68 × 0.14 × 0.06 mm³ |
| Theta range for data collection | 3.00 to 68.21° |
| Index ranges | −11 <= h <= 12, −6 <= |
| | k <= 6, −17 <= l <= 17 |
| Reflections collected | 9793 |
| Independent reflections | 2865 [R(int) = 0.1080] |
| Completeness to theta = 67.42° | 97.8% |
| Absorption correction | Empirical |
| Max. and min. transmission | 0.9504 and 0.5936 |
| Refinement method | Full-matrix least-squares on F² |
| Data/restraints/parameters | 2865/1/262 |
| Goodness-of-fit on F² | 1.041 |
| Final R indices [I > 2sigma(I)] | R1 = 0.0491, wR2 = 0.1332 |
| R indices (all data) | R1 = 0.0506, wR2 = 0.1350 |
| Absolute structure parameter | −0.1(2) |
| Largest diff. peak and hole | 0.463 and −0.274 e.Å⁻³ |

TABLE P1-2
Atomic coordinates (×10⁴) and equivalent isotropic displacement
parameters (Å² × 10³) for P1. U(eq) is defined as one third
of the trace of the orthogonalized U^{ij} tensor.

| | x | y | z | U(eq) |
|---|---|---|---|---|
| O(1) | 5327(2) | 2848(3) | 9008(1) | 27(1) |
| O(2) | 2426(2) | 124(3) | 7650(1) | 31(1) |
| O(3) | 9133(2) | 11231(3) | 7701(1) | 25(1) |
| O(4) | 10349(2) | 8161(3) | 8081(1) | 26(1) |
| O(5) | 8251(2) | 7593(3) | 9563(1) | 25(1) |
| N(1) | 1260(2) | 3839(4) | 8555(1) | 22(1) |
| N(2) | 8158(2) | 5745(4) | 8253(1) | 20(1) |
| C(1) | 4543(2) | 1485(4) | 8316(2) | 27(1) |
| C(2) | 3117(2) | 1455(4) | 8381(2) | 22(1) |
| C(3) | 2586(2) | 3832(4) | 8345(1) | 21(1) |
| C(4) | 3504(2) | 5329(4) | 9025(2) | 22(1) |
| C(5) | 4903(2) | 5129(4) | 8905(2) | 25(1) |
| C(6) | 5090(2) | 7257(5) | 6527(2) | 28(1) |
| C(7) | 3859(3) | 6289(6) | 6240(2) | 39(1) |
| C(8) | 3728(3) | 4169(6) | 5862(2) | 43(1) |
| C(9) | 4848(3) | 2993(5) | 5790(2) | 39(1) |
| C(10) | 6079(3) | 3944(5) | 6084(2) | 30(1) |
| C(11) | 6217(2) | 6102(4) | 6444(1) | 22(1) |
| C(12) | 7546(2) | 7217(5) | 6703(2) | 25(1) |
| C(13) | 7993(2) | 7793(4) | 7720(1) | 22(1) |
| C(14) | 9264(2) | 9183(4) | 7857(1) | 21(1) |
| C(15) | 8293(2) | 5795(4) | 9158(2) | 21(1) |
| C(16) | 8478(2) | 3579(5) | 9638(2) | 25(1) |
| O(99B) | 8(7) | 2369(18) | 5759(4) | 83(2) |
| O(99A) | 262(9) | 2340(30) | 4880(7) | 174(6) |
| O(99D) | 952(10) | 1010(30) | 5822(5) | 133(4) |
| O(99C) | 379(12) | 4320(30) | 6297(7) | 153(4) |

TABLE P1-3
Bond lengths [Å] and angles [°] for P1.

| | | | |
|---|---|---|---|
| O(1)—C(1) | 1.426(3) | C(1)—O(1)—C(5) | 110.47(18) |
| O(1)—C(5) | 1.427(3) | C(15)—N(2)—C(13) | 121.2(2) |
| O(2)—C(2) | 1.416(3) | O(1)—C(1)—C(2) | 111.64(19) |
| O(3)—C(14) | 1.244(3) | O(2)—C(2)—C(3) | 112.1(2) |
| O(4)—C(14) | 1.265(3) | O(2)—C(2)—C(1) | 106.89(19) |
| O(5)—C(15) | 1.240(3) | C(3)—C(2)—C(1) | 110.15(19) |
| N(1)—C(3) | 1.493(3) | N(1)—C(3)—C(2) | 110.2(2) |
| N(2)—C(15) | 1.343(3) | N(1)—C(3)—C(4) | 109.20(18) |
| N(2)—C(13) | 1.450(3) | C(2)—C(3)—C(4) | 110.62(19) |
| C(1)—C(2) | 1.518(3) | C(5)—C(4)—C(3) | 110.48(19) |
| C(2)—C(3) | 1.518(3) | O(1)—C(5)—C(4) | 110.18(19) |
| C(3)—C(4) | 1.525(3) | C(7)—C(6)—C(11) | 120.7(3) |
| C(4)—C(5) | 1.522(3) | C(8)—C(7)—C(6) | 120.4(3) |
| C(6)—C(7) | 1.388(4) | C(7)—C(8)—C(9) | 119.2(3) |
| C(6)—C(11) | 1.396(3) | C(10)—C(9)—C(8) | 120.5(3) |
| C(7)—C(8) | 1.380(5) | C(9)—C(10)—C(11) | 120.6(3) |
| C(8)—C(9) | 1.393(5) | C(10)—C(11)—C(6) | 118.5(2) |
| C(9)—C(10) | 1.384(4) | C(10)—C(11)—C(12) | 121.0(2) |
| C(10)—C(11) | 1.391(4) | C(6)—C(11)—C(12) | 120.5(2) |
| C(11)—C(12) | 1.511(3) | C(11)—C(12)—C(13) | 114.05(18) |
| C(12)—C(13) | 1.539(3) | N(2)—C(13)—C(12) | 109.7(2) |
| C(13)—C(14) | 1.541(3) | N(2)—C(13)—C(14) | 112.86(18) |
| C(15)—C(16) | 1.497(4) | O(5)—C(15)—N(2) | 121.0(2) |
| C(12)—C(13)—C(14) | 108.07(18) | O(5)—C(15)—C(16) | 122.55(19) |
| O(3)—C(14)—O(4) | 125.2(2) | N(2)—C(15)—C(16) | 116.5(2) |
| O(3)—C(14)—C(13) | 116.6(2) | | |
| O(4)—C(14)—C(13) | 118.2(2) | | |

Symmetry transformations used to generate equivalent atoms.

TABLE P1-4
Anisotropic displacement parameters (Å² × 10³) for P1.
The anisotropic displacement factor exponent takes the
form: −2π²[h² a*²U¹¹ + . . . + 2 h k a* b* U¹²].

| | U¹¹ | U²² | U³³ | U²³ | U¹³ | U¹² |
|---|---|---|---|---|---|---|
| O (1) | 24 (1) | 22 (1) | 33 (1) | 1 (1) | 5 (1) | 2 (1) |
| O (2) | 39 (1) | 27 (1) | 31 (1) | −12 (1) | 14 (1) | −12 (1) |
| O (3) | 28 (1) | 17 (1) | 29 (1) | −1 (1) | 3 (1) | −1 (1) |
| O (4) | 24 (1) | 22 (1) | 33 (1) | 4 (1) | 4 (1) | −1 (1) |
| O (5) | 28 (1) | 24 (1) | 23 (1) | −2 (1) | 6 (1) | 1 (1) |
| N (1) | 22 (1) | 16 (1) | 27 (1) | 0 (1) | 5 (1) | 1 (1) |
| N (2) | 25 (1) | 17 (1) | 20 (1) | −1 (1) | 5 (1) | −2 (1) |
| C (1) | 30 (1) | 19 (1) | 34 (1) | −2 (1) | 12 (1) | 1 (1) |
| C (2) | 29 (1) | 16 (1) | 23 (1) | −3 (1) | 8 (1) | −3 (1) |
| C (3) | 24 (1) | 18 (1) | 20 (1) | 2 (1) | 6 (1) | −1 (1) |
| C (4) | 26 (1) | 14 (1) | 27 (1) | −3 (1) | 6 (1) | 0 (1) |
| C (5) | 25 (1) | 21 (1) | 29 (1) | 0 (1) | 5 (1) | −3 (1) |
| C (6) | 35 (1) | 28 (2) | 20 (1) | 0 (1) | 5 (1) | 3 (1) |
| C (7) | 28 (1) | 61 (2) | 27 (1) | 7 (1) | 4 (1) | −1 (1) |
| C (8) | 40 (1) | 56 (2) | 28 (1) | 5 (1) | −2 (1) | −21 (1) |
| C (9) | 64 (2) | 26 (2) | 21 (1) | 0 (1) | −2 (1) | −19 (1) |
| C (10) | 44 (1) | 24 (1) | 18 (1) | 3 (1) | 1 (1) | 2 (1) |
| C (11) | 30 (1) | 21 (1) | 15 (1) | 4 (1) | 2 (1) | 0 (1) |
| C (12) | 30 (1) | 24 (1) | 19 (1) | 1 (1) | 4 (1) | −1 (1) |
| C (13) | 24 (1) | 17 (1) | 22 (1) | 0 (1) | 3 (1) | −1 (1) |
| C (14) | 24 (1) | 20 (1) | 18 (1) | −2 (1) | 4 (1) | −1 (1) |
| C (15) | 15 (1) | 22 (1) | 25 (1) | 0 (1) | 4 (1) | 0 (1) |
| C (16) | 23 (1) | 25 (2) | 26 (1) | 4 (1) | 8 (1) | 0 (1) |
| O (99B) | 70 (4) | 140 (7) | 37 (3) | 12 (4) | 7 (2) | 9 (4) |
| O (99A) | 101 (6) | 334 (18) | 99 (6) | 98 (9) | 51 (5) | 90 (9) |
| O (99D) | 104 (6) | 230 (13) | 61 (4) | −1 (6) | 9 (4) | −19 (8) |
| O (99C) | 143 (9) | 203 (13) | 110 (7) | 26 (9) | 23 (6) | 42 (9) |

TABLE P1-5
Hydrogen coordinates (×10⁴) and isotropic displacement
parameters (Å² × 10³) for P1.

| | x | y | z | U(eq) |
|---|---|---|---|---|
| H(2) | 1796 | −519 | 7803 | 47 |
| H(1X) | 1310(30) | 3470(50) | 9180(20) | 31(8) |
| H(1Y) | 690(30) | 2960(70) | 8210(20) | 41(9) |
| H(2X) | 8180(20) | 4490(50) | 7962(17) | 12(6) |
| H(1A) | 4890 | −67 | 8372 | 32 |
| H(1B) | 4599 | 2068 | 7713 | 32 |
| H(2A) | 3054 | 743 | 8968 | 26 |
| H(3) | 2510 | 4447 | 7719 | 25 |
| H(4A) | 3211 | 6909 | 8934 | 27 |
| H(4B) | 3475 | 4884 | 9651 | 27 |
| H(5A) | 4946 | 5675 | 8294 | 30 |
| H(5B) | 5493 | 6073 | 9362 | 30 |
| H(6) | 5167 | 8722 | 6782 | 34 |
| H(7) | 3101 | 7091 | 6304 | 47 |
| H(8) | 2884 | 3520 | 5654 | 51 |
| H(9) | 4768 | 1526 | 5536 | 46 |
| H(10) | 6836 | 3114 | 6040 | 36 |
| H(12A) | 7522 | 8615 | 6346 | 29 |
| H(12B) | 8204 | 6209 | 6535 | 29 |
| H(13) | 7301 | 8731 | 7898 | 26 |
| H(16A) | 9180 | 3705 | 10189 | 37 |
| H(16B) | 8714 | 2440 | 9236 | 37 |
| H(16C) | 7660 | 3143 | 9802 | 37 |
| H(1Z) | 920(30) | 5200(60) | 8501(19) | 24(7) |

### Preparation P2

### (3R,4S)-4-Aminotetrahydro-2H-pyran-3-ol (P2)

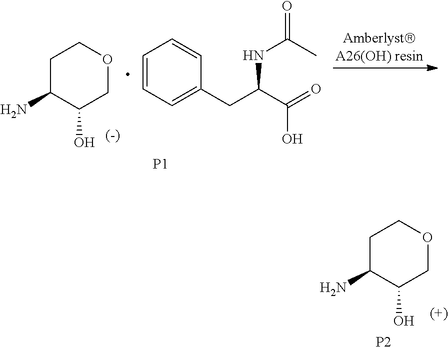

Compound P1 (3.00 g, 9.25 mmol) was suspended in a mixture of dichloromethane and methanol (1:1, 80 mL), and treated with Amberlyst® A26(OH) resin (15 g). The resulting mixture was stirred at room temperature overnight, whereupon it was filtered, and the collected resin was thoroughly washed with dichloromethane. The filtrate was concentrated in vacuo, affording the product as a light yellow solid. The product exhibited a positive (+) rotation. Yield: 1.00 g, 8.54 mmol, 92%. ¹H NMR (400 MHz, CD₃OD) δ 3.82-3.90 (m, 2H), 3.38 (ddd, J=12.0, 11.9, 2.2 Hz, 1H), 3.23 (ddd, J=10.0, 9.1, 4.8 Hz, 1H), 3.03 (dd, J=11.0, 10.1 Hz, 1H), 2.61 (ddd, J=11.4, 9.0, 4.6 Hz, 1H), 1.80-1.87 (m, 1H), 1.46 (dddd, J=13.4, 12.2, 11.5, 4.7 Hz, 1H).

### Preparation P3

### (1S,2S)-2-Methoxycyclohexanamine, hydrochloride salt (P3)

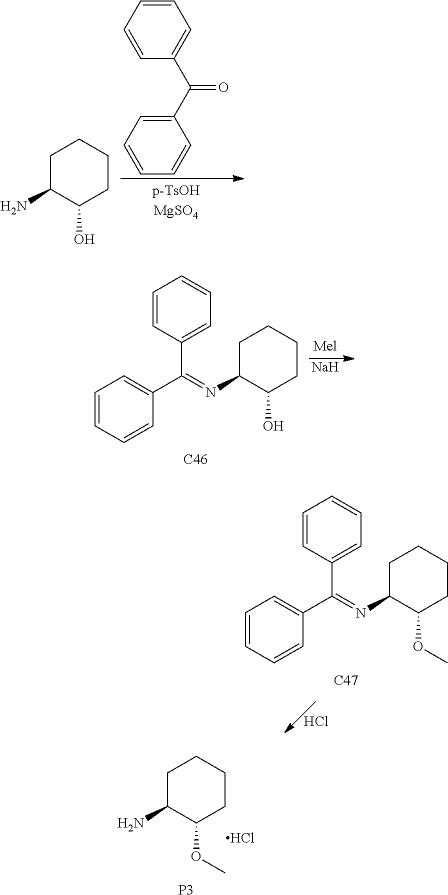

### Step 1. Synthesis of (1S,2S)-2-[(diphenylmethylidene)amino]cyclohexanol (C46)

A mixture of (1S,2S)-2-aminocyclohexanol (500 mg, 4.34 mmol), p-toluenesulfonic acid monohydrate (82.6 mg, 0.434 mmol), magnesium sulfate (1.4 g, 12 mmol), and benzophenone (775 mg, 4.25 mmol) in toluene (20 mL) was stirred at 110° C. for 42 hours. The reaction mixture was concentrated in vacuo; silica gel chromatography (Gradient: 0% to 90% ethyl acetate in petroleum ether) afforded the product as a colorless oil. Yield: 344 mg, 1.23 mmol, 29%.

### Step 2. Synthesis of N-[(1S,2S)-2-methoxycyclohexyl]-1,1-diphenylmethanimine (C47)

Iodomethane (175 mg, 1.23 mmol) was added to a 0° C. solution of C46 (344 mg, 1.23 mmol) and sodium hydride (60% in oil, 59.1 mg, 1.48 mmol) in tetrahydrofuran (20 mL), and the reaction mixture was stirred at room temperature overnight. The reaction was quenched by addition of water (10 mL), and the resulting mixture was concentrated in vacuo. Silica gel chromatography (Gradient: 0% to 7% ethyl acetate in petroleum ether) provided the product as a colorless oil. Yield: 184 mg, 0.627 mmol, 51%. ¹H NMR (400 MHz, CDCl₃) δ 7.61 (br d, J=7 Hz, 2H), 7.29-7.48 (m, 6H), 7.19-7.25 (m, 2H), 3.38 (s, 3H), 3.33-3.42 (m, 1H), 3.23-3.32 (m, 1H), 2.03-2.12 (m, 1H), 1.52-1.76 (m, 4H), 1.25-1.39 (m, 1H), 1.03-1.17 (m, 2H).

### Step 3. Synthesis of (1S,2S)-2-methoxycyclohexanamine, hydrochloride salt (P3)

A mixture of C47 (200 mg, 0.682 mmol), 1 M hydrochloric acid (20 mL), and tetrahydrofuran (20 mL) was stirred at room temperature overnight. Solvent was removed in vacuo, the residue was partitioned between ethyl acetate (15 mL) and water (15 mL), and the aqueous layer was washed with ethyl acetate (2×20 mL). The aqueous layer was then concentrated under reduced pressure to afford the product as a white solid. Yield: 130 mg, quantitative. ¹H NMR (400 MHz, CD₃OD) δ 3.40 (s, 3H), 3.12 (ddd, J=10.4, 10.3, 4.4 Hz, 1H), 2.87-2.98 (m, 1H), 2.27-2.36 (m, 1H), 2.01-2.09 (m, 1H), 1.75-1.87 (m, 2H), 1.24-1.48 (m, 3H), 1.07-1.19 (m, 1H).

### Example 1

### 5-Chloro-N-[(3R,4S)-3-hydroxytetrahydro-2H-pyran-4-yl]-4-[4-(1H-pyrazol-1-yl)benzyl]pyridine-2-carboxamide (1)

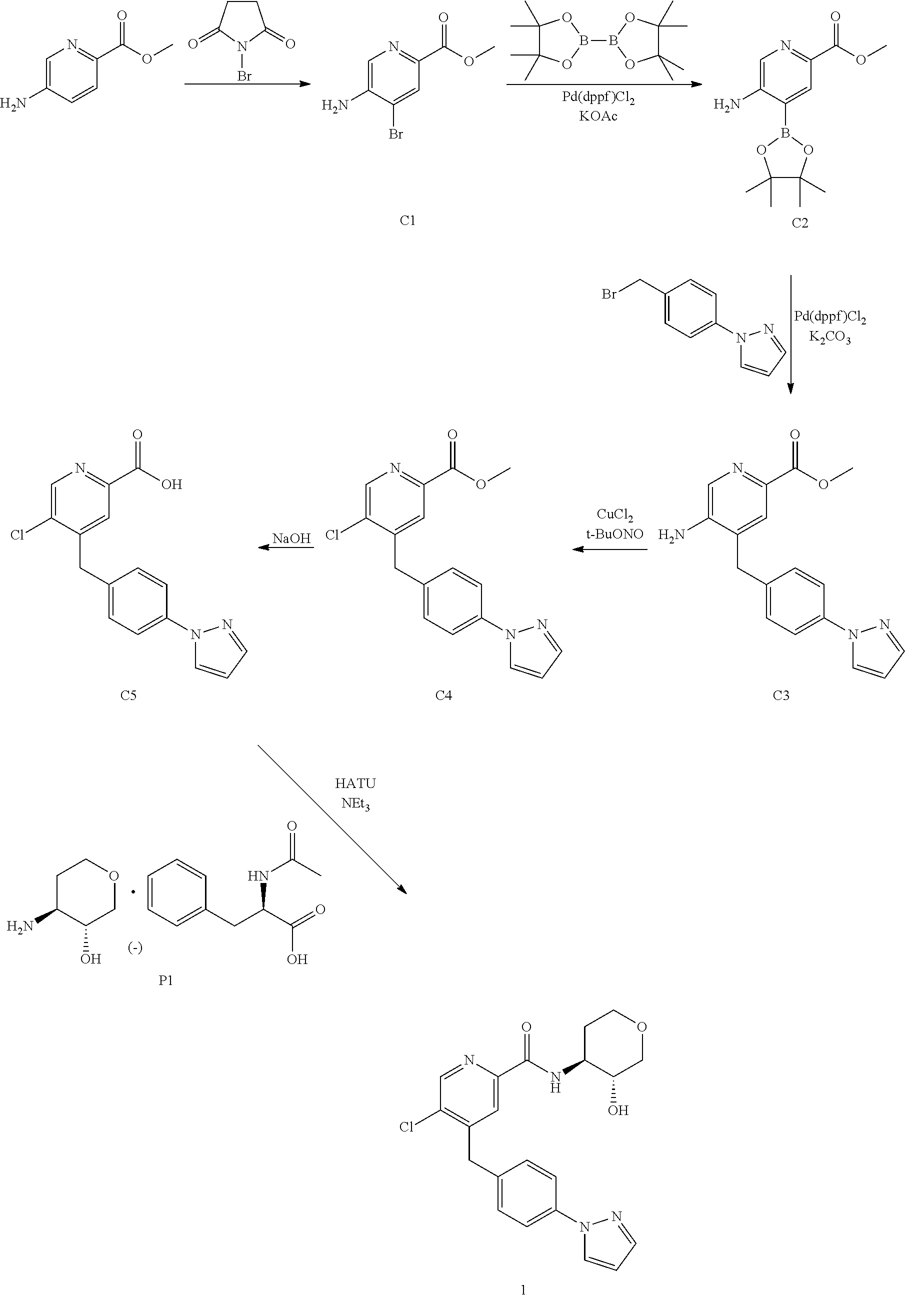

### Step 1. Synthesis of methyl 5-amino-4-bromopyridine-2-carboxylate (Cl)

N-Bromosuccinimide (468 mg, 2.63 mmol) was added portion-wise to a 50° C. solution of methyl 5-aminopyridine-2-carboxylate (400 mg, 2.6 mmol) in acetonitrile (15 mL), and the reaction mixture was heated at 50° C. overnight. Crude reaction mixtures from six additional small-scale reactions of this transformation were added (total starting material quantity: 760 mg, 5.0 mmol), and the resulting mixture was concentrated in vacuo, then purified via silica gel chromatography (Gradient: 2% to 66% ethyl acetate in petroleum ether), providing the product as a red solid. Yield: 150 mg, 0.65 mmol, 13%. ¹H NMR (400 MHz, CDCl₃) δ 8.23 (s, 1H), 8.16 (s, 1H), 4.61 (br s, 2H), 3.97 (s, 3H).

### Step 2. Synthesis of methyl 5-amino-4-(4,4,5,5-tetramethyl-1,3,2-dioxaborolan-2-yl)pyridine-2-carboxylate (C2)

A mixture of C1 (135 mg, 0.584 mmol), 4,4,4′,4′,5,5,5′,5′-octamethyl-2,2′-bi-1,3,2-dioxaborolane (223 mg, 0.878 mmol), [1,1′-bis(diphenylphosphino)ferrocene]dichloropalladium(II) (64.1 mg, 87.6 μmol), and potassium acetate (206 mg, 2.10 mmol) in toluene (10 mL) was stirred at 100° C. for 20 hours. The reaction mixture was allowed to cool, and used in the next step without purification.

### Step 3. Synthesis of methyl 5-amino-4-[4-(1H-pyrazol-1-yl)benzyl]pyridine-2-carboxylate (C3)

To the crude toluene solution of C2 from the previous step were added 1-[4-(bromomethyl)phenyl]-1H-pyrazole (155 mg, 0.654 mmol), [1,1′-bis(diphenylphosphino)ferrocene]dichloropalladium(II) (44.9 mg, 61.4 μmol), potassium carbonate (113 mg, 0.818 mmol), 1,4-dioxane (10 mL), and water (0.5 mL). The reaction mixture was stirred at 80° C. for 18 hours, whereupon it was filtered through a pad of diatomaceous earth. The filtrate was concentrated in vacuo; silica gel chromatography (Gradient: 20% to 100% ethyl acetate in petroleum ether) afforded the product as a yellow solid. Yield: 120 mg, 0.39 mmol, 67% over two steps. ¹H NMR (400 MHz, CDCl₃) δ 8.12 (s, 1H), 7.89-7.93 (m, 2H), 7.73 (d, J=1.4 Hz, 1H), 7.66 (br d, J=8.5 Hz, 2H), 7.24-7.28 (m, 2H, assumed; partially obscured by solvent peak), 6.48 (dd, J=2.4, 1.9 Hz, 1H), 4.02 (br s, 2H), 3.95-3.98 (m, 5H).

### Step 4. Synthesis of methyl 5-chloro-4-[4-(1H-pyrazol-1-yl)benzyl]pyridine-2-carboxylate (C4)

To a solution of C3 (160 mg, 0.519 mmol) and copper(II) chloride dihydrate (133 mg, 0.780 mmol) in acetonitrile (5 mL) was added tert-butyl nitrite (107 mg, 1.04 mmol). After the reaction mixture had been stirred at room temperature for 15 minutes, it was heated at 50° C. for 4 hours. The reaction mixture was filtered, and the filtrate was concentrated in vacuo; the residue was partitioned between aqueous ammonium hydroxide (50 mL) and ethyl acetate (50 mL), and the aqueous layer was extracted with ethyl acetate (2×50 mL). The combined organic layers were dried over sodium sulfate, filtered, and concentrated under reduced pressure. Purification via preparative thin layer chromatography on silica gel (Eluent: 1:1 petroleum ether/ethyl acetate) provided the product as a yellow solid. Yield: 50 mg, 0.15 mmol, 29%. LCMS m/z 327.8 [M+H]⁺. ¹H NMR (400 MHz, CDCl₃) δ 8.69 (s, 1H), 7.89-7.98 (m, 2H), 7.73 (s, 1H), 7.67 (br d, J=8.5 Hz, 2H), 7.29 (br d, J=8.5 Hz, 2H), 6.46-6.50 (m, 1H), 4.18 (s, 2H), 3.99 (s, 3H).

### Step 5. Synthesis of 5-chloro-4-[4-(1H-pyrazol-1-yl)benzyl]pyridine-2-carboxylic acid (C5)

A solution of C4 (25 mg, 76 μmol) in 1,4-dioxane (2 mL) was added to a solution of sodium hydroxide (6.1 mg, 0.15 mmol) in water (2 mL), and the reaction mixture was stirred at room temperature for 30 minutes. It was then adjusted to a pH of 4-5 via addition of a mixture of concentrated hydrochloric acid (2 mL) and water (2 mL). Removal of solvent under reduced pressure afforded the product as a yellow gum, which was employed in the next step without additional purification. LCMS m/z 313.8 [M+H]⁺.

### Step 6. Synthesis of 5-chloro-N-[(3R,4S)-3-hydroxytetrahydro-2H-pyran-4-yl]-4-[4-(1H-pyrazol-1-yl)benzyl]pyridine-2-carboxamide (1)

A solution of C5 (from the previous step, 20 mg, 64 μmol), (3R,4S)-4-aminotetrahydro-2H-pyran-3-ol, N-acetyl-D-phenylalanine salt (P1) (41.4 mg, 0.128 mmol), triethylamine (32.3 mg, 0.319 mmol), and O-(7-azabenzotriazol-1-yl)-N,N,N′,N′-tetramethyluronium hexafluorophosphate (HATU, 72.7 mg, 0.191 mmol) in acetonitrile (3 mL) was stirred at 25° C. for 4 hours. The reaction mixture was concentrated in vacuo, and the residue was purified by reversed phase HPLC (Column: Phenomenex Gemini C18, 8 μm; Mobile phase A: aqueous ammonia, pH 10; Mobile phase B: acetonitrile; Gradient: 36% to 56% B) to afford the product as a white solid. Yield: 4.0 mg, 9.7 μmol, 13% over two steps. LCMS m/z 434.9 [M+N⁺]. ¹H NMR (400 MHz, CDCl₃) δ 8.50 (s, 1H), 8.05 (s, 1H), 7.97-8.03 (m, 1H), 7.91 (d, J=2.4 Hz, 1H), 7.72 (br d, J=1 Hz, 1H), 7.65 (br d, J=8.5 Hz, 2H), 7.29 (br d, J=8.5 Hz, 2H), 6.45-6.49 (m 1H), 4.19 (s, 2H), 3.89-4.12 (m, 4H), 3.58-3.67 (m, 1H), 3.42-3.52 (m, 1H), 3.22 (dd, J=11.3, 10.0 Hz, 1H), 2.00-2.07 (m, 1H), 1.72-1.85 (m, 1H).

### Example 2

### 4-[2-Fluoro-4-(1-methyl-1H-pyrazol-4-yl)benzyl]-N-[(3R,4S)-3-hydroxytetrahydro-2H-pyran-4-yl]-5-methylpyridine-2-carboxamide (2)

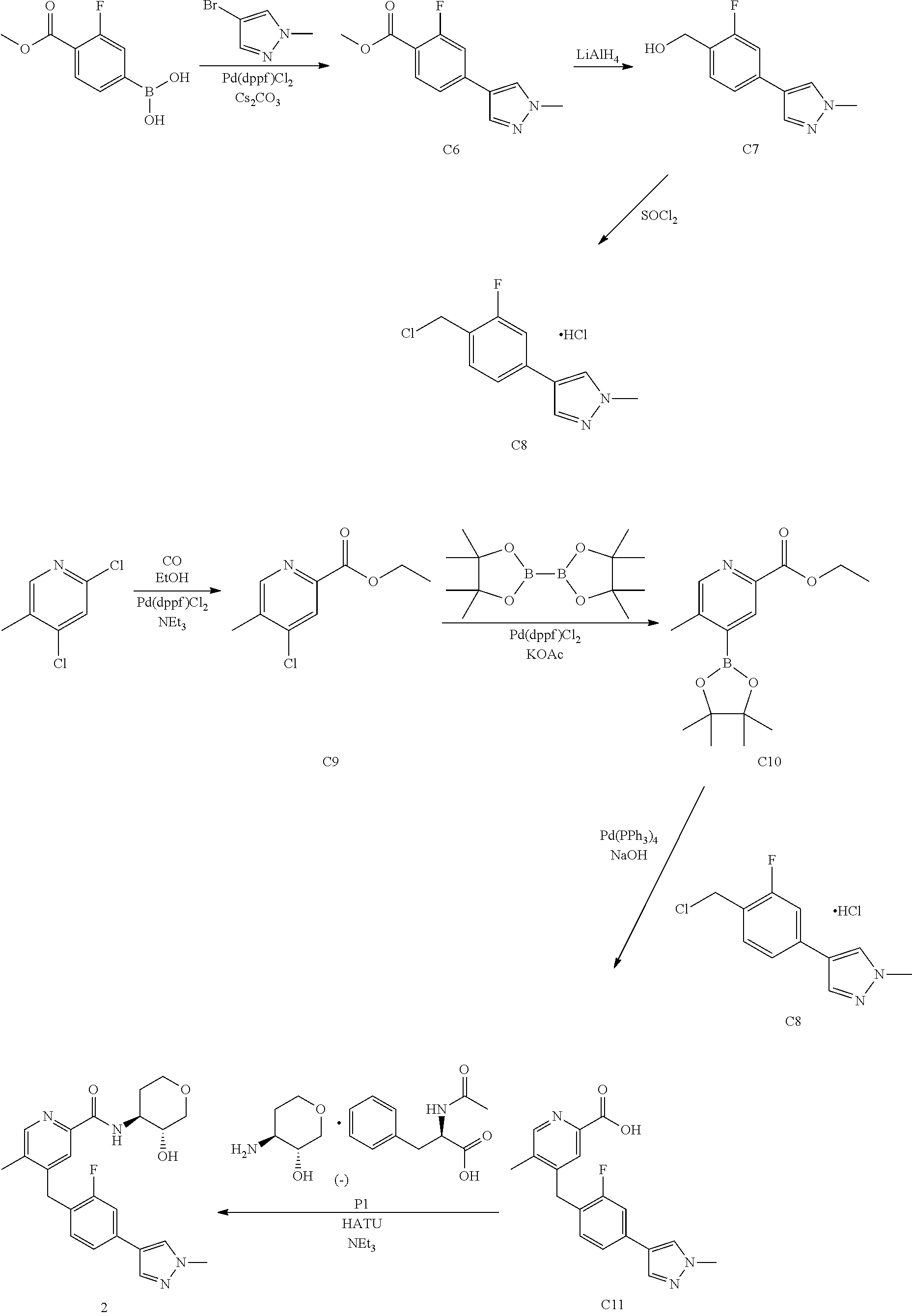

### Step 1. Synthesis of methyl 2-fluoro-4-(1-methyl-1H-pyrazol-4-yl)benzoate (C6)

4-Bromo-1-methyl-1H-pyrazole (11.5 g, 71.4 mmol), [1,1′-bis(diphenylphosphino)ferrocene]dichloropalladium(II) (1.96 g, 2.68 mmol), and cesium carbonate (31.3 g, 96.1 mmol) were added to a solution of [3-fluoro-4-(methoxycarbonyl)phenyl]boronic acid (9.5 g, 48 mmol) in 1,4-dioxane (200 mL) and water (20 mL). The reaction mixture was stirred for 3 hours at reflux, whereupon it was filtered. The filtrate was concentrated in vacuo; silica gel chromatography (Gradient: 0% to 45% ethyl acetate in petroleum ether) afforded the product as an off-white solid. Yield: 6.7 g, 29 mmol, 60%.

¹H NMR (400 MHz, CDCl₃) δ 7.93 (dd, J=8.0, 7.9 Hz, 1H), 7.80 (s, 1H), 7.69 (s, 1H), 7.29 (dd, J=8.2, 1.6 Hz, 1H), 7.21 (dd, J=12.1, 1.6 Hz, 1H), 3.97 (s, 3H), 3.93 (s, 3H).

### Step 2. Synthesis of [2-fluoro-4-(1-methyl-1H-pyrazol-4-yl)phenyl]methanol (C7)

Lithium aluminum hydride (2.72 g, 71.7 mmol) was added portion-wise to a −78° C. solution of C6 (6.7 g, 29 mmol) in tetrahydrofuran (400 mL). The reaction mixture was allowed to stir for 1 hour at −78° C., then for 3 hours in an ice-ethanol cooling bath. While still under ice-ethanol cooling, the reaction was quenched via drop-wise addition of water (3 mL) and aqueous sodium hydroxide solution (15%, 3 mL). The resulting mixture was filtered, the filtrate was concentrated in vacuo, and the residue was purified by silica gel chromatography (Gradient: 0% to 100% ethyl acetate in petroleum ether) to provide the product as a white solid. Yield: 4.0 g, 19 mmol, 66%. ¹H NMR (400 MHz, CD₃OD) δ 7.97 (s, 1H), 7.82 (s, 1H), 7.42 (dd, J=7.9, 7.8 Hz, 1H), 7.35 (dd, J=7.8, 1.2 Hz, 1H), 7.26 (dd, J=11.5, 1.2 Hz, 1H), 4.64 (s, 2H), 3.91 (s, 3H).

### Step 3. Synthesis of 4-[4-(chloromethyl)-3-fluorophenyl]-1-methyl-1H-pyrazole, hydrochloride salt (C8)

A solution of thionyl chloride (1.58 g, 13.3 mmol) in toluene (25 mL) was added drop-wise to a water bath-cooled solution of C7 (2.5 g, 12 mmol) in chloroform (53 mL) and toluene (50 mL). The reaction mixture, still in the water bath, was stirred overnight, then concentrated in vacuo, affording the product as a white solid. Yield: 2.9 g, 11 mmol, 92%. ¹H NMR (400 MHz, CD₃OD) δ 8.40 (br s, 1H), 8.34 (br s, 1H), 7.40-7.53 (m, 3H), 4.69 (s, 2H), 4.08 (s, 3H).

### Step 4. Synthesis of ethyl 4-chloro-5-methylpyridine-2-carboxylate (C9)

A mixture of 2,4-dichloro-5-methylpyridine (33 g, 0.20 mol), [1,1′-bis(diphenylphosphino)ferrocene]dichloropalladium(II) (7.45 g, 10.2 mmol), and triethylamine (61.8 g, 611 mmol) in ethanol (500 mL) was stirred under carbon monoxide (30 psi) at 60° C. for 4 hours. The reaction mixture was filtered, the filtrate was concentrated under reduced pressure, and the residue was purified by silica gel chromatography (Gradient: 10% to 50% ethyl acetate in petroleum ether), providing the product as a yellow oil. Yield: 25.0 g, 0.125 mol, 62%. ¹H NMR (400 MHz, CDCl₃) δ 8.56 (s, 1H), 8.11 (s, 1H), 4.48 (q, J=7.1 Hz, 2H), 2.44 (s, 3H), 1.44 (t, J=7.1 Hz, 3H).

### Step 5. Synthesis of ethyl 5-methyl-4-(4,4,5,5-tetramethyl-1,3,2-dioxaborolan-2-yl)pyridine-2-carboxylate (C10)

A mixture of C9 (16 g, 80 mmol), 4,4,4′,4′,5,5,5′,5′-octamethyl-2,2′-bi-1,3,2-dioxaborolane (30.5 g, 120 mmol), [1,1′-bis(diphenylphosphino)ferrocene]dichloropalladium(II) (5.86 g, 8.01 mmol), and potassium acetate (28.3 g, 288 mmol) in toluene (1.2 L) was stirred at 130° C. for 20 hours. After filtration of the reaction mixture, the filtrate was concentrated under reduced pressure. The residue was purified by chromatography on silica gel (Gradient: 10% to 50% ethyl acetate in petroleum ether) to provide a yellow solid (20 g), which was diluted with petroleum ether (50 mL) and stirred at room temperature for 20 minutes. The solid was collected via filtration to afford the product (8.8 g) as a white solid. The corresponding filtrate was concentrated in vacuo and the residue was purified by silica gel chromatography (Gradient: 0% to 30% ethyl acetate in petroleum ether); the isolated material (4.5 g) was washed with petroleum ether (5 mL) to yield additional product (3.5 g) as a white solid. Combined yield: 12.3 g, 42.2 mmol, 53%. ¹H NMR (400 MHz, CDCl₃) δ 8.57 (s, 1H), 8.39 (s, 1H), 4.48 (q, J=7.2 Hz, 2H), 2.57 (s, 3H), 1.45 (t, J=7.2 Hz, 3H), 1.37 (s, 12H).

### Step 6. Synthesis of 4-[2-fluoro-4-(1-methyl-1H-pyrazol-4-yl)benzyl]-5-methylpyridine-2-carboxylic acid (C11)

To a mixture of C10 (50 mg, 0.17 mmol), C8 (49.3 mg, 0.189 mmol), and sodium hydroxide (34.3 mg, 0.858 mmol) in a mixture of acetonitrile (5 mL) and water (0.2 mL) was added tetrakis(triphenylphosphine)palladium(0) (19.8 mg, 17.1 μmol), and the reaction mixture was stirred at 80° C. for 4 hours. It was then concentrated in vacuo and diluted with water (10 mL). The resulting mixture was acidified to pH 1 with hydrochloric acid and filtered; the filtrate was concentrated under reduced pressure to afford the product as a yellow solid (40 mg), which was used in the following step without additional purification.

### Step 7. Synthesis of 4-[2-fluoro-4-(1-methyl-1H-pyrazol-4-yl)benzyl]-N-[(3R,4S)-3-hydroxytetrahydro-2H-pyran-4-yl]-5-methylpyridine-2-carboxamide (2)

O-(7-Azabenzotriazol-1-yl)-N,N,N′,N′-tetramethyluronium hexafluorophosphate (46.7 mg, 0.123 mmol) was added to a mixture of C11 (40 mg, ≦0.12 mmol), P1 (87.7 mg, 0.270 mmol), and triethylamine (74.6 mg, 0.737 mmol) in dichloromethane (4 mL). The reaction mixture was stirred at 25° C. for 20 hours, whereupon it was treated with additional P1 (40 mg, 0.12 mmol), and stirring was continued for 20 hours. The reaction mixture was concentrated in vacuo; preparative thin layer chromatography on silica gel (Eluent: 10:1 dichloromethane/methanol) provided the product as a white solid. Yield: 17 mg, 40 μmol, 24% over two steps. LCMS m/z 425.0 [M+H]⁺. ¹H NMR (400 MHz, CDCl₃) δ 8.33 (s, 1H), 8.11 (br d, J=6 Hz, 1H), 7.94 (s, 1H), 7.73 (s, 1H), 7.60 (s, 1H), 7.14-7.19 (m, 2H), 6.99 (dd, J=8, 8 Hz, 1H), 4.40 (d, J=3.3 Hz, 1H), 4.09 (dd, J=11.4, 5.1 Hz, 1H), 4.02 (s, 2H), 3.95 (s, 3H), 3.88-4.0 (m, 2H), 3.59-3.67 (m, 1H), 3.47 (ddd, J=12, 12, 2 Hz, 1H), 3.22 (dd, J=11.3, 10.0 Hz, 1H), 2.37 (s, 3H), 1.99-2.07 (m, 1H), 1.74-1.86 (m, 1H).

### Example 3

### N-[(3R,4S)-3-Hydroxytetrahydro-2H-pyran-4-yl]-5-methyl-4-[4-(1,3-thiazol-2-yl)benzyl]pyridine-2-carboxamide (3)

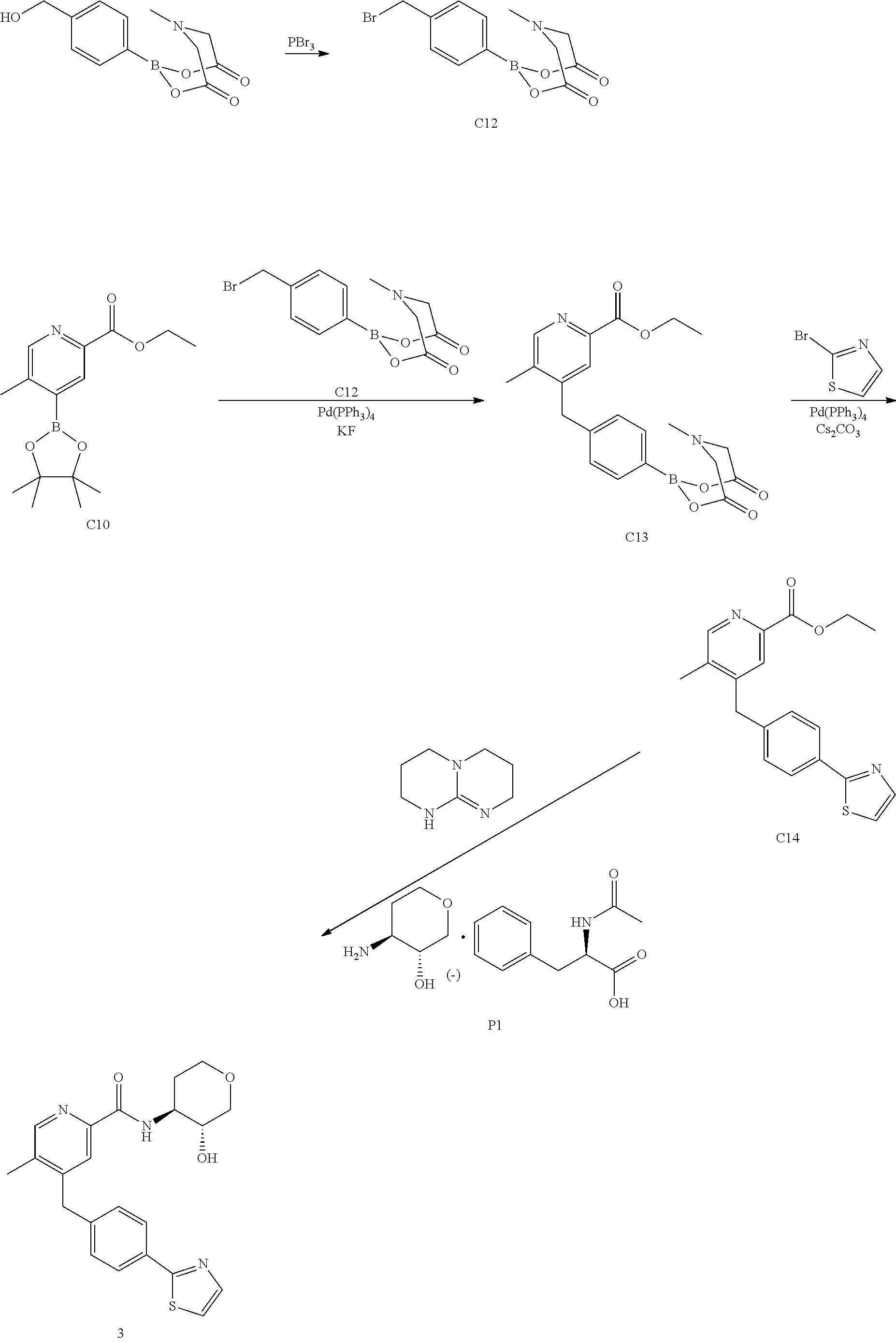

### Step 1. Synthesis of 2-[4-(bromomethyl)phenyl]-6-methyl-1,3,6,2-dioxazaborocane-4,8-dione (C12)

Phosphorus tribromide (11.3 g, 41.7 mmol) was added drop-wise to a 0° C. solution of 2-[4-(hydroxymethyl)phenyl]-6-methyl-1,3,6,2-dioxazaborocane-4,8-dione (10 g, 38 mmol) in dichloromethane (150 mL) and acetonitrile (150 mL). The reaction mixture was stirred overnight at room temperature, whereupon it was quenched via addition of saturated aqueous sodium bicarbonate solution. The aqueous layer was extracted with dichloromethane (3×200 mL), and the combined organic layers were dried, filtered, and concentrated in vacuo. The residue was washed with tert-butyl methyl ether (2×200 mL) to afford the product as a white solid. Yield: 10.7 g, 32.8 mmol, 86%. LCMS m/z 327.8 [M+H]⁺. ¹H NMR (400 MHz, CD₃OD) δ 7.47 (AB quartet, J_{AB}=8.2 Hz, □_{AB}=24.1 Hz, 4H), 4.57 (s, 2H), 4.26 (d, J=17.1 Hz, 2H), 4.06 (d, J=17.1 Hz, 2H), 2.57 (s, 3H).

### Step 2. Synthesis of ethyl 5-methyl-4-[4-(6-methyl-4,8-dioxo-1,3,6,2-dioxazaborocan-2-yl)benzyl]pyridine-2-carboxylate (C13)

To a solution of C10 (2.0 g, 6.9 mmol) and C12 (2.69 g, 8.25 mmol) in acetonitrile (100 mL) were added tetrakis(triphenylphosphine)palladium(0) (397 mg, 0.344 mmol) and potassium fluoride (2.0 g, 34 mmol). The reaction mixture was stirred for 4 hours at 80° C., whereupon it was diluted with water (400 mL) and extracted with ethyl acetate (3×200 mL). The combined organic layers were concentrated in vacuo and the residue was purified by chromatography on silica gel (Gradient: 0% to 5% methanol in dichloromethane) to provide the product as a yellow solid. Yield: 1.2 g, 2.9 mmol, 42%. LCMS m/z 410.9 [M+H]⁺. ¹H NMR (400 MHz, CD₃OD) δ 8.43 (s, 1H), 7.86 (s, 1H), 7.48 (d, J=7.8 Hz, 2H), 7.20 (d, J=7.8 Hz, 2H), 4.39 (q, J=7.1 Hz, 2H), 4.25 (d, J=17.1 Hz, 2H), 4.13 (s, 2H), 4.05 (d, J=17.1 Hz, 2H), 2.56 (s, 3H), 2.36 (s, 3H), 1.38 (t, J=7.2 Hz, 3H).

### Step 3. Synthesis of ethyl 5-methyl-4-[4-(1,3-thiazol-2-yl)benzyl]pyridine-2-carboxylate (C14)

2-Bromo-1,3-thiazole (90 mg, 0.55 mmol), tetrakis(triphenylphosphine)palladium(0) (423 mg, 0.366 mmol), and cesium carbonate (238 mg, 0.730 mmol) were added to a solution of C13 (150 mg, 0.37 mmol) in 1,4-dioxane (3 mL) and water (0.3 mL). The reaction mixture was stirred overnight at 80° C., and then filtered. The filtrate was concentrated under reduced pressure; silica gel chromatography (Gradient: 0% to 3% methanol in dichloromethane) afforded the product as a yellow gum. Yield: 50 mg, 0.15 mmol, 40%.

### Step 4. Synthesis of N-[(3R,4S)-3-hydroxytetrahydro-2H-pyran-4-yl]-5-methyl-4-[4-(1,3-thiazol-2-yl)benzyl]pyridine-2-carboxamide (3)

1,3,4,6,7,8-Hexahydro-2H-pyrimido[1,2-a]pyrimidine (95%, 350 mg, 2.39 mmol) was added to a solution of C14 (476 mg, 1.41 mmol) and P1 (456 mg, 1.41 mmol) in N,N-dimethylformamide (2.8 mL), and the reaction mixture was heated at 60° C. overnight. It was then cooled and partitioned between water and ethyl acetate. The aqueous layer was extracted with ethyl acetate, and the combined organic layers were washed with saturated aqueous sodium chloride solution, dried over magnesium sulfate, filtered, and concentrated in vacuo. Silica gel chromatography (Gradient: 50% to 100% ethyl acetate in heptane) afforded a yellow solid (497 mg). This was combined with the product (148 mg) of a similar reaction carried out on C14 (278 mg, 0.821 mmol), and the combined material was heated in a slurry with ethyl acetate. A small amount of heptane was added, and the suspension was allowed to stir and cool to room temperature over 3 hours. The resulting solid was collected via filtration to provide the product as a white powder. Yield: 300 mg, 0.73 mmol, 33%. LCMS m/z 410.1 [M+H]⁺. ¹H NMR (400 MHz, CDCl₃) δ 8.33 (s, 1H), 8.13 (br d, J=6 Hz, 1H), 8.02 (s, 1H), 7.90 (br d, J=8.1 Hz, 2H), 7.86 (d, J=3.3 Hz, 1H), 7.33 (d, J=3.2 Hz, 1H), 7.19 (br d, J=8.0 Hz, 2H), 4.35 (br s, 1H), 4.06-4.13 (m, 3H), 3.91-4.04 (m, 2H), 3.65 (ddd, J=9.5, 9.5, 5 Hz, 1H), 3.48 (ddd, J=12, 12, 2 Hz, 1H), 3.23 (dd, J=11, 10 Hz, 1H), 2.32 (s, 3H), 2.01-2.08 (m, 1H), 1.75-1.87 (m, 1H).

### Examples 4 and 5

### N-[(3,4-trans)-3-Hydroxytetrahydro-2H-pyran-4-yl]-5-methyl-4-[4-(1,3-thiazol-5-yl)benzyl]pyridine-2-carboxamide, ENT-1 (4) and N-[(3,4-trans)-3-Hydroxytetrahydro-2H-pyran-4-yl]-5-methyl-4-[4-(1,3-thiazol-5-yl)benzyl]pyridine-2-carboxamide, ENT-2 (5)

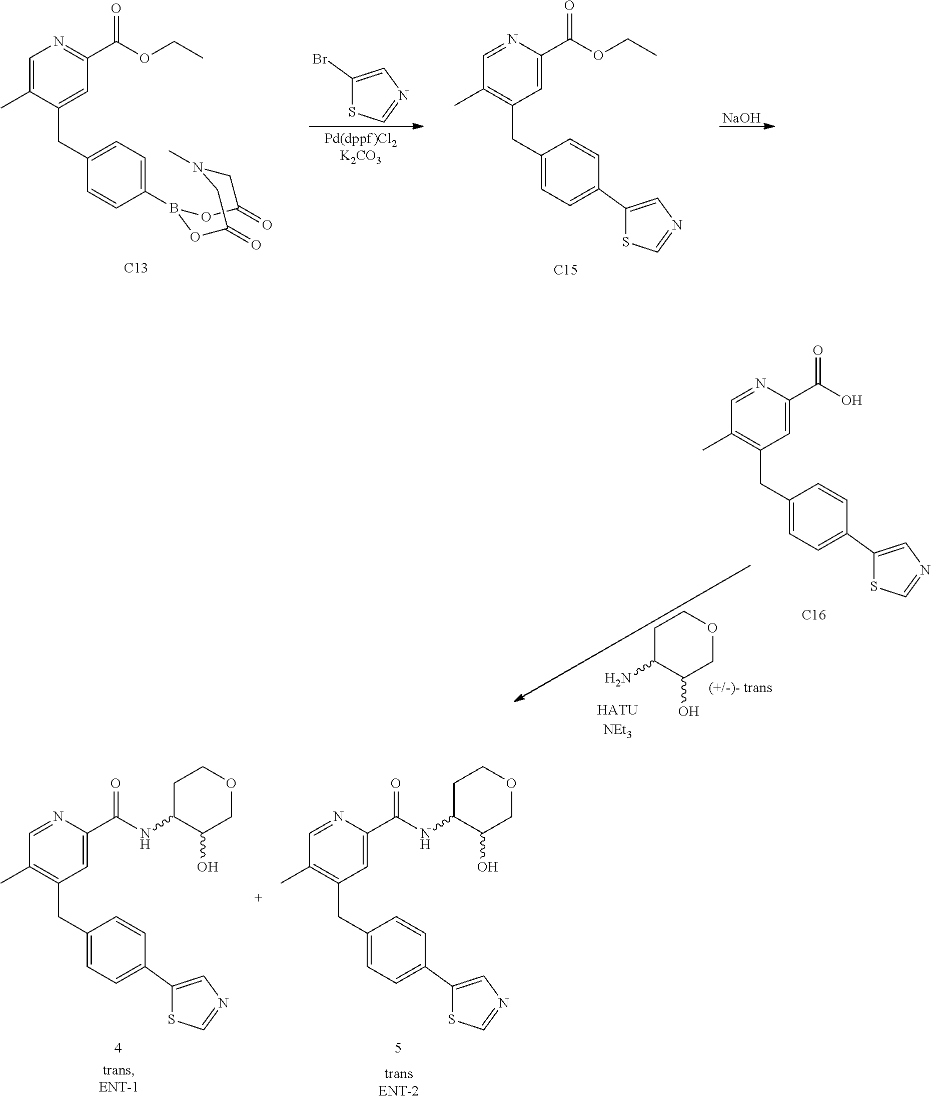

### Step 1. Synthesis of ethyl 5-methyl-4-[4-(1,3-thiazol-5-yl)benzyl]pyridine-2-carboxylate (C15)

To a solution of C13 (120 mg, 0.29 mmol) in 1,4-dioxane (3 mL) and water (0.3 mL) were added 5-bromo-1,3-thiazole (72 mg, 0.44 mmol), [1,1′-bis(diphenylphosphino)ferrocene]dichloropalladium(II) (214 mg, 0.292 mmol), and potassium carbonate (80.9 mg, 0.585 mmol). The reaction mixture was stirred overnight at 110° C., whereupon it was filtered. The filtrate was concentrated under reduced pressure, and the residue was purified by silica gel chromatography (Gradient: 0% to 3% methanol in dichloromethane) to afford the product as a yellow gum. Yield: 50 mg, 0.15 mmol, 52%.

### Step 2. Synthesis of 5-methyl-4-[4-(1,3-thiazol-5-yl)benzyl]pyridine-2-carboxylic acid (C16)

To a solution of C15 (50 mg, 0.15 mmol) in methanol (2 mL) and water (2 mL) was added sodium hydroxide (29.5 mg, 0.738 mmol), and the reaction mixture was stirred for 4 hours at reflux. It was then acidified via addition of 1 M hydrochloric acid and concentrated in vacuo to provide the product, which was used in the next step without additional purification

### Step 3. Synthesis of N-[(3,4-trans)-3-hydroxytetrahydro-2H-pyran-4-yl]-5-methyl-4-[4-(1,3-thiazol-5-yl)benzyl]pyridine-2-carboxamide, ENT-1 (4) and N-[(3,4-trans)-3-hydroxytetrahydro-2H-pyran-4-yl]-5-methyl-4-[4-(1,3-thiazol-5-yl)benzyl]pyridine-2-carboxamide, ENT-2 (5)

To a solution of C16 (from the previous step, 46 mg, 0.15 mmol) in dichloromethane (5 mL) were added O-(7-azabenzotriazol-1-yl)-N,N,N′,N′-tetramethyluronium hexafluorophosphate (56.4 mg, 0.148 mmol), trans-4-aminotetrahydro-2H-pyran-3-ol (34.7 mg, 0.296 mmol), and triethylamine (45.0 mg, 0.445 mmol), and the reaction mixture was stirred overnight at room temperature. The reaction mixture was concentrated in vacuo and the residue was purified by preparative thin layer chromatography on silica gel (Gradient: 20:1 dichloromethane/methanol) to provide the racemate of the products as a yellow gum. Yield: 35 mg, 85 μmol, 57% over two steps. This material was separated into its component enantiomers via reversed phase HPLC (Column: Chiral Technologies Chiralpak AD, 10 μm; Mobile phase: 55% ethanol in aqueous ammonia) to provide 4 and 5, both as white solids (Examples 4 and 5 are designated according to their respective retention time shown below). 4: Yield: 10.4 mg, 25.4 μmol, 30% for the chiral separation. LCMS m/z 409.9 [M+H]⁺. ¹H NMR (400 MHz, CD₃OD) δ 8.95 (s, 1H), 8.42 (s, 1H), 8.15 (s, 1H), 7.85 (s, 1H), 7.62 (d, J=8.2 Hz, 2H), 7.26 (d, J=8.2 Hz, 2H), 4.14 (s, 2H), 3.87-4.00 (m, 3H), 3.59-3.69 (m, 1H), 3.47 (ddd, J=12.0, 11.8, 2.2 Hz, 1H), 3.19 (dd, J=10.9, 10.0 Hz, 1H), 2.36 (s, 3H), 1.97-2.05 (m, 1H), 1.64-1.76 (m, 1H). Retention time: 1.23 minutes (Column: Chiral Technologies Chiralpak AD-3, 4.6×50 mm, 3 μm; Mobile phase: 3:2 [ethanol, containing 0.05% diethylamine]/carbon dioxide; Flow rate: 3 mL/minute). 5: Yield: 6.8 mg, 17 μmol, 20% for the chiral separation. LCMS m/z 409.9 [M+H]⁺. ¹H NMR (400 MHz, CD₃OD) δ 8.94 (s, 1H), 8.41 (br s, 1H), 8.15 (s, 1H), 7.85 (br s, 1H), 7.61 (d, J=8.0 Hz, 2H), 7.25 (d, J=8.0 Hz, 2H), 4.13 (s, 2H), 3.86-4.00 (m, 3H), 3.59-3.69 (m, 1H), 3.43-3.51 (m, 1H), 3.19 (dd, J=10.5, 10.5 Hz, 1H), 2.35 (s, 3H), 1.96-2.05 (m, 1H), 1.63-1.77 (m, 1H). Retention time: 2.21 minutes (Column: Chiral Technologies Chiralpak AD-3, 4.6×50 mm, 3 μm; Mobile phase: 3:2 [ethanol, containing 0.05% diethylamine]/carbon dioxide; Flow rate: 3 mL/minute).

### Example 6

### N-[(3R,4S)-3-Hydroxytetrahydro-2H-pyran-4-yl]-5-methyl-4-[4-(1-methyl-1H-pyrazol-3-yl)benzyl]pyridine-2-carboxamide (6)

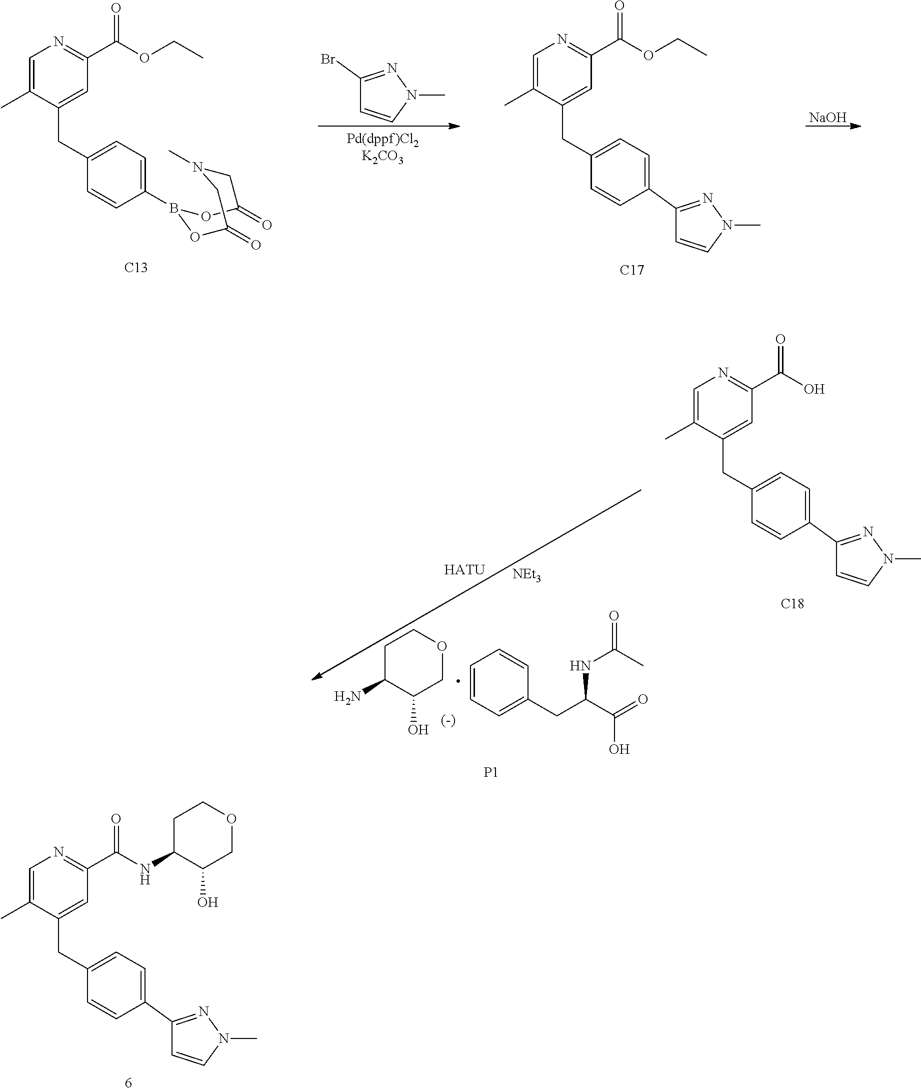

### Step 1. Synthesis of ethyl 5-methyl-4-[4-(1-methyl-1H-pyrazol-3-yl)benzyl]pyridine-2-carboxylate (C17)

[1,1′-Bis(diphenylphosphino)ferrocene]dichloropalladium(II) (10.7 mg, 14.6 μmol) was added to a mixture of C13 (60 mg, 0.15 mmol), 3-bromo-1-methyl-1H-pyrazole (28.3 mg, 0.176 mmol), and potassium carbonate (60.6 mg, 0.438 mmol) in toluene (5 mL) and water (0.2 mL), and the reaction mixture was stirred at 100° C. overnight. After removal of solvents in vacuo, the residue was purified by preparative thin layer chromatography on silica gel (Eluent: 20:1 dichloromethane/methanol) to give the crude product as a brown solid (50 mg); this was used in the next step without additional purification.

### Step 2. Synthesis of 5-methyl-4-[4-(1-methyl-1H-pyrazol-3-yl)benzyl]pyridine-2-carboxylic acid (C18)

Compound C17 (from the previous step, 50 mg, 0.15 mmol) and sodium hydroxide (23.9 mg, 0.598 mmol) were combined in a mixture of methanol (2 mL) and water (2 mL), and stirred overnight at 80° C. The reaction mixture was then concentrated in vacuo to remove methanol, and acidified to a pH of 1 with hydrochloric acid. After removal of solvent under reduced pressure, the residue (60 mg) was used directly in the following step.

### Step 3. Synthesis of N-[(3R,4S)-3-hydroxytetrahydro-2H-pyran-4-yl]-5-methyl-4-[4-(1-methyl-1H-pyrazol-3-yl)benzyl]pyridine-2-carboxamide (6)

To a solution of C18 (from the previous step, 60 mg, 0.15 mmol), P1 (83.6 mg, 0.258 mmol) and triethylamine (59.3 mg, 0.586 mmol) in dichloromethane (5 mL) was added O-(7-azabenzotriazol-1-yl)-N,N,N′,N′-tetramethyluronium hexafluorophosphate (44.5 mg, 0.117 mmol). The reaction mixture was stirred at 25° C. overnight, then at 40° C. for 3 hours, whereupon it was concentrated in vacuo. Preparative thin layer chromatography on silica gel (Eluent: 10:1 dichloromethane/methanol) provided the product as a white solid. Yield: 5.1 mg, 13 μmol, 9% over three steps. LCMS m/z 406.9 [M+H]⁺. ¹H NMR (400 MHz, CDCl₃) δ 8.31 (s, 1H), 8.08-8.15 (m, 1H), 8.02 (s, 1H), 7.71 (d, J=7.9 Hz, 2H), 7.37 (d, J=2.0 Hz, 1H), 7.13 (d, J=8.0 Hz, 2H), 6.50 (d, J=2.1 Hz, 1H), 4.41 (br s, 1H), 4.05 (s, 2H), 3.95 (s, 3H), 3.90-4.13 (m, 3H), 3.60-3.69 (m, 1H), 3.43-3.52 (m, 1H), 3.19-3.27 (m, 1H), 2.30 (s, 3H), 2.00-2.08 (m, 1H), 1.74-1.86 (m, 1H).

### Example 7

### N-[(3R,4S)-3-Hydroxytetrahydro-2H-pyran-4-yl]-5-methyl-4-[4-(1,3-thiazol-4-yl)benzyl]pyridine-2-carboxamide (7)

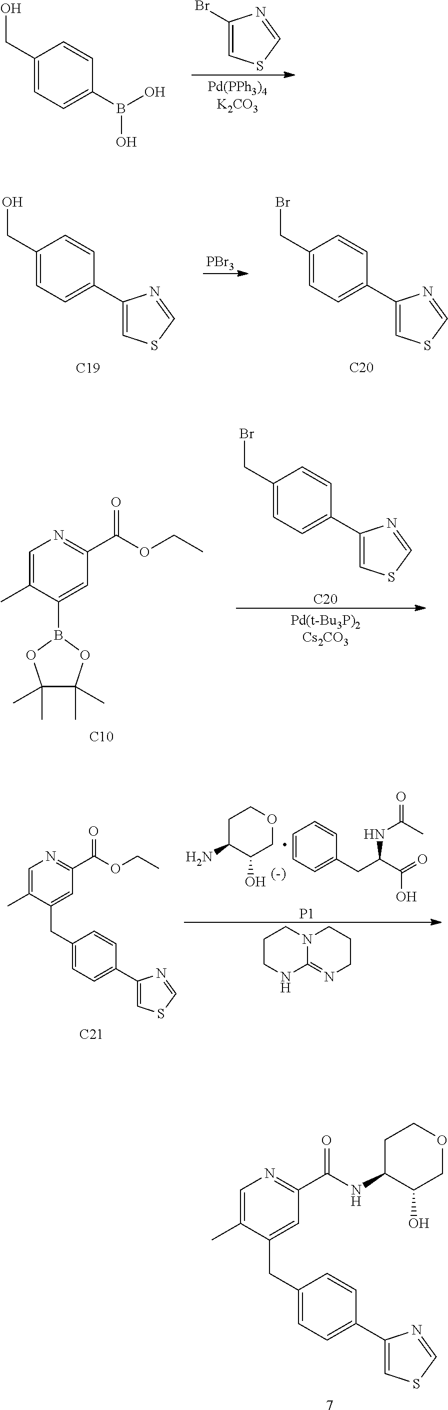

### Step 1. Synthesis of [4-(1,3-thiazol-4-yl)phenyl]methanol (C19)

Aqueous potassium carbonate solution (3.0 M, 17 mL, 51 mmol) was added to a solution of [4-(hydroxymethyl)phenyl]boronic acid (96%, 4.0 g, 25 mmol) and 4-bromo-1,3-thiazole (96%, 6.48 g, 37.9 mmol) in 1,4-dioxane (75 mL). Tetrakis(triphenylphosphine)palladium(0) (885 mg, 0.766 mmol) was added, and the reaction mixture was heated at 100° C. overnight. After cooling to room temperature, the reaction mixture was diluted with water and extracted several times with ethyl acetate. The combined organic layers were washed with saturated aqueous sodium chloride solution, dried over magnesium sulfate, filtered, and concentrated in vacuo; silica gel chromatography (Gradient: 25% to 50% ethyl acetate in heptane) provided the product as a cream-colored solid. Yield: 3.60 g, 18.8 mmol, 75%. LCMS m/z 192.0 [M+H]⁺. ¹H NMR (400 MHz, CDCl₃) δ 8.92 (d, J=2.0 Hz, 1H), 7.95 (br d, J=8.2 Hz, 2H), 7.56 (d, J=2.0 Hz, 1H), 7.46 (br d, J=8.3 Hz, 2H), 4.76 (s, 2H).

### Step 2. Synthesis of 4-[4-(bromomethyl)phenyl]-1,3-thiazole (C20)

Compound C19 (600 mg, 3.14 mmol) was dissolved in a mixture of dichloromethane (5 mL) and acetonitrile (5 mL), then treated in a drop-wise manner with phosphorus tribromide (99%, 0.298 mL, 3.14 mmol). The reaction mixture was allowed to stir at room temperature overnight, whereupon it was quenched with saturated aqueous sodium bicarbonate solution and extracted several times with ethyl acetate. The combined organic layers were washed with saturated aqueous sodium chloride solution, dried over magnesium sulfate, filtered, and concentrated in vacuo. Silica gel chromatography (Gradient: 10% to 25% ethyl acetate in heptane) afforded the product as a white solid. Yield: 525 mg, 2.07 mmol, 66%. LCMS m/z 254.0, 256.0 [M+H]⁺. ¹H NMR (400 MHz, CDCl₃) δ 8.91 (d, J=2.0 Hz, 1H), 7.93 (br d, J=8.4 Hz, 2H), 7.58 (d, J=2.0 Hz, 1H), 7.48 (br d, J=8.5 Hz, 2H), 4.55 (s, 2H).

### Step 3. Synthesis of ethyl 5-methyl-4-[4-(1,3-thiazol-4-yl)benzyl]pyridine-2-carboxylate (C21)

Aqueous cesium carbonate solution (3 M, 5.0 mL, 15 mmol) was added to a solution of C20 (1.27 g, 5.00 mmol) and C10 (1.5 g, 5.2 mmol) in tetrahydrofuran (28 mL), and the resulting solution was sparged with nitrogen gas for 50 minutes. After addition of bis(tri-tert-butylphosphine)palladium(0) (99%, 516 mg, 0.999 mmol), the reaction mixture was heated at 40° C. overnight. It was then allowed to cool to room temperature, and was partitioned between water and ethyl acetate. The aqueous layer was extracted with 30 mL portions of ethyl acetate, and the combined organic layers were washed with saturated aqueous sodium chloride solution, dried over magnesium sulfate, filtered, and concentrated in vacuo. Silica gel chromatography (Gradient: 20% to 80% ethyl acetate in heptane) provided the product as a white solid. Yield: 872 mg, 2.58 mmol, 52%. LCMS m/z 339.0 [M+H]⁺. ¹H NMR (400 MHz, CDCl₃) δ 8.86 (d, J=2.0 Hz, 1H), 8.50-8.52 (m, 1H), 7.92 (s, 1H), 7.86 (br d, J=7.8 Hz, 2H), 7.50 (d, J=2.0 Hz, 1H), 7.17 (br d, J=7.8 Hz, 2H), 4.45 (q, J=7.1 Hz, 2H), 4.06 (s, 2H), 2.31 (s, 3H), 1.42 (t, J=7.1 Hz, 3H).

### Step 4. Synthesis of N-[(3R,4S)-3-hydroxytetrahydro-2H-pyran-4-yl]-5-methyl-4-[4-(1,3-thiazol-4-yl)benzyl]pyridine-2-carboxamide (7)

Compound C21 (872 mg, 2.58 mmol) was reacted with P1 according to the method described for synthesis of 3 in Example 3. In this case, the crude product obtained after ethyl acetate extraction was taken up as a slurry in hot ethyl acetate (15 mL), which was then allowed to stir and cool for 2 hours. Collection of the precipitate via filtration afforded the product as a white solid. Yield: 525 mg, 1.28 mmol, 50%. LCMS m/z 410.2 [M+H]⁺. ¹H NMR (400 MHz, DMSO-d₆) δ 9.18 (br d, J=1 Hz, 1H), 8.51 (d, J=8.4 Hz, 1H), 8.43 (s, 1H), 8.12 (br d, J=1 Hz, 1H), 7.94 (d, J=8.0 Hz, 2H), 7.78 (s, 1H), 7.26 (d, J=8.0 Hz, 2H), 4.93 (d, J=5.7 Hz, 1H), 4.12 (s, 2H), 3.71-3.83 (m, 3H), 3.51-3.61 (m, 1H), 3.27-3.36 (m, 1H, assumed, partially obscured by solvent peak), 3.01 (dd, J=10.5, 10.5 Hz, 1H), 2.33 (s, 3H), 1.77-1.85 (m, 1H), 1.55-1.68 (m, 1H).

### Examples 8 and 9

### N-[(3S,4R)-3-Hydroxytetrahydro-2H-pyran-4-yl]-5-methyl-4-[4-(1H-pyrazol-1-yl)benzyl]pyridine-2-carboxamide (8) and N-[(3R,4S)-3-Hydroxytetrahydro-2H-pyran-4-yl]-5-methyl-4-[4-(1H-pyrazol-1-yl)benzyl]pyridine-2-carboxamide (9)

### Route 1: Preparation of Examples 8 and 9

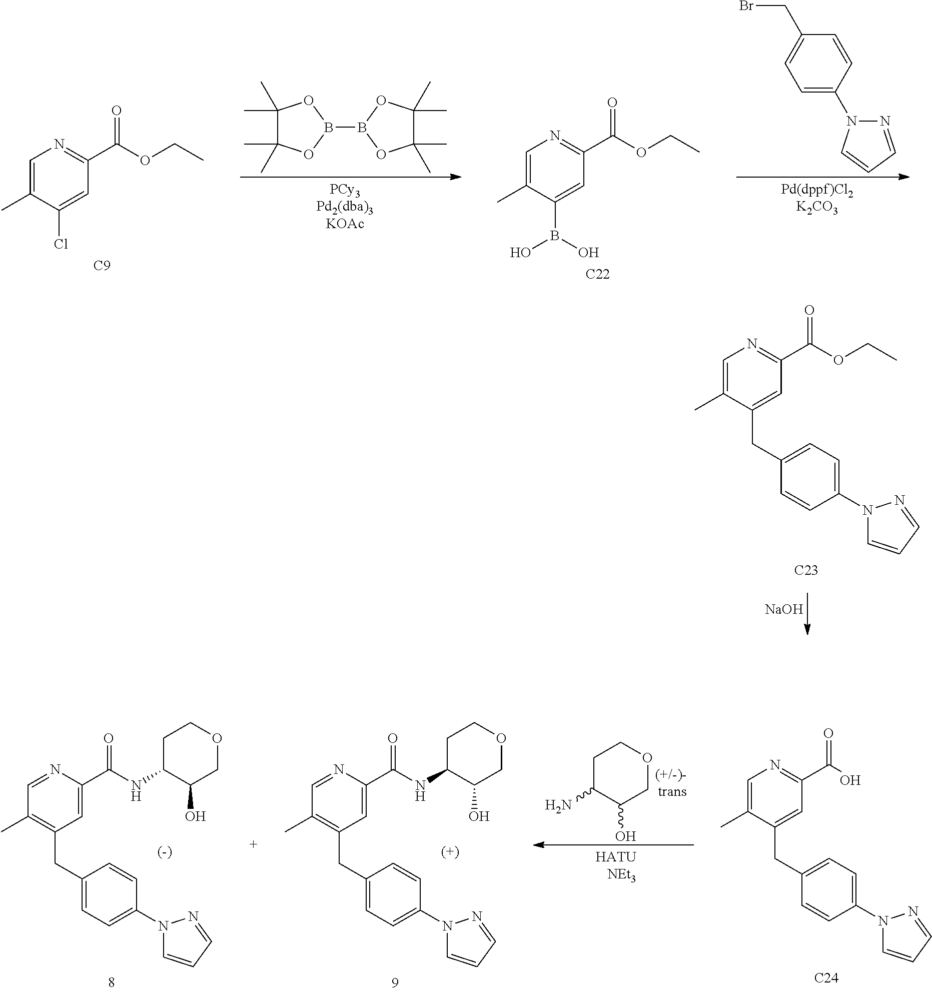

### Step 1. Synthesis of [2-(ethoxycarbonyl)-5-methylpyridin-4-yl]boronic acid (C22)

A mixture of C9 (680 mg, 3.41 mmol), 4,4,4′,4′,5,5,5′,5′-octamethyl-2,2′-bi-1,3,2-dioxaborolane (1.04 g, 4.10 mmol), tricyclohexylphosphine (48 mg, 0.17 mmol), tris(dibenzylideneacetone)dipalladium(0) (93 mg, 0.10 mmol), and potassium acetate (1.00 g, 10.2 mmol) in 1,4-dioxane (25 mL) was stirred in a sealed vial at 150° C. for 5.5 hours. The reaction mixture was filtered, and the filtrate (a 1,4-dioxane solution of C22) was used directly in the following step.

### Step 2. Synthesis of ethyl 5-methyl-4-[4-(1H-pyrazol-1-yl)benzyl]pyridine-2-carboxylate (C23)

A mixture of C22 (from the previous step, as a crude solution in 1,4-dioxane, 3.41 mmol), 1-[4-(bromomethyl)phenyl]-1H-pyrazole (1.25 g, 5.27 mmol), [1,1′-bis(diphenylphosphino)ferrocene]dichloropalladium(II) (175 mg, 0.239 mmol), and potassium carbonate (1.32 g, 9.55 mmol) in 1,4-dioxane (60 mL) and water (1 mL) was stirred at 80° C. for 20 hours. The reaction mixture was filtered through diatomaceous earth, and the filtrate was concentrated in vacuo. Silica gel chromatography (Gradient: 10% to 50% ethyl acetate in petroleum ether) afforded the product as an off-white gum. Yield: 800 mg, 2.5 mmol, 73% over two steps. ¹H NMR (400 MHz, CDCl₃) δ 8.53 (s, 1H), 7.89-7.93 (m, 2H), 7.71-7.73 (m, 1H), 7.64 (br d, J=8.5 Hz, 2H), 7.20 (br d, J=8.4 Hz, 2H), 6.47 (dd, J=2.3, 1.9 Hz, 1H), 4.47 (q, J=7.2 Hz, 2H), 4.07 (s, 2H), 2.32 (s, 3H), 1.44 (t, J=7.1 Hz, 3H).

### Step 3. Synthesis of 5-methyl-4-[4-(1H-pyrazol-1-yl)benzyl]pyridine-2-carboxylic acid (C24)

A mixture of C23 (800 mg, 2.5 mmol) and sodium hydroxide (398 mg, 9.95 mmol) in methanol (15 mL) and water (15 mL) was stirred at 80° C. for 2 hours. The reaction mixture was then diluted with water (50 mL), concentrated under reduced pressure to remove methanol, and acidified to a pH of 3-4 with concentrated hydrochloric acid. After extraction with a mixture of dichloromethane and methanol (20:1; 3×50 mL), the combined organic layers were dried over sodium sulfate, filtered, and concentrated in vacuo to provide the product as a yellow solid. Yield: 620 mg, 2.1 mmol, 84%.

### Step 4. Synthesis of N-[(3S,4R)-3-hydroxytetrahydro-2H-pyran-4-yl]-5-methyl-4-[4-(1H-pyrazol-1-yl)benzyl]pyridine-2-carboxamide (8) and N-[(3R,4S)-3-hydroxytetrahydro-2H-pyran-4-yl]-5-methyl-4-[4-(1H-pyrazol-1-yl)benzyl]pyridine-2-carboxamide (9)

A solution of C24 (800 mg, 2.73 mmol), trans-4-aminotetrahydro-2H-pyran-3-ol (383 mg, 3.27 mmol), O-(7-azabenzotriazol-1-yl)-N,N,N′,N′-tetramethyluronium hexafluorophosphate (1.24 g, 3.26 mmol), and triethylamine (828 mg, 8.18 mmol) in dichloromethane (30 mL) was stirred at room temperature for 2 hours. The reaction mixture was concentrated in vacuo, and the residue was purified via chromatography on silica gel (Gradient: 0% to 4% methanol in dichloromethane). The resulting yellow solid (950 mg, 2.4 mmol, 88%) was separated into its component enantiomers using reversed phase HPLC (Column: Chiral Technologies Chiralpak AD, 10 μm; Mobile phase: 55% ethanol in aqueous ammonia) to provide 8 and 9, both as white solids. Compound 8 was found to have a negative (−) rotation, and 9 exhibited a positive (+) rotation. The indicated absolute stereochemistry was assigned based on an X-ray crystal structure determination carried out on 9 (see below).

8: Yield: 360 mg, 0.92 mmol, 34%. LCMS m/z 392.9 [M+H]⁺. ¹H NMR (400 MHz, CD₃OD) δ 8.42 (s, 1H), 8.18 (d, J=2.5 Hz, 1H), 7.86 (s, 1H), 7.69-7.71 (m, 1H), 7.69 (br d, J=8.7 Hz, 2H), 7.30 (br d, J=8.7 Hz, 2H), 6.51 (dd, J=2, 2 Hz, 1H), 4.15 (s, 2H), 3.87-3.99 (m, 3H), 3.63 (ddd, J=9.5, 9.5, 5 Hz, 1H), 3.43-3.51 (m, 1H), 3.19 (dd, J=11, 10 Hz, 1H), 2.37 (s, 3H), 1.98-2.05 (m, 1H), 1.64-1.76 (m, 1H). Retention time: 0.91 minutes (Column: Chiral Technologies Chiralpak AD-3, 4.6×50 mm, 3 μm; Mobile phase: 2:3 [ethanol, containing 0.05% diethylamine]/carbon dioxide; Flow rate: 4 mL/minute).

9: Yield: 340 mg, 0.87 mmol, 32%. LCMS m/z 393.0 [M+H]⁺. ¹H NMR (400 MHz, CD₃OD) δ 8.42 (s, 1H), 8.18 (dd, J=2.5, 0.4 Hz, 1H), 7.86 (s, 1H), 7.70-7.71 (m, 1H), 7.69 (br d, J=8.7 Hz, 2H), 7.30 (br d, J=8.5 Hz, 2H), 6.51 (dd, J=2.5, 1.9 Hz, 1H), 4.15 (s, 2H), 3.87-3.99 (m, 3H), 3.64 (ddd, J=10, 9, 5 Hz, 1H), 3.47 (ddd, J=11.8, 11.7, 2.3 Hz, 1H), 3.19 (dd, J=11.0, 10.0 Hz, 1H), 2.37 (s, 3H), 1.98-2.06 (m, 1H), 1.64-1.76 (m, 1H). Retention time: 1.61 minutes (Column: Chiral Technologies Chiralpak AD-3, 4.6×50 mm, 3 μm; Mobile phase: 2:3 [ethanol, containing 0.05% diethylamine]/carbon dioxide; Flow rate: 4 mL/minute). A sample of 9 was crystallized from a very concentrated solution of ethyl acetate and diethyl ether; the resulting solid was slurried with 1:1 ethyl acetate/heptane and filtered. This material was subjected to X-ray structural analysis to determine its absolute configuration:

### Single-Crystal X-Ray Structural Determination of 9

### Single Crystal X-Ray Analysis

Data collection was performed on a Bruker APEX diffractometer at room temperature. Data collection consisted of omega and phi scans. The structure was solved by direct methods using SHELX software suite in the space group P2₁2₁2₁. The structure was subsequently refined by the full-matrix least squares method. All non-hydrogen atoms were found and refined using anisotropic displacement parameters.

The hydrogen atoms located on nitrogen and oxygen were found from the Fourier difference map and refined with distances and displacement parameters restrained. The remaining hydrogen atoms were placed in calculated positions and were allowed to ride on their carrier atoms. The final refinement included isotropic displacement parameters for all hydrogen atoms.

Assignment of the C20 vs N4 position on the pyrazole was done by examination of bond lengths and competitive refinement.

Analysis of the absolute structure using likelihood methods (Hooft, 2008) was performed using PLATON (Spek, 2003). The results indicate that the absolute structure has been correctly assigned. The method calculates that the probability that the structure is correct is 100.0. The Hooft parameter is reported as 0.08 with an esd of 0.04.

The final R-index was 2.9%. A final difference Fourier revealed no missing or misplaced electron density.

Pertinent crystal, data collection and refinement information is summarized in Table 1. Atomic coordinates, bond lengths, bond angles, torsion angles and displacement parameters are listed in Tables 2-5.

### SOFTWARE AND REFERENCES

SHELXTL, Version 5.1, Bruker AXS, 1997.
PLATON, A. L. Spek, J. Appl. Cryst. 2003, 36, 7-13.
MERCURY, C. F. Macrae, P. R. Edington, P. McCabe, E. Pidcock, G. P. Shields, R. Taylor, M. Towler, and J. van de Streek, J. Appl. Cryst. 2006, 39, 453-457.
OLEX2, O. V. Dolomanov, L. J. Bourhis, R. J. Gildea, J. A. K. Howard, and H. Puschmann, J. Appl. Cryst. 2009, 42, 339-341.
R. W. W. Hooft, L. H. Strayer, and A. L. Spek, J. Appl. Cryst. 2008, 41, 96-103. H. D. Flack, Acta Cryst. 1983, A39, 867-881.

TABLE 1
Crystal data and structure refinement for 9.

| Empirical formula | C₂₂H₂₄N₄O₃ | |
|---|---|---|
| Formula weight | 392.45 | |
| Temperature | 273(2) K | |
| Wavelength | 1.54178 Å | |
| Crystal system | Orthorhombic | |
| Space group | P2(1)2(1)2(1) | |
| Unit cell dimensions | a = 7.5895(2) Å | α = 90° |
| | b = 10.5562(2) Å | β = 90° |
| | c = 24.5616(6) Å | γ = 90° |

| | |
|---|---|
| Volume | 1967.78(8) Å³ |
| Z | 4 |
| Density (calculated) | 1.325 Mg/m³ |
| Absorption coefficient | 0.731 mm⁻¹ |
| F(000) | 832 |
| Crystal size | 0.21 × 0.11 × 0.05 mm³ |
| Theta range for data collection | 3.60 to 70.31° |
| Index ranges | −9 <= h <= 9, −12 <= |
| | k <= 12, −29 <= l <= 29 |
| Reflections collected | 41836 |
| Independent reflections | 3716 [R(int) = 0.0332] |
| Completeness to theta = 70.31° | 99.7% |
| Absorption correction | Empirical |
| Max. and min. transmission | 0.9644 and 0.8616 |
| Refinement method | Full-matrix least-squares on F² |
| Data/restraints/parameters | 3716/2/269 |
| Goodness-of-fit on F² | 1.013 |
| Final R indices [I > 2sigma(I)] | R1 = 0.0286, wR2 = 0.0726 |
| R indices (all data) | R1 = 0.0320, wR2 = 0.0751 |
| Absolute structure parameter | −0.03(18) |
| Largest diff. peak and hole | 0.135 and −0.086 e.Å⁻³ |

TABLE 2
Atomic coordinates (×10⁴) and equivalent isotropic displacement
parameters (Å² × 10³) for 9. U(eq) is defined as one-third
of the trace of the orthogonalized U^{ij} tensor.

| | x | y | z | U(eq) |
|---|---|---|---|---|
| C(1) | 3671(2) | −2607(1) | −872(1) | 57(1) |
| C(2) | 3664(2) | −3955(2) | −1079(1) | 62(1) |
| C(3) | 6551(2) | −3758(2) | −1411(1) | 61(1) |
| C(4) | 6710(2) | −2387(1) | −1222(1) | 49(1) |
| C(5) | 5539(2) | −2193(1) | −732(1) | 46(1) |
| C(6) | 5232(2) | −548(1) | −36(1) | 44(1) |
| C(7) | 5354(2) | 850(1) | 74(1) | 42(1) |
| C(8) | 5796(2) | 2832(1) | −255(1) | 51(1) |
| C(9) | 5636(2) | 3387(1) | 254(1) | 46(1) |
| C(10) | 5295(2) | 2600(1) | 697(1) | 44(1) |
| C(11) | 5142(2) | 1307(1) | 596(1) | 46(1) |
| C(12) | 5831(3) | 4801(1) | 316(1) | 63(1) |
| C(13) | 5160(2) | 3106(1) | 1270(1) | 55(1) |
| C(14) | 6950(2) | 3287(1) | 1533(1) | 48(1) |
| C(15) | 7456(2) | 4437(1) | 1757(1) | 51(1) |
| C(16) | 9063(2) | 4576(1) | 2019(1) | 51(1) |
| C(17) | 10200(2) | 3562(1) | 2054(1) | 48(1) |
| C(18) | 9729(2) | 2408(1) | 1832(1) | 58(1) |
| C(19) | 8118(2) | 2284(1) | 1577(1) | 58(1) |
| C(20) | 12743(2) | 4751(2) | 2444(1) | 62(1) |
| C(21) | 14216(2) | 4414(2) | 2717(1) | 70(1) |
| C(22) | 14124(3) | 3100(2) | 2751(1) | 74(1) |
| N(1) | 5592(2) | −884(1) | −547(1) | 49(1) |
| N(2) | 5682(2) | 1594(1) | −354(1) | 48(1) |
| N(3) | 11854(2) | 3684(1) | 2322(1) | 53(1) |
| N(4) | 12700(2) | 2644(1) | 2510(1) | 69(1) |
| O(1) | 4805(2) | −4113(1) | −1532(1) | 69(1) |
| O(2) | 8488(1) | −2049(1) | −1132(1) | 61(1) |
| O(3) | 4830(2) | −1304(1) | 324(1) | 59(1) |

TABLE 3
Bond lengths [Å] and angles [°] for 9.

| | | | |
|---|---|---|---|
| C(1)—C(2) | 1.511(2) | C(18)—C(19) | 1.379(2) |
| C(1)—C(5) | 1.523(2) | C(20)—N(3) | 1.347(2) |
| C(2)—O(1) | 1.420(2) | C(20)—C(21) | 1.351(2) |
| C(3)—O(1) | 1.409(2) | C(21)—C(22) | 1.391(3) |
| C(3)—C(4) | 1.525(2) | C(22)—N(4) | 1.323(2) |
| C(4)—O(2) | 1.4132(18) | N(3)—N(4) | 1.3538(17) |
| C(4)—C(5) | 1.5105(19) | C(2)—C(1)—C(5) | 110.44(12) |
| C(5)—N(1) | 1.4549(16) | O(1)—C(2)—C(1) | 111.82(13) |
| C(6)—O(3) | 1.2305(16) | O(1)—C(3)—C(4) | 113.00(13) |
| C(6)—N(1) | 1.3314(17) | O(2)—C(4)—C(5) | 113.78(11) |
| C(6)—C(7) | 1.5041(17) | O(2)—C(4)—C(3) | 111.28(12) |
| C(7)—N(2) | 1.3355(16) | C(5)—C(4)—C(3) | 108.95(12) |
| C(7)—C(11) | 1.3780(18) | N(1)—C(5)—C(4) | 111.14(11) |
| C(8)—N(2) | 1.3331(18) | N(1)—C(5)—C(1) | 111.65(11) |
| C(8)—C(9) | 1.3839(19) | C(4)—C(5)—C(1) | 109.19(11) |
| C(9)—C(10) | 1.3953(19) | O(3)—C(6)—N(1) | 123.78(12) |
| C(9)—C(12) | 1.5081(19) | O(3)—C(6)—C(7) | 121.42(12) |
| C(10)—C(11) | 1.3916(18) | N(1)—C(6)—C(7) | 114.80(11) |
| C(10)—C(13) | 1.5092(19) | N(2)—C(7)—C(11) | 123.25(11) |
| C(13)—C(14) | 1.516(2) | N(2)—C(7)—C(6) | 116.45(11) |
| C(14)—C(19) | 1.385(2) | C(11)—C(7)—C(6) | 120.31(12) |
| C(14)—C(15) | 1.3867(19) | N(2)—C(8)—C(9) | 125.05(13) |
| C(15)—C(16) | 1.387(2) | C(8)—C(9)—C(10) | 117.96(12) |
| C(16)—C(17) | 1.378(2) | C(8)—C(9)—C(12) | 120.14(13) |
| C(17)—C(18) | 1.3826(19) | C(10)—C(9)—C(12) | 121.90(13) |
| C(17)—N(3) | 1.4228(19) | C(19)—C(18)—C(17) | 119.46(14) |
| C(11)—C(10)—C(9) | 117.34(12) | C(18)—C(19)—C(14) | 122.06(14) |
| C(11)—C(10)—C(13) | 120.59(12) | N(3)—C(20)—C(21) | 107.74(16) |
| C(9)—C(10)—C(13) | 122.03(12) | C(20)—C(21)—C(22) | 104.57(16) |
| C(7)—C(11)—C(10) | 120.01(12) | N(4)—C(22)—C(21) | 112.13(17) |
| C(10)—C(13)—C(14) | 112.36(11) | C(6)—N(1)—C(5) | 122.76(11) |
| C(19)—C(14)—C(15) | 117.46(14) | C(8)—N(2)—C(7) | 116.37(11) |
| C(19)—C(14)—C(13) | 120.71(13) | C(20)—N(3)—N(4) | 111.36(13) |
| C(15)—C(14)—C(13) | 121.79(14) | C(20)—N(3)—C(17) | 128.41(13) |
| C(16)—C(15)—C(14) | 121.33(14) | N(4)—N(3)—C(17) | 120.17(12) |
| C(17)—C(16)—C(15) | 119.83(13) | C(22)—N(4)—N(3) | 104.19(14) |
| C(16)—C(17)—C(18) | 119.86(14) | C(3)—O(1)—C(2) | 112.17(11) |
| C(16)—C(17)—N(3) | 120.70(12) | | |
| C(18)—C(17)—N(3) | 119.43(13) | | |

### Symmetry Transformations Used to Generate Equivalent Atoms.

TABLE 4
Anisotropic displacement parameters (Å² × 10³) for 9.
The anisotropic displacement factor exponent takes the
form: −2π²[h² a*²U¹¹ + . . . + 2 h k a* b* U¹²].

| | U11 | U22 | U33 | U23 | U13 | U12 |
|---|---|---|---|---|---|---|
| C (1) | 55 (1) | 49 (1) | 66 (1) | −9 (1) | 1 (1) | −2 (1) |
| C (2) | 64 (1) | 53 (1) | 70 (1) | −14 (1) | −10 (1) | −6 (1) |
| C (3) | 66 (1) | 60 (1) | 58 (1) | −15 (1) | −1 (1) | 8 (1) |
| C (4) | 57 (1) | 47 (1) | 42 (1) | 1 (1) | −1 (1) | 3 (1) |
| C (5) | 59 (1) | 34 (1) | 43 (1) | −2 (1) | −2 (1) | −1 (1) |
| C (6) | 44 (1) | 41 (1) | 47 (1) | −3 (1) | −1 (1) | −3 (1) |
| C (7) | 39 (1) | 38 (1) | 47 (1) | −4 (1) | −1 (1) | −3 (1) |
| C (8) | 60 (1) | 39 (1) | 53 (1) | 2 (1) | −2 (1) | −4 (1) |
| C (9) | 42 (1) | 39 (1) | 57 (1) | −4 (1) | −4 (1) | 1 (1) |
| C (10) | 37 (1) | 45 (1) | 50 (1) | −9 (1) | 3 (1) | −2 (1) |
| C (11) | 46 (1) | 44 (1) | 46 (1) | −2 (1) | 3 (1) | −7 (1) |
| C (12) | 75 (1) | 41 (1) | 74 (1) | −8 (1) | −1 (1) | 0 (1) |
| C (13) | 54 (1) | 55 (1) | 55 (1) | −15 (1) | 11 (1) | −6 (1) |
| C (14) | 58 (1) | 48 (1) | 40 (1) | −8 (1) | 9 (1) | −5 (1) |
| C (15) | 58 (1) | 42 (1) | 53 (1) | −8 (1) | 7 (1) | −1 (1) |
| C (16) | 61 (1) | 44 (1) | 49 (1) | −7 (1) | 3 (1) | −6 (1) |
| C (17) | 60 (1) | 49 (1) | 35 (1) | 0 (1) | 4 (1) | −3 (1) |
| C (18) | 74 (1) | 46 (1) | 53 (1) | −6 (1) | −4 (1) | 8 (1) |
| C (19) | 76 (1) | 44 (1) | 56 (1) | −13 (1) | −2 (1) | −1 (1) |
| C (20) | 66 (1) | 62 (1) | 58 (1) | 3 (1) | 0 (1) | −9 (1) |
| C (21) | 60 (1) | 91 (1) | 59 (1) | 0 (1) | −4 (1) | −6 (1) |
| C (22) | 69 (1) | 88 (1) | 64 (1) | 3 (1) | −8 (1) | 9 (1) |
| N (1) | 65 (1) | 35 (1) | 47 (1) | −4 (1) | 4 (1) | −6 (1) |
| N (2) | 57 (1) | 41 (1) | 46 (1) | −3 (1) | 1 (1) | −2 (1) |
| N (3) | 61 (1) | 56 (1) | 42 (1) | 2 (1) | 1 (1) | −2 (1) |
| N (4) | 75 (1) | 68 (1) | 63 (1) | 4 (1) | −8 (1) | 11 (1) |
| O (1) | 76 (1) | 68 (1) | 63 (1) | −26 (1) | −12 (1) | 0 (1) |
| O (2) | 57 (1) | 62 (1) | 65 (1) | 3 (1) | 5 (1) | −3 (1) |
| O (3) | 79 (1) | 46 (1) | 52 (1) | 1 (1) | 7 (1) | −10 (1) |

TABLE 5
Hydrogen coordinates (×10⁴) and isotropic displacement
parameters (Å² × 10³) for 9.

| | x | y | z | U(eq) |
|---|---|---|---|---|
| H(1A) | 2932 | −2544 | −551 | 68 |
| H(1B) | 3190 | −2049 | −1149 | 68 |
| H(2A) | 4031 | −4518 | −788 | 75 |
| H(2B) | 2475 | −4186 | −1184 | 75 |
| H(3A) | 7273 | −3876 | −1733 | 73 |
| H(3B) | 7006 | −4311 | −1129 | 73 |
| H(4) | 6260 | −1847 | −1515 | 58 |
| H(5) | 5972 | −2734 | −436 | 55 |
| H(8) | 5999 | 3365 | −550 | 61 |
| H(11) | 4897 | 752 | 880 | 55 |
| H(12A) | 6230 | 5160 | −21 | 95 |
| H(12B) | 6674 | 4980 | 597 | 95 |
| H(12C) | 4714 | 5164 | 412 | 95 |
| H(13A) | 4471 | 2523 | 1489 | 66 |
| H(13B) | 4545 | 3912 | 1264 | 66 |
| H(15) | 6703 | 5129 | 1730 | 61 |
| H(16) | 9372 | 5352 | 2171 | 62 |
| H(18) | 10492 | 1720 | 1853 | 69 |
| H(19) | 7807 | 1504 | 1430 | 70 |
| H(20) | 12404 | 5573 | 2357 | 75 |
| H(21) | 15096 | 4942 | 2851 | 84 |
| H(22) | 14966 | 2601 | 2924 | 89 |
| H(98A) | 8850(30) | −2564(17) | −835(7) | 89 |
| H(99A) | 5880(30) | −216(16) | −784(7) | 89 |

### Route 2: Alternate preparation of N-[(3R,4S)-3-Hydroxytetrahydro-2H-pyran-4-yl]-5-methyl-4-[4-(1H-pyrazol-1-yl)benzyl]pyridine-2-carboxamide (9)

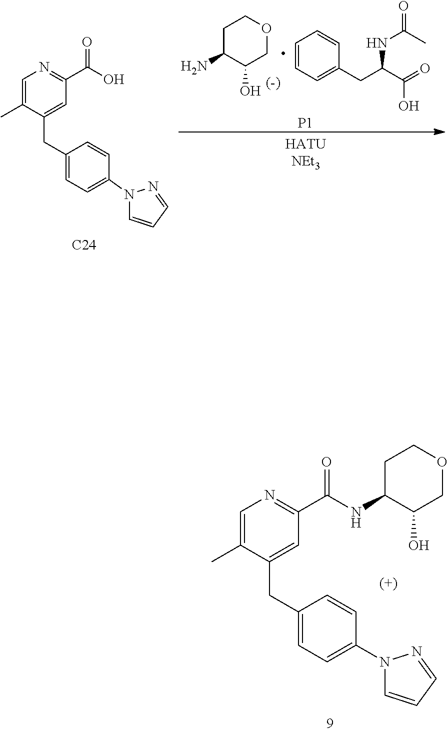

A hot solution of P1 (2.54 g, 7.83 mmol) in methanol (200 mL) was treated with Silicycle SiliaBond® carbonate resin (0.59 mmol/g, 100 g, 59 mmol), and the resulting mixture was stirred at room temperature overnight. The resin was removed via filtration, and the filter cake was thoroughly washed with methanol. The combined filtrates were concentrated in vacuo; the residue was combined with N,N-dimethylformamide (70 mL), C24 (2.00 g, 6.82 mmol), and triethylamine (1.4 mL, 10 mmol), then treated with O-(7-azabenzotriazol-1-yl)-N,N,N′,N′-tetramethyluronium hexafluorophosphate (99%, 3.93 g, 10.2 mmol). After the reaction mixture had stirred at room temperature overnight, it was diluted with half-saturated aqueous sodium bicarbonate solution, and extracted several times with ethyl acetate. The combined organic layers were washed twice with half-saturated aqueous sodium bicarbonate solution, twice with water, and once with saturated aqueous sodium chloride solution, dried over magnesium sulfate, filtered, and concentrated under reduced pressure. Silica gel chromatography (Gradient: 5% to 10% methanol in dichloromethane), followed by crystallization from a very concentrated solution of ethyl acetate and heptane, provided the product as a white solid. This material exhibited a positive (+) rotation, and was found to be crystalline via powder X-ray diffraction. Yield: 2.00 g, 5.10 mmol, 75%. LCMS m/z 393.1 [M+H]⁺. ¹H NMR (400 MHz, CD₃OD) δ 8.42 (s, 1H), 8.19 (dd, J=2.5, 0.5 Hz, 1H), 7.86 (s, 1H), 7.70-7.71 (m, 1H), 7.69 (br d, J=8.7 Hz, 2H), 7.30 (br d, J=8.7 Hz, 2H), 6.51 (dd, J=2.4, 1.9 Hz, 1H), 4.15 (s, 2H), 3.87-3.99 (m, 3H), 3.64 (ddd, J=9.7, 9.6, 4.9 Hz, 1H), 3.47 (ddd, J=11.9, 11.9, 2.2 Hz, 1H), 3.19 (dd, J=11.1, 9.8 Hz, 1H), 2.37 (s, 3H), 1.98-2.05 (m, 1H), 1.64-1.76 (m, 1H).

### Examples 10 and 11

### (−)-N-[(3,4-trans)-3-Hydroxytetrahydro-2H-pyran-4-yl]-5-methyl-4-[4-(2-methyl-1,3-oxazol-4-yl)benzyl]pyridine-2-carboxamide (10) and (+)-N-[(3,4-trans)-3-Hydroxytetrahydro-2H-pyran-4-yl]-5-methyl-4-[4-(2-methyl-1,3-oxazol-4-yl)benzyl]pyridine-2-carboxamide (11)

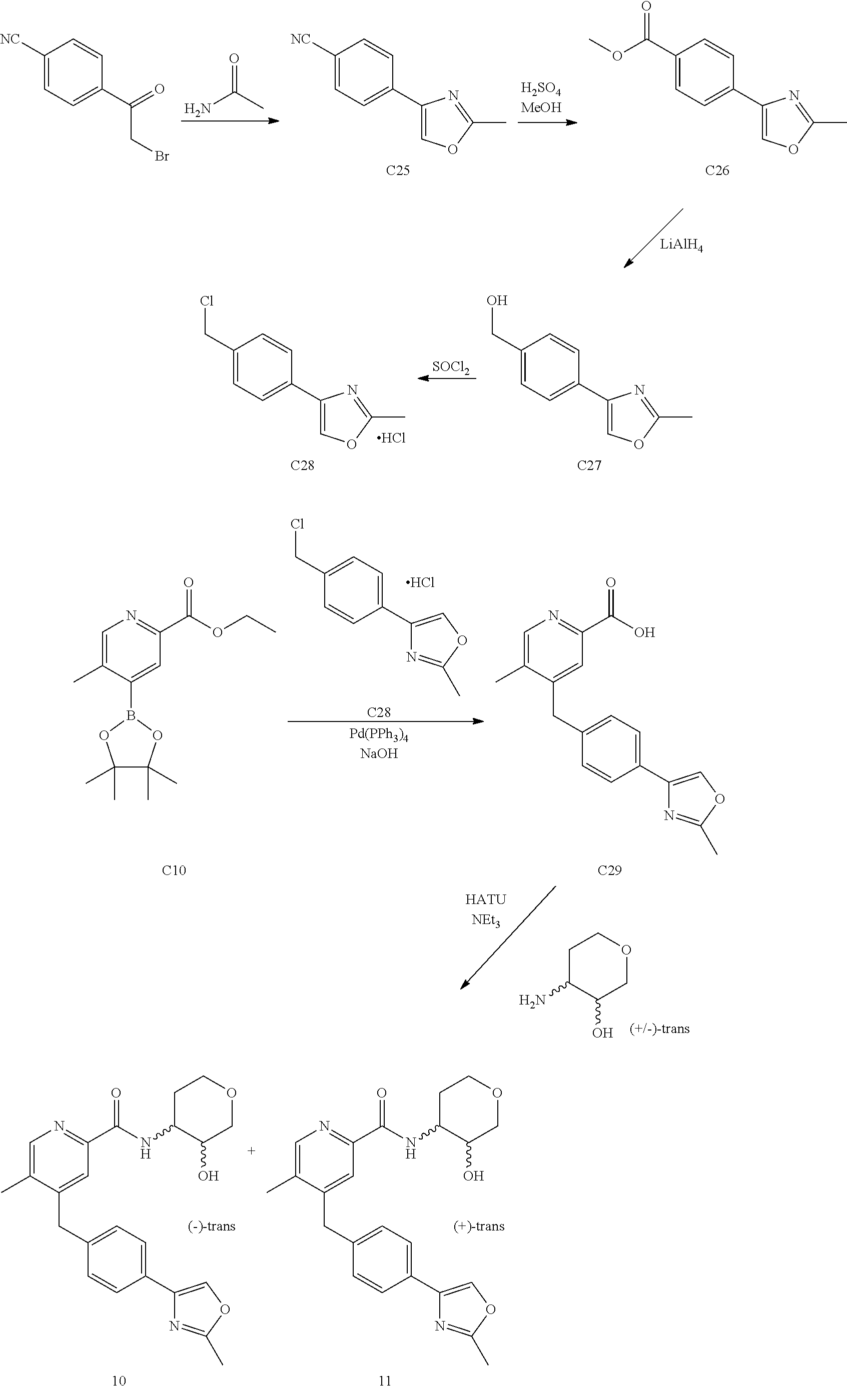

### Step 1. Synthesis of 4-(2-methyl-1,3-oxazol-4-yl)benzonitrile (C25)

A mixture of 4-(bromoacetyl)benzonitrile (9.5 g, 42 mmol) and acetamide (6.26 g, 106 mmol) in toluene (200 mL) was heated at reflux for 48 hours, whereupon it was filtered. After the filtrate had been concentrated in vacuo, silica gel chromatography (Gradient: 0% to 20% ethyl acetate in petroleum ether) afforded the product as a white solid. Yield: 7.5 g, 41 mmol, 98%. ¹H NMR (400 MHz, CDCl₃) δ 7.92 (s, 1H), 7.81 (br d, J=8.5 Hz, 2H), 7.68 (br d, J=8.7 Hz, 2H), 2.54 (s, 3H).

### Step 2. Synthesis of methyl 4-(2-methyl-1,3-oxazol-4-yl)benzoate (C26)

Compound C25 (6.0 g, 33 mmol) and concentrated sulfuric acid (50 mL) were combined in methanol (100 mL) and heated at reflux for 24 hours. The reaction mixture was slowly poured into ice water (300 mL), and the resulting mixture was adjusted to a pH of 7-8 with solid sodium hydroxide. Upon removal of methanol under reduced pressure, copious yellow solid precipitated; this was collected via filtration to provide the product as a yellow solid. Yield: 6.5 g, 30 mmol, 91%. ¹H NMR (400 MHz, CDCl₃) δ 8.06 (d, J=8.4 Hz, 2H), 7.90 (s, 1H), 7.77 (d, J=8.4 Hz, 2H), 3.92 (s, 3H), 2.53 (s, 3H).

### Step 3. Synthesis of [4-(2-methyl-1,3-oxazol-4-yl)phenyl]methanol (C27)

Lithium aluminum hydride (4.19 g, 110 mmol) was added to a −78° C. solution of C26 (6.00 g, 27.6 mmol) in tetrahydrofuran (200 mL), and the reaction mixture was allowed to stir at −30° C. for 1 hour. Water (4.5 mL) and aqueous sodium hydroxide solution (15%, 4.5 mL) were slowly added to the reaction mixture. It was then diluted with ethyl acetate (200 mL) and filtered; the filtrate was dried over sodium sulfate, filtered, and concentrated in vacuo to afford the product as a white solid. Yield: 4.0 g, 21 mmol, 76%. ¹H NMR (400 MHz, CDCl₃) δ 7.81 (s, 1H), 7.69 (d, J=8.2 Hz, 2H), 7.39 (d, J=8.0 Hz, 2H), 4.71 (s, 2H), 2.52 (s, 3H), 2.00-2.14 (br s, 1H).

### Step 4. Synthesis of 4-[4-(chloromethyl)phenyl]-2-methyl-1,3-oxazole, hydrochloride salt (C28)

Thionyl chloride (7.55 g, 63.5 mmol) was slowly added to a solution of C27 (4.0 g, 21 mmol) in dichloromethane (150 mL), and the reaction mixture was stirred at room temperature for 2 hours. Removal of solvent in vacuo provided the product as a yellow solid. Yield: 4.2 g, 17.2 mmol, 82%. ¹H NMR (400 MHz, CDCl₃) δ 8.04 (s, 1H), 7.90 (d, J=8.2 Hz, 2H), 7.51 (d, J=8.2 Hz, 2H), 4.61 (s, 2H), 2.96 (s, 3H).

### Step 5. Synthesis of 5-methyl-4-[4-(2-methyl-1,3-oxazol-4-yl)benzyl]pyridine-2-carboxylic acid (C29)

To a mixture of C28 (122 mg, 0.500 mmol), C10 (175 mg, 0.601 mmol), and sodium hydroxide (100 mg, 2.5 mmol) in acetonitrile (5 mL) and water (0.2 mL) was added tetrakis(triphenylphosphine)palladium(0) (58 mg, 50 μmol). The reaction mixture was stirred at 80° C. for 6 hours, whereupon it was diluted with water (10 mL) and washed with ethyl acetate (10 mL). The aqueous layer was acidified to a pH of 3 with hydrochloric acid, and the mixture was concentrated under reduced pressure to provide the product (160 mg), a portion of which was used in the next step without further purification

### Step 6. Synthesis of (−)-N-[(3,4-trans)-3-hydroxytetrahydro-2H-pyran-4-yl]-5-methyl-4-[4-(2-methyl-1,3-oxazol-4-yl)benzyl]pyridine-2-carboxamide (10) and (+)-N-[(3,4-trans)-3-hydroxytetrahydro-2H-pyran-4-yl]-5-methyl-4-[4-(2-methyl-1,3-oxazol-4-yl)benzyl]pyridine-2-carboxamide (11)

To a mixture of C29 (120 mg, 0.38 mmol), trans-4-aminotetrahydro-2H-pyran-3-ol (68.4 mg, 0.584 mmol) and triethylamine (118 mg, 1.17 mmol) in dichloromethane (10 mL) was added O-(7-azabenzotriazol-1-yl)-N,N,N′,N′-tetramethyluronium hexafluorophosphate (148 mg, 0.389 mmol), and the reaction mixture was stirred at room temperature overnight, then at 30° C. overnight. After the reaction mixture had been concentrated in vacuo, the residue was purified twice by preparative thin layer chromatography on silica gel (Eluent: 10:1 dichloromethane/methanol). The resulting compound was separated into its component enantiomers via reversed phase HPLC (Column: Chiral Technologies Chiralpak AD, 10 μm; Mobile phase: 55% ethanol in aqueous ammonia) to provide 10 and 11, both as white solids. Compound 10 was found to have a negative (−) rotation, and 11 exhibited a positive (+) rotation. Compounds 10 and 11 are designated according to their rotation signs.

10: Yield: 16.1 mg, 39.5 μmol, 10% over two steps. LCMS m/z 429.9 [M+N⁺]. ¹H NMR (400 MHz, CDCl₃) δ 8.30 (s, 1H), 8.12 (br d, J=6 Hz, 1H), 7.99 (s, 1H), 7.78 (s, 1H), 7.62 (d, J=8.0 Hz, 2H), 7.13 (d, J=7.8 Hz, 2H), 4.46 (br s, 1H), 4.04 (s, 2H), 3.89-4.14 (m, 3H), 3.59-3.68 (m, 1H), 3.42-3.52 (m, 1H), 3.23 (dd, J=10.8, 10.5 Hz, 1H), 2.51 (s, 3H), 2.30 (s, 3H), 1.99-2.09 (m, 1H), 1.73-1.87 (m, 1H). Retention time: 0.63 minutes (Column: Chiral Technologies Chiralpak AD-3, 4.6×50 mm, 3 μm; Mobile phase: 3:2 [methanol, containing 0.05% diethylamine]/carbon dioxide; Flow rate: 3 mL/minute).

11: Yield: 7.8 mg, 19 μmol, 5% over two steps. LCMS m/z 430.0 [M+Na⁺]. ¹H NMR (400 MHz, CDCl₃) δ 8.31 (s, 1H), 8.12 (br d, J=6 Hz, 1H), 8.00 (s, 1H), 7.78 (s, 1H), 7.63 (d, J=8.2 Hz, 2H), 7.13 (d, J=8.2 Hz, 2H), 4.45 (br s, 1H), 4.09 (dd, J=11.4, 4.8 Hz, 1H), 4.04 (s, 2H), 3.90-4.03 (m, 2H), 3.64 (br ddd, J=9.5, 9.5, 5 Hz, 1H), 3.47 (ddd, J=12.0, 11.9, 2.1 Hz, 1H), 3.23 (dd, J=11.2, 10.1 Hz, 1H), 2.51 (s, 3H), 2.30 (s, 3H), 2.00-2.08 (m, 1H), 1.74-1.86 (m, 1H). Retention time: 1.02 minutes (Column: Chiral Technologies Chiralpak AD-3, 4.6×50 mm, 3 μm; Mobile phase: 3:2 [methanol, containing 0.05% diethylamine]/carbon dioxide; Flow rate: 3 mL/minute).

### Examples 12 and 13

### (−)-N-[(1,2-cis)-2-Hydroxycyclohexyl]-5-methyl-4-[4-(1H-pyrazol-1-yl)benzyl]pyridine-2-carboxamide (12) and (+)-N-[(1,2-cis)-2-Hydroxycyclohexyl]-5-methyl-4-[4-(1H-pyrazol-1-yl)benzyl]pyridine-2-carboxamide (13)

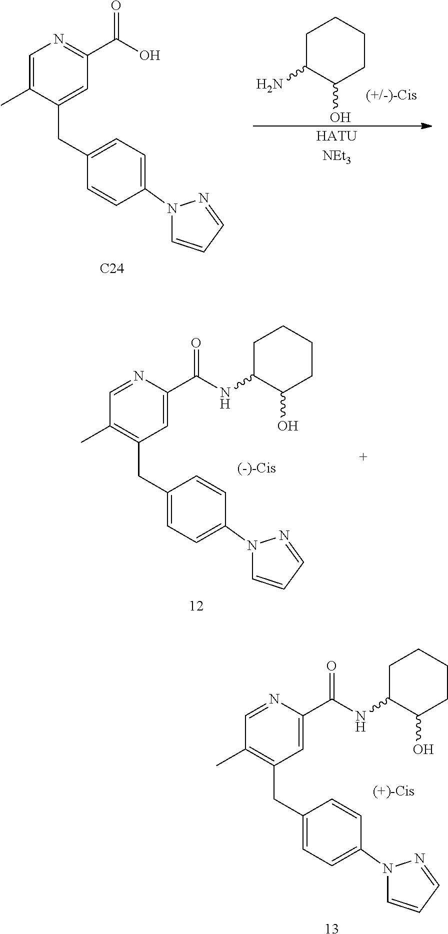

A mixture of C24 (50 mg, 0.17 mmol), cis-2-aminocyclohexanol (29.4 mg, 0.255 mmol), O-(7-azabenzotriazol-1-yl)-N,N,N′,N′-tetramethyluronium hexafluorophosphate (194 mg, 0.510 mmol), and triethylamine (86.2 mg, 0.852 mmol) in dichloromethane (5 mL) was stirred overnight at 40° C. The reaction mixture was concentrated in vacuo and the residue was purified by reversed phase HPLC (Column: DIKMA Diamonsil® C18(2), 5 μm; Mobile phase A: 0.225% formic acid in water; Mobile phase B: acetonitrile; Gradient: 40% to 60% B) to afford a racemic mixture of 12 and 13 as a white solid. Yield: 35 mg, 90 μmol, 53%. This material was separated into its component enantiomers via chiral HPLC (Column: Chiral Technologies Chiralpak AD, 10 μm; Mobile phase: 55% methanol in aqueous ammonia) to provide 12 and 13, both as white solids. Compound 12 was found to have a negative (−) rotation, and 13 exhibited a positive (+) rotation [Compounds 12 and 13 are designated according to their rotation signs].

12: Yield: 11.5 mg, 29.4 μmol, 33% from the chiral separation. LCMS m/z 390.9 [M+H]⁺. ¹H NMR (400 MHz, CD₃OD) δ 8.40 (s, 1H), 8.17-8.22 (m, 1H), 7.85 (s, 1H), 7.65-7.74 (m, 3H), 7.30 (br d, J=8.3 Hz, 2H), 6.49-6.54 (m, 1H), 4.15 (s, 2H), 3.91-3.99 (m, 2H), 2.36 (s, 3H), 1.56-1.88 (m, 6H), 1.36-1.50 (m, 2H). Retention time: 0.84 minutes (Column: Chiral Technologies Chiralpak AD-3, 4.6×50 mm, 3 μm; Mobile phase: 3:2 [methanol, containing 0.05% diethylamine]/carbon dioxide; Flow rate: 3 mL/minute).

13: Yield: 12.5 mg, 32.0 μmol, 36% from the chiral separation. LCMS m/z 391.0 [M+H]⁺. ¹H NMR (400 MHz, CD₃OD) δ 8.40 (s, 1H), 8.19 (d, J=2.4 Hz, 1H), 7.85 (s, 1H), 7.69-7.73 (m, 1H), 7.68 (d, J=8.5 Hz, 2H), 7.30 (d, J=8.4 Hz, 2H), 6.51 (dd, J=2, 2 Hz, 1H), 4.14 (s, 2H), 3.91-3.99 (m, 2H), 2.36 (s, 3H), 1.56-1.88 (m, 6H), 1.35-1.50 (m, 2H). Retention time: 1.91 minutes (Column: Chiral Technologies Chiralpak AD-3, 4.6×50 mm, 3 μm; Mobile phase: 3:2 [methanol, containing 0.05% diethylamine]/carbon dioxide; Flow rate: 3 mL/minute).

### Example 14

### 5-Chloro-N-[(3R,4S)-3-hydroxytetrahydro-2H-pyran-4-yl]-6-methyl-4-[4-(1H-pyrazol-1-yl)benzyl]pyridine-2-carboxamide (14)

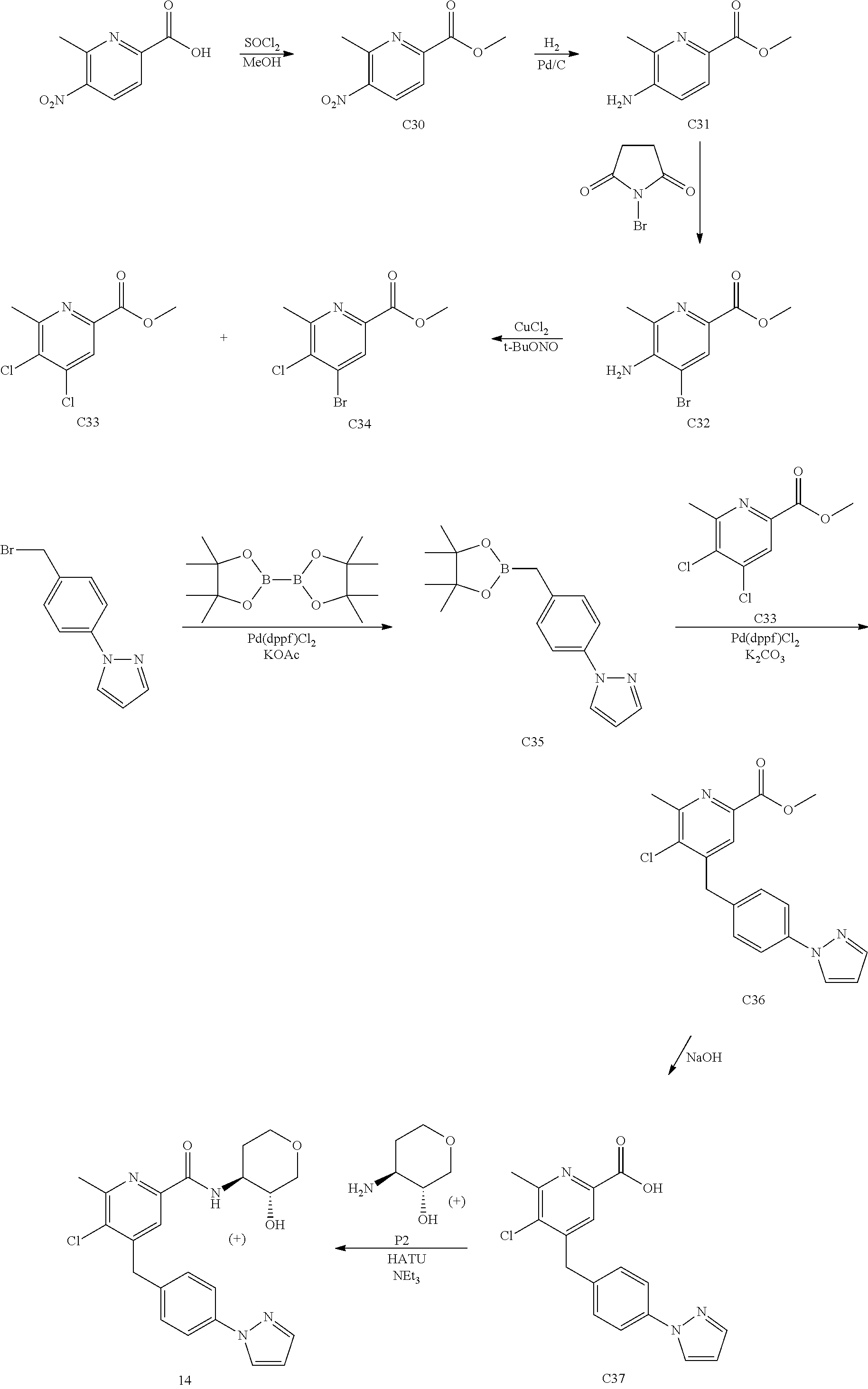

### Step 1. Synthesis of methyl 6-methyl-5-nitropyridine-2-carboxylate (C30)

A solution of 6-methyl-5-nitropyridine-2-carboxylic acid (4.0 g, 22 mmol) in methanol (50 mL) was treated with thionyl chloride (8.22 mL, 113 mmol) and heated at reflux for 17 hours. After removal of solvent in vacuo, the residue was partitioned between ethyl acetate and saturated aqueous sodium bicarbonate solution. The organic layer was dried over sodium sulfate, filtered, and concentrated under reduced pressure to afford the product. Yield: 3.7 g, 19 mmol, 86%. LCMS m/z 197.0 [M+H]⁺. ¹H NMR (400 MHz, DMSO-d₆) δ 8.57 (d, J=8.4 Hz, 1H), 8.12 (d, J=8.4 Hz, 1H), 3.92 (s, 3H), 2.77 (s, 3H).

### Step 2. Synthesis of methyl 5-amino-6-methylpyridine-2-carboxylate (C31)

An argon-purged solution of C30 (3.7 g, 19 mmol) in ethyl acetate (50 mL) was treated with 10% palladium on carbon (500 mg) and hydrogenated in a Parr shaker (40 psi hydrogen) for 4 hours. The reaction mixture was then filtered through diatomaceous earth; concentration of the filtrate in vacuo provided the product (3.1 g), which was used directly in the following step. LCMS m/z 166.9 [M+H]⁺. ¹H NMR (400 MHz, DMSO-d₆) δ 7.66 (d, J=8.3 Hz, 1H), 6.91 (d, J=8.3 Hz, 1H), 5.91 (s, 2H), 3.77 (s, 3H), 2.29 (s, 3H).

### Step 3. Synthesis of methyl 5-amino-4-bromo-6-methylpyridine-2-carboxylate (C32)

A solution of C31 (from the previous step, 3.1 g, 19 mmol) in acetonitrile (15 mL) was treated with N-bromosuccinimide (3.3 g, 19 mmol) and stirred at room temperature for 2 hours. Removal of solvent in vacuo provided a residue, which was partitioned between ethyl acetate and water. The organic layer was dried over sodium sulfate, filtered, and concentrated under reduced pressure; silica gel chromatography afforded the product as an off-white solid. Yield: 3.3 g, 13 mmol, 68% over 2 steps. LCMS m/z 245.0, 246.8 [M+H]⁺. ¹H NMR (400 MHz, DMSO-d₆) δ 7.90 (s, 1H), 6.12 (br s, 2H), 3.78 (s, 3H), 2.40 (s, 3H).

### Step 4. Synthesis of methyl 4,5-dichloro-6-methylpyridine-2-carboxylate (C33) and methyl 4-bromo-5-chloro-6-methylpyridine-2-carboxylate (C34)

A mixture of copper(II) chloride (1.7 g, 13 mmol) and tert-butyl nitrite (2.0 mL, 17 mmol) in acetonitrile (75 mL) was stirred at room temperature for 5 minutes, then heated to 60° C. Compound C32 (2.8 g, 11 mmol) was added, and stirring was continued at 60° C. for 4 hours. The reaction mixture was concentrated in vacuo and partitioned between ethyl acetate and water; the organic layer was dried over sodium sulfate, filtered, concentrated under reduced pressure, and subjected to silica gel chromatography. Further purification via reversed phase HPLC (Column: Waters XTerra Shield RP18 OBD Prep, 10 μm; Mobile phase A: 5 mM ammonium acetate in water; Mobile phase B: acetonitrile; Gradient: 10% to 40% B) afforded C33 and C34, both as white solids.

C33: Yield: 260 mg, 1.2 mmol, 11%. LCMS m/z 220.3, 222.1 [M+H]⁺. ¹H NMR (400 MHz, DMSO-d₆) δ 8.11 (s, 1H), 3.89 (s, 3H), 2.67 (s, 3H).

C34: Yield: 520 mg, 2.0 mmol, 18%. LCMS m/z 263.7, 265.7, 268.0 [M+H]⁺. ¹H NMR (400 MHz, DMSO-d₆) δ 8.20 (s, 1H), 3.89 (s, 3H), 2.68 (s, 3H).

### Step 5. Synthesis of 1-{4-[(4,4,5,5-tetramethyl-1,3,2-dioxaborolan-2-yl)methyl]phenyl}-1H-pyrazole (C35)

A mixture of 1-[4-(bromomethyl)phenyl]-1H-pyrazole (1.42 g, 5.99 mmol), 4,4,4′,4′,5,5,5′,5′-octamethyl-2,2′-bi-1,3,2-dioxaborolane (1.98 g, 7.80 mmol), [1,1′-bis(diphenylphosphino)ferrocene]dichloropalladium(II), dichloromethane complex (245 mg, 0.300 mmol), and potassium acetate (1.77 g, 18.0 mmol) in 1,4-dioxane (80 mL) was stirred at 100° C. for 6 hours. The reaction mixture was filtered through diatomaceous earth; the filtrate was concentrated in vacuo and subjected to silica gel chromatography (Gradient: 0% to 15% ethyl acetate in petroleum ether) to afford the product as an off-white solid. Yield: 1.4 g, 4.9 mmol, 82%. ¹H NMR (400 MHz, CDCl₃) δ 7.88 (d, J=2.4 Hz, 1H), 7.69-7.72 (m, 1H), 7.56 (br d, J=8.4 Hz, 2H), 7.27 (br d, J=8.2 Hz, 2H), 6.43-6.46 (m, 1H), 2.33 (s, 2H), 1.24 (s, 12H).

### Step 6. Synthesis of methyl 5-chloro-6-methyl-4-[4-(1H-pyrazol-1-yl)benzyl]pyridine-2-carboxylate (C36)

A mixture of C33 (160 mg, 0.727 mmol), C35 (310 mg, 1.09 mmol), [1,1′-bis(diphenylphosphino)ferrocene]dichloropalladium(II) (53 mg, 72 μmol) and potassium carbonate (201 mg, 1.45 mmol) in tetrahydrofuran (20 mL) and water (1 mL) was stirred at 90° C. for 40 hours. After addition of water (15 mL) to the reaction mixture, it was extracted with ethyl acetate (2×15 mL), and the combined organic layers were dried over sodium sulfate, filtered, and concentrated in vacuo. Silica gel chromatography (Gradient: 0% to 40% ethyl acetate in petroleum ether) provided the product as a white solid. Yield: 130 mg, 0.380 mmol, 52%.

### Step 7. Synthesis of 5-chloro-6-methyl-4-[4-(1H-pyrazol-1-yl)benzyl]pyridine-2-carboxylic acid (C37)

A mixture of C36 (130 mg, 0.380 mmol) and sodium hydroxide (76.1 mg, 1.90 mmol) in methanol (10 mL) and water (5 mL) was stirred at 80° C. for 2 hours. After removal of methanol under reduced pressure, water (10 mL) was added and the mixture was acidified with hydrochloric acid to a pH of 3. Filtration afforded the product as a white solid. Yield: 105 mg, 0.320 mmol, 84%. LCMS m/z 327.8 [M+H]⁺. ¹H NMR (400 MHz, CD₃OD) δ 8.20 (s, 1H), 7.87 (s, 1H), 7.66-7.76 (m, 3H), 7.37 (br d, J=8 Hz, 2H), 6.49-6.55 (m, 1H), 4.26 (s, 2H), 2.70 (s, 3H).

### Step 8. Synthesis of 5-chloro-N-[(3R,4S)-3-hydroxytetrahydro-2H-pyran-4-yl]-6-methyl-4-[4-(1H-pyrazol-1-yl)benzyl]pyridine-2-carboxamide (14)

O-(7-Azabenzotriazol-1-yl)-N,N,N′,N′-tetramethyluronium hexafluorophosphate (184 mg, 0.479 mmol) was added to a solution of C37 (105 mg, 0.320 mmol), P2 (41.2 mg, 0.352 mmol), and triethylamine (67.6 μL, 0.485 mmol) in N,N-dimethylformamide (4 mL). After the reaction mixture had been stirred at room temperature overnight, it was diluted with half-saturated aqueous sodium bicarbonate solution and extracted three times with ethyl acetate. The combined organic layers were washed twice with half-saturated aqueous sodium bicarbonate solution, twice with water, and once with saturated aqueous sodium chloride solution, then dried over magnesium sulfate, filtered, and concentrated in vacuo. Purification via chromatography on silica gel (Gradient: 0% to 50% [80:20:1 dichloromethane/methanol/concentrated ammonium hydroxide] in dichloromethane) was followed by crystallization from a very concentrated solution of warm 1:1 ethyl acetate/heptane, affording the product as a white solid. Compound 14 was found to have a positive (+) rotation. Yield: 112 mg, 0.262 mmol, 82%. LCMS m/z 427.1, 429.1 [M+H]⁺. ¹H NMR (400 MHz, CD₃OD) δ 8.64 (br d, J=8 Hz, 1H), 8.19 (dd, J=2.5, 0.6 Hz, 1H), 7.83 (br s, 1H), 7.70-7.71 (m, 1H), 7.69 (br d, J=8.7 Hz, 2H), 7.36 (br d, J=8.8 Hz, 2H), 6.51 (dd, J=2.5, 1.9 Hz, 1H), 4.25 (s, 2H), 3.87-3.99 (m, 3H), 3.65 (ddd, J=10, 10, 5 Hz, 1H), 3.47 (ddd, J=12, 12, 2 Hz, 1H), 3.18 (dd, J=11.1, 10 Hz, 1H), 2.70 (d, J=0.3 Hz, 3H), 1.96-2.03 (m, 1H), 1.66-1.78 (m, 1H).

### Example 15

### N-[(3R,4S)-3-Hydroxytetrahydro-2H-pyran-4-yl]-5-methoxy-4-[4-(1H-pyrazol-1-yl)benzyl]pyridine-2-carboxamide (15)

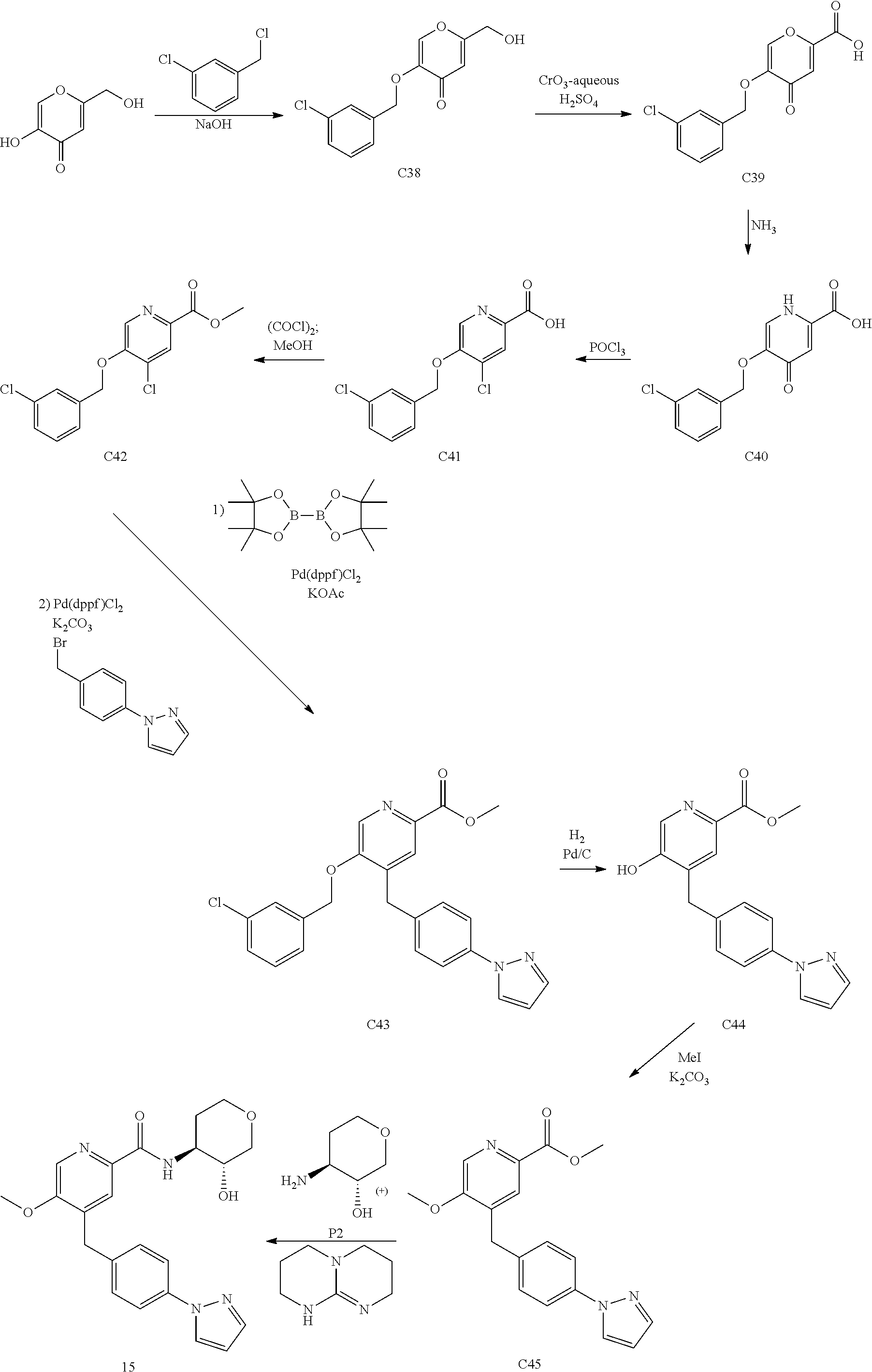

### Step 1. Synthesis of 5[(3-chlorobenzyl)oxy]-2-(hydroxymethyl)-4H-pyran-4-one (C38)

1-Chloro-3-(chloromethyl)benzene (25.7 mL, 202 mmol) was added drop-wise over a period of 10 minutes to a solution of 5-hydroxy-2-(hydroxymethyl)-4H-pyran-4-one (25.0 g, 176 mmol) and sodium hydroxide (7.74 g, 194 mmol) in aqueous methanol (1:10, 300 mL). The reaction mixture was heated to 80° C. for 5 hours, whereupon it was poured into ice-cold water. The resulting solid was isolated via filtration, then sequentially washed with water, diethyl ether (500 mL), and hexanes to afford the product as a white solid. Yield: 45 g, 170 mmol, 97%.

### Step 2. Synthesis of 5-[(3-chlorobenzyl)oxy]-4-oxo-4H-pyran-2-carboxylic acid (C39)

A suspension of C38 (10 g, 37 mmol) in acetone (200 mL) was cooled to 20° C. and slowly treated with Jones reagent (chromic acid content: 6.91 g, 69.1 mmol) over a period of 20 minutes. The reaction mixture was concentrated to half of its initial volume, whereupon it was diluted with ethyl acetate (200 mL) and extracted with saturated aqueous sodium bicarbonate solution (2×200 mL). The aqueous layer was acidified with 3 M hydrochloric acid and extracted with ethyl acetate (300 mL). This organic layer was washed with saturated aqueous sodium chloride solution, dried over sodium sulfate, filtered, and concentrated in vacuo to provide the product as a solid. Yield: 5.0 g, 18 mmol, 49%. LCMS m/z 281.2, 283.3 [M+H]⁺. ¹H NMR (300 MHz, DMSO-d₆) δ 8.39 (s, 1H), 7.4-7.5 (m, 4H), 6.95 (s, 1H), 5.01 (s, 2H).

### Step 3. Synthesis of 5[(3-chlorobenzyl)oxy]-4-oxo-1,4-dihydropyridine-2-carboxylic acid (C40)

A mixture of ammonia (25% aqueous solution, 14.6 mL, 195 mmol) and C39 (6.0 g, 21 mmol) was placed in a sealed tube and heated at 90° C. for 3 hours. The reaction mixture was then cooled to 5° C., diluted with diethyl ether (25 mL), and filtered, affording the product as a solid. Yield: 5.5 g, 20 mmol, 95%. LCMS m/z 280.2, 282.3 [M+H]⁺. ¹H NMR (300 MHz, DMSO-d₆) δ 7.50 (s, 1H), 7.3-7.4 (m, 3H), 7.25 (s, 1H), 6.58 (s, 1H), 5.02 (s, 2H).

### Step 4. Synthesis of 4-chloro-5-[(3-chlorobenzyl)oxy]pyridine-2-carboxylic acid (C41)

A suspension of C40 (5.0 g, 18 mmol) in phosphorus oxychloride (27 mL, 290 mmol) was heated at 95° C. for 30 minutes. The reaction mixture was concentrated in vacuo and quenched with water (50 mL); the resulting solid was collected via filtration. Silica gel chromatography (Eluent: 5% methanol in chloroform) provided the product as a white solid. Yield: 1 g, 3 mmol, 17%. LCMS m/z 298.3, 300.3, 302.3 [M+H]⁺. ¹H NMR (300 MHz, DMSO-d₆) δ 13.2 (br s, 1H), 8.7 (s, 1H), 8.1 (s, 1H), 7.6 (s, 1H), 7.4-7.6 (m, 3H), 5.50 (s, 2H).

### Step 5. Synthesis of methyl 4-chloro-5-[(3-chlorobenzyl)oxy]pyridine-2-carboxylate (C42)

To a 0° C. mixture of C41 (1.5 g, 5.0 mmol) in dichloromethane (20 mL) was added oxalyl chloride (1.28 g, 10.1 mmol) and N,N-dimethylformamide (184 mg, 2.52 mmol). After the reaction mixture had been stirred at room temperature for 2 hours, it was cooled to 0° C. and treated in a drop-wise manner with methanol (1 mL). The reaction mixture was then stirred at room temperature for 30 minutes, whereupon it was concentrated to dryness. The residue was washed with water (10 mL) and filtered; the filter cake was dried under vacuum. The resulting material was washed with petroleum ether (10 mL) and filtered to afford the product as a white solid. Yield: 1.5 g, 4.8 mmol, 96%. ¹H NMR (400 MHz, CDCl₃) δ 8.55 (br s, 1H), 8.29 (s, 1H), 7.48 (s, 1H), 7.37 (br s, 3H), 5.36 (br s, 2H), 4.05 (s, 3H).

### Step 6. Synthesis of methyl 5-[(3-chlorobenzyl)oxy]-4-[4-(1H-pyrazol-1-yl)benzyl]pyridine-2-carboxylate (C43)

[1,1′-Bis(diphenylphosphino)ferrocene]dichloropalladium(II) (305 mg, 0.417 mmol) was added to a mixture of C42 (1.3 g, 4.2 mmol), 4,4,4′,4′,5,5,5′,5′-octamethyl-2,2′-bi-1,3,2-dioxaborolane (2.54 g, 10.0 mmol), and potassium acetate (1.23 g, 12.5 mmol) in toluene (100 mL). The reaction mixture was heated to 120° C. for 16 hours. LCMS indicated that the desired {5-[(3-chlorobenzyl)oxy]-2-(methoxycarbonyl)pyridin-4-yl}boronic acid had been generated: LCMS m/z 321.7 [M+H]⁺. A solution of 1-[4-(bromomethyl)phenyl]-1H-pyrazole (2.47 g, 10.4 mmol) in 1,4-dioxane (100 mL) and water (10 mL) was added to the reaction mixture, followed by potassium carbonate (1.72 g, 12.4 mmol) and [1,1′-bis(diphenylphosphino)ferrocene]dichloropalladium(II) (304 mg, 0.415 mmol). After the reaction mixture had been stirred at 80° C. for 16 hours, it was filtered and the filtrate was concentrated to dryness. The residue was dissolved in ethyl acetate (100 mL), washed with water (60 mL), dried over sodium sulfate, filtered, and concentrated in vacuo. Silica gel chromatography (Gradient: 0% to 60% ethyl acetate in petroleum ether) afforded the product as an off-white solid. Yield: 850 mg, 1.96 mmol, 47%. LCMS m/z 434.0 [M+H]⁺. ¹H NMR (400 MHz, CDCl₃) δ 8.34 (s, 1H), 7.98 (s, 1H), 7.89-7.93 (m, 1H), 7.70-7.74 (m, 1H), 7.60-7.66 (m, 2H), 7.23-7.34 (m, 5H, assumed; partially obscured by solvent peak), 7.16-7.22 (m, 1H), 6.45-6.48 (m, 1H), 5.21 (s, 2H), 4.07 (s, 2H), 3.97 (s, 3H).

### Step 7. Synthesis of methyl 5-hydroxy-4-[4-(1H-pyrazol-1-yl)benzyl]pyridine-2-carboxylate (C44)

A mixture of C43 (850 mg, 1.96 mmol) and palladium on carbon (42 mg) in methanol (60 mL) was stirred for 4 hours at 30° C. under a hydrogen atmosphere (40 psi). The reaction mixture was filtered and the filtrate was concentrated in vacuo; the residue was washed with tert-butyl methyl ether (20 mL) to provide the product as a brown solid. Yield: 600 mg, 1.9 mmol, 97%. ¹H NMR (400 MHz, CDCl₃), characteristic peaks: δ 7.91-8.02 (m, 1H), 7.91 (s, 1H), 7.75 (s, 1H), 7.57-7.69 (m, 2H), 7.30-7.43 (m, 2H), 6.49 (s, 1H), 4.13 (br s, 2H), 3.97 (br s, 3H).

### Step 8. Synthesis of methyl 5-methoxy-4-[4-(1H-pyrazol-1-yl)benzyl]pyridine-2-carboxylate (C45)

To a suspension of C44 (70.0 mg, 0.226 mmol) in acetonitrile (2 mL) was added potassium carbonate (46.9 mg, 0.339 mmol) and iodomethane (33.7 mg, 0.237 mmol) at 20° C. After the mixture had been stirred for 2 hours, N,N-dimethylformamide (2 mL) and additional iodomethane (10 mg, 70 μmol) were added. Stirring was continued for 18 hours at 20° C., whereupon the reaction mixture was partitioned between dichloromethane (2 mL) and water (2 mL). The aqueous layer was extracted with dichloromethane (3×2 mL), and the combined organic layers were dried over sodium sulfate, filtered, and concentrated in vacuo. Preparative thin layer chromatography on silica gel (Eluent: 1:1 petroleum ether/ethyl acetate) afforded the product as a white solid. Yield: 20 mg, 62 μmol, 27%. LCMS m/z 323.8 [M+H]⁺.

### Step 9. Synthesis of N-[(3R,4S)-3-hydroxytetrahydro-2H-pyran-4-yl]-5-methoxy-4-[4-(1H-pyrazol-1-yl)benzyl]pyridine-2-carboxamide (15)

To a solution of C45 (15 mg, 46 μmol) in N,N-dimethylformamide (0.6 mL) was added P2 (7.61 mg, 65.0 μmol) and 1,3,4,6,7,8-hexahydro-2H-pyrimido[1,2-a]pyrimidine (6.46 mg, 46.4 μmol), and the reaction mixture was stirred for 20 hours at 50° C. Compound P2 (7.61 mg, 65.0 μmol) and 1,3,4,6,7,8-hexahydro-2H-pyrimido[1,2-a]pyrimidine (6.46 mg, 46.4 μmol) were added again, and stirring was continued for 20 hours at 50° C. After concentration of the reaction mixture in vacuo, preparative thin layer chromatography on silica gel (Eluent: ethyl acetate) provided the product as a white solid. Yield: 6.0 mg, 15 μmol, 33%. LCMS m/z 408.9 [M+H]⁺. ¹H NMR (400 MHz, CD₃OD) δ 8.32 (s, 1H), 8.16-8.19 (m, 1H), 7.88 (s, 1H), 7.69-7.71 (m, 1H), 7.66 (br d, J=8.7 Hz, 2H), 7.36 (br d, J=8.5 Hz, 2H), 6.49-6.53 (m, 1H), 4.08 (s, 2H), 4.04 (s, 3H), 3.86-3.99 (m, 3H), 3.58-3.67 (m, 1H), 3.42-3.51 (m, 1H), 3.15-3.22 (m, 1H), 1.96-2.05 (m, 1H), 1.62-1.75 (m, 1H).

### Example 16

### 5-Methyl-4-[4-(1H-pyrazol-1-yl)benzyl]-N-[(2S)-tetrahydrofuran-2-ylmethyl]pyridine-2-carboxamide, formate salt (16)

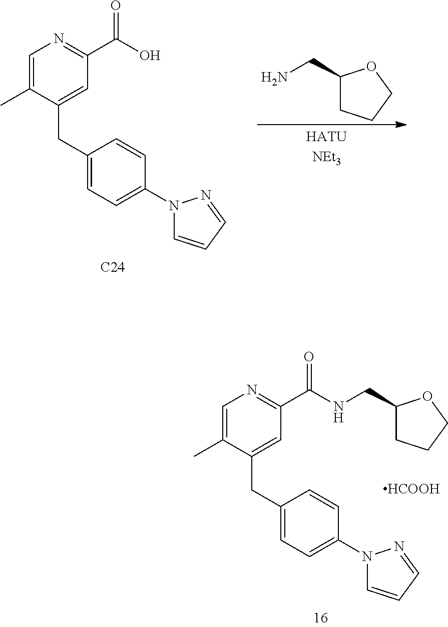 [SMILES: Cc1cnc(C(=O)O)cc1Cc1ccc(-n2cccn2)cc1 ; NC[C@@H]1CCCO1 ; "C24" ; Cc1cnc(C(=O)NC[C@@H]2CCCO2)cc1Cc1ccc(-n2cccn2)cc1 ; "·HCOOH"]

A mixture of 1-[(25)-tetrahydrofuran-2-yl]methanamine (0.38 M solution in N,N-dimethylformamide, 300 μL, 110 μmol), C24 (0.25 M solution in N,N-dimethylformamide, 300 μL, 75 μmol), and triethylamine (32 μL, 230 μmol) was treated with O-(7-azabenzotriazol-1-yl)-N,N,N′,N′-tetramethyluronium hexafluorophosphate (0.50 M solution in N,N-dimethylformamide, 150 μL, 75 μmol). The reaction vessel was sealed and shaken at 30° C. for 16 hours, whereupon solvent was removed using a Speedvac® concentrator. The residue was subjected to purification via reversed phase HPLC (Column: Phenomenex Gemini C18, 8 μm; Mobile phase A: 0.225% formic acid in water; Mobile phase B: acetonitrile; Gradient: 40% to 80% B) to afford the product. Yield: 9.1 mg, 24 μmol, 32%. LCMS m/z 377 [M+H]⁺. Retention time: 2.97 minutes (Column: Waters XBridge C18, 2.1×50 mm, 5 μm; Mobile phase A: 0.0375% trifluoroacetic acid in water; Mobile phase B: 0.01875% trifluoroacetic acid in acetonitrile; Gradient: 10% to 100% B over 4.0 minutes; Flow rate: 0.8 mL/minute).

### Example 17

### 5-Chloro-N-[(1S,2S)-2-hydroxycyclohexyl]-6-methyl-4-[4-(1H-pyrazol-1-yl)benzyl]pyridine-2-carboxamide (17)

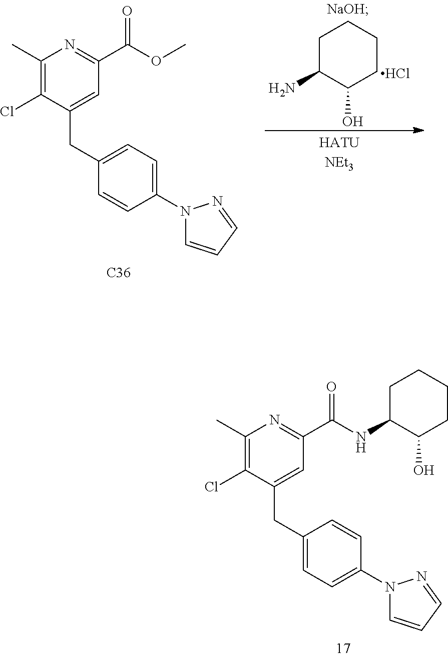 [SMILES: COC(=O)c1cc(Cc2ccc(-n3cccn3)cc2)c(Cl)c(C)n1 ; N[C@H]1CCCC[C@@H]1O ; Cl ; "C36" ; Cc1nc(C(=O)N[C@H]2CCCC[C@@H]2O)cc(Cc2ccc(-n3cccn3)cc2)c1Cl]

A mixture of C36 (25 mg, 73 μmol) and sodium hydroxide (12 mg, 0.30 mmol) in methanol (3 mL) and water (1 mL) was stirred at 70° C. for 3 hours, whereupon the pH was adjusted to approximately 7 via addition of 1 M hydrochloric acid. The resulting mixture was concentrated to dryness to provide the crude carboxylic acid as an off-white solid (25 mg). This material was combined with (1S,2S)-2-aminocyclohexanol, hydrochloride salt (23 mg, 0.15 mmol), O-(7-azabenzotriazol-1-yl)-N,N,N′,N′-tetramethyluronium hexafluorophosphate (43.3 mg, 0.114 mmol) and triethylamine (15 mg, 0.15 mmol) in N,N-dimethylformamide (12 mL), and the reaction mixture was stirred at room temperature for 20 hours. It was then concentrated to dryness, diluted with water (20 mL), and extracted with ethyl acetate (4×30 mL). The combined organic layers were dried, filtered, and concentrated under reduced pressure. Preparative thin layer chromatography (Eluent: 1:2 petroleum ether/ethyl acetate) afforded the product as an off-white solid. Yield: 7.0 mg, 16 μmol, 22%. LCMS m/z 447.0 [M+Na⁺]. ¹H NMR (400 MHz, CDCl₃) δ 7.99 (br d, J=6 Hz, 1H), 7.92 (s, 1H), 7.87-7.92 (m, 1H), 7.71 (s, 1H), 7.63 (d, J=8.3 Hz, 2H), 7.25-7.32 (m, 2H, assumed; partially obscured by solvent peak), 6.44-6.48 (m, 1H), 4.17 (s, 2H), 3.74-3.86 (m, 1H), 3.45-3.55 (m, 1H), 3.31-3.39 (m, 1H), 2.67 (s, 3H), 2.00-2.17 (m, 2H), 1.72-1.84 (m, 2H), 1.22-1.48 (m, 4H).

### Example 18

### N-[(1S,2S)-2-Hydroxycyclohexyl]-5-methyl-4-[4-(1H-pyrazol-1-yl)benzyl]pyridine-2-carboxamide (18)

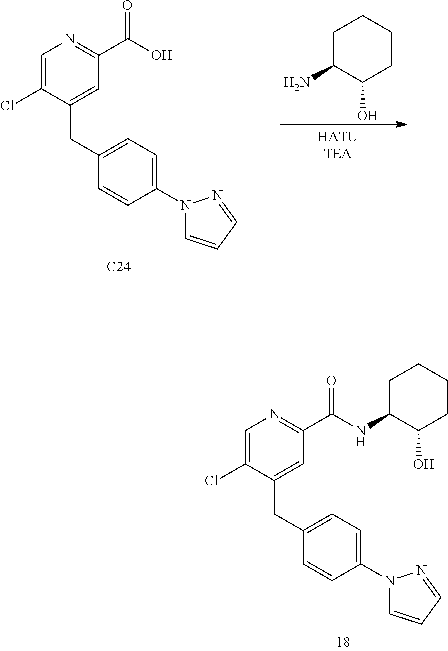 [SMILES: O=C(O)c1cc(Cc2ccc(-n3cccn3)cc2)c(Cl)cn1 ; N[C@H]1CCCC[C@@H]1O ; "C24" ; O=C(N[C@H]1CCCC[C@@H]1O)c1cc(Cc2ccc(-n3cccn3)cc2)c(Cl)cn1]

A mixture of C24 (120 mg, 0.41 mmol), (1S,2S)-2-aminocyclohexanol (56.5 mg, 0.490 mmol), O-(7-azabenzotriazol-1-yl)-N,N,N′,N′-tetramethyluronium hexafluorophosphate (187 mg, 0.492 mmol), and triethylamine (124 mg, 1.23 mmol) in dichloromethane (10 mL) was stirred overnight at room temperature. The reaction mixture was concentrated in vacuo and the residue was purified by silica gel chromatography (Gradient: 0% to 4% methanol in dichloromethane) to yield the product as a white solid. Yield: 145 mg, 0.371 mmol, 90%. LCMS m/z 391.1 [M+H]⁺. ¹H NMR (400 MHz, CD₃OD) δ 8.41 (s, 1H), 8.19 (d, J=2.5 Hz, 1H), 7.86 (s, 1H), 7.70 (d, J=1.6 Hz, 1H), 7.68 (d, J=8.5 Hz, 2H), 7.30 (d, J=8.3 Hz, 2H), 6.51 (dd, J=2.4, 1.9 Hz, 1H), 4.15 (s, 2H), 3.69-3.79 (m, 1H), 3.47-3.57 (m, 1H), 2.36 (s, 3H), 1.98-2.08 (m, 2H), 1.68-1.81 (m, 2H), 1.31-1.45 (m, 4H).

### Method A

Method A describes a specific method for preparations of certain exemplar compounds of the invention.

### Synthesis of 4-benzylpyridine-2-carboxamides or 4-(heteroarylmethyl)pyridine-2-carboxamides from 4-benzylpyridine-2-carboxylic acids or 4-(heteroarylmethyl)pyridine-2-carboxylic acids

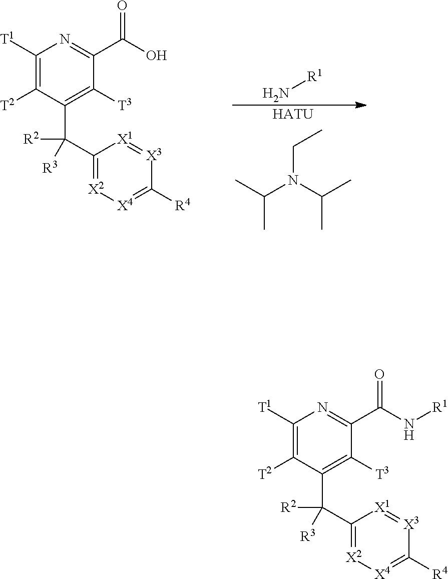

A mixture of amine R¹—NH₂ (75 μmol), the requisite 4-benzylpyridine-2-carboxylic acid or 4-(heteroarylmethyl)pyridine-2-carboxylic acid (0.15 M solution in N,N-dimethylformamide, 500 μL, 75 μmol), and N,N-diisopropylethylamine (40 μL, 230 μmol) was treated with O-(7-azabenzotriazol-1-yl)-N,N,N′,N′-tetramethyluronium hexafluorophosphate (0.375 M solution in N,N-dimethylformamide, 200 μL, 75 μmol). The reaction vessel was sealed and shaken at 50° C. for 16 hours, whereupon solvent was removed using a Speedvac® concentrator. The residue was subjected to purification via reversed phase HPLC (Column: Phenomenex Gemini C18, 8 μm; Mobile phase A: 0.225% formic acid in water; Mobile phase B: acetonitrile; Gradient 30% to 70% B) to afford the product.

Table 6 below lists some additional examples of compounds of invention (Examples 19-54) that were made using methods, starting materials or intermediates, and preparations described herein.

TABLE 6
Examples 19-54 (including Method of Preparation, Non-Commercial starting materials,
Structures and Physicochemical Data).

| | Method of | | ¹H NMR (400 MHz, CDCl₃) δ |
|---|---|---|---|
| | Preparation; | | (ppm); Mass spectrum, |
| | Non- | | observed ion m/z [M + H]⁺ or |
| | commercial | | HPLC retention time; Mass |
| Example | starting | | spectrum m/z [M + H]⁺ (unless |
| Number | materials | Structure | otherwise indicated) |
| 19 | Example 1¹; C10, P1 | 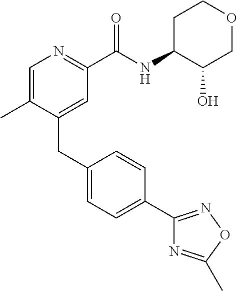 [SMILES: Cc1nc(-c2ccc(Cc3cc(C(=O)N[C@H]4CCOC[C@@H]4O)ncc3C)cc2)no1] | 8.33 (s, 1H), 8.09-8.15 (m, 1H), 7.96-8.03 (m, 3H), 7.22 (d, J = 8.2 Hz, 2H), 4.3-4.4 (br s, 1H), 4.06-4.13 (m, 3H), 3.90- 4.04 (m, 2H), 3.60-3.68 (m, 1H), 3.43-3.52 (m, 1H), 3.23 (dd, J = 11, 10 Hz, 1H), 2.66 (s, 3H), 2.31 (s, 3H), 2.00-2.08 (m, 1H), 1.74-1.87 (m, 1H); 408.9 |
| 20 | Examples 4 and 5²; C13 | 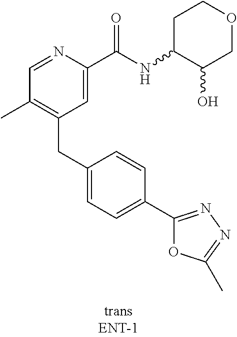 [SMILES: Cc1nnc(-c2ccc(Cc3cc(C(=O)NC4CCOCC4O)ncc3C)cc2)o1] | ¹H NMR (400 MHz, CD₃OD) δ 8.43 (s, 1H), 7.98 (br d, J = 8.4 Hz, 2H),7.85 (s,1H), 7.39 (br d, J = 8.3 Hz, 2H), 4.21 (s, 2H), 3.87-3.99 (m, 3H), 3.64 (ddd, J = 10, 10, 5 Hz, 1H), 3.47 (ddd, J = 12, 12, 2 Hz, 1H), 3.19 (dd, J = 11.0, 10.0 Hz, 1H), 2.61 (s, 3H), 2.36 (s, 3H), 1.98-2.05 (m, 1H), 1.65-1.76 (m, 1H); 408.9 |
| 21 | Examples 4 and 5²; C13 | 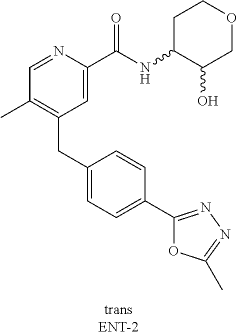 [SMILES: Cc1nnc(-c2ccc(Cc3cc(C(=O)NC4CCOCC4O)ncc3C)cc2)o1] | ¹H NMR (400 MHz, CD₃OD) δ 8.43 (3, 1H), 7.98 (br d, J = 8.4 Hz, 2H), 7.85 (s, 1H), 7.39 (br d, J = 8.4 Hz, 2H), 4.21 (s, 2H), 3.87-3.99 (m, 3H), 3.64 (ddd, J = 9.8, 9.7, 5.0 Hz, 1H), 3.47 (ddd, J = 12, 12, 2 Hz, 1H), 3.19 (dd, J = 11.0, 10.0 Hz, 1H), 2.61 (s, 3H), 2.36 (s, 3H), 1.97-2.05 (m, 1H), 1.64-1.76 (m, 1H); 408.9 |
| 22 | Example 18; C24, P3 | 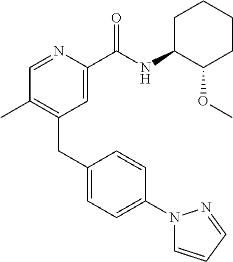 | 8.31 (s, 1H), 8.02 (s, 1H), 8.0- 8.08 (br s, 1H), 7.90 (d, J = 2 Hz, 1H), 7.71-7.73 (m, 1 H), 7.61 (br d, J = 8.5 Hz, 2H), 7.20 (br d, J = 8 Hz, 2H), 6.46 (dd, J = 2, 2 Hz, 1H), 4.06 (s, 2H), 3.93-4.03 (m, 1H), 3.38 (s, 3H), 3.20-3.27 (m, 1H), 2.29 (s, 3H), 2.15-2.23 (m, 1H), 2.06-2.14 (m, 1H), 1.75-1.83 (m, 1H), 1.64-1.72 (m, 1H), 1.3-1.47 (m, 4H); 405.0 |
| 23 | Example 18; C29 | 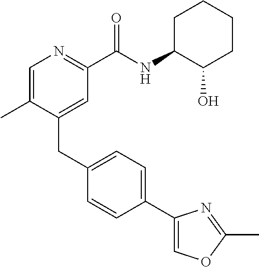 | 8.29 (s, 1H), 7.99-8.07 (m, 2H), 7.78 (s, 1H), 7.62 (d, J = 8.0 Hz, 2H), 7.13 (d, J = 7.8 Hz, 2H), 4.04 (s, 2H), 3.75-3.87 (m, 1H), 3.60-3.68 (m, 1H), 3.45-3.55 (m, 1H), 2.51 (s, 3H), 2.28 (s, 3H), 2.02-2.17 (m, 2H), 1.72- 1.82 (m, 2H), 1.23-1.48 (m, 4H); 405.9 |
| 24 | C24³ | 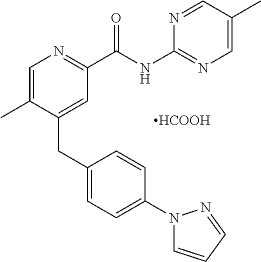 | 3.09 minutes⁴; 385 |
| 25 | Example 14; C34 | 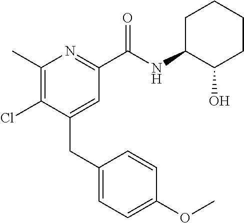 [SMILES: COc1ccc(Cc2cc(C(=O)N[C@H]3CCCC[C@@H]3O)nc(C)c2Cl)cc1] | ¹H NMR (400 MHz, CD₃OD) δ 7.74 (s, 1H), 7.13 (br d, J = 8.7 Hz, 2H), 6.86 (br d, J = 8.7 Hz, 2H), 4.11 (s, 2H), 3.76 (s, 3H), 3.67-3.76 (m, 1H), 3.48-3.57 (m, 1H), 2.67 (s, 3H), 1.95-2.08 (m, 2H), 1.67-1.80 (m, 2H), 1.30-1.44 (m, 4H); 389.0 |
| 26 | Method A; C24 | 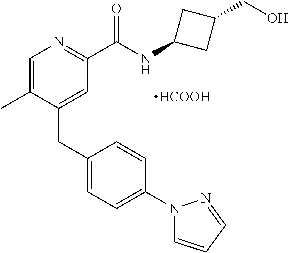 [SMILES: Cc1cnc(C(=O)N[C@H]2C[C@H](CO)C2)cc1Cc1ccc(-n2cccn2)cc1 ; "·HCOOH"] | 2.94 minutes⁴; 377 |
| 27 | Method A; C24 | 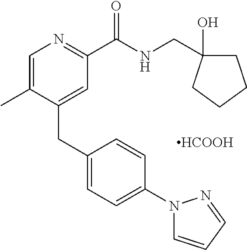 [SMILES: Cc1cnc(C(=O)NCC2(O)CCCC2)cc1Cc1ccc(-n2cccn2)cc1 ; "·HCOOH"] | 2.95 minutes⁵; 391 |
| 28 | Method A; C24 | 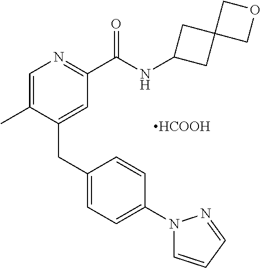 [SMILES: Cc1cnc(C(=O)NC2CC3(COC3)C2)cc1Cc1ccc(-n2cccn2)cc1 ; "·HCOOH"] | 3.03 minutes⁴; 389 |
| 29 | Method A; C24 | 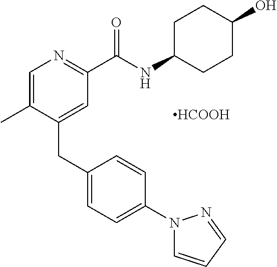 [SMILES: Cc1cnc(C(=O)N[C@H]2CC[C@@H](O)CC2)cc1Cc1ccc(-n2cccn2)cc1 ; "·HCOOH"] | 2.92 minutes⁴; 391 |
| 30 | Method A; C24 | 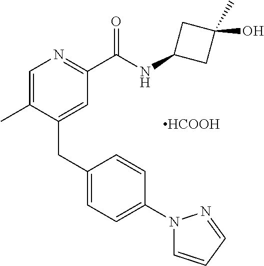 [SMILES: Cc1cnc(C(=O)N[C@H]2C[C@@](C)(O)C2)cc1Cc1ccc(-n2cccn2)cc1 ; "·HCOOH"] | 2.66 minutes⁵; 377 |
| 31 | Method A; C24 | 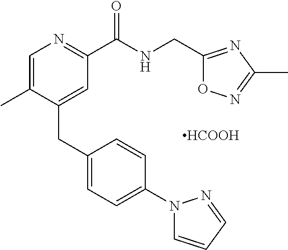 | 3.11 minutes⁴; 389 |
| 32 | Method A; C24 | 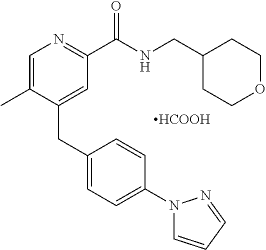 [SMILES: Cc1cnc(C(=O)NCC2CCOCC2)cc1Cc1ccc(-n2cccn2)cc1 ; "·HCOOH"] | 3.12 minutes⁴; 391 |
| 33 | Example 16; C24 | 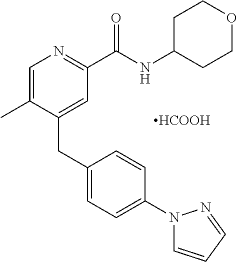 [SMILES: Cc1cnc(C(=O)NC2CCOCC2)cc1Cc1ccc(-n2cccn2)cc1 ; "·HCOOH"] | 3.05 minutes⁴; 377 |
| 34 | Example 16; C24 | 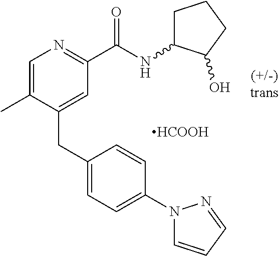 [SMILES: Cc1cnc(C(=O)NC2CCCC2O)cc1Cc1ccc(-n2cccn2)cc1 ; "·HCOOH"] | 3.03 minutes⁴; 377 |
| 35 | Example 16; C24 | 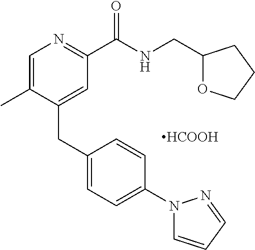 [SMILES: Cc1cnc(C(=O)NCC2CCCO2)cc1Cc1ccc(-n2cccn2)cc1 ; "·HCOOH"] | 2.97 minutes⁵; 377 |
| 36 | Example 16; C24 | 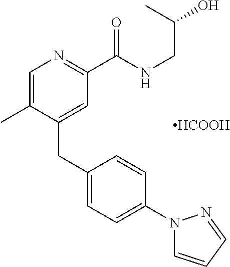 [SMILES: Cc1cnc(C(=O)NC[C@H](C)O)cc1Cc1ccc(-n2cccn2)cc1 ; "·HCOOH"] | 2.85 minutes⁴; 351 |
| 37 | Example 16⁶; C24 | 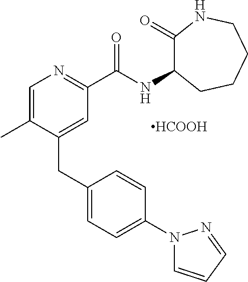 [SMILES: Cc1cnc(C(=O)N[C@@H]2CCCCNC2=O)cc1Cc1ccc(-n2cccn2)cc1 ; "·HCOOH"] | 3.05 minutes⁴; 404 |
| 38 | Example 16; C24 | 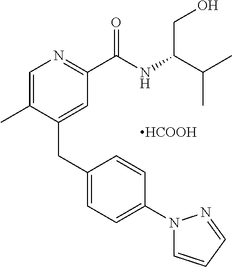 [SMILES: Cc1cnc(C(=O)N[C@H](CO)C(C)C)cc1Cc1ccc(-n2cccn2)cc1 ; "·HCOOH"] | 2.93 minutes⁵; 379 |
| 39 | Example 16; C24 | 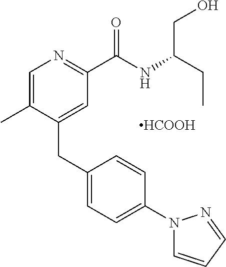 [SMILES: CC[C@@H](CO)NC(=O)c1cc(Cc2ccc(-n3cccn3)cc2)c(C)cn1 ; "·HCOOH"] | 3.00 minutes⁴; 365 |
| 40 | Example 16; C24 | 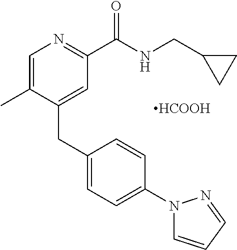 [SMILES: Cc1cnc(C(=O)NCC2CC2)cc1Cc1ccc(-n2cccn2)cc1 ; "·HCOOH"] | 3.15 minutes⁵; 347 |
| 41 | Example 16; C24 | 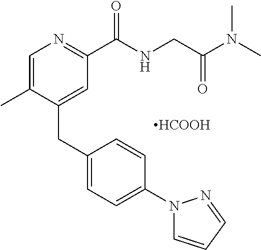 | 2.91 minutes⁴; 378 |
| 42 | Example 16⁶; C24 | 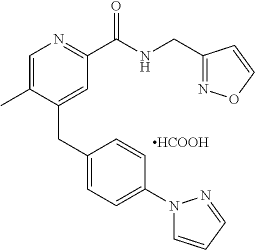 [SMILES: Cc1cnc(C(=O)NCc2ccon2)cc1Cc1ccc(-n2cccn2)cc1 ; "·HCOOH"] | 3.10 minutes⁴; 374 |
| 43 | Example 16; C24 | 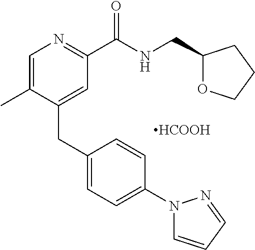 [SMILES: Cc1cnc(C(=O)NC[C@H]2CCCO2)cc1Cc1ccc(-n2cccn2)cc1 ; "·HCOOH"] | 2.97 minutes⁵; 377 |
| 44 | Example 16; C24 | 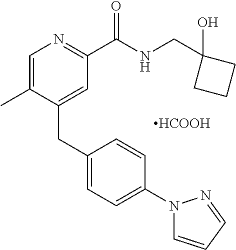 [SMILES: Cc1cnc(C(=O)NCC2(O)CCC2)cc1Cc1ccc(-n2cccn2)cc1 ; "·HCOOH"] | 3.05 minutes⁴; 377 |
| 45 | Example 16⁶; C24 | 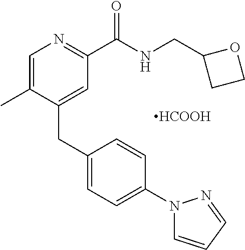 [SMILES: Cc1cnc(C(=O)NCC2CCO2)cc1Cc1ccc(-n2cccn2)cc1 ; "·HCOOH"] | 2.978 minutes⁴; 363 |
| 46 | Example 14⁷; C24 | 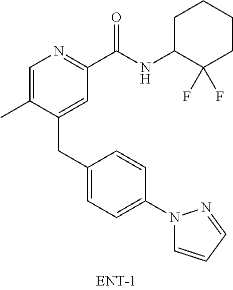 [SMILES: Cc1cnc(C(=O)NC2CCCCC2(F)F)cc1Cc1ccc(-n2cccn2)cc1] | 5.74 minutes⁸; 411.2 |
| 47 | Example 14⁷; C24 | 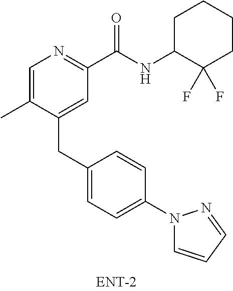 [SMILES: Cc1cnc(C(=O)NC2CCCCC2(F)F)cc1Cc1ccc(-n2cccn2)cc1] | 5.80 minutes⁸; 411.2 |
| 48 | Example 16⁶; C24 | 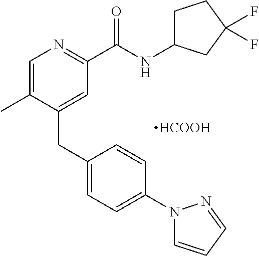 [SMILES: Cc1cnc(C(=O)NC2CCC(F)(F)C2)cc1Cc1ccc(-n2cccn2)cc1 ; "·HCOOH"] | 3.25 minutes⁵; 397 |
| 49 | Example 16⁶; C24 | 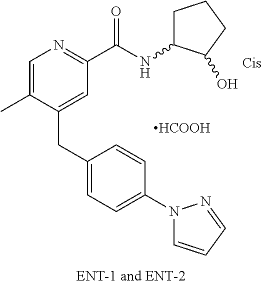 [SMILES: Cc1cnc(C(=O)NC2CCCC2O)cc1Cc1ccc(-n2cccn2)cc1 ; "·HCOOH"] | 3.045 minutes⁴; 377 |
| 50 | Example 14⁹; C9 | 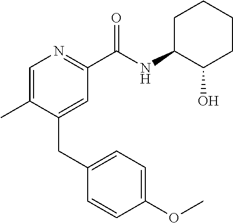 [SMILES: COc1ccc(Cc2cc(C(=O)N[C@H]3CCCC[C@@H]3O)ncc2C)cc1] | 8.28 (s, 1H), 8.02 (br d, J = 6.8 Hz, 1H), 7.96 (s, 1H), 7.03 (br d, J = 8.5 Hz, 2H), 6.82 (br d, J = 8.7 Hz, 2H), 3.96 (s, 2H), 3.79 (s, 3H), 3.76-3.85 (m, 1H), 3.49 (ddd, J = 10, 10, 4 Hz, 1H), 2.29 (s, 3H), 2.02-2.17 (m, 2H), 1.72-1.82 (m, 2H), 1.28-1.48 (m, 4H); 354.9 |
| 51 | Example 50; C9 | 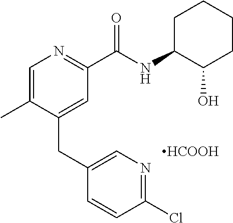 [SMILES: Cc1cnc(C(=O)N[C@H]2CCCC[C@@H]2O)cc1Cc1ccc(Cl)nc1 ; "·HCOOH"] | ¹H NMR (400 MHz, CD₃OD) δ 8.43 (s, 1H), 8.25 (d, J = 2 Hz, 1H), 7.80 (s, 1H), 7.62 (dd, J = 8, 2 Hz, 1H), 7.41 (d, J = 8.2 Hz, 1H), 4.14 (s, 2H), 3.68-3.79 (m, 1H), 3.47-3.57 (m, 1H), 2.36 (s, 3H), 1.96-2.09 (m, 2H), 1.67- 1.82 (m, 2H), 1.28-1.46 (m, 4H); 359.9 |
| 52 | Example 1; C5 | 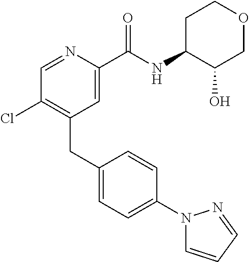 [SMILES: O=C(N[C@H]1CCOC[C@@H]1O)c1cc(Cc2ccc(-n3cccn3)cc2)c(Cl)cn1] | 8.50 (s, 1H), 8.05 (s, 1H), 8.00 (br d, J = 6 Hz, 1H), 7.91 (d, J = 2 Hz, 1H), 7.71-7.74 (m, 1H), 7.65 (br d, J = 8.5 Hz, 2H), 7.29 (br d, J = 8.5 Hz, 2H), 6.46-6.48 (m, 1H), 4.19 (s, 2H), 3.89-4.12 (m, 4H), 3.58-3.67 (m, 1H), 3.43-3.52 (m, 1H), 3.22 (dd, J = 11.3, 10.0 Hz, 1H), 2.00-2.07 (m, 1H), 1.72-1.84 (m, 1H); 434.9 [M + Na⁺] |
| 53 | Example 9¹⁰ | 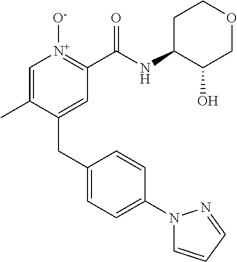 [SMILES: Cc1c[n+]([O-])c(C(=O)N[C@H]2CCOC[C@@H]2O)cc1Cc1ccc(-n2cccn2)cc1] | 11.73 (br d, J = 6 Hz, 1H), 8.20 (s, 1H), 8.11 (s, 1H), 7.90-7.92 (m, 1H), 7.71-7.74 (m, 1H), 7.65 (br d, J = 8 Hz, 2H), 7.20 (br d, J = 8 Hz, 2H), 6.46-6.49 (m, 1H), 4.05 (s, 2H), 3.94-4.10 (m, 3H), 3.68 (ddd, J = 9.3, 9.3, 4.9 Hz, 1H), 3.42-3.51 (m, 1H), 3.23 (dd, J = 10.5, 10.3 Hz, 1H), 2.27 (s, 3H), 2.02-2.11 (m, 1H), 1.74-1.86 (m, 1H)¹¹; 409.3 |
| 54 | Examples 8 and 9¹²; P2 | 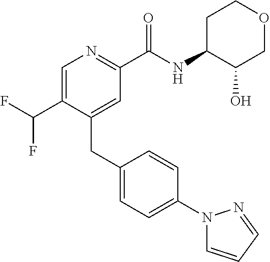 [SMILES: O=C(N[C@H]1CCOC[C@@H]1O)c1cc(Cc2ccc(-n3cccn3)cc2)c(C(F)F)cn1] | 8.71 (s, 1H), 8.10-8.16 (m, 1H), 8.08 (s, 1H), 7.89-7.93 (m, 1H), 7.71-7.74 (m, 1H), 7.67 (d, J = 8.3 Hz, 2H), 7.23 (d, J = 8.0 Hz, 2H), 6.83 (t, J_{HF} = 54.5 Hz, 1H), 6.45-6.50 (m, 1H), 4.24 (s, 2H), 4.09 (dd, J = 11, 5 Hz, 1H), 3.90-4.04 (m, 3H), 3.60-3.68 (m, 1H), 3.44-3.52 (m, 1H), 3.19-3.27 (m, 1H), 2.01-2.09 (m, 1H), 1.74-1.86 (m, 1H); 429.1 |

1. Reaction of [4-(5-methyl-1,2,4-oxadiazol-3-yl)phenyl]methanol with thionyl chloride provided 3-[4-(chloromethyl)phenyl]-5-methyl-1,2,4-oxadiazole, which was subjected to a Suzuki reaction with C10, mediated by tetrakis(triphenylphosphine)palladium(0), to afford the requisite ethyl 5-methyl-4-[4-(5-methyl-1,2,4-oxadiazol-3-yl)benzyl]pyridine-2-carboxylate.
2. Examples 20 and 21 were synthesized as the racemic mixture. Separation was carried out via reversed phase HPLC (Column: Chiral Technologies Chiralpak AD, 10 μm; Mobile phase: 55% ethanol in aqueous ammonia). The indicated absolute configurations were assigned on the basis of the relative biological activity of these two compounds (see Table 7), with reference to the known configurations and relative biological activity of Examples 8 and 9. Compound 20 exhibited a retention time of 0.73 minutes (and designated as trans, ENT-1), while 21 eluted at 1.37 minutes (and designated as trans, ENT-2), in the following supercritical fluid chromatographic system: Column: Chiral Technologies Chiralpak AD-3, 4.6 × 50 mm, 3 μm; Mobile phase: 3:2 [methanol, containing 0.05% diethylamine]/carbon dioxide; Flow rate: 3 mL/minute.
3. Compound C24 was converted to its methyl ester via treatment with hydrogen chloride in methanol at 60° C. 5-Methylpyrimidin-2-amine and trimethylaluminum were combined in toluene and tetrahydrofuran, and heated at 30° C. for 16 hours. The methyl ester was then added, and the reaction mixture was heated at 80° C. to provide the product.
4. Conditions for analytical HPLC. Column: Waters XBridge C18, 2.1 × 50 mm, 5 μm; Mobile phase A: 0.0375% trifluoroacetic acid in water; Mobile phase B: 0.01875% trifluoroacetic acid in acetonitrile; Gradient: 1% to 5% B over 0.6 minutes; 5% to 100% B over 3.4 minutes; Flow rate: 0.8 mL/minute.
5. Conditions for analytical HPLC. Column: Waters XBridge C18, 2.1 × 50 mm, 5 μm; Mobile phase A: 0.0375% trifluoroacetic acid in water; Mobile phase B: 0.01875% trifluoroacetic acid in acetonitrile; Gradient: 10% to 100% B over 4.0 minutes; Flow rate: 0.8 mL/minute.
6. In this case, the column used for purification was a Dikma Diamonsil(2) C18, 5 μm.
7. Racemic 2,2-difluorocyclohexanamine was utilized; separation of enantiomers 46 and 47 was carried out via supercritical fluid chromatography (Column: Phenomenex Amylose-2, 5 μm; Mobile phase: 4:1 carbon dioxide/methanol). Example 46 was the first-eluting enantiomer, followed by Example 47. Examples 46 and 47 are designated according to their respective retention time.
8. Conditions for analytical supercritical fluid HPLC. Column: Chiral Technologies Chiralcel OJ-H, 4.6 × 100 mm, 5 μm; Mobile phase: 4:1 carbon dioxide/methanol; Flow rate: 1.5 mL/minute.
9. Compound C9 was reacted at elevated temperature with chloro(4-methoxybenzyl)zinc in the presence of bis(tri-tert-butylphosphine)palladium(0) to provide the requisite ethyl 4-(4-methoxybenzyl)-5-methylpyridine-2-carboxylate.
10. The compound of Example 9 was oxidized with 3-chloroperoxybenzoic acid to provide Example 53.
11. This NMR data was obtained on material isolated after chromatography on silica gel, but before the final HPLC purification.
12. The requisite ethyl 5-(difluoromethyl)-4-(4,4,5,5-tetramethyl-1,3,2-dioxaborolan-2-yl)pyridine-2-carboxylate was prepared in the following manner: ethyl 4-chloro-5-methylpyridine-2-carboxylate was converted to ethyl 4-chloro-5-(hydroxymethyl)pyridine-2-carboxylate using the method described by L. F. Tietze et al., Chem. Eur. J. 2008, 14, 2527-2535. Dess-Martin oxidation to the corresponding aldehyde was followed by reaction with (diethylamino)sulfur trifluoride to afford ethyl 4-chloro-5-(difluoromethyl)pyridine-2-carboxylate. Further reaction using the conditions described for conversion of C9 to C22 in Examples 8 and 9 provided the appropriate intermediate.

### Examples 55 and 56

### 4-{(S)-Fluoro[4-(1H-pyrazol-1-yl)phenyl]methyl}-N-[(3R,4S)-3-hydroxytetrahydro-2H-pyran-4-yl]-5-methylpyridine-2-carboxamide (55) and 4-{(R)-Fluoro[4-(1H-pyrazol-1-yl)phenyl]methyl}-N-[(3R,4S)-3-hydroxytetrahydro-2H-pyran-4-yl]-5-methylpyridine-2-carboxamide (56)

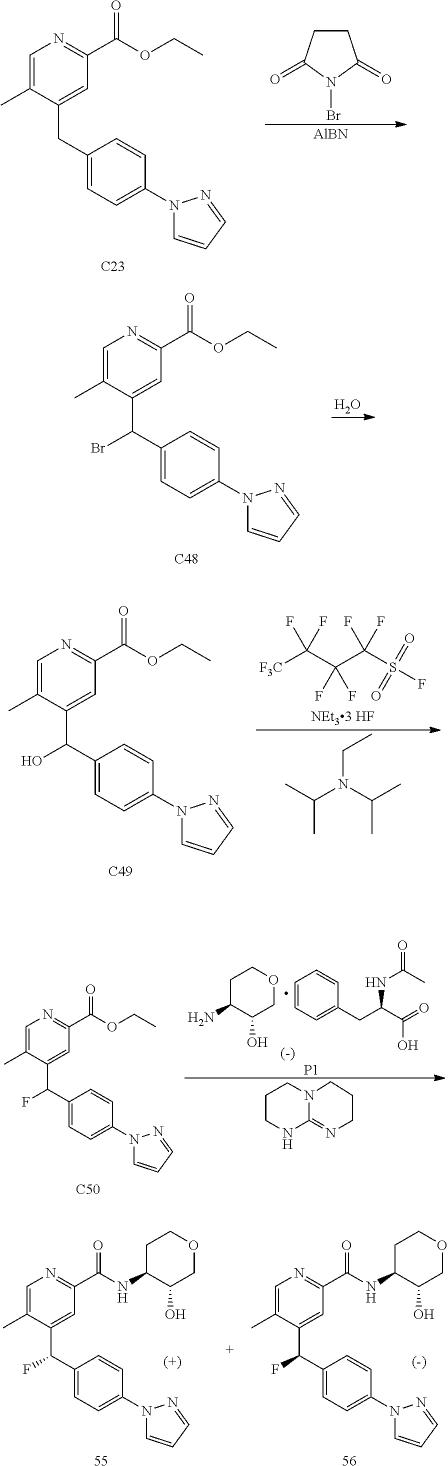

### Step 1. Synthesis of ethyl 4-{bromo[4-(1H-pyrazol-1-yl)phenyl]methyl}-5-methylpyridine-2-carboxylate (C48)

To a solution of C23 (2.00 g, 6.22 mmol) in tetrachloromethane (62 mL) was added N-bromosuccinimide (96%, 1.15 g, 6.20 mmol), followed by 2,2′-azobisisobutyronitrile (AIBN; 102 mg, 0.621 mmol). The reaction mixture was heated to 75° C. while being irradiated with a 75 watt fluorescent light bulb. After 1 hour, the reaction mixture was cooled to 0° C. and filtered; the filtrate was concentrated in vacuo and purified via chromatography on silica gel (Eluent: 35% ethyl acetate in heptane) to afford the product as a light yellow solid. Yield: 1.85 g, 4.62 mmol, 74%. ¹H NMR (400 MHz, CDCl₃) δ 8.52 (s, 1H), 8.37 (s, 1H), 7.92 (d, J=2.5 Hz, 1H), 7.71 (d, J=1.7 Hz, 1H), 7.68 (br d, J=8.7 Hz, 2H), 7.44 (br d, J=8.5 Hz, 2H), 6.46 (dd, J=2.4, 1.9 Hz, 1H), 6.35 (s, 1H), 4.47 (q, J=7.1 Hz, 2H), 2.35 (s, 3H), 1.43 (t, J=7.1 Hz, 3H).

### Step 2. Synthesis of ethyl 4-{hydroxy[4-(1H-pyrazol-1-yl)phenyl]methyl}-5-methylpyridine-2-carboxylate (C49)

Water (7 mL) was added to a solution of C48 (1.15 g, 2.87 mmol) in acetone (7 mL); the resulting white suspension was allowed to stir at room temperature for three hours. The reaction mixture was partitioned between water and ethyl acetate, and the organic layer was dried over magnesium sulfate, filtered, and concentrated in vacuo. Silica gel chromatography (Gradient: 50% to 100% ethyl acetate in heptane) afforded the product as a white solid. Yield: 706 mg, 2.09 mmol, 73%. LCMS m/z 338.1 [M+H]⁺. ¹H NMR (400 MHz, CDCl₃) δ 8.44 (s, 1H), 8.34-8.40 (m, 1H), 7.83-7.87 (m, 1H), 7.67-7.71 (m, 1H), 7.52-7.59 (m, 2H), 7.25-7.32 (m, 2H), 6.42-6.46 (m, 1H), 5.92 (s, 1H), 4.46 (q, J=7 Hz, 2H), 2.16 (s, 3H), 1.43 (t, J=7 Hz, 3H).

### Step 3. Synthesis of ethyl 4-{fluoro[4-(1H-pyrazol-1-yl)phenyl]methyl}-5-methylpyridine-2-carboxylate (C50)

1,1,2,2,3,3,4,4,4-Nonafluorobutane-1-sulfonyl fluoride (1.36 mL, 7.57 mmol) and triethylamine trihydrofluoride (1.24 mL, 7.61 mmol) were added to a solution of C49 (1.28 g, 3.79 mmol) in acetonitrile (7.6 mL). N,N-Diisopropylethylamine (4.0 mL, 23 mmol) was then introduced, and the reaction mixture was stirred at room temperature for 1 hour. After the reaction had been quenched, via addition of saturated aqueous sodium bicarbonate solution, the mixture was extracted with ethyl acetate; the organic layer was dried over magnesium sulfate, filtered, and concentrated under reduced pressure. Silica gel chromatography (Gradient: 0% to 100% ethyl acetate in heptane) provided the product as a yellow oil. Yield: 832 mg, 2.45 mmol, 65%. LCMS m/z 340.4 [M+H]⁺. ¹H NMR (400 MHz, CDCl₃) δ 8.54 (s, 1H), 8.32 (s, 1H), 7.91-7.95 (m, 1H), 7.68-7.76 (m, 3H), 7.33-7.40 (m, 2H), 6.57 (d, J_{HF}=47 Hz, 1H), 6.45-6.49 (m, 1H), 4.49 (q, J=7 Hz, 2H), 2.21 (s, 3H), 1.45 (t, J=7 Hz, 3H).

### Step 4. Synthesis of 4-{(S)-fluoro[4-(1H-pyrazol-1-yl)phenyl]methyl}-N-[(3R,4S)-3-hydroxytetrahydro-2H-pyran-4-yl]-5-methylpyridine-2-carboxamide (55) and 4-{(R)-fluoro[4-(1H-pyrazol-1-yl)phenyl]methyl}-N-[(3R,4S)-3-hydroxytetrahydro-2H-pyran-4-yl]-5-methylpyridine-2-carboxamide (56)

1,3,4,6,7,8-Hexahydro-2H-pyrimido[1,2-a]pyrimidine (95%, 289 mg, 1.97 mmol) was added to a solution of C50 (394 mg, 1.16 mmol) and P1 (377 mg, 1.16 mmol) in N,N-dimethylformamide (2.3 mL). The reaction mixture was heated to 75° C. overnight, whereupon it was cooled and partitioned between water and ethyl acetate. The aqueous layer was extracted with ethyl acetate, and the combined organic layers were washed with saturated aqueous sodium chloride solution, dried over magnesium sulfate, filtered, and concentrated in vacuo. The component diastereomers were separated using supercritical fluid chromatography [Column: Chiral Technologies Chiralpak IC, 5 μm; Mobile phase: 3:2 carbon dioxide/(25% methanol in ethyl acetate)].

The first-eluting enantiomer was 55, obtained as an off-white solid, which exhibited a positive (+) rotation. Yield: 140 mg, 0.341 mmol, 29%. LCMS m/z 411.5 [M+H]⁺. ¹H NMR (400 MHz, CDCl₃) δ 8.41 (s, 1H), 8.36 (s, 1H), 8.14 (br d, J=6.4 Hz, 1H), 7.94 (d, J=2.4 Hz, 1H), 7.71-7.76 (m, 3H), 7.37-7.41 (m, 2H), 6.58 (d, J_{HF}=47.1 Hz, 1H), 6.49 (dd, J=2.4, 1.8 Hz, 1H), 4.11 (dd, J=11.5, 5.0 Hz, 1H), 3.95-4.05 (m, 2H), 3.66 (ddd, J=9.6, 9.5, 5.0 Hz, 1H), 3.50 (ddd, J=11.9, 11.9, 2.2 Hz, 1H), 3.25 (dd, J=11.3, 10.0 Hz, 1H), 2.24 (s, 3H), 2.03-2.10 (m, 1H), 1.76-1.88 (m, 1H). This material was taken up in hot ethyl acetate and allowed to cool slowly until crystals were observed; one of these crystals of 55 was analyzed via X-ray crystallography (see below); this provided the relative configurations of the stereocenters in 55. Because the absolute configurations of the stereocenters in the (3R,4S)-4-aminotetrahydro-2H-pyran-3-ol moiety are known (see the single crystal X-ray determination of P1 above), the absolute configuration at the benzylic fluorine of 55 is thus established as shown.

The later-eluting diastereomer from the separation was therefore assigned as 56; this product was obtained as a light yellow oil. Yield: 143 mg, 0.348 mmol, 30%. LCMS m/z 411.5 [M+H]⁺. ¹H NMR (400 MHz, CDCl₃) δ 8.40 (s, 1H), 8.36 (s, 1H), 8.13 (br d, J=6 Hz, 1H), 7.94 (d, J=2.5 Hz, 1H), 7.70-7.75 (m, 3H), 7.36-7.41 (m, 2H), 6.58 (d, J_{HF}=47.1 Hz, 1H), 6.49 (dd, J=2.4, 1.8 Hz, 1H), 4.11 (dd, J=11.4, 4.8 Hz, 1H), 3.95-4.05 (m, 2H), 3.66 (ddd, J=9.6, 9.4, 5.0 Hz, 1H), 3.50 (ddd, J=11.9, 11.9, 2.2 Hz, 1H), 3.25 (dd, J=11.4, 9.8 Hz, 1H), 2.24 (s, 3H), 2.03-2.10 (m, 1H), 1.82 (dddd, J=13, 12, 12, 4.7 Hz, 1H). This material was dissolved in dichloromethane and slowly concentrated, providing an off-white solid that exhibited a negative (−) rotation.

### Single Crystal X-Ray Analysis of 55

Data collection was performed on a Bruker APEX diffractometer at room temperature. Data collection consisted of omega and phi scans.

The structure was solved by direct methods using SHELX software suite in the space group P2₁2₁2₁. The structure was subsequently refined by the full-matrix least squares method. All non-hydrogen atoms were found and refined using anisotropic displacement parameters.

The asymmetric unit was comprised of one molecule of 55.

The hydrogen atom located on nitrogen was found from the Fourier difference map and refined with distance restrained. The remaining hydrogen atoms were placed in calculated positions and were allowed to ride on their carrier atoms. The final refinement included isotropic displacement parameters for all hydrogen atoms.

The absolute configuration of the benzylic fluorine atom was determined in relation to the known stereocenters of the (3R,4S)-4-aminotetrahydro-2H-pyran-3-ol moiety (see the X-ray structure of P1 above).

The final R-index was 3%. A final difference Fourier revealed no missing or misplaced electron density.

Pertinent crystal, data collection and refinement information is summarized in Table E55-1. Atomic coordinates, bond lengths, bond angles, and displacement parameters are listed in Tables E55-2 to E55-5.

### SOFTWARE AND REFERENCES

SHELXTL, Version 5.1, Bruker AXS, 1997
PLATON, A. L. Spek, J. Appl. Cryst. 2003, 36, 7-13.
MERCURY, C. F. Macrae, P. R. Edington, P. McCabe, E. Pidcock, G. P. Shields, R. Taylor, M. Towler, and J. van de Streek, J. Appl. Cryst. 2006, 39, 453-457.
OLEX2, O. V. Dolomanov, L. J. Bourhis, R. J. Gildea, J. A. K. Howard, and H. Puschmann, J. Appl. Cryst. 2009, 42, 339-341.

TABLE E55-1
Crystal data and structure refinement for 55.

| Empirical formula | C₂₂H₂₃FN₄O₃ | |
|---|---|---|
| Formula weight | 410.44 | |
| Temperature | 296(2) K | |
| Wavelength | 1.54178 Å | |
| Crystal system | Orthorhombic | |
| Space group | P2₁2₁2₁ | |
| Unit cell dimensions | a = 7.4083(4) Å | α = 90°. |
| | b = 10.4675(5) Å | β = 90°. |
| | c = 25.6743(11) Å | γ = 90°. |

| | |
|---|---|
| Volume | 1990.95(17) Å³ |
| Z | 4 |
| Density (calculated) | 1.369 Mg/m³ |
| Absorption coefficient | 0.823 mm⁻¹ |
| F(000) | 864 |
| Crystal size | 0.18 × 0.18 × 0.06 mm³ |
| Theta range for data collection | 17.20 to 68.19° |
| Index ranges | −8 <= h <= 8, −12 <= |
| | k <= 12, −30 <= l <= 30 |
| Reflections collected | 18674 |
| Independent reflections | 3449 [R(int) = 0.0348] |
| Completeness to theta = 67.42° | 94.2% |
| Absorption correction | Empirical |
| Max. and min. transmission | 0.9523 and 0.8660 |
| Refinement method | Full-matrix least-squares on F² |
| Data/restraints/parameters | 3449/2/280 |
| Goodness-of-fit on F² | 1.048 |
| Final R indices [I > 2sigma(I)] | R1 = 0.0301, wR2 = 0.0829 |
| R indices (all data) | R1 = 0.0321, wR2 = 0.0849 |
| Absolute structure parameter | 0.12(14) |
| Largest diff. peak and hole | 0.107 and −0.095 e.Å⁻³ |

TABLE E55-2
Atomic coordinates (×10⁴) and equivalent isotropic displacement
parameters (Å² × 10³) for 55. U(eq) is defined as one third
of the trace of the orthogonalized U^{ij} tensor.

| | x | y | z | U(eq) |
|---|---|---|---|---|
| C(1) | 3013(2) | 2567(1) | 11212(1) | 50(1) |
| C(2) | 3197(3) | 1176(2) | 11388(1) | 65(1) |
| C(3) | 6179(3) | 1020(2) | 11103(1) | 63(1) |
| C(4) | 6172(2) | 2392(2) | 10906(1) | 58(1) |
| C(5) | 4266(2) | 2781(1) | 10754(1) | 45(1) |
| C(6) | 4543(2) | 4420(1) | 10083(1) | 43(1) |
| C(7) | 4438(2) | 5829(1) | 9970(1) | 42(1) |
| C(8) | 4030(2) | 7841(1) | 10276(1) | 50(1) |
| C(9) | 4247(2) | 8390(1) | 9786(1) | 46(1) |
| C(10) | 4605(2) | 7569(1) | 9370(1) | 45(1) |
| C(11) | 4694(2) | 6265(1) | 9468(1) | 45(1) |
| C(12) | 4099(3) | 9815(1) | 9718(1) | 61(1) |
| C(13) | 4880(2) | 8073(2) | 8825(1) | 54(1) |
| C(14) | 3143(2) | 8302(1) | 8532(1) | 50(1) |
| C(15) | 1885(3) | 7330(2) | 8480(1) | 60(1) |
| C(16) | 321(2) | 7495(2) | 8198(1) | 61(1) |
| C(17) | −7(2) | 8658(2) | 7959(1) | 52(1) |
| C(18) | 1225(3) | 9645(2) | 8007(1) | 59(1) |
| C(19) | 2795(2) | 9466(2) | 8295(1) | 56(1) |
| C(20) | −3984(3) | 9589(2) | 7278(1) | 80(1) |
| C(21) | −3895(3) | 8267(2) | 7227(1) | 77(1) |
| C(22) | −2503(3) | 9913(2) | 7558(1) | 70(1) |
| F(1) | 5899(2) | 7160(1) | 8551(1) | 76(1) |
| N(1) | 4195(2) | 4093(1) | 10574(1) | 48(1) |
| N(2) | 4109(2) | 6599(1) | 10376(1) | 47(1) |
| N(3) | −1615(2) | 8818(1) | 7668(1) | 58(1) |
| N(4) | −2468(2) | 7786(2) | 7462(1) | 70(1) |
| O(1) | 4981(2) | 846(1) | 11522(1) | 74(1) |
| O(2) | 1187(2) | 2877(1) | 11105(1) | 63(1) |
| O(3) | 4919(2) | 3643(1) | 9740(1) | 58(1) |

TABLE E55-3
Bond lengths [Å] and angles [°] for 55.

| | | | |
|---|---|---|---|
| C(1)—O(2) | 1.419(2) | C(8)—C(9) | 1.393(2) |
| C(1)—C(5) | 1.514(2) | C(9)—C(10) | 1.395(2) |
| C(1)—C(2) | 1.530(2) | C(9)—C(12) | 1.5063(19) |
| C(2)—O(1) | 1.408(2) | C(10)—C(11) | 1.3899(19) |
| C(3)—O(1) | 1.406(3) | C(10)—C(13) | 1.510(2) |
| C(3)—C(4) | 1.522(2) | C(13)—F(1) | 1.406(2) |
| C(4)—C(5) | 1.520(2) | C(13)—C(14) | 1.508(2) |
| C(5)—N(1) | 1.4501(16) | C(14)—C(19) | 1.386(2) |
| C(6)—O(3) | 1.2323(17) | C(14)—C(15) | 1.387(2) |
| C(6)—N(1) | 1.3311(18) | C(15)—C(16) | 1.377(3) |
| C(6)—C(7) | 1.5051(18) | C(16)—C(17) | 1.385(2) |
| C(7)—N(2) | 1.3400(18) | C(17)—C(18) | 1.384(2) |
| C(7)—C(11) | 1.3796(19) | C(17)—N(3) | 1.416(2) |
| C(8)—N(2) | 1.3262(18) | C(18)—C(19) | 1.391(2) |
| C(20)—C(22) | 1.356(3) | C(11)—C(10)—C(13) | 120.32(12) |
| C(20)—C(21) | 1.391(3) | C(9)—C(10)—C(13) | 121.31(12) |
| C(21)—N(4) | 1.316(3) | C(7)—C(11)—C(10) | 119.16(12) |
| C(22)—N(3) | 1.351(2) | F(1)—C(13)—C(14) | 108.52(13) |
| N(3)—N(4) | 1.359(2) | F(1)—C(13)—C(10) | 107.35(12) |
| O(2)—C(1)—C(5) | 113.58(12) | C(14)—C(13)—C(10) | 113.71(12) |
| O(2)—C(1)—C(2) | 111.11(13) | C(19)—C(14)—C(15) | 118.52(15) |
| C(5)—C(1)—C(2) | 108.43(12) | C(19)—C(14)—C(13) | 121.15(14) |
| O(1)—C(2)—C(1) | 112.90(14) | C(15)—C(14)—C(13) | 120.29(13) |
| O(1)—C(3)—C(4) | 112.00(14) | C(16)—C(15)—C(14) | 121.65(14) |
| C(5)—C(4)—C(3) | 109.92(14) | C(15)—C(16)—C(17) | 119.38(15) |
| N(1)—C(5)—C(1) | 111.44(11) | C(16)—C(17)—C(18) | 120.04(15) |
| N(1)—C(5)—C(4) | 111.66(12) | C(16)—C(17)—N(3) | 119.06(15) |
| C(1)—C(5)—C(4) | 109.34(12) | C(18)—C(17)—N(3) | 120.89(13) |
| O(3)—C(6)—N(1) | 123.46(12) | C(17)—C(18)—C(19) | 119.91(14) |
| O(3)—C(6)—C(7) | 121.38(12) | C(14)—C(19)—C(18) | 120.49(15) |
| N(1)—C(6)—C(7) | 115.17(11) | C(22)—C(20)—C(21) | 105.06(18) |
| N(2)—C(7)—C(11) | 123.55(12) | N(4)—C(21)—C(20) | 112.08(19) |
| N(2)—C(7)—C(6) | 116.64(11) | N(3)—C(22)—C(20) | 107.03(19) |
| C(11)—C(7)—C(6) | 119.81(12) | C(6)—N(1)—C(5) | 122.58(11) |
| N(2)—C(8)—C(9) | 124.96(13) | C(8)—N(2)—C(7) | 116.60(12) |
| C(8)—C(9)—C(10) | 117.37(12) | C(22)—N(3)—N(4) | 111.46(16) |
| C(8)—C(9)—C(12) | 120.26(14) | C(22)—N(3)—C(17) | 128.35(15) |
| C(10)—C(9)—C(12) | 122.37(13) | N(4)—N(3)—C(17) | 120.19(13) |
| C(11)—C(10)—C(9) | 118.36(12) | C(21)—N(4)—N(3) | 104.37(16) |
| | | C(3)—O(1)—C(2) | 112.01(12) |

### Symmetry Transformations Used to Generate Equivalent Atoms.

TABLE E55-4
Anisotropic displacement parameters (Å² × 10³) for 55.
The anisotropic displacement factor exponent takes the
form: −2π²[h² a*²U¹¹ + . . . + 2 h k a* b* U¹²].

| | U¹¹ | U²² | U³³ | U²³ | U¹³ | U¹² |
|---|---|---|---|---|---|---|
| C (1) | 56 (1) | 51 (1) | 44 (1) | −3 (1) | 0 (1) | −1 (1) |
| C (2) | 69 (1) | 64 (1) | 63 (1) | 17 (1) | 4 (1) | −7 (1) |
| C (3) | 62 (1) | 52 (1) | 76 (1) | 11 (1) | −11 (1) | 7 (1) |
| C (4) | 53 (1) | 49 (1) | 72 (1) | 5 (1) | −1 (1) | 3 (1) |
| C (5) | 53 (1) | 37 (1) | 44 (1) | 1 (1) | −2 (1) | 2 (1) |
| C (6) | 40 (1) | 41 (1) | 49 (1) | −1 (1) | −2 (1) | 3 (1) |
| C (7) | 36 (1) | 40 (1) | 50 (1) | 1 (1) | 0 (1) | 2 (1) |
| C (8) | 51 (1) | 42 (1) | 56 (1) | −4 (1) | 2 (1) | 2 (1) |
| C (9) | 37 (1) | 41 (1) | 60 (1) | 3 (1) | −3 (1) | 0 (1) |
| C (10) | 33 (1) | 47 (1) | 54 (1) | 6 (1) | 0 (1) | 1 (1) |
| C (11) | 41 (1) | 44 (1) | 49 (1) | −1 (1) | 1 (1) | 3 (1) |
| C (12) | 64 (1) | 41 (1) | 79 (1) | 7 (1) | 1 (1) | −2 (1) |
| C (13) | 48 (1) | 54 (1) | 60 (1) | 10 (1) | 7 (1) | −1 (1) |
| C (14) | 54 (1) | 51 (1) | 46 (1) | 9 (1) | 7 (1) | 1 (1) |
| C (15) | 66 (1) | 51 (1) | 64 (1) | 20 (1) | −3 (1) | −5 (1) |
| C (16) | 65 (1) | 55 (1) | 62 (1) | 13 (1) | −5 (1) | −9 (1) |
| C (17) | 60 (1) | 56 (1) | 40 (1) | 4 (1) | 2 (1) | 4 (1) |
| C (18) | 73 (1) | 46 (1) | 56 (1) | 11 (1) | −2 (1) | 4 (1) |
| C (19) | 63 (1) | 47 (1) | 58 (1) | 10 (1) | 1 (1) | −4 (1) |
| C (20) | 73 (1) | 105 (2) | 62 (1) | 2 (1) | −12 (1) | 20 (1) |
| C (21) | 73 (1) | 95 (1) | 64 (1) | 3 (1) | −15 (1) | −6 (1) |
| C (22) | 78 (1) | 73 (1) | 60 (1) | −5 (1) | −7 (1) | 20 (1) |
| F (1) | 65 (1) | 96 (1) | 66 (1) | 13 (1) | 22 (1) | 22 (1) |
| N (1) | 58 (1) | 37 (1) | 49 (1) | 1 (1) | 3 (1) | 5 (1) |
| N (2) | 50 (1) | 43 (1) | 50 (1) | 1 (1) | 2 (1) | 2 (1) |
| N (3) | 67 (1) | 63 (1) | 44 (1) | 3 (1) | −3 (1) | 8 (1) |
| N (4) | 79 (1) | 70 (1) | 62 (1) | 3 (1) | −12 (1) | −8 (1) |
| O (1) | 80 (1) | 71 (1) | 70 (1) | 27 (1) | −12 (1) | 4 (1) |
| O (2) | 54 (1) | 69 (1) | 66 (1) | −4 (1) | 6 (1) | 5 (1) |
| O (3) | 76 (1) | 45 (1) | 53 (1) | −3 (1) | 4 (1) | 7 (1) |

TABLE E55-5
Hydrogen coordinates (×10⁴) and isotropic displacement
parameters (Å² × 10³) for 55.

| | x | y | z | U(eq) |
|---|---|---|---|---|
| H(1) | 3422 | 3114 | 11498 | 60 |
| H(2A) | 2423 | 1036 | 11688 | 79 |
| H(2B) | 2786 | 620 | 11111 | 79 |
| H(3A) | 5842 | 452 | 10820 | 76 |
| H(3B) | 7391 | 795 | 11213 | 76 |
| H(4A) | 6620 | 2957 | 11176 | 69 |
| H(4B) | 6963 | 2466 | 10606 | 69 |
| H(5) | 3871 | 2227 | 10468 | 54 |
| H(8) | 3814 | 8388 | 10555 | 60 |
| H(11) | 4924 | 5693 | 9199 | 54 |
| H(12A) | 5265 | 10160 | 9635 | 92 |
| H(12B) | 3668 | 10192 | 10035 | 92 |
| H(12C) | 3271 | 10002 | 9441 | 92 |
| H(13) | 5563 | 8874 | 8842 | 65 |
| H(15) | 2102 | 6548 | 8640 | 73 |
| H(16) | −507 | 6831 | 8169 | 73 |
| H(18) | 1004 | 10426 | 7847 | 70 |
| H(19) | 3616 | 10133 | 8329 | 67 |
| H(20) | −4868 | 10132 | 7147 | 96 |
| H(21) | −4743 | 7780 | 7049 | 93 |
| H(22) | −2163 | 10733 | 7657 | 84 |
| H(99A) | 4050(30) | 4755(16) | 10803(7) | 57(5) |
| H(99B) | 900(50) | 2370(30) | 10807(10) | 121(10) |

### Example 57

### N-[(3R,4S)-3-Hydroxytetrahydro-2H-pyran-4-yl]-5-methyl-4-[4-(1,3-oxazol-4-yl)benzyl]pyridine-2-carboxamide (57)

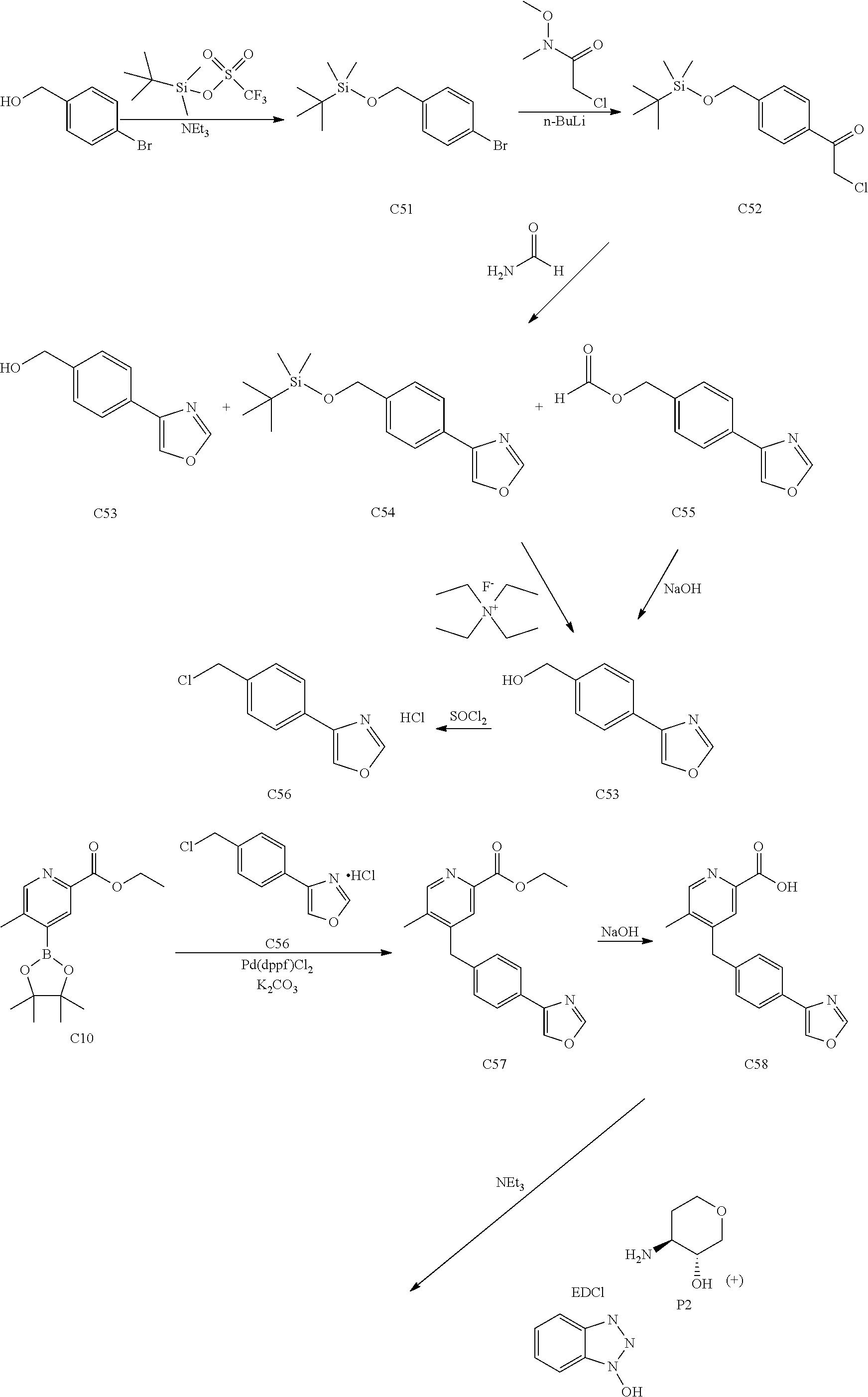

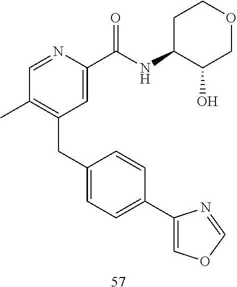 [SMILES: Cc1cnc(C(=O)N[C@H]2CCOC[C@@H]2O)cc1Cc1ccc(-c2cocn2)cc1 ; "57"]

### Step 1. Synthesis of [(4-bromobenzyl)oxy](tert-butyl)dimethylsilane (C51)

Triethylamine (27.1 g, 268 mmol) and tert-butyl(dimethyl)silyl trifluoromethanesulfonate (53 g, 200 mmol) were added to a solution of (4-bromophenyl)methanol (25.0 g, 133 mmol) in dichloromethane (500 mL), and the reaction mixture was stirred at 15° C. for 18 hours. After the addition of saturated aqueous ammonium chloride solution (500 mL), the mixture was extracted with dichloromethane (2×300 mL), and the combined organic layers were dried over sodium sulfate, filtered, and concentrated in vacuo. Chromatography on silica gel (Eluent: petroleum ether) provided the product as a colorless oil. Yield: 34.6 g, 115 mmol, 86%. ¹H NMR (400 MHz, CDCl₃) δ 7.46 (d, J=8.4 Hz, 2H), 7.21 (d, J=8.4 Hz, 2H), 4.70 (s, 2H), 0.95 (s, 9H), 0.11 (s, 6H).

### Step 2. Synthesis of 1-[4-({[tert-butyl(dimethyl)silyl]oxy}methyl)phenyl]-2-chloroethanone (C52)

To a −78° C. solution of C51 (10.0 g, 33.2 mmol) in tetrahydrofuran (120 mL) was added n-butyllithium (2.5 M in hexanes, 15.9 mL, 39.8 mmol). After the reaction mixture had stirred at −78° C. for one hour, a solution of 2-chloro-N-methoxy-N-methylacetamide (5.48 g, 39.8 mmol) in tetrahydrofuran (100 mL) was added in a drop-wise manner, while the reaction mixture was maintained at −78° C. Stirred was continued at −40° C. to −50° C. for 1 hour, whereupon the reaction was quenched by addition of saturated aqueous ammonium chloride solution (200 mL) at −40° C. to −20° C. The aqueous phase was extracted with ethyl acetate (3×200 mL), and the combined organic layers were dried over sodium sulfate, filtered, and concentrated under reduced pressure. The residue was purified by silica gel chromatography (Gradient: 1% to 15% ethyl acetate in petroleum ether) to provide the product as a colorless gum, which became a white solid upon standing. Yield: 8.50 g, 28.4 mmol, 86%. ¹H NMR (400 MHz, CDCl₃) δ 7.94 (d, J=8.3 Hz, 2H), 7.46 (d, J=8.0 Hz, 2H), 4.81 (s, 2H), 4.72 (s, 2H), 0.96 (s, 9H), 0.12 (s, 6H).

### Step 3. Synthesis of [4-(1,3-oxazol-4-yl)phenyl]methanol (C53), 4-[4-({[tert-butyl(dimethyl)silyl]oxy}methyl)phenyl]-1,3-oxazole (C54), and 4-(1,3-oxazol-4-yl)benzyl formate (C55)

A solution of C52 (3.50 g, 11.7 mmol) in formamide (20 mL) was heated at 100° C. for 18 hours. After the reaction mixture had cooled, saturated aqueous sodium bicarbonate solution (50 mL) was added, and the mixture was extracted with ethyl acetate (3×50 mL). The combined organic layers were concentrated in vacuo and purified via silica gel chromatography (Gradient: 0% to 50% ethyl acetate in petroleum ether) to provide the three products.

Compound C53 was obtained as a yellow solid. Yield: 300 mg, 1.7 mmol, 14%. ¹H NMR (400 MHz, CDCl₃) δ 7.94 (s, 1H), 7.92 (s, 1H), 7.71 (d, J=8.2 Hz, 2H), 7.39 (d, J=8.2 Hz, 2H), 4.70 (s, 2H).

Compound C54 was isolated as a red gum. Yield: 200 mg, 0.69 mmol, 6%. ¹H NMR (400 MHz, CDCl₃) δ 7.94 (s, 2H), 7.73 (br d, J=8.3 Hz, 2H), 7.38 (br d, J=8 Hz, 2H), 4.78 (s, 2H), 0.96 (s, 9H), 0.12 (s, 6H).

Compound C55 was obtained as a red solid. Yield: 600 mg, 3.0 mmol, 26%. ¹H NMR (400 MHz, CDCl₃) δ 8.17 (t, J=0.8 Hz, 1H), 7.98 (d, J=0.9 Hz, 1H), 7.96 (d, J=0.8 Hz, 1H), 7.78 (br d, J=8.4 Hz, 2H), 7.44 (br d, J=8 Hz, 2H), 5.24 (s, 2H).

### Step 4. Synthesis of [4-(1,3-oxazol-4-yl)phenyl]methanol (C53) from 4-[4-({[tert-butyl(dimethyl)silyl]oxy}methyl)phenyl]-1,3-oxazole (C54)

Tetraethylammonium fluoride hydrate (347 mg, 2.07 mmol) was added to a solution of C54 (400 mg, 1.4 mmol) in tetrahydrofuran (6 mL), and the reaction mixture was stirred at 50° C. for 3 hours. After the solvent had been removed under reduced pressure, the residue was subjected to silica gel chromatography (Gradient: 0% to 50% ethyl acetate in petroleum ether) to provide the product as a yellow solid. Yield: 180 mg, 1.0 mmol, 71%. LCMS m/z 175.8 [M+H]⁺.

### Step 5. Synthesis of [4-(1,3-oxazol-4-yl)phenyl]methanol (C53) from 4-(1,3-oxazol-4-yl)benzyl formate (C55)

To a solution of C55 (1.1 g, 5.4 mmol) in a mixture of tetrahydrofuran and water (1:1, 10 mL) was added sodium hydroxide (433 mg, 10.8 mmol). The reaction mixture was stirred at 18° C. for 1 hour, whereupon it was extracted with ethyl acetate (3×5 mL). The combined organic layers were washed with saturated aqueous sodium chloride solution, concentrated in vacuo, and purified using chromatography on silica gel (Gradient: 0% to 50% ethyl acetate in petroleum ether) to afford the product as a yellow solid. Yield: 820 mg, 4.7 mmol, 87%. LCMS m/z 175.8 [M+H]⁺. ¹H NMR (400 MHz, CDCl₃) δ 7.97 (s, 1H), 7.96 (s, 1H), 7.76 (d, J=8.2 Hz, 2H), 7.44 (d, J=7.9 Hz, 2H), 4.74 (s, 2H).

### Step 6. Synthesis of 4-[4-(chloromethyl)phenyl]-1,3-oxazole, hydrochloride salt (C56)

Thionyl chloride (2850 mg, 24.0 mmol) was added drop-wise to a solution of C53 (1.40 g, 7.99 mmol) in chloroform (10 mL) maintained in a water bath. The reaction mixture was stirred for 1 hour at 25° C., whereupon it was concentrated in vacuo to afford the product as a yellow solid. Yield: 1.50 g, 6.52 mmol, 82%. LCMS m/z 193.8 [M+H]⁺. ¹H NMR (400 MHz, CDCl₃) δ 8.03 (s, 1H), 7.99 (s, 1H), 7.77 (d, J=8.2 Hz, 2H), 7.46 (d, J=7.9 Hz, 2H), 4.63 (s, 2H).

### Step 7. Synthesis of ethyl 5-methyl-4-[4-(1,3-oxazol-4-yl)benzyl]pyridine-2-carboxylate (C57)

Compound C10 (90.2 mg, 0.310 mmol), [1,1′-bis(diphenylphosphino)ferrocene]dichloropalladium(II) (18.9 mg, 25.8 μmol), and potassium carbonate (71.4 mg, 0.517 mmol) were added to a solution of C56 (50 mg, 0.22 mmol) in a mixture of 1,4-dioxane (2 mL) and water (0.2 mL). The mixture was degassed with nitrogen for 5 minutes, whereupon it was heated to 100° C. for 18 hours. The reaction solution was taken directly into the following step. LCMS m/z 344.9 [M+Na⁺].

### Step 8. Synthesis of 5-methyl-4-[4-(1,3-oxazol-4-yl)benzyl]pyridine-2-carboxylic acid (C58)

To a solution of C57 (from the previous step, plus a second small-scale reaction, ≦0.26 mmol) in 1,4-dioxane (2.5 mL) were added water (2.5 mL) and sodium hydroxide (49.6 mg, 1.24 mmol); the reaction mixture was stirred for 20 hours at 25° C., then extracted with petroleum ether (3 mL). The aqueous layer was filtered, and the filtrate was acidified to pH 3-5 via addition of 2 M aqueous hydrochloric acid. It was then extracted with dichloromethane (3×10 mL), and the combined dichloromethane layers were dried over sodium sulfate, filtered, and concentrated in vacuo to afford the product as an off-white solid. Yield: 62 mg, 0.21 mmol, 81% over 2 steps. LCMS m/z 294.9 [M+H]⁺.

### Step 9. Synthesis of N-[(3R,4S)-3-hydroxytetrahydro-2H-pyran-4-yl]-5-methyl-4-[4-(1,3-oxazol-4-yl)benzyl]pyridine-2-carboxamide (57)

1-[3-(Dimethylamino)propyl]-3-ethylcarbodiimide hydrochloride (EDCI; 56.5 mg, 0.295 mmol), 1H-benzotriazol-1-ol (42.7 mg, 0.316 mmol), and triethylamine (64.0 mg, 0.632 mmol) were added to a solution of C58 (62 mg, 0.21 mmol) in a mixture of dichloromethane (5 mL) and N,N-dimethylformamide (3 mL). The mixture was stirred for 4 hours at 25° C., whereupon P2 (29.6 mg, 0.253 mmol) was added and stirring was continued for 18 hours at 25° C. Additional 1-[3-(dimethylamino)propyl]-3-ethylcarbodiimide hydrochloride (56.5 mg, 0.295 mmol), 1H-benzotriazol-1-ol (42.7 mg, 0.316 mmol), and triethylamine (64.0 mg, 0.632 mmol) were introduced, and the reaction mixture was stirred for another 30 minutes; additional P2 (29.6 mg, 0.253 mmol) was then added, and stirring was carried out for another 18 hours at 25° C. The reaction mixture was diluted with dichloromethane (20 mL), washed sequentially with saturated aqueous citric acid solution (20 mL) and aqueous sodium hydroxide solution (1 M, 20 mL), and concentrated in vacuo. The residue was subjected to preparative thin layer chromatography on silica gel (Eluent: 1:2 petroleum ether/ethyl acetate), followed by reversed phase HPLC purification (Column: Phenomenex Gemini C18, 5 μm; Mobile phase A: water containing 0.225% formic acid; Mobile phase B: acetonitrile containing 0.225% formic acid; Gradient: 23% to 43% B). The product was obtained as a white solid. Yield: 20 mg, 51 μmol, 24%. LCMS m/z 394.1 [M+H]⁺. ¹H NMR (400 MHz, CDCl₃) δ 8.32 (s, 1H), 8.12 (br d, J=6 Hz, 1H), 8.00 (s, 1H), 7.92-7.96 (m, 2H), 7.68 (d, J=8.2 Hz, 2H), 7.17 (d, J=8.2 Hz, 2H), 4.33-4.46 (br m, 1H), 4.09 (dd, J=12, 5 Hz, 1H), 4.06 (s, 2H), 3.90-4.04 (m, 2H), 3.59-3.68 (m, 1H), 3.43-3.52 (m, 1H), 3.23 (dd, J=10.9, 10.2 Hz, 1H), 2.32 (s, 3H), 2.00-2.08 (m, 1H), 1.74-1.87 (m, 1H).

### Example 58

### N-[(3R,4S)-3-Hydroxytetrahydro-2H-pyran-4-yl]-5-methoxy-4-[4-(2-methyl-1,3-oxazol-4-yl)benzyl]pyridine-2-carboxamide (58)

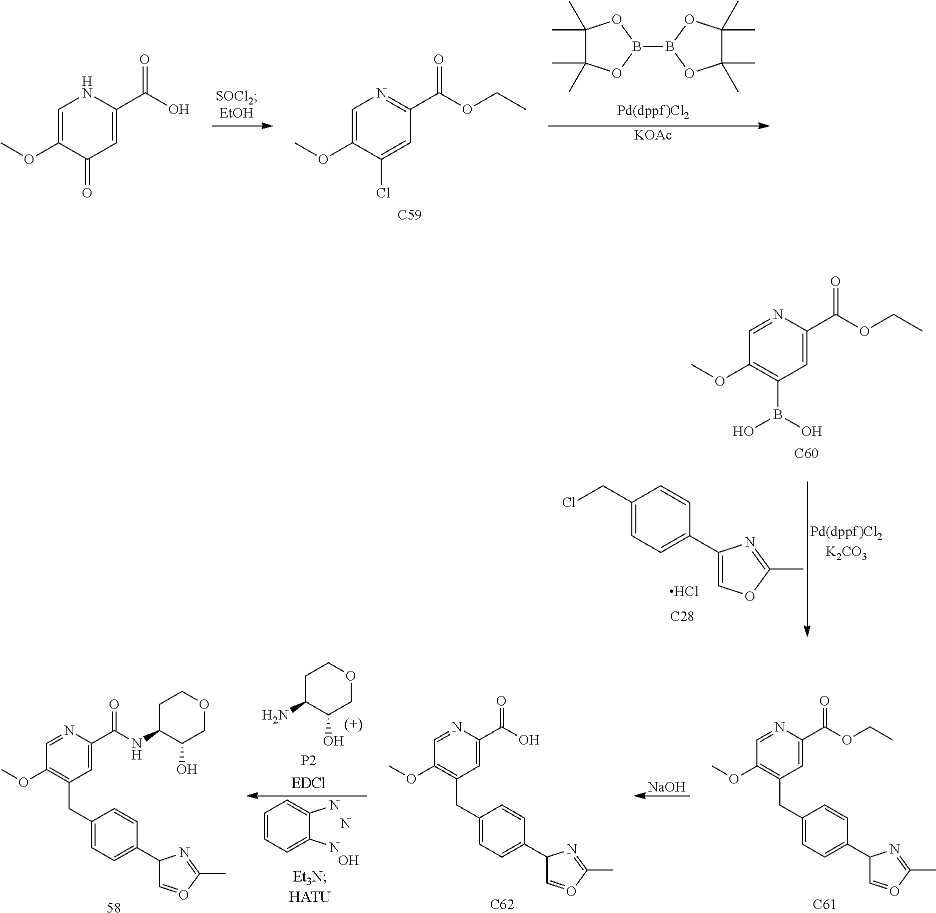

### Step 1. Synthesis of ethyl 4-chloro-5-methoxypyridine-2-carboxylate (C59)

A mixture of 5-methoxy-4-oxo-1,4-dihydropyridine-2-carboxylic acid (30.0 g, 177 mmol) and thionyl chloride (250 mL) was stirred at 100° C. for 18 hours, whereupon the reaction mixture was concentrated in vacuo. The residue was dissolved in anhydrous ethanol (200 mL); the resulting solution was heated at reflux for 20 minutes and then cooled to 20° C. After the mixture had been neutralized by addition of anhydrous sodium carbonate, it was filtered. The filtrate was cooled in an ice-ethanol bath, and stirred for 30 minutes; the precipitate was collected via filtration to afford the product as an off-white solid. The resulting filtrate was concentrated to a smaller volume under reduced pressure and cooled in an ice-ethanol bath. The precipitate was collected via filtration, providing additional product. Combined yield: 14.2 g, 65.8 mmol, 37%. LCMS m/z 215.8 [M+H]⁺. ¹H NMR (400 MHz, DMSO-d₆) δ 8.58 (s, 1H), 8.09 (s, 1H), 4.32 (q, J=7.1 Hz, 2H), 4.08 (s, 3H), 1.32 (t, J=7.1 Hz, 3H).

### Step 2. Synthesis of [2-(ethoxycarbonyl)-5-methoxypyridin-4-yl]boronic acid (C60)

A mixture of C59 (100 mg, 0.46 mmol), 4,4,4′,4′,5,5,5′,5′-octamethyl-2,2′-bi-1,3,2-dioxaborolane (177 mg, 0.697 mmol), potassium acetate (114 mg, 1.16 mmol), and [1,1′-bis(diphenylphosphino)ferrocene]dichloropalladium(11) (33.9 mg, 46.3 μmol) in toluene (10 mL) was stirred at 130° C. for 18 hours. The reaction mixture was used directly in the following step. LCMS m/z 225.9 [M+H]⁺.

### Step 3. Synthesis of ethyl 5-methoxy-4-[4-(2-methyl-1,3-oxazol-4-yl)benzyl]pyridine-2-carboxylate (C61)

1,4-Dioxane (10 mL) and water (2 mL) were added to C60 (as a toluene solution from the previous step, ≦0.46 mmol). Compound C28 (169 mg, 0.692 mmol), [1,1′-bis(diphenylphosphino)ferrocene]dichloropalladium(11) (33.8 mg, 46.2 μmol), and potassium carbonate (160 mg, 1.16 mmol) were then introduced, and the reaction mixture was stirred at 80° C. for 4 hours. After removal of solvents in vacuo, the residue was purified by preparative thin layer chromatography on silica gel (Eluent: 1:1 petroleum ether/ethyl acetate), affording the product as a light yellow gum. Yield: 150 mg, 0.426 mmol, 93% over two steps. LCMS m/z 353.0 [M+H]⁺. ¹H NMR (400 MHz, CDCl₃) δ 8.33 (s, 1H), 7.91 (s, 1H), 7.78 (s, 1H), 7.64 (d, J=8.3 Hz, 2H), 7.23 (d, J=8.0 Hz, 2H), 4.43 (q, J=7.1 Hz, 2H), 4.01 (s, 2H), 4.00 (s, 3H), 2.52 (s, 3H), 1.42 (t, J=7.1 Hz, 3H).

### Step 4. Synthesis of 5-methoxy-4-[4-(2-methyl-1,3-oxazol-4-yl)benzyl]pyridine-2-carboxylic acid (C62)

Sodium hydroxide (32 mg, 0.80 mmol) and C61 (150 mg, 0.426 mmol) were combined in a mixture of methanol (3 mL) and water (3 mL) and stirred at 30° C. for 2 hours. The reaction mixture was then acidified to pH 2 via addition of 1 M aqueous hydrochloric acid. Removal of solvent in vacuo afforded the product as a light yellow gum. Yield: 130 mg, 0.40 mmol, 95%. LCMS m/z 324.9 [M+H]⁺.

### Step 5. Synthesis of N-[(3R,4S)-3-hydroxytetrahydro-2H-pyran-4-yl]-5-methoxy-4-[4-(2-methyl-1,3-oxazol-4-yl)benzyl]pyridine-2-carboxamide (58)

To a solution of C62 (65 mg, 0.20 mmol) in dichloromethane (3 mL) were added triethylamine (77 μL, 0.55 mmol), 1-[3-(dimethylamino)propyl]-3-ethylcarbodiimide hydrochloride (53.0 mg, 0.277 mmol), and 1H-benzotriazol-1-ol (37.4 mg, 0.277 mmol). After the reaction mixture had been stirred at 10° C. for 1 hour, P2 (30.2 mg, 0.258 mmol) was added, and stirring was continued at 10° C. for 16 hours. It was then warmed to 30° C. for another 5 hours, whereupon it was treated with O-(7-azabenzotriazol-1-yl)-N,N,N′,N′-tetramethyluronium hexafluorophosphate (40 mg, 0.11 mmol). After 1 hour, the reaction mixture was filtered through Amberlyst® A-26 (hydroxide form) ion exchange resin; the filtrate was concentrated in vacuo and purified via reversed phase HPLC (Column: Agela Durashell C18, 5 μm; Mobile phase A: water containing 0.225% formic acid; Mobile phase B: acetonitrile; Gradient: 28% to 48%). The product was isolated as a white solid. Yield: 7.0 mg, 8%. LCMS m/z 445.9 [M+N⁺]. ¹H NMR (400 MHz, CD₃OD) δ 8.31 (s, 1H), 8.10 (s, 1H), 7.85 (s, 1H), 7.64 (br d, J=8.2 Hz, 2H), 7.27 (br d, J=8.2 Hz, 2H), 4.04 (s, 2H), 4.03 (s, 3H), 3.86-3.99 (m, 3H), 3.58-3.66 (m, 1H), 3.42-3.51 (m, 1H), 3.18 (dd, J=10.7, 10.2 Hz, 1H), 2.49 (s, 3H), 1.96-2.04 (m, 1H), 1.62-1.75 (m, 1H).

### Examples 59 and 60

### (+)-4-{Fluoro[4-(1,3-thiazol-4-yl)phenyl]methyl}-N-[(3R,4S)-3-hydroxytetrahydro-2H-pyran-4-yl]-5-methylpyridine-2-carboxamide (diastereomer 1) (59) and (−)-{Fluoro[4-(1,3-thiazol-4-yl)phenyl]methyl}-N-[(3R,4S)-3-hydroxytetrahydro-2H-pyran-4-yl]-5-methylpyridine-2-carboxamide (diastereomer 2) (60)

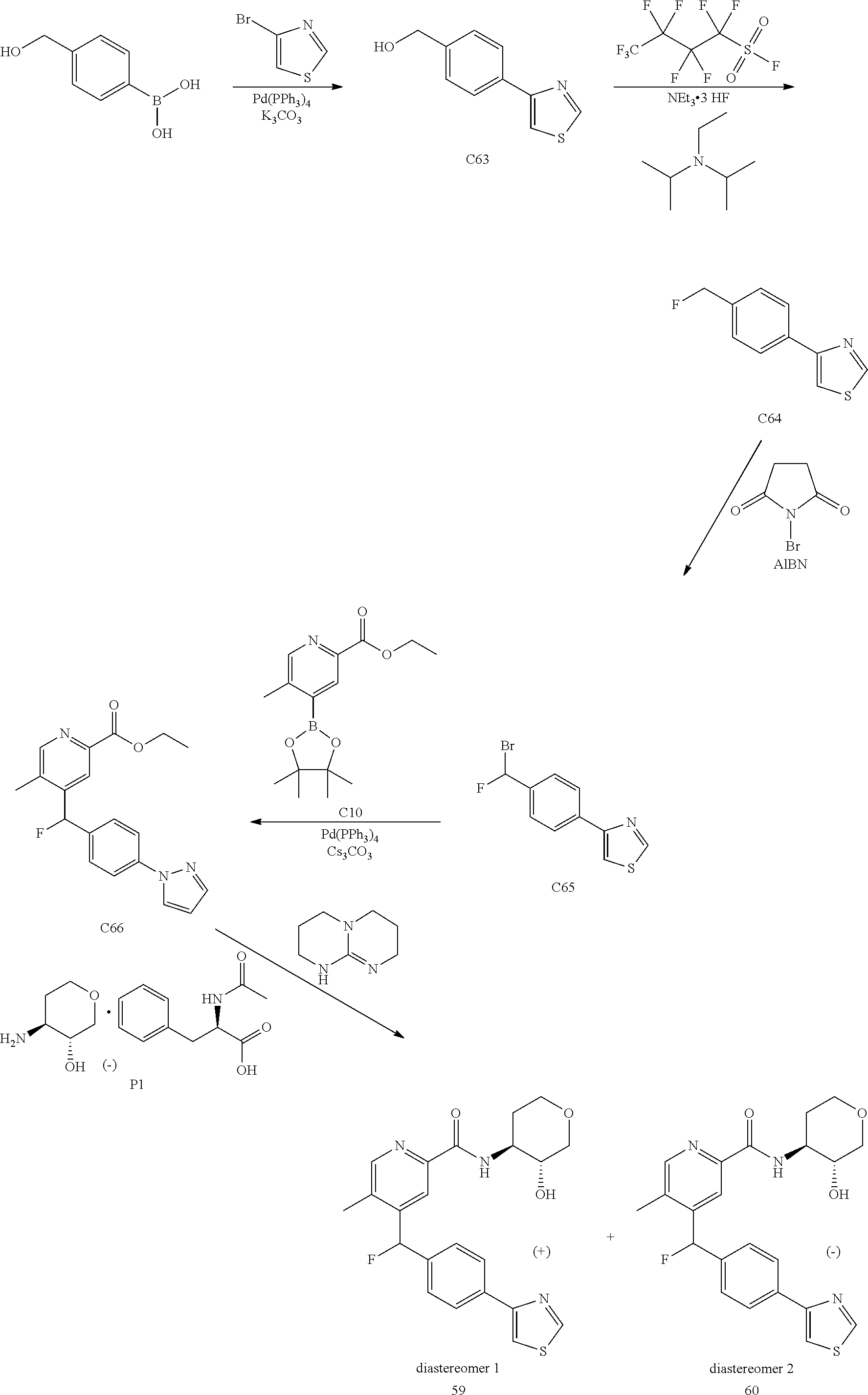

### Step 1. Synthesis of [4-(1,3-thiazol-4-yl)phenyl]methanol (C63)

[4-(Hydroxymethyl)phenyl]boronic acid (96%, 4.0 g, 25 mmol) and 4-bromo-1,3-thiazole (96%, 6.48 g, 37.9 mmol) were dissolved in 1,4-dioxane (75 mL). Aqueous potassium carbonate solution (3 M, 17 mL, 51 mmol) was added, followed by tetrakis(triphenylphosphine)palladium(0) (880 mg, 0.76 mmol), and the reaction mixture was heated overnight at 100° C. It was then cooled to room temperature, diluted with water, and extracted several times with ethyl acetate. The combined organic layers were washed with saturated aqueous sodium chloride solution, dried over magnesium sulfate, filtered, and concentrated under reduced pressure. Purification via chromatography on silica gel (Gradient: 25% to 50% ethyl acetate in heptane) afforded the product as a cream-colored solid. Yield: 3.60 g, 18.8 mmol, 75%. LCMS m/z 192.0 [M+H]⁺. ¹H NMR (400 MHz, CDCl₃) δ 8.92 (d, J=2.0 Hz, 1H), 7.95 (br d, J=8.2 Hz, 2H), 7.56 (d, J=2.0 Hz, 1H), 7.46 (br d, J=8.3 Hz, 2H), 4.76 (s, 2H).

### Step 2. Synthesis of 4-[4-(fluoromethyl)phenyl]-1,3-thiazole (C64)

To a solution of C63 (2.00 g, 10.5 mmol) in acetonitrile (40 mL) were added 1,1,2,2,3,3,4,4,4-nonafluorobutane-1-sulfonyl fluoride (2.1 mL, 11.7 mmol) and triethylamine trihydrofluoride (1.88 mL, 11.5 mmol), followed by N,N-diisopropylethylamine (3.64 mL, 20.9 mmol). The reaction mixture was stirred for six hours, whereupon the reaction was quenched via addition of saturated aqueous sodium bicarbonate solution. The mixture was extracted several times with ethyl acetate, and the combined organic layers were dried over magnesium sulfate, filtered, and concentrated in vacuo. Chromatography on silica gel (Eluent: 10% ethyl acetate in heptane, followed by 25% ethyl acetate in heptane) provided the product as a white solid. Yield: 800 mg, 4.1 mmol, 39%. ¹H NMR (400 MHz, CDCl₃) δ 8.93 (s, 1H), 7.98 (d, J=8.1 Hz, 2H), 7.58-7.61 (m, 1H), 7.47 (d, J=7.5 Hz, 2H), 5.43 (d, J_{HF}=47.7 Hz, 2H).

### Step 3. Synthesis of 4-{4-[bromo(fluoro)methyl]phenyl}-1,3-thiazole (C65)

N-Bromosuccinimide (96%, 1.16 g, 6.26 mmol) was added to a solution of C64 (1.10 g, 5.69 mmol) in tetrachloromethane (40 mL). 2,2′-Azobisisobutyronitrile (96%, 97 mg, 0.57 mmol) was added, and the reaction mixture was heated at reflux for two hours. After it had been cooled to room temperature, the reaction mixture was quenched with water, and extracted several times with dichloromethane. The combined organic layers were washed with saturated aqueous sodium chloride solution, dried over magnesium sulfate, filtered, and concentrated in vacuo. Silica gel chromatography (Eluent: 10% ethyl acetate in heptane) afforded the product as a light pink solid (1.25 g). By ¹H NMR analysis, this material was contaminated with a small amount of unreacted C64. Yield, corrected for C64 remaining in the isolated product: 1.08 g, 3.97 mmol, 70%. ¹H NMR (400 MHz, CDCl₃) δ 8.92 (d, J=2.0 Hz, 1H), 7.98-8.03 (m, 2H), 7.63 (d, J=2.0 Hz, 1H), 7.57-7.61 (m, 2H), 7.46 (d, J_{HF}=49.4 Hz, 1H).

### Step 4. Synthesis of ethyl 4-{fluoro[4-(1,3-thiazol-4-yl)phenyl]methyl}-5-methylpyridine-2-carboxylate (C66)

1,4-Dioxane (10 mL) was added to a mixture of C10 (400 mg, 1.37 mmol), C65 (449 mg, 1.65 mmol), and tetrakis(triphenylphosphine)palladium(0) (159 mg, 0.138 mmol) in a sealable reaction vessel. Aqueous cesium carbonate solution (3 M, 1.4 mL, 4.2 mmol) was introduced, the reaction vessel was sealed, and the reaction mixture was heated at 50° C. for two hours. After the reaction mixture had cooled to room temperature, it was diluted with ethyl acetate, washed sequentially with water and with saturated aqueous sodium chloride solution, dried over magnesium sulfate, filtered, and concentrated in vacuo. Purification via silica gel chromatography (Eluent: 25% ethyl acetate in heptane, followed by 50% and then 75% ethyl acetate in heptane) provided the product as a yellow oil. Yield: 350 mg, 0.98 mmol, 72%. LCMS m/z 357.4 [M+H]⁺. ¹H NMR (400 MHz, CDCl₃) δ 8.89 (d, J=2.0 Hz, 1H), 8.55-8.56 (m, 1H), 8.35 (s, 1H), 7.94-7.98 (m, 2H), 7.59 (d, J=2.0 Hz, 1H), 7.34-7.39 (m, 2H), 6.59 (d, J_{HF}=47.1 Hz, 1H), 4.51 (qd, J=7.1, 0.6 Hz, 2H), 2.23 (s, 3H), 1.47 (t, J=7.1 Hz, 3H).

### Step 5. Synthesis of (+)-4-{fluoro[4-(1,3-thiazol-4-yl)phenyl]methyl}-N-[(3R,4S)-3-hydroxytetrahydro-2H-pyran-4-yl]-5-methylpyridine-2-carboxamide (diastereomer 1) (59) and ( )-{fluoro[4-(1,3-thiazol-4-yl)phenyl]methyl}-N-[(3R,4S)-3-hydroxytetrahydro-2H-pyran-4-yl]-5-methylpyridine-2-carboxamide (diastereomer 2) (60)

A mixture of C66 (350 mg, 0.98 mmol), P1 (414 mg, 1.28 mmol), and 1,3,4,6,7,8-hexahydro-2H-pyrimido[1,2-a]pyrimidine (98%, 237 mg, 1.67 mmol) in N,N-dimethylformamide (5 mL) was stirred at 60° C. overnight. The reaction mixture was diluted with water and extracted several times with ethyl acetate. The combined organic extracts were washed sequentially with saturated aqueous sodium bicarbonate solution and saturated aqueous sodium chloride solution, dried over magnesium sulfate, filtered, and concentrated under reduced pressure. The residue was subjected to chromatography on silica gel (Gradient: 75% to 100% ethyl acetate in heptane) to afford the racemic product as a colorless oil, which slowly solidified upon standing. Yield: 350 mg, 0.82 mmol, 84%. This material was separated into its component diastereomers using supercritical fluid chromatography (Column: Chiral Technologies Chiralcel OD-H, 5 μm; Mobile phase: 65:35 carbon dioxide/methanol). The first-eluting diastereomer was 59 (diastereomer 1), isolated as a solid; this material exhibited a positive (+) rotation. Yield: 120 mg, 0.281 mmol, 34% for the purification. LCMS m/z 428.5 [M+H]⁺. ¹H NMR (400 MHz, CDCl₃) δ 8.89 (d, J=2.0 Hz, 1H), 8.43 (s, 1H), 8.35-8.36 (m, 1H), 8.16 (br d, J=6 Hz, 1H), 7.94-7.98 (m, 2H), 7.59 (d, J=2.0 Hz, 1H), 7.35-7.40 (m, 2H), 6.59 (d, J_{HF}=47.1 Hz, 1H), 4.11 (br dd, J=11.4, 5.0 Hz, 1H), 3.95-4.05 (m, 2H), 3.67 (ddd, J=9.7, 9.4, 5.0 Hz, 1H), 3.50 (ddd, J=11.9, 11.9, 2.2 Hz, 1H), 3.25 (dd, J=11.4, 9.9 Hz, 1H), 2.25 (s, 3H), 2.04-2.11 (m, 1H), 1.82 (dddd, J=13, 12, 12, 5 Hz, 1H).

The second-eluting product was 60 (diastereomer 2), obtained as a colorless oil that slowly solidified. This material exhibited a negative (−) rotation. Yield: 120 mg, 0.281 mmol, 34% for the purification. LCMS m/z 428.5 [M+H]⁺. ¹H NMR (400 MHz, CDCl₃) δ 8.89 (d, J=2.0 Hz, 1H), 8.41 (s, 1H), 8.35 (s, 1H), 8.17 (br d, J=6 Hz, 1H), 7.95 (br d, J=8 Hz, 2H), 7.58 (d, J=2.0 Hz, 1H), 7.34-7.39 (m, 2H), 6.58 (d, J_{HF}=47.1 Hz, 1H), 4.11 (br dd, J=11.4, 5.1 Hz, 1H), 3.95-4.05 (m, 2H), 3.67 (ddd, J=9.6, 9.5, 5.0 Hz, 1H), 3.49 (ddd, J=11.9, 11.9, 2.2 Hz, 1H), 3.25 (dd, J=11.4, 9.9 Hz, 1H), 2.24 (s, 3H), 2.03-2.10 (m, 1H), 1.75-1.87 (m, 1H).

### Example 61

### 4-[4-(1,3-Dimethyl-1H-pyrazol-4-yl)benzyl]-N-[(3R,4S)-3-hydroxytetrahydro-2H-pyran-4-yl]-5-methylpyridine-2-carboxamide (61)

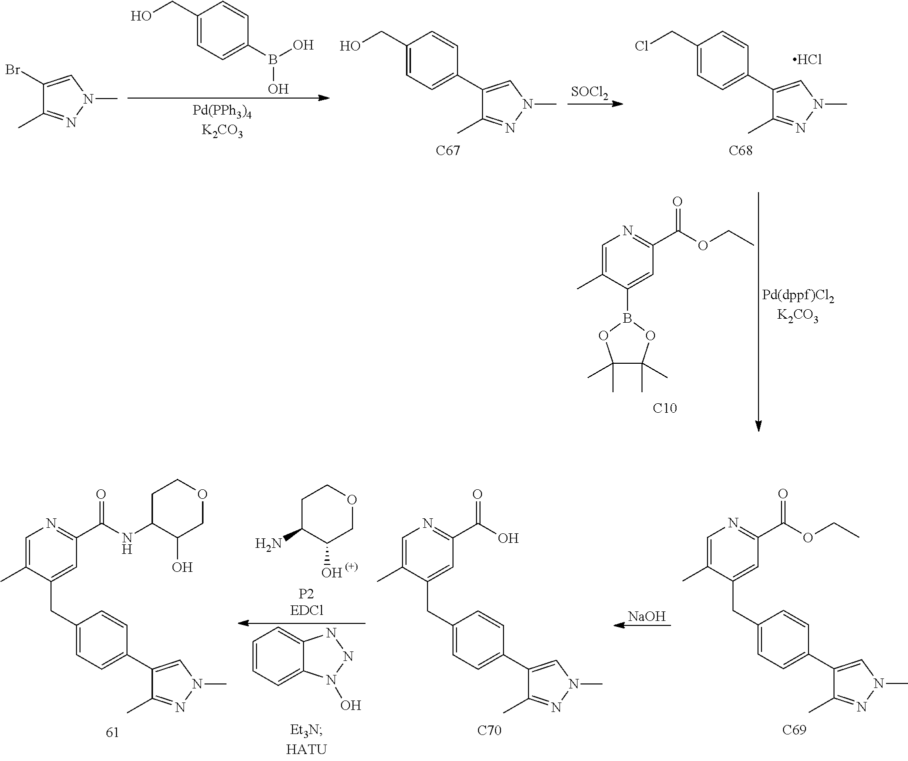

### Step 1. Synthesis of [4-(1,3-dimethyl-1H-pyrazol-4-yl)phenyl]methanol (C67)

A mixture of 4-bromo-1,3-dimethyl-1H-pyrazole (200 mg, 1.14 mmol), [4-(hydroxymethyl)phenyl]boronic acid (260 mg, 1.71 mmol), potassium carbonate (474 mg, 3.43 mmol) and tetrakis(triphenylphosphine)palladium(0) (132 mg, 0.114 mmol) in 1,4-dioxane (12 mL) and water (3 mL) was heated at 100° C. for 16 hours. The reaction mixture was concentrated in vacuo; purification using silica gel chromatography (Gradient: 0% to 100% ethyl acetate in petroleum ether) provided the product (190 mg) as a yellow solid. By ¹H NMR and mass spectroscopic analysis, this material was contaminated with triphenylphosphine oxide. Yield, corrected for triphenylphospine oxide content: 120 mg, 0.59 mmol, 52%. LCMS m/z 202.9 [M+H]⁺. ¹H NMR (400 MHz, CDCl₃), product peaks only: δ 7.43 (s, 1H), 7.39 (br s, 4H), 4.72 (s, 2H), 3.89 (s, 3H), 2.40 (s, 3H).

### Step 2. Synthesis of 4-[4-(chloromethyl)phenyl]-1,3-dimethyl-1H-pyrazole, hydrochloride salt (C68)

Thionyl chloride (1.0 mL, 14 mmol) was added to a solution of C67 (280 mg; when corrected for triphenylphospine oxide contamination: 180 mg, 0.9 mmol) in chloroform (20 mL), and the reaction mixture was stirred at 16° C. for 3 hours. Removal of solvent in vacuo provided the crude product as a yellow solid (380 mg). A portion of this material was used in the following step.

### Step 3. Synthesis of ethyl 4-[4-(1,3-dimethyl-1H-pyrazol-4-yl)benzyl]-5-methylpyridine-2-carboxylate (C69)

Potassium carbonate (161 mg, 1.16 mmol) and [1,1′-bis(diphenylphosphino)ferrocene]dichloropalladium(II) (28.4 mg, 38.8 μmol) were added to a solution of C68 (from the previous step, 100 mg, 0.24 mmol) and C10 (136 mg, 0.467 mmol) in 1,4-dioxane (18 mL) and water (2 mL). The reaction mixture was stirred at 80° C. for 8 hours, whereupon it was cooled to room temperature and filtered. The filtrate was concentrated to dryness under reduced pressure and purified via chromatography on silica gel (Gradient: 85% to 100% ethyl acetate in petroleum ether), affording the product as a light yellow oil. Yield: 60 mg, 0.17 mmol, 70% over 2 steps. LCMS m/z 349.9 [M+H]⁺.

### Step 4. Synthesis of 4-[4-(1,3-dimethyl-1H-pyrazol-4-yl)benzyl]-5-methylpyridine-2-carboxylic acid (C70)

Sodium hydroxide (27.5 mg, 0.688 mmol) was added to a solution of C69 (60 mg, 0.17 mmol) in a mixture of tetrahydrofuran (5 mL) and water (5 mL). The reaction mixture was stirred at 50° C. for 16 hours, then concentrated to dryness under reduced pressure to provide the product (74 mg) as a light yellow oil; this material was used in the following step without further purification. LCMS m/z 321.9 [M+H]⁺.

### Step 5. Synthesis of 4-[4-(1,3-dimethyl-1H-pyrazol-4-yl)benzyl]-N-[(3R,4S)-3-hydroxytetrahydro-2H-pyran-4-yl]-5-methylpyridine-2-carboxamide (61)

1-[3-(Dimethylamino)propyl]-3-ethylcarbodiimide hydrochloride (66.2 mg, 0.345 mmol) and 1H-benzotriazol-1-ol (46.7 mg, 0.346 mmol) were added to a solution of C70 (from the previous step, 74 mg, ≦0.17 mmol) in dichloromethane (3 mL), and the reaction mixture was stirred at 18° C. for 1 hour. Compound P2 (27.0 mg, 0.230 mmol) was then introduced, and stirring was continued at 18° C. for 18 hours. O-(7-Azabenzotriazol-1-yl)-N,N,N′,N′-tetramethyluronium hexafluorophosphate (131 mg, 0.344 mmol) was then added, and stirring was continued at 18° C. for 20 hours. The reaction mixture was filtered and subjected to preparative thin layer chromatography on silica gel (Eluent: ethyl acetate), then purified by reversed phase HPLC purification (Column: Agela Durashell C18, 5 μm; Mobile phase A: water containing 0.225% formic acid; Mobile phase B: acetonitrile containing 0.225% formic acid; Gradient: 25% to 45% B). The product was obtained as a white solid. Yield: 22 4 mg, 53.2 μmol, 31% over 2 steps. LCMS m/z 421.0 [M+H]⁺. ¹H NMR (400 MHz, CDCl₃) δ 8.32 (s, 1H), 8.08-8.18 (br m, 1H), 8.00 (s, 1H), 7.40 (s, 1H), 7.30 (br d, J=7.8 Hz, 2H), 7.12 (br d, J=8.0 Hz, 2H), 4.04 (s, 2H), 3.90-4.13 (m, 3H), 3.88 (s, 3H), 3.58-3.69 (m, 1H), 3.42-3.53 (m, 1H), 3.23 (dd, J=10.5, 10.4 Hz, 1H), 2.38 (s, 3H), 2.34 (s, 3H), 1.99-2.08 (m, 1H), 1.73-1.87 (m, 1H).

Table 6-1 below lists some additional examples of compounds of the invention (Examples 62-72) that were made using methods, starting materials or intermediates, and preparations described herein.

TABLE 6-1
Examples 62-72 (including Method of Preparation, Non-Commercial starting materials, Structures and
Physicochemical Data).

| | | | ¹H NMR (400 MHz, CDCl₃) δ |
|---|---|---|---|
| | Method of | | (ppm); Mass spectrum, |
| | Preparation; | | observed ion m/z [M + H]⁺ or |
| | Non-commercial | | HPLC retention time; Mass |
| Example | starting | | spectrum m/z [M + H]⁺ (unless |
| Number | materials | Structure | otherwise indicated) |
| 62 | Example 14¹; C44, P2 | 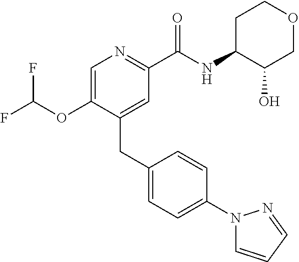 [SMILES: O=C(N[C@H]1CCOC[C@@H]1O)c1cc(Cc2ccc(-n3cccn3)cc2)c(OC(F)F)cn1] | ¹H NMR (400 MHz, CD₃OD) δ 8.48 (s, 1H), 8.20 (d, J = 2.4 Hz, 1H), 8.00 (s, 1H), 7.67-7.72 (m, 3H), 7.38 (br d, J = 8.7 Hz, 2H), 7.08 (t, J_{HF} = 72.7 Hz, 1H), 6.52 (dd, J = 2, 2 Hz, 1H), 4.17 (s, 2H), 3.87-3.99 (m, 3H), 3.60-3.67 (m, 1H), 3.42-3.51 (m, 1H), 3.18 (dd, J = 11, 10 Hz, 1H), 1.95-2.02 (m, 1H), 1.64-1.77 (m, 1H); 444.9 |
| 63 | Example 3^{2, 3}; C50 | 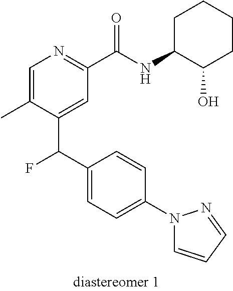 [SMILES: Cc1cnc(C(=O)N[C@H]2CCCC[C@@H]2O)cc1C(F)c1ccc(-n2cccn2)cc1] | ¹H NMR (600 MHz, DMSO-d₆) δ 8.49 (d, J = 2.4 Hz, 1H), 8.48 (s, 1H), 8.35 (br d, J = 7.9 Hz, 1H), 8.14 (s, 1H), 7.90 (d, J = 8.5 Hz, 2H), 7.74-7.76 (m, 1H), 7.47 (br d, J = 8 Hz, 2H), 6.97 (d, J_{HF} = 46.2 Hz, 1H), 6.54-6.56 (m, 1H), 4.64 (br d, J = 5 Hz, 1H), 3.56-3.64 (m, 1H), 3.42-3.50 (m, 1H), 2.21 (s, 3H), 1.86-1.96 (m, 2H), 1.58-1.69 (m, 2H), 1.19-1.36 (m, 4H); 409.4 |
| 64 | Example 3^{2, 3}; C50 | 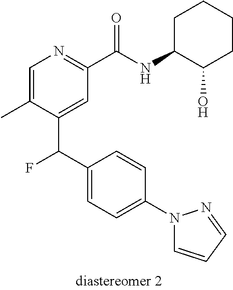 [SMILES: Cc1cnc(C(=O)N[C@H]2CCCC[C@@H]2O)cc1C(F)c1ccc(-n2cccn2)cc1] | ¹H NMR (600 MHz, DMSO-d₆) δ 8.50 (d, J = 2.5 Hz, 1H), 8.48 (s, 1H), 8.36 (br d, J = 8.0 Hz, 1H), 8.13 (s, 1H), 7.90 (br d, J = 8.4 Hz, 2H), 7.74-7.76 (m, 1H), 7.48 (br d, J = 8.3 Hz, 2H), 6.97 (d, J_{HF} = 46.3 Hz, 1H), 6.55 (dd, J = 2.0, 1.8 Hz, 1H), 4.66 (d, J = 5.4 Hz, 1H), 3.56- 3.63 (m, 1H), 3.43-3.50 (m, 1H), 2.21 (s, 3H), 1.87-1.97 (m, 2H), 1.59-1.68 (m, 2H), 1.21-1.33 (m, 4H); 409.3 |
| 65 | Example 6⁴; C10, P2 | 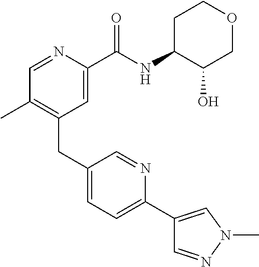 | 8.38 (br s, 1H), 8.33 (s, 1H), 8.11 (br d, J = 6 Hz, 1H), 7.99 (s, 1H), 7.92 (s, 1H), 7.90 (s, 1H), 7.34-7.41 (m, 2H), 4.28- 4.37 (br m, 1H), 4.09 (dd, J = 11.5, 4.8 Hz, 1H), 4.02 (s, 2H), 3.96 (s, 3H), 3.90-4.01 (m, 1H), 3.59-3.68 (m, 1H), 3.44-3.52 (m, 1H), 3.23 (dd, J = 11.2, 10.2 Hz, 1H), 2.34 (s, 3H), 2.01-2.08 (m, 1H), 1.74- 1.86 (m, 1H); 408.0 |
| 66 | Example 3⁵; C13, P1 | 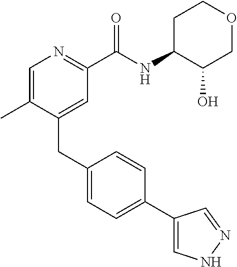 [SMILES: Cc1cnc(C(=O)N[C@H]2CCOC[C@@H]2O)cc1Cc1ccc(-c2cn[nH]c2)cc1] | 8.32 (s, 1H), 8.14 (br d, J = 6 Hz, 1H), 8.00 (d, 1H), 7.82- 7.90 (br s, 2H), 7.44 (br d, J = 8.2 Hz, 2H), 7.13 (br d, J = 8.2 Hz, 2H), 4.09 (dd, J = 11, 5 Hz, 1H), 4.05 (s, 2H), 3.91- 4.04 (m, 3H), 3.64 (ddd, J = 9.6, 9.5, 5.0 Hz, 1H), 3.48 (ddd, J = 11.9, 11.9, 2.2 Hz, 1H), 3.23 (dd, J = 11.4, 9.9 Hz, 1H), 2.34 (s, 3H), 1.99-2.08 (m, 1H), 1.80 (dddd, J = 13, 12, 12, 5 Hz, 1H); 393.5 |
| 67 | Example 3; C13, P1 | 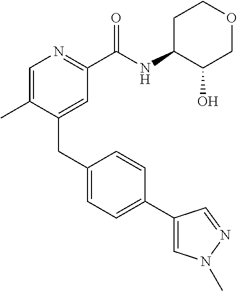 [SMILES: Cc1cnc(C(=O)N[C@H]2CCOC[C@@H]2O)cc1Cc1ccc(-c2cnn(C)c2)cc1] | 8.32 (s, 1H), 8.16 (br d, J = 6 Hz, 1H), 8.01 (s, 1H), 7.73 (d, J = 0.7 Hz, 1H), 7.58-7.59 (m, 1H), 7.39 (br d, J = 8.3 Hz, 2H), 7.10 (br d, J = 8.3 Hz, 2H), 4.32-4.45 (br s, 1H), 4.09 (br dd, J = 11.4, 5.0 Hz, 1H), 4.04 (s, 2H), 3.98-4.04 (m, 1H), 3.95 (s, 3H), 3.90-3.98 (m, 1H), 3.65 (ddd, J = 9.6, 9.6, 5.0 Hz, 1H), 3.48 (ddd, J = 11.9, 11.9, 2.2 Hz, 1H), 3.23 (dd, J = 11.4, 9.9 Hz, 1H), 2.33 (s, 3H), 2.01-2.08 (m, 1H), 1.80 (dddd, J = 13.0, 11.9, 11.9, 4.8 Hz, 1H); 407.5 |
| 68 | Example 58; C60, C20, P2 | 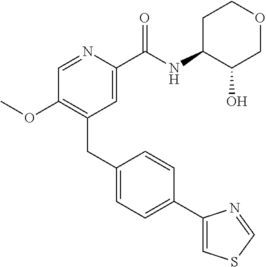 [SMILES: COc1cnc(C(=O)N[C@H]2CCOC[C@@H]2O)cc1Cc1ccc(-c2cscn2)cc1] | 8.84-8.90 (m, 1H), 8.13 (s, 1H), 8.00 (s, 1H), 7.96 (br d, J = 6 Hz, 1H), 7.85 (d, J = 8.2 Hz, 2H), 7.49-7.52 (m, 1H), 7.25-7.31 (m, 2H, assumed; partially obscured by solvent peak), 4.43-4.54 (br m, 1H), 4.08 (dd, J = 11.5, 4.9 Hz, 1H), 4.03 (s, 2H), 4.00 (s, 3H), 3.96-4.0 (m, 1H), 3.87-3.96 (m, 1H), 3.57-3.66 (m, 1H), 3.42-3.51 (m, 1H), 3.22 (dd, J = 10.9, 10.3 Hz, 1H), 1.98- 2.06 (m, 1H), 1.72-1.84 (m, 1H); 448.0 [M + Na⁺] |
| 69 | Example 58⁶; C10, P2 | 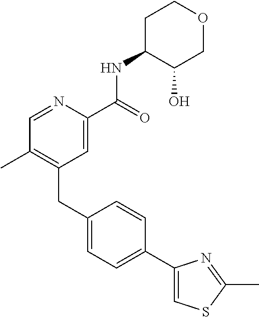 | 8.31 (s, 1H), 8.12 (br d, J = 6 Hz, 1H), 8.01 (s, 1H), 7.80 (d, J = 8.2 Hz, 2H), 7.28 (s, 1H), 7.15 (d, J = 8.2 Hz, 2H), 4.39- 4.47 (br m, 1H), 4.10 (dd, J = 12, 5 Hz, 1H), 4.06 (s, 2H), 3.90-4.04 (m, 2H), 3.60-3.68 (m, 1H), 3.43-3.52 (m, 1H), 3.23 (dd, J = 11.2, 9.8 Hz, 1H), 2.77 (s, 3H), 2.30 (s, 3H), 2.00-2.08 (m, 1H), 1.75-1.87 (m, 1H); 423.9 |
| 70 | Example 58²; C60 | 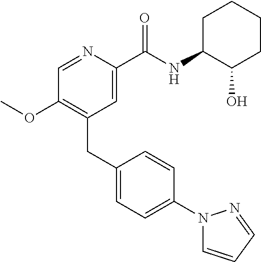 [SMILES: COc1cnc(C(=O)N[C@H]2CCCC[C@@H]2O)cc1Cc1ccc(-n2cccn2)cc1] | ¹H NMR (400 MHz, CD₃OD) δ 8.30 (s, 1H), 8.17 (s, 1H), 7.87 (s, 1H), 7.60-7.74 (m, 3H), 7.35 (br d, J = 8 Hz, 2H), 6.47- 6.54 (m, 1H), 4.06 (s, 2H), 4.03 (s, 3H), 3.65-3.77 (br m, 1H), 3.45-3.56 (br m, 1H), 1.95-2.09 (br m, 2H), 1.65- 1.82 (br m, 2H), 1.25-1.45 (br m, 4H); 407.0 |
| 71 | Example 58²; C60, C56 | 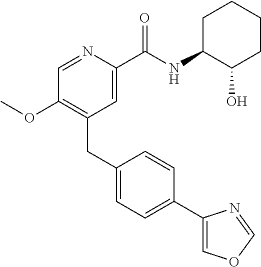 [SMILES: COc1cnc(C(=O)N[C@H]2CCCC[C@@H]2O)cc1Cc1ccc(-c2cocn2)cc1] | characteristic peaks: 8.13 (s, 1H), 8.00 (s, 1H), 7.91-7.94 (m, 2H), 7.87 (br d, J = 7 Hz, 1H), 7.67 (br d, J = 8.3 Hz, 2H), 7.23-7.29 (m, 2H, assumed; partially obscured by solvent peak), 4.02 (s, 2H), 3.99 (s, 3H), 2.01-2.16 (m, 2H), 1.72- 1.81 (m, 2H), 1.22-1.47 (m, 4H); 407.9 |
| 72 | Example 58²; C62 | 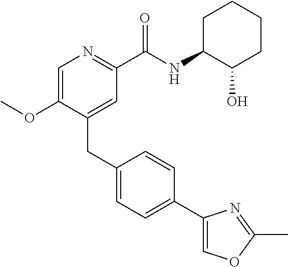 | ¹H NMR (400 MHz, CD₃OD) δ 8.30 (s, 1H), 8.10 (s, 1H), 7.84 (s, 1H), 7.64 (br s, 2H), 7.27 (br s, 2H), 4.04 (s, 2H), 4.03 (s, 3H), 3.66-3.76 (m, 1H), 3.46-3.55 (m, 1H), 2.49 (s, 3H), 1.97-2.07 (m, 2H), 1.68- 1.80 (m, 2H), 1.27-1.43 (m, 4H); 422.0 |

1. The requisite 5-(difluoromethoxy)-4-[4-(1H-pyrazol-1-yl)benzyl]pyridine-2-carboxylic acid was prepared via reaction of C44 with sodium chloro(difluoro)acetate and potassium carbonate at elevated temperature.
2. (1S,2S)-2-Aminocyclohexanol was used in the final coupling step.
3. The diastereomeric Examples 63 and 64 were separated using supercritical fluid chromatography (Column: Chiral Technologies Chiralcel OD-H, 5 μm; Mobile phase: 3:1 carbon dioxide/methanol). Example 63 was the first-eluting diastereomer, and Example 64 was the second-eluting diastereomer.
4. Suzuki reaction of (6-chloropyridin-3-yl)methanol with 1-methyl-4-(4,4,5,5-tetramethyl-1,3,2-dioxaborolan-2-yl)-1H-pyrazole afforded [6-(1-methyl-1H-pyrazol-4-yl)pyridin-3-yl]methanol. Subsequent bromination with phosphorus tribromide provided the requisite 5-(bromomethyl)-2-(1-methyl-1H-pyrazol-4-yl)pyridine.
5. Suzuki reaction of C13 with 4-bromo-1-trityl-1H-pyrazole provided ethyl 5-methyl-4-[4-(1-trityl-1H-pyrazol-4-yl)benzyl]pyridine-2-carboxylate; removal of the trityl group with 1 M hydrochloric acid in methanol afforded the requisite ethyl 5-methyl-4-[4-(1H-pyrazol-4-yl)benzyl]pyridine-2-carboxylate.
6. The requisite 4-[4-(chloromethyl)phenyl]-2-methyl-1,3-thiazole, hydrochloride salt, was prepared via chlorination of [4-(2-methyl-1,3-thiazol-4-yl)phenyl]methanol with thionyl chloride.

### Example AA

### M1 FLIPR Assay

This assay was designed to select and characterize compounds that affect the activity of human M1 muscarinic acetylcholine receptors (Similar M1 PAM FLIPR assays can be found, for example, at U.S. Pat. No. 8,664,234). Human M1 receptors were stably expressed in Chinese hamster ovary (CHO) cells (HD Bioscience). The effect of test compounds on intracellular calcium was measured on an FLIPR Tetra (Molecular Devices) using the Fluo-8, AM calcium dye (Molecular Probes) with a red dye quenching agent (Sigma).

Cells:

CHO cells expressing hM1 cells had been previously cultured and frozen in assay ready vials. Cell vials were thawed, then plated at a density of 10,000 cells per well in a 384 well black wall, clear bottom plate (Greiner #781090) and incubated overnight at 37 degrees C. with 5% CO₂. Cells were grown and plated in F12 nutrient media (Gibco BRL #21700-075) supplemented with 10% FBS (Hyclone #CH30160.03) and Pen/Strep (Gibco #15070-063).

Dye Loading:

After overnight incubation, cell plates were removed from the incubator and the growth media was discarded and replaced with loading solution containing the following: 2 μM Fluo-8-AM (Molecular Probes #F14242), 2 mM Probenecid (Sigma #P8761), lx Acid Red 1 (Sigma #210633), in HBSS buffer containing (grams/L): 0.1 CaCl₂, 0.1 MgCl₂*6H₂O, 0.049 MgSO₄, 0.4 KCl, 0.06 KH₂PO₄, 8.06 NaCl, 0.12 Na₂HPO₄*12H₂O, 1.1 D-glucose*H₂O, 0.35 NaHCO₃, 4.766 HEPES, pH 7.4. The plate was incubated in the loading solution at 37 degrees C. in the dark for 1 hour.

Compound Preparation:

Test compounds were initially prepared as 100% DMSO stock solutions, then transferred and serially diluted in 384-well compound plates (Greiner #784201). Each compound was tested at 10 concentrations in duplicate per experiment. Positive and negative controls for positive allosteric modulator evaluation were 30 μM acetylcholine (Ach) and an EC₁₀-EC₃₀ concentration of acetylcholine, approximately 2 nM but could be adjusted for each experiment to maintain the EC₁₀-EC₃₀ range.

FLIPR Reading:

After the 1-hour dye loading incubation, test compounds were added to the cell plate containing Fluo-8. Approximately 10 minutes after compound addition, an EC₁₀-EC₃₀ concentration of acetylcholine was added to each well and the fluorescence measured to determine the PAM potentiation of the compound.

Data Analysis:

Data was exported from the FLIPR Tetra as maximum fluorescence/minimum fluorescence for each well. The percent effect for each compound well was determined using the mean values for the positive and negative controls on each plate for each read. Percent effect was 100*(compound negative control)/(positive control negative control). Dose response curves were fitted to the compound percent effect data using a 4-parameter logistic fit model to determine PAM (positive allosteric modulator) Inflection Point values. Compounds with inverted U dose response curves had the concentrations greater than the concentration giving the peak response excluded from the fit. Data was reported as Inflection Point. The compounds of Examples 1-72 had activity according to this assay, generally with an Inflection Point (IP) of 10 μM or less (using Inflection Point as a measure of activity). Such a result is indicative of the intrinsic activity of the compounds of the invention as M1 allosteric modulators.

TABLE 7
Biological Data and Compound Name for Examples 1-72.

| | M1 PAM Inflection | |
|---|---|---|
| | Point (μM) Geometric | |
| Example | mean of 2-6 determinations | |
| Number | (unless otherwise indicated) | Compound Name |
| 1 | 0.187^{a} | 5-chloro-N-[(3R,4S)-3-hydroxytetrahydro-2H-pyran-4-yl]-4-[4- |
| | | (1H-pyrazol-1-yl)benzyl]pyridine-2-carboxamide |
| 2 | 0.125^{a} | 4-[2-fluoro-4-(1-methyl-1H-pyrazol-4-yl)benzyl]-N-[(3R,4S)-3- |
| | | hydroxytetrahydro-2H-pyran-4-yl]-5-methylpyridine-2- |
| | | carboxamide |
| 3 | 0.101^{a} | N-[(3R,4S)-3-hydroxytetrahydro-2H-pyran-4-yl]-5-methyl-4- |
| | | [4-(1,3-thiazol-2-yl)benzyl]pyridine-2-carboxamide |
| 4 | 8.11 | N-[(3,4-trans)-3-hydroxytetrahydro-2H-pyran-4-yl]-5-methyl- |
| | | 4-[4-(1,3-thiazol-5-yl)benzyl]pyridine-2-carboxamide, ENT-1 |
| 5 | 0.180 | N-[(3,4-trans)-3-hydroxytetrahydro-2H-pyran-4-yl]-5-methyl- |
| | | 4-[4-(1,3-thiazol-5-yl)benzyl]pyridine-2-carboxamide, ENT-2 |
| 6 | 0.142 | N-[(3R,4S)-3-hydroxytetrahydro-2H-pyran-4-yl]-5-methyl-4- |
| | | [4-(1-methyl-1H-pyrazol-3-yl)benzyl]pyridine-2-carboxamide |
| 7 | 0.060^{a} | N-[(3R,4S)-3-hydroxytetrahydro-2H-pyran-4-yl]-5-methyl-4- |
| | | [4-(1,3-thiazol-4-yl)benzyl]pyridine-2-carboxamide |
| 8 | 4.86^{a} | N-[(3S,4R)-3-hydroxytetrahydro-2H-pyran-4-yl]-5-methyl-4- |
| | | [4-(1H-pyrazol-1-yl)benzyl]pyridine-2-carboxamide |
| 9 | 0.109^{a} | N-[(3R,4S)-3-hydroxytetrahydro-2H-pyran-4-yl]-5-methyl-4- |
| | | [4-(1H-pyrazol-1-yl)benzyl]pyridine-2-carboxamide |
| 10 | 6.34 | (−)-N-[(3,4-trans)-3-hydroxytetrahydro-2H-pyran-4-yl]-5- |
| | | methyl-4-[4-(2-methyl-1,3-oxazol-4-yl)benzyl]pyridine-2- |
| | | carboxamide |
| 11 | 0.146^{a} | (+)-N-[(3,4-trans)-3-hydroxytetrahydro-2H-pyran-4-yl]-5- |
| | | methyl-4-[4-(2-methyl-1,3-oxazol-4-yl)benzyl]pyridine-2- |
| | | carboxamide |
| 12 | 0.960 | (−)-N-[(1,2-cis)-2-hydroxycyclohexyl]-5-methyl-4-[4-(1H- |
| | | pyrazol-1-yl)benzyl]pyridine-2-carboxamide |
| 13 | 1.46 | (+)-N-[(1,2-cis)-2-hydroxycyclohexyl]-5-methyl-4-[4-(1H- |
| | | pyrazol-1-yl)benzyl]pyridine-2-carboxamide |
| 14 | 0.116^{a} | 5-chloro-N-[(3R,4S)-3-hydroxytetrahydro-2H-pyran-4-yl]-6- |
| | | methyl-4-[4-(1H-pyrazol-1-yl)benzyl]pyridine-2-carboxamide |
| 15 | 0.065 | N-[(3R,4S)-3-hydroxytetrahydro-2H-pyran-4-yl]-5-methoxy-4- |
| | | [4-(1H-pyrazol-1-yl)benzyl]pyridine-2-carboxamide |
| 16 | 1.82 | 5-methyl-4-[4-(1H-pyrazol-1-yl)benzyl]-N-[(2S)- |
| | | tetrahydrofuran-2-ylmethyl]pyridine-2-carboxamide, formate |
| | | salt |
| 17 | 0.191 | 5-chloro-N-[(1S,2S)-2-hydroxycyclohexyl]-6-methyl-4-[4-(1H- |
| | | pyrazol-1-yl)benzyl]pyridine-2-carboxamide |
| 18 | 0.092^{a} | N-[(1S,2S)-2-hydroxycyclohexyl]-5-methyl-4-[4-(1H-pyrazol- |
| | | 1-yl)benzyl]pyridine-2-carboxamide |
| 19 | 5.57 | N-[(3R,4S)-3-hydroxytetrahydro-2H-pyran-4-yl]-5-methyl-4- |
| | | [4-(5-methyl-1,2,4-oxadiazol-3-yl)benzyl]pyridine-2- |
| | | carboxamide |
| 20 | >9.94 | N-[(3,4-trans)-3-hydroxytetrahydro-2H-pyran-4-yl]-5-methyl- |
| | | 4-[4-(5-methyl-1,3,4-oxadiazol-2-yl)benzyl]pyridine-2- |
| | | carboxamide, ENT-1 |
| 21 | 1.95 | N-[(3,4-trans)-3-hydroxytetrahydro-2H-pyran-4-yl]-5-methyl- |
| | | 4-[4-(5-methyl-1,3,4-oxadiazol-2-yl)benzyl]pyridine-2- |
| | | carboxamide, ENT-2 |
| 22 | >5.99 | N-[(1S,2S)-2-methoxycyclohexyl]-5-methyl-4-[4-(1H-pyrazol- |
| | | 1-yl)benzyl]pyridine-2-carboxamide |
| 23 | 0.363^{a} | N-[(1S,2S)-2-hydroxycyclohexyl]-5-methyl-4-[4-(2-methyl-1,3- |
| | | oxazol-4-yl)benzyl]pyridine-2-carboxamide |
| 24 | 4.39 | 5-methyl-N-(5-methylpyrimidin-2-yl)-4-[4-(1H-pyrazol-1- |
| | | yl)benzyl]pyridine-2-carboxamide, formate salt |
| 25 | 1.14 | 5-chloro-N-[(1S,2S)-2-hydroxycyclohexyl]-4-(4- |
| | | methoxybenzyl)-6-methylpyridine-2-carboxamide |
| 26 | 5.04 | N-[trans-3-(hydroxymethyl)cyclobutyl]-5-methyl-4-[4-(1H- |
| | | pyrazol-1-yl)benzyl]pyridine-2-carboxamide, formate salt |
| 27 | 1.55 | N-[(1-hydroxycyclopentyl)methyl]-5-methyl-4-[4-(1H-pyrazol- |
| | | 1-yl)benzyl]pyridine-2-carboxamide, formate salt |
| 28 | 4.77 | 5-methyl-N-(2-oxaspiro[3.3]hept-6-yl)-4-[4-(1H-pyrazol-1- |
| | | yl)benzyl]pyridine-2-carboxamide, formate salt |
| 29 | 3.82 | N-(cis-4-hydroxycyclohexyl)-5-methyl-4-[4-(1H-pyrazol-1- |
| | | yl)benzyl]pyridine-2-carboxamide, formate salt |
| 30 | 1.12 | N-(cis-3-hydroxy-3-methylcyclobutyl)-5-methyl-4-[4-(1H- |
| | | pyrazol-1-yl)benzyl]pyridine-2-carboxamide, formate salt |
| 31 | 8.63 | 5-methyl-N-[(3-methyl-1,2,4-oxadiazol-5-yl)methyl]-4-[4-(1H- |
| | | pyrazol-1-yl)benzyl]pyridine-2-carboxamide, formate salt |
| 32 | 4.60 | 5-methyl-4-[4-(1H-pyrazol-1-yl)benzyl]-N-(tetrahydro-2H- |
| | | pyran-4-ylmethyl)pyridine-2-carboxamide, formate salt |
| 33 | 3.44 | 5-methyl-4-[4-(1H-pyrazol-1-yl)benzyl]-N-(tetrahydro-2H- |
| | | pyran-4-yl)pyridine-2-carboxamide, formate salt |
| 34 | 1.02 | N-[trans-2-hydroxycyclopentyl]-5-methyl-4-[4-(1H-pyrazol-1- |
| | | yl)benzyl]pyridine-2-carboxamide, formate salt |
| 35 | 2.73 | 5-methyl-4-[4-(1H-pyrazol-1-yl)benzyl]-N-(tetrahydrofuran-2- |
| | | ylmethyl)pyridine-2-carboxamide, formate salt |
| 36 | 6.65 | N-[(2S)-2-hydroxypropyl]-5-methyl-4-[4-(1H-pyrazol-1- |
| | | yl)benzyl]pyridine-2-carboxamide, formate salt |
| 37 | 3.56 | 5-methyl-N-[(3R)-2-oxoazepan-3-yl]-4-[4-(1H-pyrazol-1- |
| | | yl)benzyl]pyridine-2-carboxamide, formate salt |
| 38 | 3.63 | N-[(2S)-1-hydroxy-3-methylbutan-2-yl]-5-methyl-4-[4-(1H- |
| | | pyrazol-1-yl)benzyl]pyridine-2-carboxamide, formate salt |
| 39 | 4.83 | N-[(2S)-1-hydroxybutan-2-yl]-5-methyl-4-[4-(1H-pyrazol-1- |
| | | yl)benzyl]pyridine-2-carboxamide, formate salt |
| 40 | 9.64 | N-(cyclopropylmethyl)-5-methyl-4-[4-(1H-pyrazol-1- |
| | | yl)benzyl]pyridine-2-carboxamide, formate salt |
| 41 | 8.03 | N-[2-(dimethylamino)-2-oxoethyl]-5-methyl-4-[4-(1H-pyrazol- |
| | | 1-yl)benzyl]pyridine-2-carboxamide, formate salt |
| 42 | 8.13 | 5-methyl-N-(1,2-oxazol-3-ylmethyl)-4-[4-(1H-pyrazol-1- |
| | | yl)benzyl]pyridine-2-carboxamide, formate salt |
| 43 | 9.25 | 5-methyl-4-[4-(1H-pyrazol-1-yl)benzyl]-N-[(2R)- |
| | | tetrahydrofuran-2-ylmethyl]pyridine-2-carboxamide, formate |
| | | salt |
| 44 | 2.70 | N-[(1-hydroxycyclobutyl)methyl]-5-methyl-4-[4-(1H-pyrazol-1- |
| | | yl)benzyl]pyridine-2-carboxamide, formate salt |
| 45 | 7.21 | 5-methyl-N-(oxetan-2-ylmethyl)-4-[4-(1H-pyrazol-1- |
| | | yl)benzyl]pyridine-2-carboxamide, formate salt |
| 46 | 1.22 | N-(2,2-difluorocyclohexyl)-5-methyl-4-[4-(1H-pyrazol-1- |
| | | yl)benzyl]pyridine-2-carboxamide, ENT-1 |
| 47 | 0.57 | N-(2,2-difluorocyclohexyl)-5-methyl-4-[4-(1H-pyrazol-1- |
| | | yl)benzyl]pyridine-2-carboxamide, ENT-2 |
| 48 | 3.50 | N-(3,3-difluorocyclopentyl)-5-methyl-4-[4-(1H-pyrazol-1- |
| | | yl)benzyl]pyridine-2-carboxamide, formate salt |
| 49 | 1.63 | N-[cis-2-hydroxycyclopentyl]-5-methyl-4-[4-(1H-pyrazol-1- |
| | | yl)benzyl]pyridine-2-carboxamide, formate salt |
| 50 | 3.48 | N-[(1S,2S)-2-hydroxycyclohexyl]-4-(4-methoxybenzyl)-5- |
| | | methylpyridine-2-carboxamide |
| 51 | 2.83 | 4-[(6-chloropyridin-3-yl)methyl]-N-[(1S,2S)-2- |
| | | hydroxycyclohexyl]-5-methylpyridine-2-carboxamide, formate |
| | | salt |
| 52 | 0.187^{a} | 5-chloro-N-[(3R,4S)-3-hydroxytetrahydro-2H-pyran-4-yl]-4-[4- |
| | | (1H-pyrazol-1-yl)benzyl]pyridine-2-carboxamide |
| 53 | 0.271 | N-[(3R,4S)-3-hydroxytetrahydro-2H-pyran-4-yl]-5-methyl-4- |
| | | [4-(1H-pyrazol-1-yl)benzyl]pyridine-2-carboxamide 1-oxide |
| 54 | 0.198 | 5-(difluoromethyl)-N-[(3R,4S)-3-hydroxytetrahydro-2H-pyran- |
| | | 4-yl]-4-[4-(1H-pyrazol-1-yl)benzyl]pyridine-2-carboxamide |
| 55 | >9.12 | 4-{(S)-fluoro[4-(1H-pyrazol-1-yl)phenyl]methyl}-N-[(3R,4S)-3- |
| | | hydroxytetrahydro-2H-pyran-4-yl]-5-methylpyridine-2- |
| | | carboxamide |
| 56 | 0.030^{a} | 4-{(R)-fluoro[4-(1H-pyrazol-1-yl)phenyl]methyl}-N-[(3R,4S)-3- |
| | | hydroxytetrahydro-2H-pyran-4-yl]-5-methylpyridine-2- |
| | | carboxamide |
| 57 | 0.144 | N-[(3R,4S)-3-hydroxytetrahydro-2H-pyran-4-yl]-5-methyl-4- |
| | | [4-(1,3-oxazol-4-yl)benzyl]pyridine-2-carboxamide |
| 58 | 0.086 | N-[(3R,4S)-3-hydroxytetrahydro-2H-pyran-4-yl]-5-methoxy-4- |
| | | [4-(2-methyl-1,3-oxazol-4-yl)benzyl]pyridine-2-carboxamide |
| 59 | 1.83^{a} | (+)-4-{fluoro[4-(1,3-thiazol-4-yl)phenyl]methyl}-N-[(3R,4S)-3- |
| | | hydroxytetrahydro-2H-pyran-4-yl]-5-methylpyridine-2- |
| | | carboxamide (diastereomer 1) |
| 60 | 0.023^{a} | (−)-4-{fluoro[4-(1,3-thiazol-4-yl)phenyl]methyl}-N-[(3R,4S)-3- |
| | | hydroxytetrahydro-2H-pyran-4-yl]-5-methylpyridine-2- |
| | | carboxamide (diastereomer 2) |
| 61 | 0.116 | 4-[4-(1,3-dimethyl-1H-pyrazol-4-yl)benzyl]-N-[(3R,4S)-3- |
| | | hydroxytetrahydro-2H-pyran-4-yl]-5-methylpyridine-2- |
| | | carboxamide |
| 62 | 0.129^{a} | 5-(difluoromethoxy)-N-[(3R,4S)-3-hydroxytetrahydro-2H- |
| | | pyran-4-yl]-4-[4-(1H-pyrazol-1-yl)benzyl]pyridine-2- |
| | | carboxamide |
| 63 | 8.76 | 4-{fluoro[4-(1H-pyrazol-1-yl)phenyl]methyl}-N-[(1S,2S)-2- |
| | | hydroxycyclohexyl]-5-methylpyridine-2-carboxamide |
| | | (diastereomer 1) |
| 64 | 0.033^{a} | 4-{fluoro[4-(1H-pyrazol-1-yl)phenyl]methyl}-N-[(1S,2S)-2- |
| | | hydroxycyclohexyl]-5-methylpyridine-2-carboxamide |
| | | (diastereomer 2) |
| 65 | 0.180 | N-[(3R,4S)-3-hydroxytetrahydro-2H-pyran-4-yl]-5-methyl-4- |
| | | {[6-(1-methyl-1H-pyrazol-4-yl)pyridin-3-yl]methyl}pyridine-2- |
| | | carboxamide |
| 66 | 0.114^{a} | N-[(3R,4S)-3-hydroxytetrahydro-2H-pyran-4-yl]-5-methyl-4- |
| | | [4-(1H-pyrazol-4-yl)benzyl]pyridine-2-carboxamide |
| 67 | 0.052^{a} | N-[(3R,4S)-3-hydroxytetrahydro-2H-pyran-4-yl]-5-methyl-4- |
| | | [4-(1-methyl-1H-pyrazol-4-yl)benzyl]pyridine-2-carboxamide |
| 68 | 0.074^{a} | N-[(3R,4S)-3-hydroxytetrahydro-2H-pyran-4-yl]-5-methoxy-4- |
| | | [4-(1,3-thiazol-4-yl)benzyl]pyridine-2-carboxamide |
| 69 | 0.048^{a} | N-[(3R,4S)-3-hydroxytetrahydro-2H-pyran-4-yl]-5-methyl-4- |
| | | [4-(2-methyl-1,3-thiazol-4-yl)benzyl]pyridine-2-carboxamide |
| 70 | 0.104^{a} | N-[(1S,2S)-2-hydroxycyclohexyl]-5-methoxy-4-[4-(1H- |
| | | pyrazol-1-yl)benzyl]pyridine-2-carboxamide |
| 71 | 0.125 | N-[(1S,2S)-2-hydroxycyclohexyl]-5-methoxy-4-[4-(1,3-oxazol- |
| | | 4-yl)benzyl]pyridine-2-carboxamide |
| 72 | 0.142 | N-[(1S,2S)-2-hydroxycyclohexyl]-5-methoxy-4-[4-(2-methyl- |
| | | 1,3-oxazol-4-yl)benzyl]pyridine-2-carboxamide |

^{a}Reported EC₅₀ value is the geometric mean of ≧7 determinations

Various modifications of the invention, in addition to those described herein, will be apparent to those skilled in the art from the foregoing description. Such modifications are also intended to fall within the scope of the appendant claims. Each reference (including all patents, patent applications, journal articles, books, and any other publications) cited in the present application is hereby incorporated by reference in its entirety.

1. A compound of Formula I: 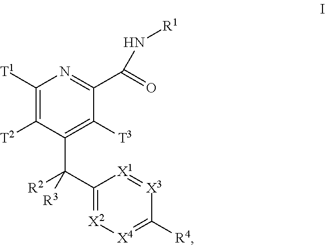
or an N-oxide thereof, or a pharmaceutically acceptable salt of the compound or the N-oxide, wherein:
R¹ is selected from the group consisting of C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted one or more independently selected R⁵, and wherein each of the C₁₋₈ alkyl, C₃₋₁₀ cycloalkyl, 4- to 10-membered heterocycloalkyl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is further optionally substituted one or more oxo;
each of R² and R³ is independently selected from the group consisting of H, halogen, OH, methyl, and methoxy, wherein each of the methyl and methoxy is optionally substituted with one or more substituents each independently selected from OH and halogen;
R⁴ is selected from the group consisting of H, halogen, OR⁶, CN, C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted with one or more independently selected R⁷, and wherein each of the C₁₋₈ alkyl, C₃₋₁₀ cycloalkyl, 4- to 10-membered heterocycloalkyl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is further optionally substituted one or more oxo;
T¹ is selected from the group consisting of H, halogen, N(R^{c})₂, —NR^{e}R^{f}, —CN, C₁₋₆ alkyl, C₂₋₆ alkenyl, C₂₋₆ alkynyl, C₃₋₆ cycloalkyl, (C₃₋₆ cycloalkyl)-C₁₋₂ alkyl-, and C₁₋₆ alkoxy, wherein each of the C₁₋₆ alkyl, C₂₋₆ alkenyl, C₂₋₆ alkynyl, C₃₋₆ cycloalkyl, (C₃₋₆ cycloalkyl)-C₁₋₂ alkyl-, and C₁₋₆ alkoxy of T¹ is optionally substituted with one or more substituents independently selected from the group consisting of halogen, —CN, —C(═O)C₁₋₄ alkyl, —C(═O)OH, —C(═O)O—C₁₋₄ alkyl, —C(═O)NHC₁₋₄ alkyl, —C(═O)N(C₁₋₄ alkyl)₂, oxo, —OH, —OC(═O)—C₁₋₄ alkyl, —OC(═O)O—C₁₋₄ alkyl, —NH₂, —NH(C₁₋₄ alkyl), —N(C₁₋₄ alkyl)₂, —NHC(═O)C₁₋₄ alkyl, —NHC(═O)OC₁₋₄ alkyl, —NHC(═O)NHC₁₋₄ alkyl, and C₁₋₄ alkoxy, and wherein R^{e} and R^{f} together with the N atom to which they are attached form a 4- to 7-membered heterocycloalkyl optionally substituted with one or more substituents each independently selected from the group consisting of halogen, —OH, oxo, —C(═O)H, —C(═O)OH, —C(═O)—C₁₋₄ alkyl, —C(═O)—NH₂, —C(═O)—N(C₁₋₄ alkyl)₂, —CN, C₁₋₄ alkyl, C₁₋₄ alkoxy, C₁₋₄ hydroxylalkyl, C₁₋₄ haloalkyl, and C₁₋₄ haloalkoxy;
T² is selected from the group consisting of halogen, —N(R^{c})₂, —NR^{e}R^{f}, —CN, C₁₋₆ alkyl, C₂₋₆ alkenyl, C₂₋₆ alkynyl, C₃₋₆ cycloalkyl, (C₃₋₆ cycloalkyl)-C₁₋₂ alkyl-, and C₁₋₆ alkoxy, wherein each of the C₁₋₆ alkyl, C₂₋₆ alkenyl, C₂₋₆ alkynyl, C₃₋₆ cycloalkyl, (C₃₋₆ cycloalkyl)-C₁₋₂ alkyl-, and C₁₋₆ alkoxy of T² is optionally substituted with one or more substituents independently selected from the group consisting of halogen, —CN, —C(═O)C₁₋₄ alkyl, —C(═O)OH, —C(═O)O—C₁₋₄ alkyl, —C(═O)NHC₁₋₄ alkyl, —C(═O)N(C₁₋₄ alkyl)₂, oxo, —OH, —OC(═O)—C₁₋₄ alkyl, —OC(═O)O—C₁₋₄ alkyl, —NH₂, —NH(C₁₋₄ alkyl), —N(C₁₋₄ alkyl)₂, —NHC(═O)C₁₋₄ alkyl, —NHC(═O)OC₁₋₄ alkyl, —NHC(═O)NHC₁₋₄ alkyl, and C₁₋₄ alkoxy;
T³ is selected from the group consisting of H, halogen, CH₃, and C₁ fluoroalkyl;
each of X¹, X², X³, and X⁴ is independently selected from the group consisting of CR⁹ and N, provided that at most two of X¹, X², X³, and X⁴ are N;
each R⁵ is independently selected from the group consisting of halogen, —OH, —NO₂, —CN, —SF₅, C₁₋₆ alkyl, C₁₋₆ haloalkyl, C₁₋₆ alkoxy, C₁₋₆ haloalkoxy, C₂₋₆ alkenyl, C₂₋₆ alkynyl, C₃₋₇ cycloalkyl, a 4- to 10-membered heterocycloalkyl, —N(R^{a})(R^{b}), —N(R^{c})(C(═O)R^{d}), —O(═O)—N(R^{a})(R^{b}), —C(═O)—R^{d}, —C(═O)—OR^{d}, —OC(═O)—R^{d}, —N(R^{c})(S(═O)₂R^{d}), —S(═O)₂—N(R^{a})(R^{b}), —SR^{d}, and —OR^{d}, wherein each of the C₁₋₆ alkyl, C₃₋₇ cycloalkyl, and heterocycloalkyl is optionally substituted with one or more substituents each independently selected from the group consisting of halogen, —CN, —OH, C₁₋₄ alkyl, C₁₋₄ alkoxy, C₁₋₄ haloalkyl, C₁₋₄ haloalkoxy, C₃₋₆ cycloalkyl, —N(R^{a})(R^{b}), —N(R^{c})(C(═O)R^{d}), —C(═O)—OR^{d}, —C(═O)H, —C(═O)R^{d}, —C(═O)N(R^{a})(R^{b}), —N(R^{c})(S(═O)₂R^{d}), —S(═O)₂—N(R^{a})(R^{b}), —SR^{d}, and —OR^{d};
R⁶ is selected from the group consisting of H, C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the C₁₋₈ alkyl, C₃₋₁₀ cycloalkyl, 4- to 10-membered heterocycloalkyl, C₆₋₁₀ aryl, 5- to 10-membered heteroaryl, (C₃₋₁₀ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl- is optionally substituted with one or more substituents independently selected from the group consisting of halogen, —CN, —C(═O)C₁₋₄ alkyl, —C(═O)OH, —C(═O)O—C₁₋₄ alkyl, —C(═O)NHC₁₋₄ alkyl, —C(═O)N(C₁₋₄ alkyl)₂, oxo, —OH, —OC(═O)—C₁₋₄ alkyl, —OC(═O)O—C₁₋₄ alkyl, —NH₂, —NH(C₁₋₄ alkyl), —N(C₁₋₄ alkyl)₂, —NHC(═O)C₁₋₄ alkyl, —NHC(═O)OC₁₋₄ alkyl, —NHC(═O)NHC₁₋₄ alkyl, and C₁₋₄ alkoxy;
each R⁷ is independently selected from the group consisting of halogen, —OH, —NO₂, —CN, —SF₅, C₁₋₆ alkyl, C₁₋₆ haloalkyl, C₁₋₆ haloalkoxy, C₂₋₆ alkenyl, C₂₋₆ alkynyl, C₃₋₇ cycloalkyl, a 4- to 10-membered heterocycloalkyl, —N(R^{a})(R^{b}), —N(R^{c})(C(═O)R^{d}), —C(═O)—N(R^{a})(R^{b}), —C(═O)—R^{d}, —C(═O)—OR^{d}, —OC(═O)—R^{d}, —N(R^{c})(S(═O)₂R^{d}), —S(═O)₂—N(R^{a})(R^{b}), —SR^{d}, and —OR^{d}, wherein each of eth C₁₋₆ alkyl, C₃₋₇ cycloalkyl, and heterocycloalkyl is optionally substituted with one or more substituents each independently selected from the group consisting of halogen, —CN, —OH, C₁₋₄ alkyl, C₁₋₄ alkoxy, C₁₋₄ haloalkyl, C₁₋₄ haloalkoxy, C₃₋₆ cycloalkyl, —N(R^{a})(R^{b}), —N(R^{c})(C(═O)R^{d}), —C(═O)—OR^{d}, —C(═O)H, —C(═O)R^{d}, —C(═O)N(R^{a})(R^{b}), —N(R^{c})(S(═O)₂R^{d}), —S(═O)₂—N(R^{a})(R^{b}), —SR^{d}, and —OR^{d};
each R⁹ is independently selected from the group consisting of H, halogen, —OH, —NO₂, —CN, —SF₅, C₁₋₆ alkyl, C₁₋₆ haloalkyl, C₁₋₆ haloalkoxy, C₂₋₆ alkenyl, C₂₋₆ alkynyl, C₃₋₇ cycloalkyl, C₃₋₆ cycloalkyl-C₁₋₂ alkyl-, 4- to 10-membered heterocycloalkyl, —N(R^{a})(R^{b}), —N(R^{c})(C(═O)R^{d}), —C(═O)—N(R^{a})(R^{b}), —C(═O)—R^{d}, —C(═O)—OR^{d}, —OC(═O)—R^{d}, —N(R^{c})(S(═O)₂R^{d}), —S(═O)₂—N(R^{a})(R^{b}), —SR^{d}, and OR^{d}, wherein each of the C₁₋₆ alkyl, C₃₋₇ cycloalkyl, C₃₋₆ cycloalkyl-C₁₋₂ alkyl-, and heterocycloalkyl is optionally substituted with one or more substituents each independently selected from the group consisting of halogen, —CN, —OH, C₁₋₄ alkyl, C₁₋₄ alkoxy, C₁₋₄ haloalkyl, C₁₋₄ haloalkoxy, C₃₋₆ cycloalkyl, —N(R^{a})(R^{b}), —N(R^{c})(C(═O)R^{d}), —C(═O)—OR^{d}, —C(═O)H, —C(═O)R^{d}, —C(═O)N(R^{a})(R^{b}), —N(R^{c})(S(═O)₂R^{d}), —S(═O)₂—N(R^{a})(R^{b}), —SR^{d}, and OR^{d};
each R^{a} is independently H, C₁₋₄ alkyl, C₁₋₄ haloalkyl, C₃₋₇ cycloalkyl, or (C₃₋₇ cycloalkyl)-C₁₋₄ alkyl-;
each R^{b} is independently H or selected from the group consisting of C₁₋₄ alkyl, C₁₋₄ haloalkyl, C₃₋₇ cycloalkyl, a 4- to 10-membered heterocycloalkyl, C₆₋₁₀ aryl, a 5- to 10-membered heteroaryl, (C₃₋₇ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the selections from the group is optionally substituted with one or more substituents each independently selected from the group consisting of —OH, —CN, C₁₋₄ alkyl, C₃₋₇ cycloalkyl, C₁₋₄ hydroxylalkyl, —S—C₁₋₄ alkyl, —C(═O)H, —C(═O)—C₁₋₄ alkyl, —C(═O)—O—C₁₋₄ alkyl, —C(═O)—NH₂, —C(═O)—N(C₁₋₄ alkyl)₂, C₁₋₄ haloalkyl, C₁₋₄ alkoxy, and C₁₋₄ haloalkoxy;
or R^{a} and R^{b} together with the N atom to which they are attached form a 4- to 10-membered heterocycloalkyl or a 5- to 10-membered heteroaryl, each optionally substituted with one or more substituents each independently selected from the group consisting of halogen, —OH, oxo, —C(═O)H, —C(═O)OH, —C(═O)—C₁₋₄ alkyl, —C(═O)—NH₂, —C(═O)—N(C₁₋₄ alkyl)₂, —CN, C₁₋₄ alkyl, C₃₋₆ cycloalkyl, (C₃₋₆ cycloalkyl)-C₁₋₂ alkyl-, C₁₋₄ alkoxy, C₁₋₄ hydroxylalkyl, C₁₋₄ haloalkyl, and C₁₋₄ haloalkoxy;
each R^{c} is independently selected from the group consisting of H, C₁₋₄ alkyl, C₃₋₇ cycloalkyl, and (C₃₋₇ cycloalkyl)-C₁₋₄ alkyl-;
each R^{d} is independently selected from the group consisting of C₁₋₆ alkyl, C₃₋₇ cycloalkyl, a 4- to 14-membered heterocycloalkyl, C₆₋₁₀ aryl, a 5- to 10-membered heteroaryl, (C₃₋₇ cycloalkyl)-C₁₋₄ alkyl-, (4- to 10-membered heterocycloalkyl)-C₁₋₄ alkyl-, (C₆₋₁₀ aryl)-C₁₋₄ alkyl-, and (5- to 10-membered heteroaryl)-C₁₋₄ alkyl-, wherein each of the selections from the group is optionally substituted with one or more substituents each independently selected from the group consisting of halogen, —CF₃, —CN, —OH, oxo, —S—C₁₋₄ alkyl, C₁₋₄ alkyl, C₁₋₄ haloalkyl, C₂₋₆ alkenyl, C₂₋₆ alkynyl, C₃₋₇ cycloalkyl, C₁₋₄ alkoxy, and C₁₋₄ haloalkoxy; and
R^{e} and R^{f} together with the N atom to which they are attached form a 4- to 7-membered heterocycloalkyl optionally substituted with one or more substituents each independently selected from the group consisting of halogen, —OH, oxo, —C(═O)H, —C(═O)OH, —C(═O)—C₁₋₄ alkyl, —C(═O)—NH₂, —C(═O)—N(C₁₋₄ alkyl)₂, —CN, C₁₋₄ alkyl, C₃₋₆ cycloalkyl, (C₃₋₆ cycloalkyl)-C₁₋₂ alkyl-, C₁₋₄ alkoxy, C₁₋₄ hydroxylalkyl, C₁₋₄ haloalkyl, and C₁₋₄ haloalkoxy,
provided that when R¹ is optionally substituted (4- to 10-membered heterocycloalkyl)-C₁₋₄ alkyl-, then the 4- to 10-membered heterocycloalkyl moiety comprises one oxygen ring-form atom.

2. (canceled)

3. The compound, N-oxide, or pharmaceutically acceptable salt of claim 1 wherein each of R² and R³ is independently selected from the group consisting of H and F.

4-7. (canceled)

8. The compound, N-oxide, or pharmaceutically acceptable salt of claim 3 wherein R¹ is a moiety of Formula b-1 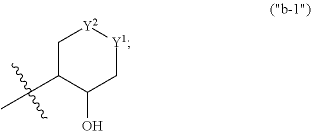
Y¹ is O; and Y² is CH₂.

9. The compound, N-oxide, or pharmaceutically acceptable salt of claim 3 wherein R¹ is a moiety of Formula b-1 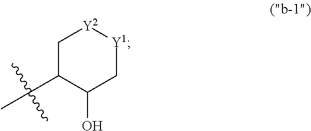
Y¹ is CH₂; and Y² is CH₂.

10. (canceled)

11. The compound, N-oxide, or pharmaceutically acceptable salt of claim 8 wherein the OH group in Formula b 1 or b 2 is trans to the NH—C(═O) moiety of Formula I.

12. (canceled)

13. The compound, N-oxide, or pharmaceutically acceptable salt of claim 3 wherein R¹ is a moiety of Formula b-3:

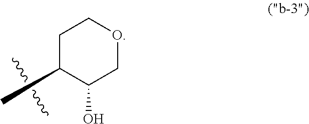

14. The compound, N-oxide, or pharmaceutically acceptable salt of claim 3 wherein R¹ is a moiety of Formula b-4:

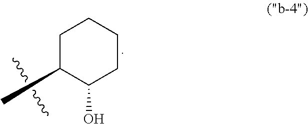

15-17. (canceled)

18. The compound, N-oxide, or pharmaceutically acceptable salt of claim 13 wherein T¹ is H, Cl, or methyl.

19. The compound, N-oxide, or pharmaceutically acceptable salt of claim 18 wherein T² is selected from the group consisting of Cl, —CN, C₁₋₄ alkyl, C₁₋₄ alkoxy, C₁ haloalkyl, and C₁₋₄ haloalkoxy.

20-21. (canceled)

22. The compound, N-oxide, or pharmaceutically acceptable salt of claim 19 wherein T³ is H.

23-24. (canceled)

25. The compound, N-oxide, or pharmaceutically acceptable salt of claim 22 wherein 0 or 1 of X¹, X², X³, and X⁴ is N and each of the rest of X¹, X², X³, and X⁴ is CR⁹.

26. The compound, N-oxide, or pharmaceutically acceptable salt of claim 22 wherein each of X¹, X², X³, and X⁴ is independently CR⁹.

27-28. (canceled)

29. The compound, N-oxide, or pharmaceutically acceptable salt of claim 26 wherein each R⁹ is independently H, halogen, C₁₋₄ alkyl, C₁₋₄ haloalkyl, C₁₋₄ alkoxy, or C₁₋₄ haloalkoxy.

30. The compound, N-oxide, or pharmaceutically acceptable salt of claim 26 wherein each R⁹ is H.

31-32. (canceled)

33. The compound, N-oxide, or pharmaceutically acceptable salt of claim 29 wherein R⁴ is 5- to 6-membered heteroaryl optionally substituted with one or more independently selected R⁷.

34-35. (canceled)

36. The compound, N-oxide, or pharmaceutically acceptable salt of claim 33 wherein R⁴ is selected from pyrazolyl, oxazoly, and thiazolyl, each of the selections is optionally substituted with one or more independently selected R⁷.

37-39. (canceled)

40. The compound, N-oxide, or pharmaceutically acceptable salt of claim 33 wherein each R⁷ is independently selected from the group consisting of OH, halogen, —CN, C₁₋₂ alkyl, C₁₋₂ haloalkyl, C₁₋₂ alkoxy, and C₁₋₂ haloalkoxy.

41. The compound, N-oxide, or pharmaceutically acceptable salt of claim 29 wherein:
R⁴ is a moiety of Formula c-1, c-2, c-3, c-4, c-5, or c-6: 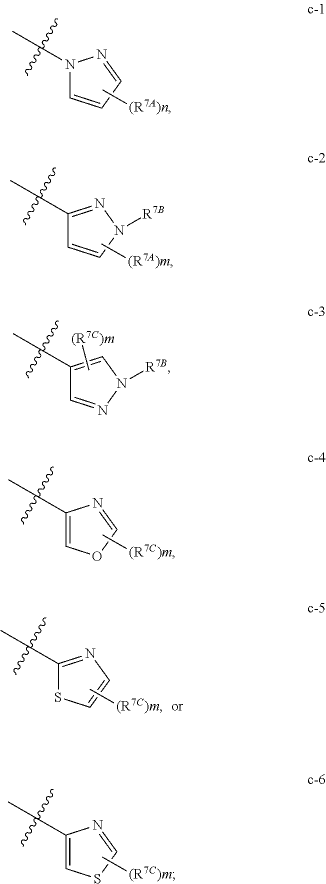
each R^{7A} is independently halogen, —CN, —OH, C₁₋₂ alkyl, C₁₋₂ haloalkyl, C₁₋₂ alkoxy, or C₁₋₂ haloalkoxy;
R^{7B} is C₁₋₂ alkyl;
each R^{7C} is independently C₁₋₂ alkyl, C₁₋₂ haloalkyl, C₁₋₂ alkoxy, or C₁₋₂ haloalkoxy;
n is 0, 1, 2, or 3; and
m is 0, 1, or 2.

42. The compound, or N-oxide, or pharmaceutically acceptable salt of claim 41 wherein R⁴ is a moiety of Formula c-1.

43. The compound, or N-oxide, or pharmaceutically acceptable salt of claim 41 wherein R⁴ is a moiety of Formula c-4.

44. The compound, or N-oxide, or pharmaceutically acceptable salt of claim 41 wherein R⁴ is a moiety of Formula c-6.

45. A compound or N-oxide of claim 1 selected from the group consisting of:
4-[2-fluoro-4-(1-methyl-1H-pyrazol-4-yl)benzyl]-N-[(3R,4S)-3-hydroxytetrahydro-2H-pyran-4-yl]-5-methylpyridine-2-carboxamide;
N-[(3R,4S)-3-hydroxytetrahydro-2H-pyran-4-yl]-5-methyl-4-[4-(1,3-thiazol-2-yl)benzyl]pyridine-2-carboxamide;
N-[(3,4-trans)-3-hydroxytetrahydro-2H-pyran-4-yl]-5-methyl-4-[4-(1,3-thiazol-5-yl)benzyl]pyridine-2-carboxamide, ENT-2;
N-[(3R,4S)-3-hydroxytetrahydro-2H-pyran-4-yl]-5-methyl-4-[4-(1-methyl-1H-pyrazol-3-yl)benzyl]pyridine-2-carboxamide;
N-[(3R,4S)-3-hydroxytetrahydro-2H-pyran-4-yl]-5-methyl-4-[4-(1,3-thiazol-4-yl)benzyl]pyridine-2-carboxamide;
N-[(3R,4S)-3-hydroxytetrahydro-2H-pyran-4-yl]-5-methyl-4-[4-(1H-pyrazol-1-yl)benzyl]pyridine-2-carboxamide;
(+)-N-[(3,4-trans)-3-hydroxytetrahydro-2H-pyran-4-yl]-5-methyl-4-[4-(2-methyl-1,3-oxazol-4-yl)benzyl]pyridine-2-carboxamide;
(−)-N-[(1,2-cis)-2-hydroxycyclohexyl]-5-methyl-4-[4-(1H-pyrazol-1-yl)benzyl]pyridine-2-carboxamide;
5-chloro-N-[(3R,4S)-3-hydroxytetrahydro-2H-pyran-4-yl]-6-methyl-4-[4-(1H-pyrazol-1-yl)benzyl]pyridine-2-carboxamide;
N-[(3R,4S)-3-hydroxytetrahydro-2H-pyran-4-yl]-5-methoxy-4-[4-(1H-pyrazol-1-yl)benzyl]pyridine-2-carboxamide;
5-chloro-N-[(1S,2S)-2-hydroxycyclohexyl]-6-methyl-4-[4-(1H-pyrazol-1-yl)benzyl]pyridine-2-carboxamide;
N-[(1S,2S)-2-hydroxycyclohexyl]-5-methyl-4-[4-(1H-pyrazol-1-yl)benzyl]pyridine-2-carboxamide;
N-[(1S,2S)-2-hydroxycyclohexyl]-5-methyl-4-[4-(2-methyl-1,3-oxazol-4-yl)benzyl]pyridine-2-carboxamide;
N-[trans-2-hydroxycyclopentyl]-5-methyl-4-[4-(1H-pyrazol-1-yl)benzyl]pyridine-2-carboxamide;
N-(2,2-difluorocyclohexyl)-5-methyl-4-[4-(1H-pyrazol-1-yl)benzyl]pyridine-2-carboxamide, ENT-2;
5-chloro-N-[(3R,4S)-3-hydroxytetrahydro-2H-pyran-4-yl]-4-[4-(1H-pyrazol-1-yl)benzyl]pyridine-2-carboxamide;
N-[(3R,4S)-3-hydroxytetrahydro-2H-pyran-4-yl]-5-methyl-4-[4-(1H-pyrazol-1-yl)benzyl]pyridine-2-carboxamide 1-oxide;
5-(difluoromethyl)-N-[(3R,4S)-3-hydroxytetrahydro-2H-pyran-4-yl]-4-[4-(1H-pyrazol-1-yl)benzyl]pyridine-2-carboxamide;
4-{(R)-fluoro[4-(1H-pyrazol-1-yl)phenyl]methyl}-N-[(3R,4S)-3-hydroxytetrahydro-2H-pyran-4-yl]-5-methylpyridine-2-carboxamide;
N-[(3R,4S)-3-Hydroxytetrahydro-2H-pyran-4-yl]-5-methoxy-4-[4-(2-methyl-1,3-oxazol-4-yl)benzyl]pyridine-2-carboxamide;
N-[(3R,4S)-3-hydroxytetrahydro-2H-pyran-4-yl]-5-methoxy-4-[4-(1,3-thiazol-4-yl)benzyl]pyridine-2-carboxamide; and
N-[(3R,4S)-3-hydroxytetrahydro-2H-pyran-4-yl]-5-methyl-4-[4-(2-methyl-1,3-thiazol-4-yl)benzyl]pyridine-2-carboxamide,
or an N-oxide thereof, or a pharmaceutically acceptable salt of the compound or N-oxide.

46. A compound of claim 1 that is N-[(3R,4S)-3-hydroxytetrahydro-2H-pyran-4-yl]-5-methyl-4-[4-(1,3-thiazol-4-yl)benzyl]pyridine-2-carboxamide, or an N-oxide thereof, or a pharmaceutically acceptable salt of the compound or the N-oxide.

47. A compound of claim 1 that is N-[(3R,4S)-3-hydroxytetrahydro-2H-pyran-4-yl]-5-methyl-4-[4-(1H-pyrazol-1-yl)benzyl]pyridine-2-carboxamide, or an N-oxide thereof, or a pharmaceutically acceptable salt of the compound or the N-oxide.

48. A compound of claim 1 that is (+)-N-[(3,4-trans)-3-hydroxytetrahydro-2H-pyran-4-yl]-5-methyl-4-[4-(2-methyl-1,3-oxazol-4-yl)benzyl]pyridine-2-carboxamide, or an N-oxide thereof, or a pharmaceutically acceptable salt of the compound or the N-oxide.

49. A compound of claim 1 that is N-[(3R,4S)-3-hydroxytetrahydro-2H-pyran-4-yl]-5-methoxy-4-[4-(1H-pyrazol-1-yl)benzyl]pyridine-2-carboxamide, or an N-oxide thereof, or a pharmaceutically acceptable salt of the compound or the N-oxide.

50. A compound of claim 1 that is 4-{(R)-fluoro[4-(1H-pyrazol-1-yl)phenyl]methyl}-N-[(3R,4S)-3-hydroxytetrahydro-2H-pyran-4-yl]-5-methylpyridine-2-carboxamide, or an N-oxide thereof, or a pharmaceutically acceptable salt of the compound or the N-oxide.

51. A compound of claim 1 that is N-[(3R,4S)-3-Hydroxytetrahydro-2H-pyran-4-yl]-5-methoxy-4-[4-(2-methyl-1,3-oxazol-4-yl)benzyl]pyridine-2-carboxamide; or an N-oxide thereof, or a pharmaceutically acceptable salt of the compound or the N-oxide.

52. A compound of claim 1 that is N-[(3R,4S)-3-hydroxytetrahydro-2H-pyran-4-yl]-5-methoxy-4-[4-(1,3-thiazol-4-yl)benzyl]pyridine-2-carboxamide, or an N-oxide thereof, or a pharmaceutically acceptable salt of the compound or the N-oxide.

53. A compound of claim 1 that is N-[(3R,4S)-3-hydroxytetrahydro-2H-pyran-4-yl]-5-methyl-4-[4-(2-methyl-1,3-thiazol-4-yl)benzyl]pyridine-2-carboxamide, or an N-oxide thereof, or a pharmaceutically acceptable salt of the compound or the N-oxide.

54. A pharmaceutical composition comprising a therapeutically effective amount of a compound, or N-oxide, or pharmaceutically acceptable salt of claim 1, and a pharmaceutically acceptable carrier.

55. (canceled)

56. A method for treating an M1-mediated (or M1-associated) disease or disorder in a patient, said method comprising administering to the patient a therapeutically effective amount of a compound, or N-oxide, or pharmaceutically acceptable salt of claim 1, wherein the M1-mediated (or M1-associated) disease or disorder is a disease or disorder selected from the group consisting of Alzheimer's disease, schizophrenia or psychosis, pain, addiction, a sleep disorder, a cognitive disorder, Parkinson's Disease, dyskinesia, dry mouth, pulmonary hypertension, chronic obstructive pulmonary disease (COPD), asthma, urinary incontinence, glaucoma, Trisomy 21 (Down Syndrome), cerebral amyloid angiopathy, dementia, Hereditary Cerebral Hemorrhage with Amyloidosis of the Dutch-Type (HCHWA-D), Creutzfeld-Jakob disease, prion disorders, amyotrophic lateral sclerosis, progressive supranuclear palsy, head trauma, stroke, pancreatitis, inclusion body myositis, other peripheral amyloidoses, diabetes, autism, and atherosclerosis.

57-58. (canceled)

59. A method for modulating an activity of an M1 receptor, said method comprising contacting the M1 receptor with a compound, or N-oxide, or pharmaceutically acceptable salt of claim 1.
